(19) United States
(12) Patent Application Publication
(10) Pub. No.: US 2024/0307522 A1
(43) Pub. Date: Sep. 19, 2024

(54) COMPOSITIONS AND METHODS FOR THE PREVENTION AND/OR TREATMENT OF COVID-19

(71) Applicant: PROVIDENCE THERAPEUTICS HOLDINGS INC., Calgary (CA)

(72) Inventors: Eric G. Marcusson, Calgary (CA); Jumai Adeola Abioye, Calgary (CA); Natalia Martin Orozco, Calgary (CA); Yuko Arita, Calgary (CA)

(21) Appl. No.: 18/248,334

(22) PCT Filed: Oct. 8, 2021
(86) PCT No.: PCT/CA2021/051419

§ 371 Date: Apr. 7, 2023

(30) Foreign Application Priority Data
Oct. 9, 2020 (CA) 3096009
Jan. 26, 2021 (CA) 3107232
Mar. 23, 2021 (CA) 3113094
Apr. 23, 2021 (CA) 3116284
Apr. 30, 2021 (CA) 3116932
May 12, 2021 (CA) 3118329
Aug. 9, 2021 (CA) 3128078
Aug. 19, 2021 (CA) 3128660
Sep. 28, 2021 (CA) 3132188

(51) Int. Cl. A61K 39/215; A61K 9/00; A61K 39/00; A61P 31/14; C07K 14/005

(52) U.S. Cl. CPC A61K 39/215; A61K 9/0019; A61P 31/14; C07K 14/005; A61K 2039/53; A61K 2039/54; A61K 2039/545; A61K 2039/575; C12N 2770/20022; C12N 2770/20034

(57) ABSTRACT

The present disclosure generally relates to compositions, formulations, methods, and/or uses of nucleic acid vaccines, specifically nucleic acid vaccines (e.g., RNA, mRNA, DNA vaccines) encoding one or more proteins, peptides, fragments or variants thereof of SARS-CoV-2 for the prevention, alleviation and/or treatment and/or prevention of COVID-19, including mitigation of physiologic effects of infection and/or symptoms.

### CROSS REFERENCE TO RELATED APPLICATIONS

This application claims priority to Canadian Application Nos.: 3,096,009 filed Oct. 9, 2020; U.S. Pat. No. 3,107,232 filed Jan. 26, 2021; U.S. Pat. No. 3,113,094 filed Mar. 23, 2021; U.S. Pat. No. 3,116,284 filed Apr. 23, 2021; U.S. Pat. No. 3,116,932 filed Apr. 30, 2021; U.S. Pat. No. 3,118,329 filed May 12, 2021; U.S. Pat. No. 3,128,078 filed Aug. 9, 2021; U.S. Pat. No. 3,128,660 filed Aug. 19, 2021; U.S. Pat. No. 3,132,188 filed Sep. 28, 2021, the contents of each of which are incorporated by reference in their entirety.

### SEQUENCE LISTING

The present application is being filed along with a Sequence Listing in electronic format. The Sequence Listing file, entitled 2092_1004PCT_SL.txt, was created on Oct. 1, 2021, and is 193,800 bytes in size. The information in electronic format of the Sequence Listing is incorporated herein by reference in its entirety.

### FIELD

The present disclosure generally relates to compositions, formulations, methods, and/or uses of nucleic acid vaccines, specifically nucleic acid vaccines (e.g., RNA, mRNA, DNA vaccines) encoding one or more proteins, peptides, fragments or variants thereof of SARS-CoV-2 for the prevention, alleviation and/or treatment and/or prevention of COVID-19, including mitigation of physiologic effects of infection and/or symptoms.

### BACKGROUND

Severe acute respiratory syndrome coronavirus 2 (SARS-CoV-2) is a new strain of coronavirus which began infecting mammals in 2019 in China and has spread to a pandemic. SARS-CoV-2 infection causes coronavirus disease 2019 (termed “COVID-19”), which affects mammals in different ways including individuals who are asymptomatic to individuals who have a wide range of symptoms that range from mild symptoms to severe illness or death.

Vaccines are an effective way to provide prophylactic protection against infective diseases. Currently, there are limited vaccines available to prevent, alleviate and/or treat COVID-19. Treatment of COVID-19 has been limited to the management of symptoms and/or the side effects of the disease. Thus, there remains a strong need for COVID-19 vaccines including formulations for delivering the vaccines to a range of different target T-cells.

### SUMMARY

The present disclosure provides nucleic acid vaccines, compositions and formulations comprising nucleic acid vaccines, and methods of using same for preventing infection of coronavirus for the prevention, alleviation and treatment of COVID-19. The nucleic acid vaccines may include polynucleotides which encode at least one antigen protein, fragment or variant thereof of SARS-CoV-2. The SARS-CoV-2 antigen protein is a structural protein of SARS-CoV-2. The structural protein may be the spike protein, the membrane protein, the nucleocapsid phosphoprotein or the envelope protein. Non-limiting examples of the amino acid sequences of these structural proteins are shown in Table 1 (SEQ ID Nos: 1-6 and 15-19).

Provided herein are nucleic acid vaccines for COVID-19 for use in a method of vaccinating a subject for COVID-19, wherein the nucleic acid vaccine may include at least one polynucleotide encoding at least one structural protein or a fragment thereof of SARS-CoV-2.

Provided herein are methods of inducing an immune response in a subject by administering the nucleic acid vaccines described herein in an effective amount to produce an immune response. The immune response may be, but is not limited to, a T-cell response or a B cell response. As a non-limiting example, the immune response may be produced by a single administration of the nucleic acid vaccines described herein. As another non-limiting example, the immune response may be produced by a booster administration of the nucleic acid vaccines described herein. The administering the pharmaceutical composition may produce a dose-responsive immune response in the subject. As a non-limiting example, the dose-responsive immune response may comprise induction of one or more of SARS-CoV-2 spike protein specific IgG, IgG1, IgG2a, IgG2b, IgM and IgA antibodies in the subject. As another non-limiting example, the dose-responsive immune response may comprise induction of one or more of IL-2+ T-cells, IL-4+ T-cells, and IFN-gamma+ T-cells. In some embodiments, the administering the pharmaceutical composition does not induce significant adverse reactions in the subject.

Provided herein are methods of treating and/or preventing COVID-19 in a subject by administering the nucleic acid vaccines described herein.

Provided herein are pharmaceutical compositions and formulations of the nucleic acid vaccines for the treatment and prevention of COVID-19.

The nucleic acid vaccines described herein may be formulated in one or more lipid nanoparticles (LNPs).

Also provided herein are nucleic acid vaccines for COVID-19, comprising about 0.2 mg/mL mRNA, wherein the mRNA comprises a coding region with a nucleic acid sequence that is at least 95% identical to SEQ ID NO: 7. In some embodiments, the mRNA of the nucleic acid vaccines disclosed herein comprises a coding region with a nucleic acid sequence as set forth in SEQ ID NO: 7. The nucleic acid vaccines may be formulated as a 2 mL fill in a 3 mL glass vial.

In some embodiments, administering the nucleic acid vaccines to a subject comprises administering about 5 μg to about 100 μg of the mRNA to the subject. For instance, the methods may comprise administering about 16 μg of the mRNA to the subject. Alternatively, the methods may comprise administering about 40 μg of the mRNA to the subject. Or the methods may comprise administering about 100 μg of the mRNA to the subject.

In some embodiments, administering the nucleic acid vaccines to a subject comprises administering about 0.025 mL to about 0.5 mL of the nucleic acid vaccine to the subject. For instance, the methods may comprise administering about 0.025 mL of the nucleic acid vaccine to the subject, about 0.05 mL of the nucleic acid vaccine to the subject, about 0.08 mL of the nucleic acid vaccine to the subject, about 0.2 mL of the nucleic acid vaccine to the subject, or about 0.5 mL of the nucleic acid vaccine to the subject.

In some embodiments of the methods provided, the administering comprises an intramuscular (IM) injection of the nucleic acid vaccine to the subject.

The nucleic acid vaccines may be administered to a subject in a first dose of the nucleic acid vaccine followed by a second dose of the nucleic acid vaccine after between about 1 and about 5 weeks. In some embodiments, the second dose of the nucleic acid vaccine is administered about 4 weeks after the first dose.

In some embodiments, anti-Spike protein IgG antibodies are detected in the subject by day 28 after receiving a first dose of the nucleic acid vaccine.

In some embodiments, anti-Spike protein IgG antibodies are detected in the subject by day 28 after receiving a first dose of the nucleic acid vaccine and are enhanced after receiving a second dose of the nucleic acid vaccine in the subject by day 42.

In some embodiments, anti-Spike protein IgG antibodies in the subject are increased to 10-fold higher than the average values of anti-Spike protein IgG antibodies from serum samples from SARS-CoV-2 convalescent patients.

In some embodiments, the SARS-CoV-2 neutralizing antibodies are detected in the subject by day 28 after a first dose of the nucleic acid vaccine.

In some embodiments, the SARS-CoV-2 neutralizing antibodies are enhanced after a second dose of the nucleic acid vaccine in the subject by day 42.

Thus, the disclosure provides nucleic acid vaccines for COVID-19 for use in a method of vaccinating a subject for COVID-19, wherein the nucleic acid vaccine comprises about 0.2 mg/mL mRNA, wherein the mRNA comprises a nucleic acid sequence that is at least 95% identical to SEQ ID NO: 7, and wherein the nucleic acid vaccine is formulated for intramuscular (IM) injection and formulated in a lipid nanoparticle (LNP).

The details of various embodiments are set forth in the description below. Other features, objects and advantages will be apparent from the description, and from the claims.

### BRIEF DESCRIPTION OF THE DRAWINGS

FIG. 1 shows results from a SARS-CoV-2 neutralization assay using virus isolated from a patient in Ontario. Groups 1-5 correlate to the vaccine formulation administered (see Table 6).

FIG. 2 shows results from a neutralization assay using a SARS-CoV-2 pseudotyped lentivirus that encodes a luciferase gene and can infect HEK293T-cells. Groups 1-5 correlate to the vaccine formulation administered (see Table 6).

FIG. 3 shows the ID50 (dilution at which 50% inhibition of infectivity is seen) for both the SARS-CoV-2 clinical isolate and pseudovirus neutralization assays.

FIG. 4 shows IFN-γ analysis by ELISpot to determine the T-cell response to immunization with PTX-B.

FIG. 5 shows cytokine profiling by Luminex in mice vaccinated with a prime and boost of PTX-B at Days 1 and 22.

FIG. 6A-FIG. 6B show cytokine profiling by flow cytometry in mice vaccinated with a prime and boost of PTX-B at Days 1 and 22.

FIG. 7 shows change in body weight in mice challenged with SARS-CoV-2.

FIG. 8 shows protective efficacy in AAV6-hACE2 transduction mouse model.

FIG. 9 shows lung histopathology scores in a AAV6-hACE2 transduction mouse model.

FIG. 10 shows IFN-γ and IL-4 ELISpot analysis of splenocytes from PTX-B immunized mice.

FIG. 11 shows protection from infection with a SARS-CoV-2 clinical isolate in a SARS-CoV-2 neutralization assay.

FIG. 12 shows protection from infection in a pseudovirus neutralization assay.

FIG. 13A-FIG. 13C show anti-SARS-CoV-2 anti-Spike protein antibody profiles.

FIG. 14 shows levels of infectious virus was significantly lower in vaccinated animals in a SARS-CoV-2 challenge study in hamsters.

FIG. 15 shows calculations of T-cell stimulation index in a co-culture experiment using SARS-CoV-2 N, M, and N/M protein nucleic acid vaccines.

FIG. 16 shows characterization of a serum/antibody neutralization assay using pseudovirus encoding S protein variants from SARS-CoV-2 variants of concern (VOCs) and other variants.

FIG. 17 shows protection efficiency (ID50) of PTX-B against infection from SARS-CoV-2 VOC pseudovirus and other pseudovirus variants.

FIG. 18 shows protection efficiency (ID50) of PTX-B against infection from individual SARS-CoV-2 VOC pseudovirus and other pseudovirus variants.

FIG. 19 shows anti-Spike protein IgG levels in PTX-B vaccinated subjects at days 8, 28, and 42 after vaccination with 16, 40, or 100 μg doses.

FIG. 20 shows anti-Spike protein IgG levels in placebo-treated control subjects compared to levels in SARS-CoV-2 convalescent patient plasma.

FIG. 21 shows neutralizing activity in samples from PTX-B vaccinated subjects at days 8, 28, and 42 after vaccination with 16, 40, or 100 μg doses.

FIG. 22 shows anti-COVID-19 neutralizing antibody levels in samples from PTX-B vaccinated subjects at days 8, 28, and 42 after vaccination with 16, 40, or 100 μg doses.

FIG. 23 shows anti-COVID-19 neutralizing antibody concentrations from PTX-B vaccinated subjects at days 28, and 42 after vaccination with 16, 40, or 100 μg doses.

FIG. 24 shows pseudotyped virus neutralization of anti-COVID-19 neutralizing antibody from PTX-B vaccinated subjects at days 28, and 42 after vaccination with 16, 40, or 100 μg doses.

FIG. 25 shows the prediction of protective efficacy of PTX-B based on Khoury model.

FIG. 26 shows the PTX-B induced neutralization activity against the SAR-CoV-2 original strain, and the Alpha, Beta and Delta VOCs.

### DETAILED DESCRIPTION

### I. Introduction

The following description sets forth exemplary compositions, methods, parameters and the like. It should be recognized, however, that such description is not intended as a limitation on the scope of the present disclosure but is instead provided as a description of exemplary embodiments.

Described herein are polynucleotides (e.g., mRNAs), compositions, formulations, methods, and/or use of nucleic acid vaccines, specifically nucleic acid vaccines comprising polynucleotides encoding one or more antigen proteins, fragments or variants thereof of SARS-CoV-2 for the prevention, alleviation and/or treatment of COVID-19. The antigen protein may be a structural protein of SARS-CoV-2. The structural protein may be the spike(S) protein, the membrane(M) protein, the nucleocapsid(N) phosphoprotein or the envelope(E) protein.

In some embodiments, at least one component of the nucleic acid vaccine is a polynucleotide encoding at least one of the antigen proteins or the fragments or variants of the antigen proteins of SARS-CoV-2. The antigen protein may be a structural protein of SARS-CoV-2. The polynucleotide may be a RNA polynucleotide such as an mRNA polynucleotide.

In some embodiments, the nucleic acid vaccine includes at least one mRNA polynucleotide encoding at least one of the structural proteins or the fragments or variants of the structural proteins of SARS-CoV-2.

In some embodiments, the polynucleotide may be designed to encode one or more polypeptides of interest from SARS-CoV-2, or fragments or variants thereof. Such polypeptide of interest of SARS-CoV-2 may include, but is not limited to, whole polypeptides, a plurality of polypeptides or fragments of polypeptides or variants of polypeptides, which independently may be encoded by one or more regions or parts or the whole of a polynucleotide from SARS-CoV-2. As used herein, the term “polypeptides of interest” refer to any polypeptide which is selected to be encoded within, or whose function is affected by, the polynucleotides described herein. Any of the peptides or polypeptides described herein may be antigenic (also referred to as immunogenic).

As used herein, “polypeptide” means a polymer of amino acid residues (natural or unnatural) linked together most often by peptide bonds. The term, as used herein, refers to proteins, polypeptides, and peptides of any size, structure, or function, or origin. In some embodiments, the polypeptides of interest are antigens encoded by the polynucleotides as described herein.

In some embodiments, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, or at least about 85%, more preferably at least about 90%, even more preferably at least about 95% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gap and penalties introduced in the calculation.

By “homologs” as it applies to polypeptide sequences means the corresponding sequence of other species having substantial identity to a second sequence of a second species.

“Analogs”, as used herein, is meant to include polypeptide variants which differ by one or more amino acid alterations, e.g., substitutions, additions or deletions of amino acid residues that still maintain one or more of the properties of the parent or starting polypeptide.

In some embodiments, the present disclosure contemplates several types of compositions which are polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

For example, sequence tags or amino acids, such as one or more lysines, can be added to the peptide sequences described herein (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of nonconservative substitutions include the substitution of a nonpolar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side chains or terminal residues, or by harnessing mechanisms of post-translational modifications that function in selected recombinant hosT-cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

“Features” when referring to polypeptides are defined as distinct amino acid sequence-based components of a molecule. Features of the polypeptides encoded by the polynucleotides described herein include surface manifestations, local conformational shape, folds, loops, half-loops, domains, half-domains, sites, termini or any combination thereof.

As used herein when referring to polypeptides the term “surface manifestation” refers to a polypeptide-based component of a protein appearing on an outermost surface.

As used herein when referring to polypeptides the term “local conformational shape” means a polypeptide based structural manifestation of a protein which is located within a definable space of the protein.

As used herein when referring to polypeptides the term “fold” refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to polypeptideconformation means a bend which alters the direction of the backbone of a peptide or polypeptide and may involve one, two, three or more amino acid residues.

As used herein when referring to polypeptides the term “loop” refers to a structural feature of a polypeptide which may serve to reverse the direction of the backbone of a peptide or polypeptide. Where the loop is found in a polypeptide and only alters the direction of the backbone, it may comprise four or more amino acid residues. Oliva et al. have identified at least 5 classes of protein loops (J. MolBiol., 266 (4): 814-830; 1997). Loops may be open or closed. Closed loops or “cyclic” loops may comprise 2, 3, 4, 5, 6, 7, 8, 9, 10 or more amino acids between the bridging moieties. Such bridging moieties may comprise a cysteine-cysteine bridge (Cys-Cys) typical in polypeptides having disulfide bridges or alternatively bridging moieties may be non-protein based such as the dibromozylyl agents used herein.

As used herein when referring to polypeptides the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids).

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the term “half-domain” means a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+1−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, when referring to polypeptides the term “site” as it pertains to amino acid-based embodiments is used synonymously with “amino acid residue” and “amino acid side chain.” A site represents a position within a peptide or polypeptide that may be modified, manipulated, altered, derivatized or varied within the polypeptide-based molecules described herein.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide-based molecules described herein may be characterized as having both an N-terminus (terminated by an amino acid with a free amino group (NH2)) and a C-terminus (terminated by an amino acid with a free carboxyl group (COOH)). Proteins described herein are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a desired component of a polypeptide to be encoded by a polynucleotide described herein, any of several manipulations and/or modifications of these features may be performed by moving, swapping, inverting, deleting, randomizing or duplicating. Furthermore, it is understood that manipulation of features may result in the same outcome as a modification to the molecules described herein. For example, a manipulation which involved deleting a domain would result in the alteration of the length of a molecule just as modification of a nucleic acid to encode less than a full-length molecule would.

In a polypeptide, the term “modification” refers to a modification as compared to the canonical set of 20 amino acids. The modifications may be various distinct modifications. In some embodiments, the regions may contain one, two, or more (optionally different) modifications.

Modifications and manipulations can be accomplished by methods known in the art such as, but not limited to, site directed mutagenesis or a priori incorporation during chemical synthesis. The resulting modified molecules may then be tested for activity using in vitro or in vivo assays such as those described herein or any other suitable screening assay known in the art.

In some embodiments, the polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical to a reference protein. The protein fragment may contain 10, 20, 30, 40, 50, 60, 70, 80, 90, 100, or greater than 100 amino acids in length. In another example, any protein that includes a stretch of about 20, about 30, about 40, about 50, or about 100 amino acids, or more, which are about 40%, about 50%, about 60%, about 70%, about 80%, about 85%, about 90%, about 95%, or about 100% identical to any of the sequences described herein can be utilized in accordance with the nucleic acid vaccines described herein. In certain embodiments, a polypeptide to be utilized in accordance with the nucleic acid vaccines described herein includes 2, 3, 4, 5, 6, 7, 8, 9, 10, or more mutations as shown in any of the sequences provided or referenced herein.

As such, polynucleotides of the present disclosure encode peptides or polypeptides containing substitutions, insertions and/or additions, deletions and covalent modifications with respect to reference sequences, in particular the peptide or polypeptide sequences disclosed herein. The polynucleotides may also contain substitutions, insertions and/or additions, deletions and covalent modifications with respect to the polynucleotide reference sequences.

Reference molecules (polypeptides or polynucleotides) may share a certain identity with the designed molecules (polypeptides or polynucleotides). The term “identity” as known in the art, refers to a relationship between the sequences of two or more peptides, polypeptides or polynucleotides, as determined by comparing the sequences. In the art, identity also means the degree of sequence relatedness between them as determined by the number of matches between strings of two or more amino acid residues or nucleosides. Identity measures the percent of identical matches between the smaller of two or more sequences with gap alignments (if any) addressed by a particular mathematical model or computer program (e.g., “algorithms”). Identity of related peptides can be readily calculated by known methods. Such methods include, but are not limited to, those described in Computational Molecular Biology, Lesk, A. M., ed., Oxford University Press, N.Y., 1988; Biocomputing: Informatics and Genome Projects, Smith, D. W., ed., Academic Press, N.Y., 1993; Computer Analysis of Sequence Data, Part 1, Griffin, A. M., and Griffin, H. G., eds., Humana Press, N.J., 1994; Sequence Analysis in Molecular Biology, von Heinje, G., Academic Press, 1987; Sequence Analysis Primer, Gribskov, M. and Devereux, J., eds., M. Stockton Press, N.Y, 1991; and Carillo et al., SIAM J. Applied Math. 48: 1073; 1988).

In some embodiments, the encoded polypeptide variant may have the same or a similar activity as the reference polypeptide. Alternatively, the variant may have an altered activity (e.g., increased or decreased) relative to a reference polypeptide. Generally, variants of a particular polynucleotide or polypeptide described herein will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul et al., Gapped BLAST and PSLBLAST: a new generation of protein database search programs, Nucleic Acids Res. 1997, 25:3389-3402.) Other tools are described herein, specifically in the definition of “Identity.”

### II. Compositions of the Present Disclosure

### SARS-Cov-2

Severe acute respiratory syndrome coronavirus 2 (SARS-CoV-2) is a new strain of coronavirus which causes coronavirus disease 2019 termed “COVID-19.” COVID-19 affects mammals in different ways including individuals who are asymptomatic to individuals who have a wide span of symptoms that range from mild symptoms to severe illness or death. To date, about 80% of COVID-19 patients have mild to moderate symptoms whereas about 20% may develop complications such as sever pneumonia, acute respiratory distress syndrome, sepsis and even death. The list of symptoms associated with COVID-19 is constantly changing as doctors and scientists learn more about COVID-19 and how it affects the body, but some of the symptoms recognized to date include fever or chills, cough, shortness of breath or difficulty breathing, fatigue, body aches, muscle aches, headaches, sore throat, congestion or runny nose, nausea and/or vomiting, diarrhea, and a new loss of taste or smell.

The genome of SARS-CoV-2 encodes four structural proteins: spike (S), envelope (E), membrane (M), and nucleocapsid (N), and nonstructural proteins (named nsp1 to nsp16) and accessory proteins. The viral surface spike protein is generally the leading mediator for viral entry into cells. SARS-CoV-2 spike binds to its receptor human ACE2 (hACE2) through its receptor-binding domain (RBD) and is proteolytically activated by human proteases. Another feature of the spike protein of SARS-CoV-2 is that the protein has a functional furin cleavage site at the S1-S2 boundary (S1 is the receptor binding unit and S2 is the membrane fusion unit), which can preactivate the entry of many viruses including SARS-CoV-2. SARS-CoV-2 spike has been used as a protective antigen that elicits neutralizing antibodies in various vaccine developing strategies. The membrane protein and the envelope protein are for viral assembly. The envelop protein (E) can forms a homopentameric cation channel that is important for virus pathogenicity Mandala et al., Nature Structural and Molecular Bio. 2020, 27: 1202-1208). The nucleocapsid protein packages the viral genome into a helical ribonucleocapsid (RNP) and has a role in viral self-assembly (Chang et al.; The SARS coronavirus nucleocapsid protein—Forms and functions; Antiviral Res. 2014; 103:39-50; the contents of which are herein incorporated by reference in their entirety). Additionally, the nucleocapsid protein in SARS-CoV-2 can modulate the hosT-cell machinery and may be included in regulatory roles in the viral life cycle.

While not wishing to be bound by theory, it appears that SARS-CoV-2 binds to the human receptor ACE2 (hACE2). The receptor-binding domain (RBD) in the spike protein appears to be the most variable part of the coronavirus genome. There are six RBD amino acids have been shown to be critical for binding to ACE2 receptors and the SARS-CoV-2 genome appears to have a RBD that has a high affinity binding to ACE2 for humans, ferrets, cats and other species with high receptor homology (Anderson et. al.; The Proximal Origin of SARS-CoV-2; Nature Medicine, 2020; 26(4): 450-452; the contents of which are herein incorporated by reference in their entirety).

In some embodiments, the polynucleotides of the nucleic acid vaccine described herein encode the full-length polypeptide of a structural protein, or a fragment or variant of the structural protein of SARS-CoV-2, such as the spike protein, the nucleocapsid protein, the envelop protein or the membrane protein.

In some embodiments, the polynucleotides of the nucleic acid vaccine described herein encode more than one fragment or variant of a structural protein of SARS-CoV-2, such as the spike protein, the nucleocapsid protein, the envelop membrane and/or the membrane protein.

In some embodiments, the polynucleotides of the nucleic acid vaccine described herein encode a mutated variant of one of the structural proteins, or a fragment of the mutated variant of the structural proteins of SARS-CoV-2. As a non-limiting example, the variant may be a single amino acid change from Aspartic Acid to Glycine in one of the structural proteins of SARS-CoV-2.

In some embodiments, the polynucleotides of the nucleic acid vaccine described herein encode a full-length polypeptide of the spike protein, or a fragment, or a variant of the spike protein of SARS-CoV-2. As a non-limiting example, the variant may be a single amino acid change from Aspartic Acid to Glycine in the spike protein of SARS-CoV-2. As a non-limiting example, the variant may be a single amino acid change from Aspartic Acid to Glycine at position 614 (D614G) in the spike protein of SARS-CoV-2 (Korber et al.; Tracking Changes in SARS-CoV-2 Spike: Evidence that D614G Increases Infectivity of the COVID-19 Virus; Cell; 2020, 182(4): 812-827; the contents of which is herein incorporated by reference in its entirety).

In some embodiments, the nucleic acid vaccine described herein may encode one or more proteins, peptides, fragments or variants thereof of the structural proteins of SARS-CoV-2. Non-limiting examples of proteins, peptides, fragments or variants of the structural proteins of SARS-CoV-2 are provided in Table 1. In the table, the NCBI reference number is also provided if known.

TABLE 1
Structural Protein Sequences of SARS-CoV-2

| Sequence | | |
|---|---|---|
| Identifier | | |
| (SEQ | | Sequence |
| ID NO) | Description | Type |
| 1 | Spike protein (NCBI Ref.: | Protein |
| | YP_009724390.1) (“S protein”) | |
| 2 | Spike protein with D614G mutation | Protein |
| 3 | Envelope protein (NCBI Ref.: | Protein |
| | YP_009724392.1) | |
| 4 | Membrane protein (NCBI Ref.: | Protein |
| | YP_009724393.1) | |
| 5 | Nucleocapsid phosphoprotein | Protein |
| | (NCBI Ref.: YP_009724397.2) | |
| 6 | B.1.351 (South African) Variant | Protein |
| | Spike protein | |
| 15 | B.1.17 (UK) Variant Spike protein | Protein |
| 16 | Spike protein with D614G and | Protein |
| | L452R mutation | |
| 17 | B.1.17 (UK) Variant Spike protein | Protein |
| | with L452R mutation | |
| 18 | B.1.351 (South African) Variant | Protein |
| | Spike protein with L452R mutation | |
| 19 | P.1 (Brazil) Variant Spike Protein | Protein |

In some embodiments, the nucleic acid vaccine described herein may encode at least one structural protein with at least 75%, 80%, 85%, 90%, 95%, 96%, 97%, 98%, 99% or 100% of any of the sequences in Table 1 or fragments of any of the sequences in Table 1 or variants of any of the sequences in Table 1.

In some embodiments, the nucleic acid vaccine may be an mRNA vaccine that, when translated, produces one or more proteins, peptides, fragments or variants thereof of the structural proteins of SARS-CoV-2. Accordingly, the polynucleotides of the mRNA vaccine are mRNA polynucleotides encoding one or more proteins, peptides, fragments or variants thereof of the structural proteins of SARS-CoV-2.

In one embodiment, the coding sequences of mRNA vaccines described herein may be based on the coding sequence of the spike(S) protein from the genome of SARS-CoV-2 Wuhan-Hu-1 isolate (GenBank: NM908947.3, complete genome sequence). In some embodiments, a change of the code for a single amino acid change from D614 to G614 is introduced to match the amino acid of the current dominant circulating strains.

Non-limiting examples of a RNA sequence encoding proteins, peptides, fragments or variants of the structural proteins of SARS-CoV-2 are provided in Table 2.

TABLE 2
Sequences of Spike protein of SARS-CoV-2

| Sequence | | |
|---|---|---|
| Identifier | | |
| (SEQ | | Sequence |
| ID NO) | Description | Type |
| 7 | Coding region for the Spike protein | RNA |
| | with D614G mutation | |
| 20 | Coding region for the SARS-CoV-2 | RNA |
| | variant B.1.351 (South African variant) | |
| | Spike protein | |
| 21 | Coding region for the M protein | RNA |
| 22 | Coding region for the N and M protein | RNA |
| 23 | Coding region for the N protein | RNA |
| 24 | Coding region for the signal peptide | RNA |
| | and encoding N and M protein | |
| 25 | Sequence encoding S protein receptor | RNA |
| | binding domain (RBD) | |
| 26 | Sequence encoding S protein with | RNA |
| | mutated furin site mRNA | |
| 27 | Sequence encoding the Spike protein | RNA |
| | with D614G mutation | |
| 28 | Sequence encoding M protein | RNA |
| 29 | Sequence encoding N and M protein | RNA |
| 30 | Sequence encoding N protein | RNA |
| 31 | Sequence with a signal peptide and | RNA |
| | encoding N and M protein | |
| 32 | Sequence encoding SARS-CoV-2 | RNA |
| | variant B.1.351 (South African variant) | |
| | Spike protein | |

In some embodiments, the mRNA sequence encoding the spike protein with D614G mutation of SARS-CoV-2 comprises the coding region of SEQ ID NO: 7, or a fragment or variant thereof.

In some embodiments, the mRNA sequence encoding the spike protein with D614G mutation of SARS-CoV-2 comprises SEQ ID NO: 27, or a fragment or variant thereof.

In some embodiments, the nucleic acid vaccines may comprise a region encoding any of the sequences listed in Table 1 or a fragment or variant thereof. The nucleic acid vaccines may comprise hybrid or chimeric regions, or mimics or variants. In some embodiments, the nucleic acid vaccines may comprise any of the polynucleotide sequences listed in Table 3.

TABLE 3
Exemplary Sequences to be used in the Nucleic Acid
Vaccines for treating or preventing COVID-19

| Sequence | | |
|---|---|---|
| Identifier | | |
| (SEQ | | Sequence |
| ID NO) | Description | Type |
| 8 | Sequence encoding M protein | DNA |
| 9 | Sequence encoding N and M protein | DNA |
| 10 | Sequence encoding N protein | DNA |
| 11 | Sequence with a signal peptide and | DNA |
| | encoding N and M protein | |
| 12 | Sequence encoding SARS-CoV-2 | DNA |
| | variant B.1.351 (South African variant) | |
| | Spike protein | |
| 33 | Coding region for SARS-CoV-2 | DNA |
| | variant B.1.351 (South African variant) | |
| | Spike protein | |
| 34 | Coding region for the M protein | DNA |
| 35 | Coding region for the N and M protein | DNA |
| 36 | Coding region for the N protein | DNA |
| 37 | Coding region for the signal peptide | DNA |
| | and encoding N and M protein | |
| 38 | Sequence encoding M protein | DNA |
| 39 | Sequence encoding N and M protein | DNA |
| 40 | Sequence encoding N protein | DNA |
| 41 | Sequence with a signal peptide and | DNA |
| | encoding N and M protein | |
| 42 | Sequence encoding the Spike protein | DNA |
| | with D614G mutation | |
| 43 | Sequence encoding SARS-CoV-2 | DNA |
| | variant B.1.351 (South African variant) | |
| | Spike protein | |
| 50 | Sequence encoding S protein receptor | DNA |
| | binding domain (RBD) | |
| 51 | Sequence encoding S protein with | DNA |
| | mutated furin site | |

Any of the sequences referred to in Tables 1-3 or variants thereof may also be used in a memory booster vaccine described herein.

In some embodiments, the nucleic acid vaccine described herein encodes a protein or fragment or variant thereof that is at least 80%, at least 85%, at least 90%, at least 91%, at least 92%, at least 93%, at least 94%, at least 95%, at least 96%, at least 97%, at least 98%, or at least 99% identical to a protein provided by an amino acid sequence in Table 1. The terms “identical” or percent “identity” in the context of two or more polypeptide sequences refer to two or more sequences that are the same. The percent identity between polypeptide sequences may be performed using algorithms known in the art, such as BLAST and CLUSTAL.

The sequence of the SARS-CoV-2 protein or fragment or variant thereof may be obtained from any source. In some embodiments, the sequence of the SARS-CoV-2 protein or fragment or variant thereof is from a strain that is capable of or at risk of infecting human subjects.

In some embodiments, the sequence of the SARS-CoV-2 protein or fragment or variant thereof may be modified or optimized (such as codon optimized) for expression in a particular cell or host organism.

In some embodiments, the nucleic acid vaccine described herein may be a multivalent vaccine. The multivalent vaccine may include polynucleotides that encodes at least two different one or more proteins, peptides, fragments or variants thereof of SARS-CoV-2. As a non-limiting example, the polynucleotides may encode the same or a different structural protein. As a non-limiting example, the polynucleotides may encode the same structural protein but different variants of the structural protein.

In some embodiments, the nucleic acid vaccine encodes the full-length S protein of SARS-CoV-2. In some embodiments, the nucleic acid vaccine encodes a fragment of the S protein of SARS-CoV-2. In some embodiments, the nucleic acid vaccine encodes the receptor binding domain (RBD) fragment of the spike protein of SARS-CoV-2. In some embodiments, the nucleic acid vaccine encodes a variant of the spike protein of SARS-CoV-2. In some embodiments, the nucleic acid vaccine encodes a S protein sequence of SARS-CoV-2 (e.g., full-length, fragment or variant) where the S protein has a mutated furin cleavage site. The S protein furin cleavage site mutant will remove or disable the furin cleavage site(s) in S protein (e.g., between the S1 and S2 boundary). In some viral envelope proteins, disruption of a furin cleavage site was found to enhance expression and stability. In some embodiments, the nucleic acid vaccine encodes a S protein sequence of SARS-CoV-2 (e.g., full-length, fragment or variant) where the S protein includes the D614G mutation. The nucleic acid vaccine encoding the S protein of SARS-CoV-2, a fragment or variant thereof may also include a signal peptide and/or at least one linker (e.g., GSG linker) sequence and one or more sequences in the nucleic acid vaccine may be codon optimized.

In some embodiments, the nucleic acid vaccine encodes the full-length M protein of SARS-CoV-2. In some embodiments, the nucleic acid vaccine encodes a fragment of the M protein of SARS-CoV-2. In some embodiments, the nucleic acid vaccine encodes the topological domain (e.g., virion surface or intravirion region) of the M protein of SARS-CoV-2. In some embodiments, the nucleic acid vaccine encodes the transmembrane domain of the M protein of SARS-CoV-2. In some embodiments, the nucleic acid vaccine encodes a variant of the M protein (e.g., full-length protein or fragment) of SARS-CoV-2. The nucleic acid vaccine encoding the M protein of SARS-CoV-2, a fragment or variant thereof may also include a signal peptide and/or at least one linker (e.g., GSG linker) sequence and one or more sequences in the nucleic acid vaccine may be codon optimized.

In some embodiments, the nucleic acid vaccine encodes the full-length N protein of SARS-CoV-2. In some embodiments, the nucleic acid vaccine encodes a fragment of the N protein of SARS-CoV-2. In some embodiments, the nucleic acid vaccine encodes the RNA binding domain of the N protein of SARS-CoV-2. In some embodiments, the nucleic acid vaccine encodes the dimerization domain of the N protein of SARS-CoV-2. In some embodiments, the nucleic acid vaccine encodes a variant of the N protein (e.g., full-length protein or fragment) of SARS-CoV-2. The nucleic acid vaccine encoding the N protein of SARS-CoV-2, a fragment or variant thereof may also include a signal peptide and/or at least one linker (e.g., GSG linker) sequence and one or more sequences in the nucleic acid vaccine may be codon optimized.

In some embodiments, the nucleic acid vaccine encodes the full-length E protein of SARS-CoV-2. In some embodiments, the nucleic acid vaccine encodes a fragment of the E protein of SARS-CoV-2. In some embodiments, the nucleic acid vaccine encodes the topological domain (e.g., virion surface or intravirion region) of the E protein of SARS-CoV-2. In some embodiments, the nucleic acid vaccine encodes the transmembrane domain of the E protein of SARS-CoV-2. In some embodiments, the nucleic acid vaccine encodes a variant of the E protein (e.g., full-length protein or fragment) of SARS-CoV-2. The nucleic acid vaccine encoding the E protein of SARS-CoV-2, a fragment or variant thereof may also include a signal peptide and/or at least one linker (e.g., GSG linker) sequence and one or more sequences in the nucleic acid vaccine may be codon optimized.

In some embodiments, the nucleic acid vaccine encodes two different structural proteins of SARS-CoV-2. In some embodiments, the nucleic acid vaccine encodes a S protein, fragment or variant thereof of SARS-CoV-2 and a M protein, fragment or variant thereof of SARS-CoV-2. In some embodiments, the nucleic acid vaccine encodes a S protein, fragment or variant thereof of SARS-CoV-2 and a N protein, fragment or variant thereof of SARS-CoV-2. In some embodiments, the nucleic acid vaccine encodes a S protein, fragment or variant thereof of SARS-CoV-2 and an E protein, fragment or variant thereof of SARS-CoV-2. In some embodiments, the nucleic acid vaccine encodes a M protein, fragment or variant thereof of SARS-CoV-2 and a N protein, fragment or variant thereof of SARS-CoV-2. In some embodiments, the nucleic acid vaccine encodes a M protein, fragment or variant thereof of SARS-CoV-2 and an E protein, fragment or variant thereof of SARS-CoV-2. In some embodiments, the nucleic acid vaccine encodes a N protein, fragment or variant thereof of SARS-CoV-2 and an E protein, fragment or variant thereof of SARS-CoV-2. The nucleic acid vaccine encoding two different structural proteins, fragment or variant thereof of SARS-CoV-2, may also include a signal peptide and/or at least one linker (e.g., GSG linker) sequence and one or more sequences in the nucleic acid vaccine may be codon optimized. In some embodiments, the sequences encoding the two different structural proteins or fragments or variants thereof of SARS-Cov-2 of the nucleic acid vaccine are constructed as a single polynucleotide.

In some embodiments, the nucleic acid vaccine encodes at least three different sequences of the structural proteins fragment or variant thereof for SARS-CoV-2. In some embodiments, the nucleic acid vaccine encodes two different S proteins, fragments or variants sequences for SARS-CoV-2 and a M protein, fragment or variant sequence for SARS-CoV-2. In some embodiments, the nucleic acid vaccine encodes two different S proteins, fragments or variants sequences for SARS-CoV-2 and a N protein, fragment or variant sequence for SARS-CoV-2. In some embodiments, the nucleic acid vaccine encodes two different S proteins, fragments or variants sequences for SARS-CoV-2 and an E protein, fragment or variant sequence for SARS-CoV-2. In some embodiments, the nucleic acid vaccine encodes two different M proteins, fragments or variants sequences for SARS-CoV-2 and a S protein, fragment or variant sequence for SARS-CoV-2. In some embodiments, the nucleic acid vaccine encodes two different N proteins, fragments or variants sequences for SARS-CoV-2 and a S protein, fragment or variant sequence for SARS-CoV-2. In some embodiments, the nucleic acid vaccine encodes two different E proteins, fragments or variants sequences for SARS-CoV-2 and a S protein, fragment or variant sequence for SARS-CoV-2. In some embodiments, the nucleic acid vaccine encodes two different M proteins, fragments or variants sequences for SARS-CoV-2 and a N protein, fragment or variant sequence for SARS-CoV-2. In some embodiments, the nucleic acid vaccine encodes two different M proteins, fragments or variants sequences for SARS-CoV-2 and an E protein, fragment or variant sequence for SARS-CoV-2. In some embodiments, the nucleic acid vaccine encodes two different N proteins, fragments or variants sequences for SARS-CoV-2 and a M protein, fragment or variant sequence for SARS-CoV-2. In some embodiments, the nucleic acid vaccine encodes two different N proteins, fragments or variants sequences for SARS-CoV-2 and an E protein, fragment or variant sequence for SARS-CoV-2. In some embodiments, the nucleic acid vaccine encodes two different E proteins, fragments or variants sequences for SARS-CoV-2 and a N protein, fragment or variant sequence for SARS-CoV-2. In some embodiments, the nucleic acid vaccine encodes a S protein, fragment or variant sequence for SARS-CoV-2, a M protein, fragment or variant sequence for SARS-CoV-2, and a N protein, fragment or variant sequence for SARS-CoV-2. In some embodiments, the nucleic acid vaccine encodes a S protein, fragment or variant sequence for SARS-CoV-2, a M protein, fragment or variant sequence for SARS-CoV-2, and an E protein, fragment or variant sequence for SARS-CoV-2. In some embodiments, the nucleic acid vaccine encodes a S protein, fragment or variant sequence for SARS-CoV-2, a N protein, fragment or variant sequence for SARS-CoV-2, and an E protein, fragment or variant sequence for SARS-CoV-2. In some embodiments, the nucleic acid vaccine encodes a M protein, fragment or variant sequence for SARS-CoV-2, a N protein, fragment or variant sequence for SARS-CoV-2, and an E protein, fragment or variant sequence for SARS-CoV-2. The nucleic acid vaccine encoding at least three different sequences of the structural proteins fragment or variant thereof for SARS-CoV-2, may also include a signal peptide and/or at least one linker (e.g., GSG linker) sequence and one or more sequences in the nucleic acid vaccine may be codon optimized.

### SARS-CoV-2 Variants

SARS-CoV-2 is a member of the large coronavirus family of viruses. Multiple variants (sometimes referred to as “strains” or “lineages”) of SARS-CoV-2 have been identified globally. The nomenclature for SARS-CoV-2 variants used in this description is consistent with the PANGO nomenclature for new virus lineages (Rambaut, Andrew, et al., A dynamic nomenclature proposal for SARS-CoV-2 lineages to assist genomic epidemiology. Nature microbiology, 2020, 5: 1403-1407, the contents of which are incorporated herein by reference in their entirety). Near real-time data relating to PANGO SARS-CoV-2 lineages or variants can be accessed online using Nextstrain's SARs-CoV-2 analysis user interface (nextstrain.org/ncov/global).

As of this disclosure, numerous PANGO lineage variants of SARS-CoV-2 have been identified, including the following (number in parentheses represents number of cases per each submitted PANGO lineage: A (37); A.1 (8); A.11 (2); A.12 (1); A.19 (5); A.2 (6); A.2.2 (9); A.2.4 (5); A.2.5 (12); A.21 (8); A.22 (1); A.23 (2); A.23.1 (40); A.24 (2); A.25 (1); A.28 (4); A.3 (3); A.5 (5); A.6 (1); AD.2 (1); AE.1 (1); AE.2 (2); AE.4 (1); AE.5 (1); AE.7 (1); AE.8 (1); AG.1 (1); AY.1; AY.2; AY.3; B (47); B.1 (374); B.1.1 (237); B.1.1.1 (40); B.1.1.10 (2); B.1.1.111 (2); B.1.1.121 (1); B.1.1.133 (2); B.1.1.141 (5); B.1.1.142 (6); B.1.1.153 (6); B.1.1.157 (1); B.1.1.159 (3); B.1.1.160 (1); B.1.1.161 (1); B.1.1.163 (8); B.1.1.170 (1); B.1.1.174 (1); B.1.1.176 (2); B.1.1.180 (1); B.1.1.186 (2); B.1.1.189 (4); B.1.1.198 (2); B.1.1.200 (1); B.1.1.204 (2); B.1.1.207 (6); B.1.1.214 (22); B.1.1.216 (9); B.1.1.219 (1); B.1.1.222 (32); B.1.1.226 (1); B.1.1.230 (1); B.1.1.231 (4); B.1.1.232 (1); B.1.1.241 (1); B.1.1.243 (1); B.1.1.25 (26); B.1.1.263 (2); B.1.1.265 (1); B.1.1.27 (6); B.1.1.273 (1); B.1.1.274 (7); B.1.1.28 (34); B.1.1.280 (3); B.1.1.284 (5); B.1.1.294 (7); B.1.1.297 (1); B.1.1.300 (1); B.1.1.301 (1); B.1.1.304 (1); B.1.1.306 (5); B.1.1.312 (3); B.1.1.315 (2); B.1.1.316 (4); B.1.1.317 (8); B.1.1.318 (1); B.1.1.326 (1); B.1.1.328 (3); B.1.1.33 (14); B.1.1.330 (6); B.1.1.331 (1); B.1.1.333 (4); B.1.1.337 (2); B.1.1.344 (2); B.1.1.345 (1); B.1.1.348 (29); B.1.1.350 (1); B.1.1.351 (2); B.1.1.354 (7); B.1.1.355 (2); B.1.1.359 (2); B.1.1.365 (1); B.1.1.366 (1); B.1.1.368 (1); B.1.1.37 (1); B.1.1.372 (2); B.1.1.374 (5); B.1.1.375 (9); B.1.1.381 (1); B.1.1.383 (1); B.1.1.388 (1); B.1.1.389 (17); B.1.1.139 (3); B.1.1.394 (3); B.1.1.397 (4); B.1.1.398 (2); B.1.1.40 (2); B.1.1.404 (1); B.1.1.410 (3); B.1.1.411 (3); B.1.1.413 (3); B.1.1.416 (6); B.1.1.419 (2); B.1.1.420 (4); B.1.1.428 (2); B.1.1.429 (2); B.1.1.430 (1); B.1.1.432 (8); B.1.1.434 (1); B.1.1.447 (1); B.1.1.448 (2); B.1.1.451 (1); B.1.1.464 (2); B.1.1.485 (1); B.1.1.487 (5); B.1.1.50 (16); B.1.1.514 (2); B.1.1.516 (2); B.1.1.517 (1); B.1.1.519 (106); B.1.1.521 (1); B.1.1.54 (2); B.1.1.56 (1); B.1.1.57 (1); B.1.1.63 (7); B.1.1.7 (534); B.1.1.70 (10); B.1.1.71 (1); B.1.1.99 (1); B.1.108 (1); B.1.110.3 (1); B.1.111 (29); B.1.116 (1); B.1.126 (2); B.1.128 (3); B.1.13 (1); B.1.139 (2); B.1.146 (1); B.1.149 (1); B.1.153 (2); B.1.160 (65); B.1.160.14 (1); B.1.160.15 (1); B.1.160.25 (1); B.1.160.28 (1); B.1.160.8 (1); B.1.160.9 (1); B.1.164 (2); B.1.170 (2); B.1.177 (71); B.1.177.11 (1); B.1.177.12 (1); B.1.177.15 (1); B.1.177.18 (1); B.1.177.21 (7); B.1.177.32 (4); B.1.177.35 (1); B.1.177.4 (1); B.1.177.40 (2); B.1.177.42 (1); B.1.177.43 (1); B.1.177.44 (2); B.1.177.46 (3); B.1.177.49 (1); B.1.177.51 (1); B.1.177.52 (3); B.1.177.53 (1); B.1.177.54 (2); B.1.177.59 (1); B.1.177.6 (1); B.1.177.60 (23); B.1.177.68 (1); B.1.177.73 (6); B.1.177.76 (1); B.1.177.77 (1); B.1.177.78 (1); B.1.177.79 (1); B.1.177.81 (5); B.1.177.82 (1); B.1.177.83 (1); B.1.177.86 (3); B.1.189 (2); B.1.192 (7); B.1.195 (4); B.1.2 (222); B.1.210 (2); B.1.214 (6); B.1.214.2 (1); B.1.219 (5); B.1.22 (3); B.1.22.1 (16); B.1.220 (1); B.1.221 (27); B.1.221.1 (1); B.1.223 (1); B.1.229 (1); B.1.23 (2); B.1.232 (1); B.1.234 (20); B.1.236 (3); B.1.237 (3); B.1.240 (7); B.1.240.1 (14); B.1.241 (1); B.1.243 (34); B.1.256 (1); B.1.258 (61); B.1.258.11 (1); B.1.258.17 (17); B.1.258.2 (1); B.1.258.22 (1); B.1.258.23 (1); B.1.260 (2); B.1.273 (1); B.1.277 (1); B.1.279 (1); B.1.281 (4); B.1.289 (1); B.1.291 (2); B.1.3 (1); B.1.306 (1); B.1.308 (1); B.1.311 (5); B.1.324 (1); B.1.329 (1); B.1.334 (1); B.1.338 (1); B.1.346 (1); B.1.349 (2); B.1.351 (199); B.1.356 (3); B.1.357 (1); B.1.36 (56); B.1.36.1 (5); B.1.36.10 (2); B.1.36.16 (33); B.1.36.17 (1); B.1.36.18 (12); B.1.36.19 (1); B.1.36.21 (1); B.1.36.22 (6); B.1.36.29 (6); B.1.36.31 (5); B.1.36.34 (3); B.1.36.38 (1); B.1.36.8 (3); B.1.360 (1); B.1.361 (3); B.1.362 (8); B.1.367 (3); B.1.369 (12); B.1.369.1 (1); B.1.370 (1); B.1.371 (1); B.1.375 (1); B.1.379 (1); B.1.380 (9); B.1.393 (1); B.1.396 (2); B.1.398 (11); B.1.399 (1); B.1.400 (4); B.1.404 (2); B.1.409 (5); B.1.411 (19); B.1.416 (16); B.1.420 (6); B.1.426 (1); B.1.427 (25); B.1.428 (4); B.1.429 (58); B.1.438 (4); B.1.441 (4); B.1.451 (1); B.1.456 (4); B.1.459 (15); B.1.462 (1); B.1.465 (1); B.1.466 (4); B.1.466.1 (1); B.1.466.2 (34); B.1.468 (7); B.1.469 (1); B.1.470 (11); B.1.471 (5); B.1.476 (1); B.1.478 (1); B.1.479 (1); B.1.480 (2); B.1.492 (1); B.1.497 (28); B.1.499 (24); B.1.504 (1); B.1.505 (1); B.1.509 (3); B.1.517 (6); B.1.517.1 (16); B.1.523 (3); B.1.524 (21); B.1.525 (16); B.1.526 (8); B.1.526.1 (6); B.1.526.2 (2); B.1.527 (2); B.1.530 (9); B.1.535 (2); B.1.540 (1); B.1.541 (1); B.1.544 (8); B.1.547 (1); B.1.551 (1); B.1.558 (3); B.1.560 (1); B.1.561 (6); B.1.564 (2); B.1.565 (6); B.1.568 (3); B.1.575 (4); B.1.576 (1); B.1.577 (5); B.1.581 (1); B.1.582 (7); B.1.587 (1); B.1.588 (4); B.1.595.4 (1); B.1.596 (17); B.1.596.1 (1); B.1.600 (8); B.1.603 (2); B.1.605 (1); B.1.609 (6); B. 1.617.1; B.1.617.2; B.1.617.3; B.1.619 (1); B.1.620 (12); B.1.621 (65); B.1.67 (1); B.1.84 (1); B.1.91 (4); B.1.94 (1); B.12 (1); B.27 (3); B.28 (1); B.3 (6); B.31 (2); B.35 (4); B.4 (13); B.4.1 (1); B.4.2 (1); B.4.6 (2); B.4.7 (2); B.40 (3); B.42 (2); B.43 (1); B.45 (1); B.53 (2); B.55 (2); B.56 (1); B.6 (23); B.6.3 (1); B.6.6 (5); B.6.7 (1); B.6.8 (29); C.1 (2); C.1.1 (1); C.11 (5); C.12 (3); C.13 (1); C.14 (4); C.16 (18); C.17 (2); C.18 (1); C.2 (5); C.2.1 (11); C.23 (2); C.26 (5); C.29 (1); C.30 (1); C.32 (1); C.35 (10); C.36 (14); C.36.1 (1); C.36.3 (10); C.4 (3); C.8 (2); C. 37 (54); D.2 (33); L.3 (8); N.2 (1); N.3 (4); N.4 (14); N.5 (8); N.6 (3); N.7 (4); N.9 (4); P.1 (57); P.2 (47); P.6 (2); P.7 (3); Q.1 (6), Q.3 (7); Q.4 (2); Q.5 (1); Q.6 (1). Q.8 (7), R.1 (9); S.1 (1); U.2 (1); U.3 (1); W.1 (1); Y.1 (2); and Z.1 (1).

From an epidemiological perspective, variants are typically categorized as Variants of Interest (VOIs), Variants of Concern (VOCs), and Variants of High Consequence (VOHCs). For information relevant to categorizing specific variants as VOIs, VOCs, or VOHCs see, for example, cdc.gov/coronavirus/2019-ncov/cases-updates/variant-surveillance/variant-info.html.

VOIs may have certain genetic markers associated with changes to receptor binding, reduced neutralization by antibodies generated against previous infection or vaccination, reduced efficacy of treatments, potential diagnostic impact, or predicted increase in transmissibility or disease severity. In some instances, VOIs have specific genetic markers that are predicted to affect transmission, diagnostics, therapeutics, or immune escape, or cause an increased proportion of cases or unique outbreak clusters. SARS-CoV-2 VOIs include, for example, PANGO lineage B. 1.1.7 (Alpha), B.1.351 (Beta); B.1.427/429 (Epsilon); B.1.526 (Iota); B.1.525 (Eta); B.1.617.1 (Kappa); B.1.617.2 (Delta); B.1.621 (Mu); C37 (Lamba); P.1 (Gamma) and P.2.

VOCs may include variants for which there is evidence of an increase in transmissibility, more severe disease (increased hospitalizations or deaths), significant reduction in neutralization by antibodies generated during previous infection or vaccination, reduced effectiveness of treatments or vaccines, or diagnostic detection failures. In some instances, VOCs have evidence of impact on diagnostics, treatments, and vaccines, widespread interference with diagnostic test targets, evidence of substantially increased resistance to one or more class of therapies, evidence of significant decreased neutralization by antibodies generated during previous infection or vaccination, evidence of reduced vaccine-induced protection from severe disease, evidence of increased transmissibility, or evidence of increased disease severity. SARS-CoV-2 VOCs may include, for example, PANGO lineage B.1.1.7 (Alpha), P.1 (Gamma), B.1.351(Beta), B.1.427 and B.1.429 ((Epsilon), B.1.526 (Iota), B.1.525 (Eta), B.1.617.1 (Kappa), B.1.617.2 (Delta), B.1.621 (Mu), and C37 (Lamba).

VOHCs may have clear evidence that prevention measures or medical countermeasures (MCMs) have significantly reduced effectiveness relative to previously circulating variants. In some instances, VOHCs have impact on Medical Countermeasures (MCM), demonstrated failure of diagnostics, evidence to suggest a significant reduction in vaccine effectiveness, a disproportionately high number of vaccine breakthrough cases, very low vaccine-induced protection against severe disease, significantly reduced susceptibility to multiple Emergency Use Authorization (EUA) or approved therapeutics, more severe clinical disease and increased hospitalizations.

The nucleic acid vaccines disclosed herein may encode one or more polypeptides, e.g., one or more proteins, peptides, fragments or variants thereof, of any of the SARS-CoV-2 variants described herein. In some embodiments, the nucleic acid vaccines disclosed herein may encode one or more polypeptides, e.g., one or more proteins, peptides, fragments or variants thereof, of a SARS-CoV-2 VOI, VOC, and/or VOHC. In some embodiments, the nucleic acid vaccines encode a polypeptide comprising the specific mutation called D614G.

In some embodiments, the nucleic acid vaccines encode one or more polypeptide comprising one or more mutations or substitutions present in the B.1.526 SARS-CoV-2 variant (i.e., Iota variant), such as one or more of: Spike protein substitutions L5F, T95I, D253G, S477N, E484K, D614G, and/or A701V; ORF1a substitutions L3201P, T265I, and/or Δ3675/3677; ORF1b substitutions P314L and/or Q1011H; ORF3a substitutions P42L, Q57H; ORF8 substitution Ti 11; and/or 5′UTR substitution R81C.

In some embodiments, the nucleic acid vaccines encode one or more polypeptide comprising one or more mutations or substitutions present in the B.1.525 SARS-CoV-2 variant (i.e., Eta variant), such as one or more of: Spike protein substitutions A67V, A69/70, A144, E484K, D614G, Q677H and/or F888L; ORF1b substitution P314F; ORF1a substitution T2007I; M protein substitution I82T; N protein substitutions A12G and/or T205I; and/or 5′UTR substitution R81C.

In some embodiments, the nucleic acid vaccines encode one or more polypeptide comprising one or more mutations or substitutions present in the P.2 SARS-CoV-2 variant, such as one or more of: Spike protein substitutions E484K, D614G, and/or V1176F; ORF1a substitutions L3468V and/or L3930F; ORF1b substitution P314L; N protein substitutions A119S, R203K, G204R, and/or M234I; 5′UTR substitution R81C.

In some embodiments, the nucleic acid vaccines encode one or more polypeptide comprising one or more mutations or substitutions present in the B.1.1.7 SARS-CoV-2 variant (i.e., Alpha variant), such as one or more of: Spike protein substitutions A69/70, A144Y, E484K, S494P, N501Y, A570D, D614G, and/or P681H.

In some embodiments, the nucleic acid vaccines encode one or more polypeptide comprising one or more mutations or substitutions present in the P.1 SARS-CoV-2 variant (i.e., Gamma variant), such as one or more of: Spike protein substitutions K417N/T, E484K, N501Y, and/or D614G.

In some embodiments, the nucleic acid vaccines encode one or more polypeptide comprising one or more mutations or substitutions present in the B.1.351 SARS-CoV-2 variant (i.e., Beta variant), such as one or more of: Spike protein substitutions K417N, E484K, N501Y, and/or D614G. The B.1.351 variant is also referred to as the South African variant, as it first originated in South Africa.

In some embodiments, the nucleic acid vaccines encode one or more polypeptide comprising one or more mutations or substitutions present in the B.1.427 SARS-CoV-2 variant, such as one or more of: Spike protein substitutions L452R and/or D614G.

In some embodiments, the nucleic acid vaccines encode one or more polypeptide comprising one or more mutations or substitutions present in the B.1.429 SARS-CoV-2 variant, such as one or more of: Spike protein substitutions S13I, W152C, L452R, and/or D614G.

In some embodiments, the nucleic acid vaccines encode one or more polypeptide comprising one or more mutations or substitutions present in the B.1.617.1 SARS-CoV-2 variant, such as one or more of: Spike protein substitutions G142D, E154K, L452R, E484Q, D614G, P681R, and/or Q1071H.

In some embodiments, the nucleic acid vaccines encode one or more polypeptide comprising one or more mutations or substitutions present in the B.1.617.2 SARS-CoV-2 variant (i.e., Delta variant), such as one or more of: Spike protein substitutions T19R, T95I, G142D, A156/157, R158G, L452R, T478K, D614G, P681R, and/or D950N. In other embodiments, the nucleic acid vaccines encode one or more polypeptide comprising one or more further mutations or substitutions present in the B.1.617.2 SARS-CoV-2 variant, such as one or more of: Spike protein substitutions V70F, A222V, W258L, and/or K417N.

In some embodiments, the nucleic acid vaccines encode one or more polypeptide comprising one or more mutations or substitutions present in the B.1.617.3 SARS-CoV-2 variant, such as one or more of: Spike protein substitutions T19R, G142D, L452R, E484Q, D614G, P681R, and/or D950N.

In some embodiments, the nucleic acid vaccines encode a SARS-CoV-2 Spike protein, e.g., protein, peptide, fragment, or variant, comprising one or more substitutions and/or deletions selected from: A570D, A67V, A701V, D253G, D614G, E484K, F888L, K417N/T, L452R, L5F, N501Y, P681H, Q677H, S13I, S477N, S494P, T95I, V1176F, W152C, Δ144, Δ144Y, and Δ69/70.

In some embodiments, the nucleic acid vaccines encode a SARS-CoV-2 ORF1a comprising one or more substitutions and/or deletions selected from: L3201P, T265I, T2007I, L3468V, A3675-3677, and L3930F.

In some embodiments, the nucleic acid vaccines encode a SARS-CoV-2 ORF1b comprising one or more substitutions selected from: P314F, P314L, and Q1011H.

In some embodiments, the nucleic acid vaccines encode a SARS-CoV-2 ORF3a comprising one or more substitutions selected from: P42L and Q57H.

In some embodiments, the nucleic acid vaccines encode a SARS-CoV-2 ORF8 comprising a T11I substitution.

In some embodiments, the nucleic acid vaccines encode a SARS-CoV-2 5′UTR comprising a R81C substitution.

In some embodiments, the nucleic acid vaccines encode a SARS-CoV-2 M protein, e.g., protein, peptide, fragment, or variant, comprising I82T substitution.

In some embodiments, the nucleic acid vaccines encode a SARS-CoV-2 N protein, e.g., protein, peptide, fragment, or variant, comprising one or more substitutions selected from: A12G, A119S, R203K, G204R, T205I, and M234I.

### Components of Nucleic Acid Vaccines

In some embodiments, the polynucleotides described herein encode at least one polypeptide of interest, e.g., one or more proteins, peptides, fragments or variants thereof of SARS-CoV-2. The proteins, peptides, fragments or variants thereof of SARS-CoV-2 of the present disclosure may be wild type where they are derived from the infectious agent, or modified (e.g., the structural proteins or fragments and variants thereof are engineered, designed or artificial). They may have any combination of the features described herein.

In some embodiments, the polynucleotides of the nucleic acid vaccines described herein encode one or more peptides or polypeptides of interest. Such peptides or polypeptides are structural proteins, or fragments or variants thereof of SARS-CoV-2 for the prevention, alleviation and/or treatment of COVID-19. As a non-limiting example, these peptides or polypeptides may serve as an antigen or antigenic molecule (also preferred to as immunogenic molecule). The term “nucleic acid,” in its broadest sense, includes any compound and/or substance that comprise a polymer of nucleotides. These polymers are often referred to as polynucleotides.

Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof.

In some embodiments, in vitro transcription (IVT) enzymatic synthesis methods may be used to make linear polynucleotides (referred to as “IVT polynucleotides”) encoding one or more proteins, peptides, fragments or variants thereof of SARS-CoV-2 of the present disclosure.

In some embodiment, the nucleic acid vaccines may include “chimeric polynucleotides” which have portions or regions which differ in size and/or encoded protein (e.g., structural protein of SARS-CoV-2). A “chimera” is an entity having two or more incongruous or heterogeneous parts or regions. As used herein a “part” or “region” of a polynucleotide is defined as any portion of the polynucleotide which is less than the entire length of the polynucleotide.

In some embodiments, the nucleic acid vaccine includes polynucleotides from about 30 to about 100,000 nucleotides in length(e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some embodiments, the nucleic acid vaccine includes at least one polynucleotide encoding at least one peptide or polypeptide of interest. In another embodiment, the polynucleotides may be non-coding.

In some embodiments, the length of a region encoding at least one peptide or polypeptide of interest of the polynucleotides of the nucleic acid vaccine is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides). As used herein, such a region may be referred to as a “coding region” or “region encoding.”

In some embodiments, the polynucleotides of the nucleic acid vaccine is or functions as a messenger RNA (mRNA). As used herein, the term “messenger RNA (mRNA)” refers to any polynucleotide which encodes at least one peptide or polypeptide of interest and which is capable of being translated to produce the encoded peptide or polypeptide of interest in vitro, in vivo, in situ or ex vivo.

The shortest length of a region of the polynucleotide of the nucleic acid vaccine can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g., 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 40 amino acids, e.g., no longer than 35, 30, 25, 20, 17, 15, 14, 13, 12, 11 or 10 amino acids. Examples of dipeptides that the polynucleotide sequences can encode or include, but are not limited to, carnosine and anserine.

The region of the polynucleotide of the nucleic acid vaccine encoding one or more proteins, peptides, fragments or variants thereof of SARS-CoV-2 for the prevention, alleviation and/or treatment of COVID-19 may be greater than about 30 nucleotides in length. The length may be, but is not limited to, at least or greater than about 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides. In some embodiments, the region includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

mRNA Components

The nucleic acid vaccines described herein may be an mRNA vaccine. The mRNA vaccine includes at least one mRNA molecule which, when translated, produce at least one peptide or polypeptide of interest for the prevention, alleviation and/or treatment of COVID-19. In general, an mRNA molecule generally includes at least a coding region, a 5′ untranslated region (UTR), a 3′ UTR, a 5′ cap and a poly-A tail. mRNA Components: Start Codon and Stop Codon

In some embodiments, the mRNA includes a region to initiate translation. This region may include any translation initiation sequence or signal including a Start codon. As a non-limiting example, the region includes a Start codon. In some embodiments, the Start codon may be “ATG,” “ACG,” “AGG,” “ATA,” “ATT,” “CTG,” “GTG,” “TTG,” “AUG,” “AUA,” “AUU,” “CUG,” “GUG,” or “UUG”.

In some embodiments, the mRNA includes a region to stop translation. This region may include any translation termination sequence or signal including a Stop codon. As a non-limiting example, the region includes a Stop codon. In some embodiments, the Stop codon may be “TGA,” “TAA,” “TGA,” “TAG,” “UGA,” “UAA,” “UGA” or “UAG.”

In some embodiments, the regions to initiate or terminate translation may independently range from 3 to 40, e.g., 5-30, 10-20, 15, or at least 4, or 30 or fewer nucleotides in length. Additionally, these regions may comprise, in addition to a Start and/or Stop codon, one or more signal and/or restriction sequences.

In some embodiments, a masking agent may be used to mask a first start codon or alternative start codon in order to increase the chance that translation will initiate on a start codon or alternative start codon downstream to the masked start codon or alternative start codon.

In some embodiments, the start codon may be removed from the polynucleotide sequence in order to have the translation of the polynucleotide begin on a codon which is not the start codon. Translation of the polynucleotide may begin on the codon following the removed start codon or on a downstream start codon or an alternative start codon. The polynucleotide sequence where the start codon is removed may further comprise at least one masking agent for the downstream start codon and/or alternative start codons in order to control or attempt to control the initiation of translation, the length of the polynucleotide and/or the structure of the polynucleotide.

mRNA Components: Coding Region

In some embodiments, the coding region of the polynucleotide of the nucleic acid vaccine may encode at least one peptide or polypeptide of interest. Non-limiting examples of peptides or polypeptides of interest include one or more proteins, peptides, fragments or variants thereof of SARS-CoV-2 for the prevention, alleviation and/or treatment of COVID-19.

mRNA Components: Untranslated Region

The polynucleotides of the nucleic acid vaccines described herein may comprise one or more regions or parts which act or function as an untranslated region (UTR). Wild type UTRs of a gene are transcribed but not translated. In mRNA, the 5 ‘UTR starts at the transcription start site and continues to the start codon but does not include the start codon; whereas, the 3′ UTR starts immediately following the stop codon and continues until the transcriptional termination signal. While not wishing to be bound by theory, UTRs may have a role in terms of stability and translation of the nucleic acid molecule and translation. Variants of UTRs may be utilized wherein one or more nucleotides are added or removed to the termini, including A, T, C or G.

In some embodiments, the UTRs of the polynucleotide of the nucleic acid vaccine may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

Wild type 5′ UTRs include features which play roles in translation initiation as these 5′ UTRs include sequences such as Kozak sequences which are known to be involved in how the ribosome initiates translation of many genes. 5′ UTRs also have been known to form secondary structures which are involved in elongation factor binding. Other non-UTR sequences (e.g., introns or portions of intron sequences) may also be used as regions or subregions which may increase protein production as well as polynucleotide levels.

Natural or wild type 3′ UTRs are known to have stretches of Adenosines and Uridines embedded in them. These AU rich signatures are particularly prevalent in genes with high rates of turnover. Introduction, removal or modification of 3′ UTR AU rich elements (AREs) can be used to modulate the stability of polynucleotides of the nucleic acid vaccines.

The UTR from any gene may be incorporated into the regions of the polynucleotides of the nucleic acid vaccines. Alternatively, artificial UTRs, which are not variants of wild type regions, may also be used in the polynucleotides of the nucleic acid vaccines. These UTRs or portions thereof may be placed in the same orientation as in the transcript from which they were selected or may be altered in orientation or location. As used herein, the term “altered” as it relates to a UTR sequence, means that the UTR has been changed in some way in relation to a reference sequence. As a non-limiting example, a 5′ or 3′ UTR may be inverted, shortened, lengthened, made with one or more other 5′ UTRs or 3′ UTRs from a different parental sequence.

In some embodiments, flanking regions are selected from a family of transcripts whose proteins share a common function, structure, feature of property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The polynucleotides of the nucleic acid vaccines disclosed herein may comprise a 5′UTR having a sequence of SEQ ID NO: 13 (DNA) or SEQ ID NO: 47 (RNA). In some embodiments, the 5′ UTR of the polynucleotides of the nucleic acid vaccines disclosed herein consist of the nucleic acid sequence of SEQ ID NO: 13 (DNA) or SEQ ID NO: 47 (RNA). In some embodiments, the 5′UTR is directly 5′ of the start codon of the sequence encoding the SARs-CoV-2 polypeptide of the nucleic acid vaccine. In some embodiments, the 5′UTR is 1, 2, 3, 4, 5, 6 or more nucleotides 5′ of the start codon of the sequence encoding the SARs-CoV-2 polypeptide of the nucleic acid vaccine; e.g., a spacer sequence of 1, 2, 3, 4, 5, 6 or more nucleotides separates the 5′UTR from the start codon of the sequence encoding the SARs-CoV-2 polypeptide of the nucleic acid vaccine. The polynucleotides of the nucleic acid vaccines disclosed herein may comprise a 5′UTR having a sequence with at least 80% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 85% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 90% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 91% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 92% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 93% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 94% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 95% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 96% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 97% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 98% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 99% sequence identity to the nucleic acid sequence of SEQ ID NO: 13 (DNA) or SEQ ID NO: 47 (RNA). The polynucleotides of the nucleic acid vaccines disclosed herein may comprise a 5′UTR having a sequence with at least 100% sequence identity to the nucleic acid sequence of SEQ ID NO: 13 (DNA) or SEQ ID NO: 47 (RNA).

The polynucleotides of the nucleic acid vaccines disclosed herein may comprise a 3′UTR having a sequence of SEQ ID NO: 14 (DNA) or SEQ ID NO: 48 (RNA). In some embodiments, the 3′ UTR of the polynucleotides of the nucleic acid vaccines disclosed herein consist of the nucleic acid sequence of SEQ ID NO: 14 (DNA) or SEQ ID NO: 48 (RNA). In some embodiments, the 3′UTR is directly 3′ of the start codon of the sequence encoding the SARs-CoV-2 polypeptide of the nucleic acid vaccine. In some embodiments, the 3′UTR is 1, 2, 3, 4, 5, 6 or more nucleotides 3′ of the start codon of the sequence encoding the SARs-CoV-2 polypeptide of the nucleic acid vaccine; e.g., a spacer sequence of 1, 2, 3, 4, 5, 6 or more nucleotides separates the 3′UTR from the start codon of the sequence encoding the SARs-CoV-2 polypeptide of the nucleic acid vaccine. The polynucleotides of the nucleic acid vaccines disclosed herein may comprise a 3′UTR having a sequence with at least 80% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 85% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 90% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 91% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 92% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 93% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 94% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 95% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 96% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 97% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 98% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 99% sequence identity to the nucleic acid sequence of SEQ ID NO: 14 (DNA) or SEQ ID NO: 48 (RNA). The polynucleotides of the nucleic acid vaccines disclosed herein may comprise a 3′UTR having a sequence with at least 100% sequence identity to the nucleic acid sequence of SEQ ID NO: 14 (DNA) or SEQ ID NO: 48 (RNA).

The polynucleotides of the nucleic acid vaccines disclosed herein may comprise a 3′UTR having a sequence of SEQ ID NO: 52 (DNA) or SEQ ID NO: 53 (RNA). In some embodiments, the 3′ UTR of the polynucleotides of the nucleic acid vaccines disclosed herein consist of the nucleic acid sequence of SEQ ID NO: 52 (DNA) or SEQ ID NO: 53 (RNA). In some embodiments, the 3′UTR is directly 3′ of the start codon of the sequence encoding the SARs-CoV-2 polypeptide of the nucleic acid vaccine. In some embodiments, the 3′UTR is 1, 2, 3, 4, 5, 6 or more nucleotides 3′ of the start codon of the sequence encoding the SARs-CoV-2 polypeptide of the nucleic acid vaccine; e.g., a spacer sequence of 1, 2, 3, 4, 5, 6 or more nucleotides separates the 3′UTR from the start codon of the sequence encoding the SARs-CoV-2 polypeptide of the nucleic acid vaccine. The polynucleotides of the nucleic acid vaccines disclosed herein may comprise a 3′UTR having a sequence with at least 80% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 85% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 90% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 91% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 92% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 93% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 94% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 95% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 96% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 97% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 98% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 99% sequence identity to the nucleic acid sequence of SEQ ID NO: 52 (DNA) or SEQ ID NO: 53 (RNA). The polynucleotides of the nucleic acid vaccines disclosed herein may comprise a 3′UTR having a sequence with at least 100% sequence identity to the nucleic acid sequence of SEQ ID NO: 52 (DNA) or SEQ ID NO: 53 (RNA).

mRNA Components: Cap and IRES Sequences

In some embodiments, the polynucleotides of the nucleic acid vaccines disclosed herein may comprise a 5′ cap structure. The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

In some embodiments, the 5′ terminal capping region of the polynucleotide of the nucleic acid vaccine may comprise a single cap or a series of nucleotides forming the cap. The capping region may be from 1 to 10, e.g., 2-9, 3-8, 4-7, 1-5, 5-10, or at least 2, or 10 or fewer nucleotides in length. In some examples, the capping region may comprise 1, 2, 3, 4, 5, 6, 7, 8, 9 or 10 nucleotides. In some embodiments, the cap is absent.

In some embodiments, cap analogs, which herein are also referred to as synthetic cap analogs, chemical caps, chemical cap analogs, or structural or functional cap analogs may be used in the nucleic acid vaccines. Cap analogs, which may be chemically (e.g., non-enzymatically) or enzymatically synthesized, differ from natural (e.g., endogenous, wild-type or physiological) 5′-caps in their chemical structure, but they retain cap function.

In some embodiments, the 5′ terminal caps of the polynucleotides of the nucleic acid vaccines may include endogenous caps or cap analogs. As a non-limiting example, 5′ terminal caps may comprise a guanine analog. Useful guanine analogs include, but are not limited to, inosine, N1-methyl-guanosine (mlG), 2′fluoro-guanosine, 7-deaza-guanosine, 8-oxo-guanosine, 2-amino-guanosine, LNA-guanosine, and 2-azido-guanosine.

The skilled artisan will appreciate that 5′ capping can be generated via enzymatic or other synthetic processes. Endogenous mRNA molecules are 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

Polynucleotides, e.g., mRNAs, of the nucleic acid vaccine described herein may be modified to include a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a vaccinia virus capping enzyme available from, e.g., New England Biolabs (Ipswich, MA) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-ante-terminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs may be chemically (e.g., non-enzymatically) or enzymatically synthesized and linked to a nucleic acid molecule, such as an mRNA molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivalently be designated 3′ 0-Me-m7G(5′)ppp(5′)G). The 3′-O atom of the other, unmodified, guanine becomes linked to the 5′-terminal nucleotide of the capped nucleic acid molecule (e.g., an mRNA). The N7- and 3′-O-methlyated guanine provide the terminal moiety of the capped nucleic acid molecule (e.g., mRNA).

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

While cap analogs allow for the concomitant capping of a nucleic acid molecule in an in vitro transcription reaction, up to 20% of transcripts can remain uncapped. This, as well as the structural differences of cap analogs from endogenous 5′-cap structures may lead to reduced translational competency and reduced cellular stability.

In exemplary aspects of the present disclosure, polynucleotides, e.g., mRNAs, can be capped post-transcriptionally, using enzymes. For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap 1 structure. In some embodiments, the Cap 1 structure provides a higher translational-competency and cellular stability and a reduced activation of cellular pro-inflammatory cytokines, as compared, e.g., to other 5′cap analog structures known in the art. Cap structures include 7mG(5′)ppp(5′)N,pN2p (Cap 0), 7mG(5′)ppp(5′)N1mpNp (Cap 1), and 7mG(5′)-ppp(5′)N1mpN2mp (Cap 2).

In one embodiment, the polynucleotide of the nucleic acid vaccine described herein comprises a Cap 1 structure.

Because the polynucleotides, e.g., mRNA, may be capped post-transcriptionally, and because this process is more efficient, up to 100% of the polynucleotides, e.g., mRNA, may be capped. This is in contrast to ˜₈₀% when a cap analog is linked to an mRNA in the course of an in vitro transcription reaction.

In some embodiments, the polynucleotides of the nucleic acid vaccines may contain an internal ribosome entry site (IRES) sequence. While not wishing to be bound by theory, IRES plays an important role in initiating protein synthesis in absence of the 5′ cap structure. An IRES may act as the sole ribosome binding site or may serve as one of multiple ribosome binding sites of an mRNA.

mRNA Components: Tailing Region

In some embodiments, the polynucleotide of the nucleic acid vaccine, e.g., the mRNA includes a tailing region. Non-liming examples of a tailing region include a poly-A sequence, a poly-C sequence, and/or a polyA-G quartet.

In some embodiments the mRNA includes a chain terminating nucleoside. Non-limiting examples of chain terminating nucleosides include 2′-O methyl, F and locked nucleic acids (LNA).

In some embodiments, the sequence of the tailing region of the polynucleotide of the nucleic acid vaccine may range from absent to 500 nucleotides in length (e.g., at least 60, 70, 80, 90, 120, 140, 160, 180, 200, 250, 300, 350, 400, 450, or 500 nucleotides). If the tailing region is a poly-A tail, the length may be described in units of or as a function of poly-A Binding Protein binding.

In some embodiments, poly-A tails may also be added after the construct is exported from the nucleus.

In some embodiments, a long chain of adenine nucleotides (poly-A tail) may be added to a polynucleotide such as an mRNA molecule during RNA processing in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

In some embodiments, the length of a poly-A tail, when present, is greater than 30 nucleotides in length (e.g., at least or greater than about 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A tail region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000 nucleotides).

In some embodiments, the poly-A tail is approximately 99 nucleotides in length (SEQ ID NO: 44).

In some embodiments, the poly-A tail is designed relative to the length of the overall polynucleotide or the length of a particular region of the polynucleotide. This design may be based on the length of a coding region, the length of a particular feature or region or based on the length of the ultimate product expressed from the polynucleotides.

In this context the poly-A tail may be 10, 20, 30, 40, 50, 60, 70, 80, 90, or 100% greater in length than the polynucleotide or feature thereof. The poly-A tail may also be designed as a fraction of the polynucleotides to which it belongs. In this context, the poly-A tail may be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein may enhance expression.

### Signal Sequences

In some embodiments, the polynucleotides of the nucleic acid vaccines may also encode additional features which may facilitate the trafficking of the polypeptides to therapeutically relevant sites. One such feature which aids in protein trafficking is the signal sequence. As used herein, a “signal sequence” or “signal peptide” is a polynucleotide or polypeptide, respectively, which is from about 9 to 200 nucleotides (3-60 amino acids) in length which is incorporated at the 5′ terminus of the coding region or the N-terminus polypeptide encoded, respectively. In some embodiments, addition of these sequences result in trafficking of the encoded polypeptide to the endoplasmic reticulum through one or more secretory pathways. Some signal peptides are cleaved from the protein by signal peptidase after the proteins are transported.

In some embodiments, the polynucleotides of the nucleic acid vaccines described herein include a signal sequence comprising SEQ ID NO: 45 (DNA) or SEQ ID NO: 49 (RNA).

### Codon Optimization

The polynucleotides of the nucleic acid vaccines, their regions or parts or subregions may be codon optimized. Codon optimization methods are known in the art and may be useful in efforts to achieve one or more of several goals. These goals include, but are not limited to, match codon frequencies in target and host organisms to ensure proper folding, alter GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include, but are not limited to, services from GeneArt (Life Technologies), DNA2.0 (Menlo Park Calif) and/or proprietary methods. In some embodiments, the ORF sequence is optimized using optimization algorithms. Codon options for each amino acid are given in Table 4.

TABLE 4
Codon Options

| Single Letter | Amino | |
|---|---|---|
| Nomenclature | Acid Name | Codon Options |
| A | Alanine | GCT, GCC, GCA, GCG |
| C | Cysteine | TGT, TGC |
| D | Aspartic acid | GAT, GAC |
| E | Glutamic acid | GAA, GAG |
| F | Phenylalanine | TTT, TTC |
| G | Glycine | GGT, GGC, GGA, GGG |
| H | Histidine | CAT, CAC |
| I | Isoleucine | ATT, ATC, ATA |
| K | Lysine | AAA, AAG |
| L | Leucine | CTT, CTC, CTA, CTG, |
| | | TTA, TTG |
| M | Methionine | ATG |
| N | Asparagine | AAT, AAC |
| P | Proline | CCT, CCC, CCA, CCG |
| Q | Glutamine | CAA, CAG |
| R | Arginine | CGT, CGC, CGA, CGG, |
| | | AGA, AGG |
| s | Serine | TCT, TCC, TCA, TCG, |
| | | AGT, AGC |
| Sec | Selenocysteine | UGA in mRNA in presence |
| | | of Selenocysteine insertion |
| | | element (SECTS) |
| Stop | Stop codons | TAA, TAG, TGA |
| T | Threonine | ACT, ACC, AC A, ACG |
| V | Valine | GTT, GTC, GTA, GTG |
| w | Tryptophan | TGG |
| γ | Tyrosine | TAT, TAC |

In some embodiments, the nucleic acid vaccine is vectorized after codon optimization. Non-limiting examples of vectors include, but are not limited to, plasmids, viruses, cosmids, and artificial chromosomes.

### Modifications

Nucleic acid vaccines of the present disclosure, including mRNA vaccines, may include one or more modifications. The terms “modification” or, as appropriate, “modified” refer to modification with respect to A, G, U or C ribonucleotides. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids.

As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group or other backbone linkage (internucleoside linkage).

The modifications may be various distinct modifications. In some embodiments, the coding region(s), the untranslated region(s), the flanking region(s), and/or the terminal or tailing regions may contain one, two, or more (optionally different) nucleoside or nucleotide modifications. In some embodiments, nucleic acid vaccines of the present disclosure comprise one or more modifications which render the nucleic acid molecules, when introduced to a cell, more resistant to degradation in the cell and/or more stable in the cell as compared to unmodified polynucleotides.

The polynucleotides of the nucleic acid vaccines described herein can include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted, for example, with optionally substituted amino, optionally substituted thiol, optionally substituted alkyl (e.g., methyl or ethyl), optionally substituted or halo (e.g., chloro or fluoro) atoms or groups. In certain embodiments, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. Additional modifications are described herein.

In some embodiments, the modifications include 2′-O-Methyl-modified or 2′-O-Methoxyethyl-modified nucleotides (2′-OMe and 2′-MOE modifications, respectively).

In some embodiments, the polynucleotides of the nucleic acid vaccines described herein may comprise at least one modification described herein.

The polynucleotides of the nucleic acid vaccines described herein can include a combination of modifications to the sugar, the nucleobase, and/or the internucleoside linkage.

Modifications of polynucleotides (e.g., RNA polynucleotides, such as mRNA polynucleotides) that are useful in the vaccines of the present disclosure include, but are not limited to, any modifications as described in PCT Publication WO2017070626, the contents of which are incorporated herein by reference in their entirety, including, for example, modification or deletion of nucleotides (or codons) encoding one or more N-linked glycosylation site in a translated polypeptide. Modifications that are useful in the vaccines of the present disclosure may also comprise any modifications as described in PCT Publication WO2018200892, the contents of which are incorporated herein by reference in their entirety. The vaccines of the present disclosure may further comprise features or modifications as described in PCT patent application publications WO2020255063, WO2020182869, WO2016011222, WO2016011226, WO2016005004, WO2016000792, WO2015176737, WO2015085318, WO2015048744, and WO2015034925, and United States patent application publications US20200254086, US20200206362, US20180311336 and US20180303929; the contents of each of which are incorporated herein by reference in their entireties.

For example, the polynucleotides, including the mRNA molecules of the nucleic acid vaccines described herein, can include modifications as follows. The internucleoside linkages of the polynucleotides may be partially or fully modified. The polynucleotides may comprise modifications to one or more nucleobases. The polynucleotides may comprise 5-methylcytosines in place of all cytosine nucleobases/cytidine nucleotides. Further the polynucleotides may have one or more modifications to one or more of the sugar subunits of a nucleoside. The sugar modification can be one or more locked nucleic acids (LNAs) or 2′-O-Methoxyethyl-modified (“2′-MOE”) modifications. The polynucleotides can be designed with a patterned array of sugar, nucleobase or linkage modifications. In some embodiments, the polynucleotides can comprise modifications to maximize stability. In some embodiments, the polynucleotides can be fully 2′-MOE-sugar modified.

### Modified Nucleobases

The modified nucleosides and nucleotides can include a modified nucleobase. Examples of nucleobases found in RNA include, but are not limited to, adenine, guanine, cytosine, and uracil. Examples of nucleobases found in DNA include, but are not limited to, adenine, guanine, cytosine, and thymine.

In some embodiments, the modified nucleobase is a modified uracil. Exemplary nucleobases and nucleosides having a modified uracil include pseudouridine (W), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 5-methylaminomethyl-2-thio-uridine (mnm⁵s2U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (rm⁵U), 1-taurinomethyl-pseudouridine, 5-taurinomethyl-2-thio-uridine(m⁵s2U), 1-taurinomethyl-4-thio-pseudouridine, 5-methyl-uridine (m⁵U, i.e., having the nucleobase deoxythymine), 1-methylpseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ)), 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′□F□ara□uridine, 2′□F□uridine, 2′□OH□ara□uridine, 5□(2□carbomethoxyvinyl) uridine, and 5□[3□(1□E□propenylamino)uridine.

In some embodiments, the modified nucleobase is a modified cytosine.

Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (frCm), N4,N4,2′-O-trimethyl-cytidine (m⁴2Cm), 1-thio-cytidine, 2′□F□ara□cytidine, 2′□F□cytidine, and 2′□OH□ara□cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m6A), N6-isopentenyl-adenosine (i⁶A), 2-methylthio-N6-isopentenyl-adenosine (ms²i6A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g6A), N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′□F□ara□adenosine, 2′□F□adenosine, 2′□OH□ara□adenosine, and N6□(19□amino□pentaoxanonadecyl)-adenosine.

In some embodiments, the modified nucleobase is a modified guanine. Exemplary nucleobases and nucleosides having a modified guanine include inosine (I), 1-methyl-inosine (miI), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQi), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²2G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), and 2′-O-ribosylguanosine (phosphate) (Gr(p)).

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

Different sugar modifications, nucleotide modifications, and/or internucleoside linkages (e.g., backbone structures) may be introduced at various positions in a polynucleotide described herein. One of ordinary skill in the art will appreciate that the nucleotide analogs or other modification(s) may be located at any position(s) of a polynucleotide such that the function of the polynucleotide is not substantially decreased. The polynucleotides of the present disclosure may contain from about 1% to about 100% modified nucleotides (either in relation to overall nucleotide content, or in relation to one or more types of nucleotide, i.e. any one or more of A, G, T/U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the polynucleotides of the nucleic acid vaccines described herein may be modified to be a circular nucleic acid. The termini of the polynucleotides may be linked by chemical reagents or enzymes, producing circular polynucleotides that have no free ends. Circular polynucleotides are expected to be more stable than linear counterparts and to be resistant to digestion with exonucleases. Circular polynucleotides may further comprise other structural and/or chemical modifications with respect to A, G, T/U or C ribonucleotides/deoxyribonucleotides.

In some embodiments, the polynucleotides are at least 50% modified, e.g., at least 50% of the nucleotides are modified. In some embodiments, the polynucleotides are at least 75% modified, e.g., at least 75% of the nucleotides are modified. It is to be understood that since a nucleotide (sugar, base and phosphate moiety, e.g., linkage) may each be modified, any modification to any portion of a nucleotide, or nucleoside, will constitute a modification.

In some embodiments, the polynucleotides are at least 10% modified in only one component of the nucleotide, with such component being the nucleobase, sugar, or linkage between nucleosides. For example, modifications may be made to at least 10%, 20%, 30%, 40%, 50%, 60%, 70%, 80%, 90% or 100% of the nucleobases, sugars, or linkages of a polynucleotide described herein.

As non-limiting examples, the uracil nucleosides of the polynucleotide of the nucleic acid vaccine are all modified. The modifications may be the same or different. In some embodiments, the guanine nucleosides of the polynucleotide of the nucleic acid vaccine are all modified. The modifications may be the same or different. In some embodiments, the guanine nucleosides of the polynucleotide of the nucleic acid vaccine are all modified. The modifications may be the same or different. In some embodiments, the cytosine nucleosides of the polynucleotide of the nucleic acid vaccine are all modified. The modifications may be the same or different. In some embodiments, the adenine nucleosides of the polynucleotide of the nucleic acid vaccine are all modified. The modifications may be the same or different.

In one embodiment of the disclosure, the polynucleotide of the nucleic acid vaccine is modified to comprise N1-methyl-pseudouridine nucleotides.

### Sugar Modifications

The modified nucleosides and nucleotides which may be incorporated into polynucleotides (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); a polyethyleneglycol (PEG), —O(CH2CH2O)nCH2CH2OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges include methylene, propylene, ether, or amino bridges; aminoalkyl; aminoalkoxy; amino; and amino acid.

In some embodiments, the polynucleotide, such as the mRNA of the nucleic acid vaccine described herein comprises at least one sugar modification. Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, polynucleotide molecules as described herein, including mRNAs, can include nucleotides containing, e.g., arabinose, as the sugar.

Nonlimiting examples of the sugar modification may include the modifications provided in Table 5. The polynucleotides of the present disclosure can have one or more nucleotides carrying a modification as provided in Table 5. In some embodiments, each of the nucleotides of a polynucleotide described herein carries any one of the modifications as provided in Table 5, or none of the modifications as provided in Table 5.

TABLE 5
Nucleotide Sugar Modifications

| Nucleotide | Structure Depiction |
|---|---|
| DNA | 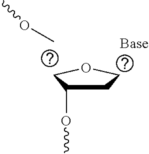 |
| 2′-O-Methyl (2′-OMe) | 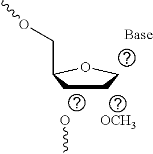 |
| 2′F-RNA | 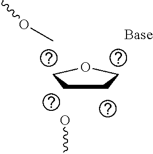 |
| 2′F-ANA | 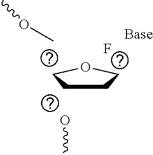 |
| 4′S-RNA | 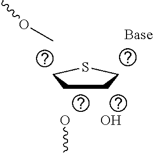 |
| UNA | 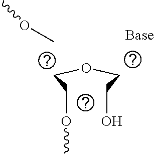 |
| LNA | 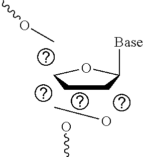 |
| 4′S-FANA | 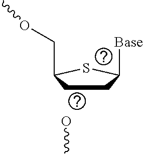 |
| 2′-O-Methoxyethyl (2′-MOE) | 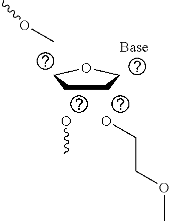 |
| 2′-O-Allyl | 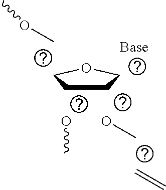 |
| 2′-O-Ethylamine | 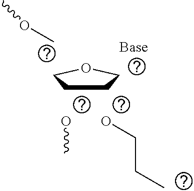 |
| 2′-O-Cyanoethyl | 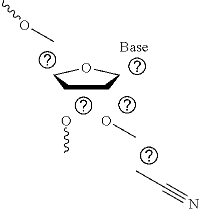 |
| 2′-O-Acetalester | 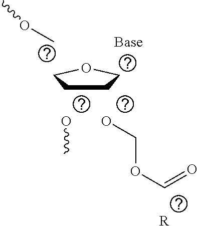 |
| 4′-C-aminomethyl-2′-O-methyl RNA | 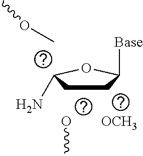 |
| 2′-azido | 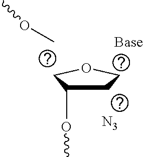 |
| Methylene-cLNA | 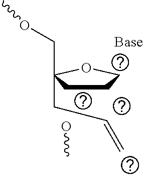 |
| N-MeO-amino BNA | 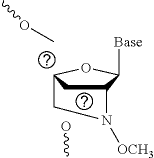 |
| N-Me-aminooxy BNA | 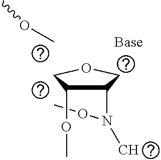 |
| 2′,4′-BNANC[NMe] | 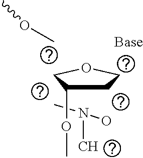 |
| MC | 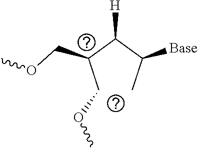 |
| ONA | 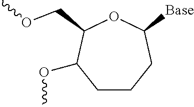 |
| tc-DNA | 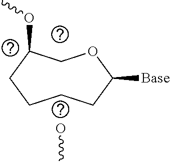 |
| CeNA | 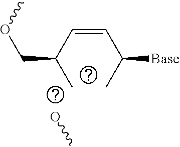 |
| ANA | 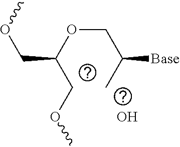 |
| HNA | 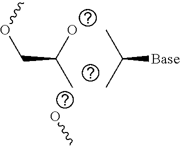 |

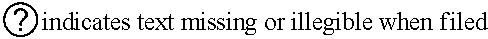

In some embodiments, at least one of the 2′ positions of the sugar (OH in RNA or H in DNA) of a nucleotide of the polynucleotides is substituted with —OMe, referred to as 2′-OMe. In some embodiments, at least one of the 2′ positions of the sugar (OH in RNA or H in DNA) of a nucleotide of the polynucleotides is substituted with —F, referred to as 2′-F.

### Internucleoside Linkages

The polynucleotides of the present disclosure can include any modification to the internucleoside linkage (e.g., to a linking phosphate/to a phosphodiester linkage/to the phosphodiester backbone). In the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, methylphosphonates phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polynucleotides through the unnatural phosphorothioate backbone linkages. Phosphorothioate DNA and RNA have increased nuclease resistance and subsequently a longer half-life in a cellular environment. Phosphorothioate linked polynucleotide molecules are expected to also reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

In some embodiments, the polynucleotides comprise at least one phosphorothioate linkage or methylphosphonate linkage between nucleotides.

In some embodiments, the polynucleotides comprise at least one 5′-(E)-vinylphosphonate (5′-E-VP), a phosphate mimic, as a modification.

In one embodiment of the present disclosure, the polynucleotide (e.g., mRNA) of the nucleic acid vaccine for COVID-19 may be modified.

### Valency

Nucleic acid vaccines of the present disclosure may vary in their valency. “Valency” refers to the number of antigenic components in the nucleic acid vaccine or the polynucleotide of the nucleic acid vaccines. The antigenic components of the nucleic acid vaccine may be on the same polynucleotide or they may be on different polynucleotides. In some embodiments, the nucleic acid vaccine may be monovalent. In some embodiments, the nucleic acid vaccine may be divalent. In some embodiments, the nucleic acid vaccine may be trivalent. In some embodiments, the nucleic acid vaccine may be multivalent which may comprise 2, 3, 4, 5, 6, 7, 8, 9, 10, 11, 12, 13, 14, 15, 16, 17, 18, 19, 20, 21, 22, 23, 24, 25 or more than 25 antigens or antigenic moieties such as, but not limited to, antigenic peptides. As a non-limiting example, antigenic peptides may be one or more fragments or variants of the structural proteins of SARS-CoV-2.

### Synthesis

### Enzymatic Methods

### In Vitro Transcription-Enzymatic Synthesis

cDNA encoding the polynucleotides of the nucleic acid vaccines described herein may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and polymerase variants.

In some embodiments, the DNA template is removed from the IVT reaction, using a DNase I enzyme. The digested DNA and nucleotides are then removed during oligo dT purification of the mRNA. This purification method is based on affinity of the poly-A tail of the mRNA to the poly-dT column bed. Centrifugation may be used but may not be required to remove the digested DNA and nucleotides. After purification by a reverse phase column (e.g., SDVB) to remove double stranded RNA from the mRNA, ultrafiltration may be utilized, followed by one or more filtration steps. Following purification, residual DNA may be measured to confirm that the DNA has been removed by using PCR for a region of the plasmid outside of the region transcribed into mRNA. In some embodiments, where concentration of the product is desired, diafiltration methods may be used followed by one or more filtration steps to remove any bioburden (e.g., biomolecules, or other biomaterial).

Any number of RNA polymerases or variants may be used in the synthesis of the polynucleotides of the nucleic acid vaccine described herein. RNA polymerases may be modified by inserting or deleting amino acids of the RNA polymerase sequence.

Polynucleotide or nucleic acid synthesis reactions may be carried out by enzymatic methods utilizing polymerases. Polymerases catalyze the creation of phosphodiester bonds between nucleotides in a polynucleotide or nucleic acid chain. Currently known DNA polymerases can be divided into different families based on amino acid sequence comparison and crystal structure analysis. DNA polymerase I (pol I) or A polymerase family, including the Klenow fragments of E. coli, Bacillus DNA polymerase I, Thermus aquaticus (Taq) DNA polymerases, and the T7 RNA and DNA polymerases, is among the best studied of these families. Another large family is DNA polymerase a (pol a) or B polymerase family, including all eukaryotic replicating DNA polymerases and polymerases from phages T4 and RB69. Although they employ similar catalytic mechanism, these families of polymerases differ in substrate specificity, substrate analog-incorporating efficiency, degree and rate for primer extension, mode of DNA synthesis, exonuclease activity, and sensitivity against inhibitors.

### Solid-Phase Chemical Synthesis

In some embodiments, polynucleotides of the nucleic acid vaccines described herein may be manufactured in whole or in part using solid phase techniques. Solid-phase chemical synthesis of polynucleotides or nucleic acids is an automated method wherein molecules are immobilized on a solid support and synthesized step by step in a reactant solution. Impurities and excess reagents are washed away and no purification is required after each step. The automation of the process is amenable on a computer-controlled solid-phase synthesizer. Solid-phase synthesis allows rapid production of polynucleotides or nucleic acids in a relatively large scale that leads to the commercial availability of some polynucleotides or nucleic acids.

In some embodiments, automated solid-phase synthesis is used where the chain is synthesized in 3′ to 5′ direction. The hydroxyl group in the 3′ end of a nucleoside is tethered to a solid support via a chemically cleavable or light-cleavable linker. Activated nucleoside monomers, such as 2′-deoxynucleosides (dA, dC, dG and dT), ribonucleosides (A, C, G, and U), or chemically modified nucleosides, are added to the support-bound nucleoside sequentially. At the end of the synthesis, a cleaving agent such as ammonia or ammonium hydroxide is added to remove all the protecting groups and release the polynucleotide chains from the solid support. Light may also be applied to cleave the polynucleotide chain. The product can then be further purified with high pressure liquid chromatography (HPLC) or electrophoresis.

### Liquid Phase Chemical Synthesis

The synthesis of polynucleotides of the nucleic acid vaccines described herein by the sequential addition of monomer building blocks may be carried out in a liquid phase. A covalent bond is formed between the monomers or between a terminal functional group of the growing chain and an incoming monomer. Functional groups not involved in the reaction must be temporarily protected. After the addition of each monomer building block, the reaction mixture has to be purified before adding the next monomer building block. The functional group at one terminal of the chain has to be deprotected to be able to react with the next monomer building blocks. A liquid phase synthesis is labor- and time-consuming and cannot not be automated. Despite the limitations, liquid phase synthesis is still useful in preparing short polynucleotides in a large scale. Because the system is homogenous, it does not require a large excess of reagents and is cost-effective in this respect.

### Quantification and Purification

In some embodiments, the polynucleotides of the nucleic acid vaccines described herein may be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, bronchoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alter natively, exosomes may be retrieved from an organ selected from the group consisting of lung, heart, pancreas, stomach, intestine, bladder, kidney, ovary, testis, skin, colon, breast, prostate, brain, esophagus, liver, and placenta.

In the exosome quantification method, a sample of not more than 2 mL is obtained from the subject and the exosomes isolated by size exclusion chromatography, density gradient centrifugation, differential centrifugation, nanomembrane ultrafiltration, immunosorbent capture, affinity purification, microfluidic separation, or combinations thereof. In the analysis, the level or concentration of a polynucleotide may be an expression level, presence, absence, truncation or alteration of the administered construct. It is advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunosorbent capture, affinity purification, microfluidic separation, or combinations thereof.

These methods afford the investigator the ability to monitor, in real time, the level of polynucleotides remaining or delivered. This is possible because the polynucleotides described herein differ from the endogenous forms due to the structural modifications.

In some embodiments, the polynucleotide may be quantified using methods such as, but not limited to, ultraviolet visible spectroscopy (UV/Vis). Anon-limiting example of a UV/Vis spectrometer is a NANODROP® spectrometer (ThermoFisher, Waltham, Mass.). The quantified polynucleotide may be analyzed in order to determine if the polynucleotide may be of proper size, check that no degradation of the polynucleotide has occurred. Degradation of the polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

Purification of the polynucleotides of the nucleic acid vaccines described herein may include, but is not limited to, polynucleotide clean-up, quality assurance and quality control. Clean-up may be performed by methods known in the arts such as, but not limited to, AGEN-COURT® beads (Beckman Coulter Genomics, Danvers, Mass.), poly-T beads, LNA™ oligo-T capture probes (EX-IQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

### III. Pharmaceutical Compositions and Delivery

The nucleic acid vaccines described herein may be used as therapeutic or prophylactic agents. In some embodiments, the present disclosure provides pharmaceutical compositions comprising at least one pharmaceutically acceptable carrier and a nucleic acid vaccine, i.e., a nucleic acid vaccine for COVID-19. In accordance, the pharmaceutical compositions comprising the nucleic acid vaccine described herein can be used for preventing, alleviating and/or treating COVID-19.

Provided herein are nucleic acid vaccines and pharmaceutical compositions thereof which may be used in combination with one or more pharmaceutically acceptable excipients. Pharmaceutical compositions may optionally comprise one or more additional active substances, e.g. therapeutically and/or prophylactically active substances. Pharmaceutical compositions of the nucleic acid vaccines described herein may be sterile and/or pyrogen-free.

In some embodiments, compositions are administered to humans, human patients or subjects. For the purposes of the present disclosure, the phrase “active ingredient” generally refers to the nucleic acid vaccines or the polynucleotides contained therein, e.g., polynucleotides encoding one or more proteins, peptides, fragments or variants thereof of SARS-CoV-2 for the prevention, alleviation and/or treatment of COVID-19, to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

### Formulations

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one nucleic acid vaccine composition, e.g., nucleic acid vaccine for COVID-19, e.g., one mRNA vaccine for COVID-19. As a non-limiting example, the formulations may contain 1, 2, 3, 4 or 5 nucleic acid vaccine compositions with different sequences, e.g., 1, 2, 3, 4 or 5 mRNA vaccine compositions with different sequences. In some embodiments, the formulation contains at least two nucleic acid vaccine (e.g., mRNA vaccine) compositions with different sequences. In some embodiments, the formulation contains at least three nucleic acid vaccine (e.g., mRNA vaccine) compositions with different sequences. In some embodiments, the formulation contains at least four nucleic acid vaccine (e.g., mRNA vaccine) compositions with different sequences. In some embodiments, the formulation contains at least five nucleic acid vaccine (e.g., mRNA vaccine) compositions with different sequences.

The nucleic acid vaccine compositions of the present disclosure can be formulated using one or more excipients to: (1) increase stability; (2) increase cell transfection; (3) permit the sustained or delayed release (e.g., from a depot formulation of the nucleic acid vaccine composition); (4) alter the biodistribution (e.g., target the nucleic acid vaccine composition to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with nucleic acid vaccine compositions (e.g., for transplantation into a subject), hyaluronidase, nanoparticle mimics and combinations thereof. Accordingly, the formulations of the present disclosure can include one or more excipients, each in an amount that together increases the stability of the nucleic acid vaccine compositions and/or increases cell transfection by the nucleic acid vaccine compositions. Further, the nucleic acid vaccine compositions of the present disclosure may be formulated using self-assembled nucleic acid nanoparticles. Pharmaceutically acceptable carriers, excipients, and delivery agents for nucleic acids that may be used in the formulation with the nucleic acid vaccine compositions of the present disclosure are disclosed in PCT Patent Application Publication WO 2013/090648, the contents of which are incorporated herein by reference in their entirety.

### Lipidoids

The nucleic acid vaccine compositions of the disclosure can be formulated using one or more lipidoids.

The synthesis of lipidoids has been extensively described and formulations containing these compounds are particularly suited for delivery of oligonucleotides or nucleic acids (see Mahon et al., Bioconjug Chem. 2010, 21:1448-1454; Schroeder et al., J Intern Med. 2010, 267:9-21; Akinc et al., Nat Biotechnol. 200, 826:561-569; Love et al., Proc Natl Acad Sci USA. 2010, 107:1864-1869; Siegwart et al., Proc Natl Acad Sci US A. 2011, 108:12996-3001; the contents of all of which are incorporated herein by references in their entirety).

While these lipidoids have been used to effectively deliver double-stranded small interfering RNA molecules in rodents and non-human primates (see Akinc et al., Nat Biotechnol. 2008, 26:561-569; Frank-Kamenetsky et al., Proc Natl Acad Sci USA. 2008, 105:11915-11920; Akinc et al., Mol Ther. 2009, 17:872-879; Love et al., Proc Natl Acad Sci USA. 2010, 107:1864-1869; Leuschner et al., Nat Biotechnol. 2011, 29:1005-1010; the contents of all of which is incorporated herein by reference in their entirety), the present disclosure contemplates their formulation and use in delivering at least one pharmaceutically acceptable carrier, including nucleic acid vaccines. Complexes, micelles, liposomes or particles can be prepared containing these lipidoids and therefore, can result in an effective delivery of the nucleic acid vaccine compositions following the injection of a lipidoid formulation via localized and/or systemic routes of administration. Lipidoid complexes containing nucleic acid vaccine compositions can be administered by various means including, but not limited to, intravenous (IV), intramuscular (IM), subcutaneous (SC), intraparenchymal (IPa), intrathecal (IT), or intracerebroventricular (ICV) administration.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009, 17:872-879; the contents of which are herein incorporated by reference in their entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 2010, 401:61; the contents of which are herein incorporated by reference in their entirety), C12-200 (including derivatives and variants), and MD1, can be tested for in vivo activity.

The lipidoid referred to herein as “98N12-5” is disclosed by Akinc et al., Mol Ther. 2009, 17:872-879 and the contents of which is incorporated herein by reference in their entirety.

The lipidoid referred to herein as “C12-200” is disclosed by Love et al., Proc Natl Acad Sci USA. 2010, 107:1864-1869 and Liu and Huang, Molecular Therapy. 2010, 669-670; the contents of both of which are herein incorporated herein by reference in their entirety. The lipidoid formulations can include particles comprising either 3 or 4 or more components in addition to the nucleic acid vaccine compositions. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In some embodiments, nucleic acid vaccine compositions formulated with a lipidoid for systemic intravenous administration. For example, a final optimized intravenous formulation using nucleic acid vaccine compositions and comprising a lipid molar composition of 42% 98N12-5, 48% cholesterol, and 10% PEG-lipid with a final weight ratio of about 7.5 to 1 total lipid to nucleic acid vaccine compositions and a C14 alkyl chain length on the PEG lipid, with a mean particle size of roughly 50-60 nm, can result in the distribution of the formulation to be greater than 90% to the liver. (see, Akinc et al., Mol Ther. 2009, 17:872-879; the contents of which are herein incorporated by reference herein in their entirety). In another example, an intravenous formulation using a C12-200 lipidoid (see PCT Patent Application Publication WO2010129709, the contents of which are herein incorporated by reference in their entirety) may have a molar ratio of 50/10/38.5/1.5 of C12-200/disteroylphosphatidyl choline/cholesterol/PEG-DMG, with a weight ratio of 7 to 1 total lipid to nucleic acid and a mean particle size of 80 nm may be effective to deliver nucleic acid vaccine compositions (see, Love et al., Proc Natl Acad Sci USA. 2010, 107:1864-1869, the contents of which are herein incorporated by reference herein in their entirety).

In some embodiments, an MD1 lipidoid-containing formulation may be used to effectively deliver nucleic acid vaccine compositions to hepatocytes in vivo. The characteristics of optimized lipidoid formulations for intramuscular or subcutaneous routes may vary significantly depending on the targeT-cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009,17:872-879, the contents of which are herein incorporated by reference in their entirety), use of a lipidoid-formulated nucleic acid vaccine compositions to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008, 26:561-569; Leuschner et al., Nat Biotechnol. 2011, 29:1005-1010; Cho et al. Adv. Funct. Mater. 2009, 19:3112-3118; 8^{th} International Judah Folkman Conference, Cambridge, MA Oct. 8-9, 2010; the contents of each of which are herein incorporated by reference herein in their entirety). For effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of nucleic acid vaccine compositions for delivery to differenT-cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011, 29:1005-1010; the contents of which are herein incorporated by reference in their entirety). The use of lipidoid formulations for the localized delivery of nucleic acids to cells via either subcutaneous or intramuscular delivery, may not require all of the formulation components desired for systemic delivery, and as such may comprise only the lipidoid and nucleic acid vaccine compositions.

### Liposomes

The nucleic acid vaccine compositions of the disclosure can be formulated using one or more liposomes.

In some embodiments, pharmaceutical compositions of nucleic acid vaccine compositions include liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions comprising the nucleic acid vaccines described herein may include, without limitation, liposomes such as those formed from 1,2-dioleyloxy-N,N-dimethylaminopropane (DODMA) liposomes, DiLa2 liposomes from Marina Biotech (Bothell, WA), SMARTICLES®/NOV340 (Marina Biotech, Bothell), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US Patent Application Publication US20100324120; the contents of which are herein incorporated by reference in their entirety), neutral DOPC (1,2-dioleoyl-sn-glycero-3-phosphocholine) based liposomes (e.g., siRNA delivery for ovarian cancer (Landen et al. Cancer Biology & Therapy 2006, 5(12): 1708-1713); the contents of which is herein incorporated by reference in its entirety), hyaluronan-coated liposomes (Quiet Therapeutics, Israel), and liposomes which may deliver small molecule drugs such as, but not limited to, DOXIL® from Janssen Biotech, Inc. (Horsham, PA).

In some embodiments, pharmaceutical compositions comprising the nucleic acid vaccines described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999, 6:271-281; Zhang et al. Gene Therapy. 1999, 6:1438-1447; Jeffs et al. Pharm Res. 2005, 22:362-372; Morrissey et al., Nat Biotechnol. 2005, 2:1002-1007; Zimmermann et al., Nature. 2006, 441:111-114; Heyes et al. J Contr Rel. 2005, 107:276-287; Semple et al. Nature Biotech. 2010, 28:172-176; Judge et al. J Clin Invest. 2009, 119:661-673; deFougerolles Hum Gene Ther. 2008, 19:125-132; the contents of each of which are incorporated herein in their entireties). The original manufacturing method by Wheeler et al. was a detergent dialysis method, which was later improved by Jeffs et al. and is referred to as the spontaneous vesicle formation method. The liposome formulations may be composed of 3 to 4 lipid components in addition to the nucleic acid vaccine compositions. As a non-limiting example, a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In another example, the nucleic acid-lipid particle may comprise a cationic lipid comprising from about 50 mol % to about 85 mol % of the total lipid present in the particle; a non-cationic lipid comprising from about 13 mol % to about 49.5 mol % of the total lipid present in the particle; and a conjugated lipid that inhibits aggregation of particles comprising from about 0.5 mol % to about 2 mol % of the total lipid present in the particle as described in WO2009127060 to Maclachlan et al; the contents of which are incorporated herein by reference in their entirety. In another example, the nucleic acid-lipid particle may be any nucleic acid-lipid particle disclosed in US2006008910 to Maclachlan et al.; the contents of which are incorporated herein by reference in their entirety. As a non-limiting example, the nucleic acid-lipid particle may comprise a cationic lipid of Formula I, a non-cationic lipid, and a conjugated lipid that inhibits aggregation of particles.

In some embodiments, the nucleic acid vaccine compositions of the present disclosure may be formulated in a lipid vesicle which may have crosslinks between functionalized lipid bilayers.

In some embodiments, the liposome may contain a sugar-modified lipid disclosed in U.S. Pat. No. 5,595,756 to Bally et al., the contents of which are incorporated herein by reference in their entirety. The lipid may be a ganglioside and cerebroside in an amount of about 10 mol percent.

In some embodiments, the nucleic acid vaccine compositions of the present disclosure may be formulated in a liposome comprising a cationic lipid. The liposome may have a molar ratio of nitrogen atoms in the cationic lipid to the phosphates in the nucleic acid vaccine compositions (N:P ratio) of between 1:1 and 20:1 as described in PCT Patent Application Publication No. WO2013006825, the contents of which are herein incorporated by reference in their entirety. In some embodiments, the liposome may have a N:P ratio of greater than 20:1 or less than 1:1.

In some embodiments, the nucleic acid vaccine compositions of the present disclosure may be formulated in a lipid-polycation complex. The formation of the lipid-polycation complex may be accomplished by methods known in the art and/or as described in U.S. Pub. No. 20120178702, the contents of which are herein incorporated by reference in their entirety. As a non-limiting example, the polycation may include a cationic peptide or a polypeptide such as, but not limited to, polylysine, polyornithine and/or polyarginine and the cationic peptides described in PCT Patent Application Publication No. WO2012013326; the contents of which are herein incorporated by reference in their entirety. In some embodiments, the nucleic acid vaccine compositions may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010, 28:172-176; the contents of which are herein incorporated by reference in their entirety), the liposome formulation was composed of 57.1% cationic lipid, 7.1% dipalmitoylphosphatidylcholine, 34.3% cholesterol, and 1.4% PEG-c-DMA.

In some embodiments, the pharmaceutical compositions may be formulated with any amphoteric liposome disclosed in PCT Patent Application Publication No.:WO 2008043575 to Panzner and U.S. Pat. No. 8,580,297 to Essler et al. (Marina Biotech), the contents of which are incorporated herein by reference in their entirety. The amphoteric liposome may comprise a mixture of lipids including a cationic amphiphile, an anionic amphiphile and optional one or more neutral amphiphiles. The amphoteric liposome may comprise amphoteric compounds based on amphiphilic molecules, the head groups of which being substituted with one or more amphoteric groups. In some embodiments, the pharmaceutical compositions may be formulated with an amphoteric lipid comprising one or more amphoteric groups having an isoelectric point between 4 and 9, as disclosed in US Patent Application Publication No.: US20140227345 to Essler et al. (Marina Biotech), the contents of which are incorporated herein by reference in their entirety.

In some embodiments, the pharmaceutical composition may be formulated with liposomes comprising a sterol derivative as disclosed in U.S. Pat. No. 7,312,206 to Panzner et al. (Novosom), the contents of which are incorporated herein by reference in their entirety. In some embodiments, the pharmaceutical composition may be formulated with amphoteric liposomes comprising at least one amphipathic cationic lipid, at least one amphipathic anionic lipid, and at least one neutral lipid, or liposomes comprise at least one amphipathic lipid with both a positive and a negative charge, and at least one neutral lipid, wherein the liposomes are stable at pH 4.2 and pH 7.5, as disclosed in U.S. Pat. No. 7,780,983 to Panzner et al. (Novosom), the contents of which are incorporated herein by reference in their entirety. In some embodiments, the pharmaceutical composition may be formulated with liposomes comprising a serum-stable mixture of lipids taught in US Patent Application Publication No.: US 20110076322 to Panzner et al, the contents of which are incorporated herein by reference in their entirety, capable of encapsulating the nucleic acid vaccine compositions of the present disclosure. The lipid mixture comprises phosphatidylcholine and phosphatidylethanolamine in a ratio in the range of about 0.5 to about 8. The lipid mixture may also include pH sensitive anionic and cationic amphiphiles, such that the mixture is amphoteric, being negatively charged or neutral at pH 7.4 and positively charged at pH 4. The drug/lipid ratio may be adjusted to target the liposomes to particular organs or other sites in the body. In some embodiments, liposomes loaded with the nucleic acid vaccine compositions of the present disclosure as cargo, are prepared by the method disclosed in US Patent Application Publication No.: US 20120021042 to Panzner et al., the contents of which are incorporated herein by reference in their entirety. The method comprises steps of admixing an aqueous solution of a polyanionic active agent and an alcoholic solution of one or more amphiphiles and buffering said admixture to an acidic pH, wherein the one or more amphiphiles are susceptible of forming amphoteric liposomes at the acidic pH, thereby to form amphoteric liposomes in suspension encapsulating the active agent.

### Lipoplexes

The nucleic acid vaccine compositions of the disclosure can be formulated using one or more lipoplexes.

In some embodiments, the nucleic acid vaccine compositions may be formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008, 68:9788-9798; Strumberg et al. Int J Clin Pharmacol Ther, 2012, 50:76-78; Santel et al., Gene Ther, 2006, 13:1222-1234; Santel et al., Gene Ther.,2006, 13:1360-1370; Gutbier et al., Pulm Pharmacol. Ther. 2010, 23:334-344; Kaufmann et al. Microvasc Res., 2010, 80:286-293 Weide et al. J Immunother., 2009, 32:498-507; Weide et al. J Immunother., 2008, 31:180-188; Pascolo., Expert Opin. Biol. Ther. 4:1285-1294; Fotin-Mleczek et al., J. Immunother., 2011, 34:1-15; Song et al., Nature Biotechnol. 2005, 23:709-717; Peer et al., Proc Natl Acad Sci USA. 2007, 6; 104:4095-4100; deFougerolles Hum Gene Ther. 2008, 19:125-132; the contents of each of which are incorporated herein by reference in their entirety).

### Lipid Nanoparticles (LNPs)

In some embodiments, the nucleic acid vaccine compositions of the present disclosure may be formulated in a lipid nanoparticle (LNP). In general, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments, LNPs may comprise a cargo or a payload into their interior space, into the inter membrane space, onto their exterior surface, or any combination thereof.

LNPs useful herein are known in the art and generally comprise cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition.

The components of the LNP may be selected based on the desired target, tropism, cargo, size, or other desired feature or property.

The LNP may be the lipid nanoparticles described in PCT Patent Application Publication No. WO2012170930, the contents of which are herein incorporated by reference in their entirety.

In some embodiments, the nucleic acid vaccine compositions of the present disclosure may be formulated in a LNP that comprises at least one cationic lipid.

In some embodiments, the cationic lipid which may be used in formulations of the present disclosure may be selected from, but not limited to, a cationic lipid described in PCT Patent Application Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; the contents of each of which are herein incorporated by reference in their entirety. The cationic lipid may be also selected from, but not limited to, formula A described in PCT Patent Application Publication Nos. WO2012040184, WO2011153120, WO2011149733, WO2011090965, WO2011043913, WO2011022460, WO2012061259, WO2012054365 and WO2012044638; the contents of each of which are herein incorporated by reference in their entirety. Alternatively, the cationic lipid may be selected from, but not limited to, formula CLI-CLXXIX of PCT Patent Application No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; the contents of each of which are herein incorporated by reference in their entirety. The cationic lipid may be a multivalent cationic lipid such as the cationic lipid disclosed in U.S. Pat. No. 7,223,887 to Gaucheron et al., the contents of which are incorporated herein by reference in their entirety. The cationic lipid may have a positively-charged head group including two quaternary amine groups and a hydrophobic portion including four hydrocarbon chains as described in U.S. Pat. No. 7,223,887 to Gaucheron et al. The cationic lipid may be biodegradable as the biodegradable lipids disclosed in US Patent Application Publication No.: US20130195920 to Maier et al., the contents of which are incorporated herein by reference in their entirety. The cationic lipid may have one or more biodegradable groups located in a lipidic moiety of the cationic lipid as described in formula I-IV in US20130195920 to Maier et al.

In some embodiments, the cationic lipid may also be the cationic lipids disclosed in US20130156845 to Manoharan et al. and US 20130129785 to Manoharan et al., WO 2012047656 to Wasan et al., WO 2010144740 to Chen et al., WO 2013086322 to Ansell et al., or WO 2012016184 to Manoharan et al., the contents of each of which are incorporated herein by reference in their entirety.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-1 O-amine, (15Z)—N,N-dimethyl eptacos-15-en-1 O-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-1 O-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl} dodecan-1-amine, 1-[(1R,2S)-2-hepty lcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof. In some embodiments, the lipid may be a cleavable lipid such as those described in PCT Patent Application Publication No. WO2012170889, the contents of which are herein incorporated by reference in their entirety.

In some embodiments, the nanoparticles described herein may comprise at least one cationic polymer described herein and/or known in the art.

In some embodiments, the cationic lipid may be synthesized by methods known in the art and/or as described in PCT Patent Application Publication Nos. WO2012040184, WO2011153120, WO2011149733, WO2011090965, WO2011043913, WO2011022460, WO2012061259, WO2012054365, WO2012044638, WO2010080724 and WO201021865; the contents of each of which are herein incorporated by reference in their entirety.

In some embodiments, the pharmaceutical compositions of the nucleic acid vaccine compositions may include at least one of the PEGylated lipids described in PCT Patent Application Publication No. WO2012099755, the contents of which are herein incorporated by reference in their entirety.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In some embodiments, the LNP formulations of the nucleic acid vaccine compositions may contain PEG-c-DOMG at 3% lipid molar ratio. In some embodiments, the LNP formulations of the nucleic acid vaccine compositions may contain PEG-c-DOMG at 1.5% lipid molar ratio.

In some embodiments, the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the LNP formulation may contain PEG-DMG 2000 (1,2-dimyristoyl-sn-glycero-3-phophoethanolamine-N-[methoxy(polyethylene glycol)-2000), a cationic lipid known in the art. In some embodiments, the LNP formulation may contain PEG-DMG 2000 and at least one other component. In some embodiments, the LNP formulation may contain PEG-DMG 2000, DSPC and cholesterol. As a non-limiting example, the LNP formulation may contain PEG-DMG 2000, DLin-DMA, DSPC and cholesterol. As another non-limiting example, the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS, 2012, 109(36): 14604-14609; herein incorporated by reference in its entirety).

As another non-limiting example, the nucleic acid vaccine compositions described herein may be formulated in a nanoparticle to be delivered by a parenteral route as described in U.S. Patent Application Publication No. US20120207845; the contents of which are herein incorporated by reference in their entirety.

In some embodiments, the nucleic acid vaccine compositions of the present disclosure may be formulated with a plurality of cationic lipids, such as a first and a second cationic lipid as described in US Patent Application Publication No.: US20130017223 to Hope et al., the contents of which are incorporated herein by reference in their entirety. The first cationic lipid can be selected on the basis of a first property and the second cationic lipid can be selected on the basis of a second property, where the properties may be determined as outlined in US20130017223. In some embodiments, the first and second properties are complementary.

The nucleic acid vaccine compositions described herein may be formulated with a lipid particle comprising one or more cationic lipids and one or more second lipids, and one or more nucleic acids, wherein the lipid particle comprises a solid core, as described in US Patent Publication No. US20120276209 to Cullis et al., the contents of which are incorporated herein by reference in their entirety.

In some embodiments, the nucleic acid vaccine compositions of the present disclosure may be complexed with a cationic amphiphile in an oil-in-water (o/w) emulsion such as described in European Publication No.: EP2298358 to Satishchandran et al., the contents of which are incorporated herein by reference in their entirety. The cationic amphiphile may be a cationic lipid, modified or unmodified spermine, bupivacaine, or benzalkonium chloride and the oil may be a vegetable or an animal oil. As a non-limiting example, at least 10% of the nucleic acid-cationic amphiphile complex is in the oil phase of the oil-in-water emulsion (see e.g., the complex described in. EP2298358 to Satishchandran et al.), the contents of which are incorporated herein by reference in its entirety.

In some embodiments, the nucleic acid vaccine compositions of the present disclosure may be formulated with a composition comprising a mixture of cationic compounds and neutral lipids. As a non-limiting example, the cationic compounds may be formula (I) disclosed in PCT Patent Application Publication No.: WO 1999010390 to Ansell et al., the contents of which are described herein by reference in their entirety, and the neutral lipid may be selected from the group consisting of diacylphosphatidylcholine, diacylphosphatidylethanolamine, ceramide and sphingomyelin. In another non-limiting example, the lipid formulation may comprise a cationic lipid of formula A, a neutral lipid, a sterol and a PEG or PEG-modified lipid disclosed in US Patent Publication No.: US 20120101148 to Akinc et al., the contents of which are incorporated herein by reference in their entirety.

In some embodiments, the LNP formulation may be formulated by the methods described in International Publication Nos. WO2011127255 or WO2008103276. As a non-limiting example, the nucleic acid vaccine compositions of the present disclosure may be encapsulated in any of the lipid nanoparticle (LNP) formulations described in WO2011127255 and/or WO2008103276; the contents of each of which are herein incorporated by reference in their entirety.

In some embodiments, the LNP formulations described herein may comprise a polycationic composition. As a non-limiting example, the polycationic composition may be selected from formula 1-60 of US Patent Publication No. US20050222064; the contents of which are herein incorporated by reference in their entirety. The LNP formulations comprising a polycationic composition may be used for the delivery of the nucleic acid vaccine compositions described herein in vivo and/or in vitro.

In some embodiments, the LNP formulations described herein may additionally comprise a permeability enhancer molecule. Non-limiting permeability enhancer molecules are described in US Patent Publication No. US20050222064; the contents of which are herein incorporated by reference in their entirety.

The nanoparticle formulations may be a carbohydrate nanoparticle comprising a carbohydrate carrier and a nucleic acid vaccine composition (e.g., a nucleic acid vaccine for COVID-19). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., PCT Patent Application Publication No. WO2012109121; the contents of which are herein incorporated by reference in their entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the nucleic acid vaccine compositions is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. The lipid nanoparticle may be a self-assembly lipid-polymer nanoparticle (see Zhang et al., ACS Nano, 2008, 2 (8):1696-1702; the contents of which are herein incorporated by reference in their entirety).

In some embodiments, formulations comprising the nucleic acid vaccine compositions described herein may also be constructed or altered such that they passively or actively are directed to differenT-cell types in vivo, including but not limited to immune cells, endothelial cells, antigen presenting cells, and leukocytes (Akinc et al. Mol Ther. 2010, 18:1357-1364; Song et al., Nat Biotechnol. 2005, 23:709-717; Judge et al., J Clin Invest. 2009, 119:661-673; Kaufmann et al., Microvasc Res, 2010, 80:286-293; Santel et al., Gene Ther 2006, 13:1222-1234; Santel et al., Gene Ther, 2006, 13:1360-1370; Gutbier et al., Pulm Pharmacol. Ther. 2010, 23:334-344; Basha et al., Mol. Ther. 2011, 19:2186-2200; Fenske and Cullis, Expert Opin Drug Deliv. 2008, 5:25-44; Peer et al., Science. 2008, 319:627-630; Peer and Lieberman, Gene Ther. 2011, 18:1127-1133; the contents of each of which are incorporated herein by reference in their entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010, 18:1357-1364; the contents of which are herein incorporated by reference in their entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011, 8:197-206; Musacchio and Torchilin, Front Biosci. 2011, 16:1388-1412; Yu et al., Mol Membr Biol. 2010, 27:286-298; Patil et al., Crit Rev Ther Drug Carrier Syst. 2008, 25:1-61; Benoit et al., Biomacromolecules. 2011, 12:2708-2714; Zhao et al., Expert Opin Drug Deliv. 2008, 5:309-319; Akinc et al., Mol Ther. 2010, 18:1357-1364; Srinivasan et al., Methods Mol Biol. 2012, 820:105-116; Ben-Arie et al., Methods Mol Biol. 2012, 757:497-507; Peer J Control Release. 2010, 20:63-68; Peer et al., Proc Natl Acad Sci US A. 2007, 104:4095-4100; Kim et al., Methods Mol Biol. 2011, 721:339-353; Subramanya et al., Mol Ther. 2010, 18:2028-2037; Song et al., Nat Biotechnol. 2005, 23:709-717; Peer et al., Science. 2008, 319:627-630; Peer and Lieberman, Gene Ther. 2011, 18:1127-1133; the contents of each of which are incorporated herein by reference in their entirety).

In some embodiments, the nucleic acid vaccine compositions of the present disclosure can be formulated for controlled release and/or targeted delivery. As used herein, “controlled release” refers to a pharmaceutical composition or compound release profile that conforms to a particular pattern of release to affect a therapeutic outcome. In some embodiments, the nucleic acid vaccine compositions may be encapsulated into a delivery agent described herein and/or known in the art for controlled release and/or targeted delivery. As used herein, the term “encapsulate” means to enclose, surround, or encase. As it relates to the formulation of the compositions of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition of the disclosure may be enclosed, surrounded or encased within the delivery agent. “Partially encapsulated” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition of the disclosure are encapsulated in the delivery agent.

The nucleic acid vaccine compositions may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc., Deerfield, IL), PEG-based sealants, and COSEAL® (Baxter International, Inc., Deerfield, IL).

In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the formulations comprising the nucleic acid vaccine compositions for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the nucleic acid vaccine compositions of the present disclosure may be formulated with a targeting lipid with a targeting moiety such as the targeting moieties disclosed in US Patent Application Publication No.: US20130202652 to Manoharan et al., the contents of which are incorporated herein by reference in their entirety. As a non-limiting example, the targeting moiety of formula I of US 20130202652 to Manoharan et al. may be selected in order to favor the lipid being localized with a desired organ, tissue, cell, cell type or subtype, or organelle. Non-limiting targeting moieties that are contemplated in the present disclosure include transferrin, anisamide, an RGD peptide, prostate specific membrane antigen (PSMA), fucose, an antibody, or an aptamer.

In some embodiments, the nucleic acid vaccine compositions of the present disclosure may be encapsulated in a therapeutic nanoparticle. Therapeutic nanoparticles may be formulated by methods described herein and known in the art such as, but not limited to, PCT Patent Application Publication Nos. WO2010005740, WO2010030763, WO2010005721, WO2010005723, and WO2012054923, US Pub. Nos. US20110262491, US20100104645, US20100087337, US20100068285, US20110274759, US20100068286 and US20120288541 and U.S. Pat. Nos. 8,206,747, 8,293,276, 8,318,208 and 8,318,211; the contents of each of which are herein incorporated by reference in their entirety. Therapeutic polymer nanoparticles may be identified by the methods described in US Pub No. US20120140790, the contents of which are herein incorporated by reference in their entirety.

In some embodiments, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the nucleic acid vaccine compositions of the present disclosure (see PCT Patent Application Publication No. WO2010075072 and US Pub No. US20100216804, US20110217377 and US20120201859, the contents of each of which are herein incorporated by reference in their entirety).

In some embodiments, the therapeutic nanoparticles may be formulated to be target specific. As a non-limiting example, the therapeutic nanoparticles may include a corticosteroid (see PCT Patent Application Publication No. WO2011084518; the contents of which are herein incorporated by reference in their entirety). In some embodiments, the therapeutic nanoparticles may be formulated to be cancer specific. As a non-limiting example, the therapeutic nanoparticles may be formulated in nanoparticles described in PCT Patent Application Publication No. WO2008121949, WO2010005726, WO2010005725, and WO2011084521, and US Pub No. US20100069426, US20120004293 and US20100104655, the contents of each of which are herein incorporated by reference in their entirety.

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the therapeutic nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example, the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and PCT Patent Application Publication No. WO2012166923, the contents of each of which are herein incorporated by reference in their entirety).

In some embodiments, the therapeutic nanoparticle may comprise a multiblock copolymer such as, but not limited to the multiblock copolymers described in U.S. Pat. Nos. 8,263,665 and 8,287,910; the contents of each of which are herein incorporated by reference in their entirety.

In some embodiments, the block copolymers described herein may be included in a polyion complex comprising a non-polymeric micelle and the block copolymer. (See e.g., U.S. Pub. No. US20120076836; the contents of which are herein incorporated by reference in their entirety).

In some embodiments, the nanoparticles for delivery of the nucleic acid vaccines described herein include block co-polymers. Non-limiting examples of block co-polymers include those of formula I, formula II, formula III, formula IV, formula V, formula VI and formula VII of PCT Patent Application Publication No. WO2015017519, the contents of which are herein incorporated by reference in their entirety.

In some embodiments, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; the contents of which are herein incorporated by reference in their entirety) and combinations thereof.

In some embodiments, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. The degradable polyesters may include a PEG conjugation to form a PEGylated polymer.

In some embodiments, the therapeutic nanoparticle may include a conjugation of at least one targeting ligand. The targeting ligand may be any ligand known in the art such as, but not limited to, a monoclonal antibody. (Kirpotin et al, Cancer Res. 2006, 66:6732-6740; the contents of which are herein incorporated by reference in their entirety).

In some embodiments, the therapeutic nanoparticle may be formulated in an aqueous solution which may be used to target cancer (see PCT Patent Application Publication No. WO2011084513 and US Pub No. US20110294717, the contents of each of which are herein incorporated by reference in their entirety).

In some embodiments, the nucleic acid vaccine compositions may be encapsulated in, linked to and/or associated with synthetic nanocarriers. Synthetic nanocarriers include, but are not limited to, those described in PCT Patent Application Publication Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, the contents of each of which are herein incorporated by reference in their entirety. The synthetic nanocarriers may be formulated using methods known in the art and/or described herein. As a non-limiting example, the synthetic nanocarriers may be formulated by the methods described in PCT Patent Application Publication Nos. WO2010005740, WO2010030763 and WO201213501 and US Pub. Nos. US20110262491, US20100104645, US20100087337 and US2012024422, the contents of each of which are herein incorporated by reference in their entirety. The synthetic nanocarrier formulations may be lyophilized by methods described in PCT Patent Application Publication Pub. No. WO2011072218 and U.S. Pat. No. 8,211,473; the contents of each of which are herein incorporated by reference in their entirety.

In some embodiments, the synthetic nanocarriers may contain reactive groups to release the nucleic acid vaccine compositions described herein (see PCT Patent Application Publication No. WO20120952552 and US Pub No. US20120171229, the contents of each of which are herein incorporated by reference in their entirety).

In some embodiments, the synthetic nanocarriers may be formulated for targeted release. In some embodiments, the synthetic nanocarrier may be formulated to release the nucleic acid vaccine compositions at a specified pH and/or after a desired time interval. As a non-limiting example, the synthetic nanoparticle may be formulated to release the nucleic acid vaccine compositions after 24 hours and/or at a pH of 4.5 (see PCT Patent Application Publication Nos. WO2010138193 and WO2010138194 and US Pub Nos. US20110020388 and US20110027217, the contents of each of which are herein incorporated by reference in their entireties).

In some embodiments, the synthetic nanocarriers may be formulated for controlled and/or sustained release of the nucleic acid vaccine compositions described herein. As a non-limiting example, the synthetic nanocarriers for sustained release may be formulated by methods known in the art, described herein and/or as described in PCT Patent Application Publication No. WO2010138192 and US Pub No. US20100303850, the contents each of which are herein incorporated by reference in their entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No.

US20120282343; the contents of which are herein incorporated by reference in their entirety.

In some embodiments, the nucleic acid vaccine compositions of the present disclosure may be formulated in a modular composition such as described in U.S. Pat. No. 8,575,123 to Manoharan et al., the contents of which are herein incorporated by reference in their entirety. As a non-limiting example, the modular composition may comprise a nucleic acid, e.g., the nucleic acid vaccine compositions of the present disclosure, at least one endosomolytic component, and at least one targeting ligand. The modular composition may have a formula such as any formula described in U.S. Pat. No. 8,575,123 to Manoharan et al.

In some embodiments, the nucleic acid vaccine compositions of the present disclosure may be encapsulated in the lipid formulation to form a stable nucleic acid-lipid particle (SNALP) such as described in U.S. Pat. No. 8,546,554 to de Fougerolles et al., the contents of which are incorporated here by reference in their entirety. The lipid may be cationic or non-cationic. In one non-limiting example, the lipid to nucleic acid ratio (mass/mass ratio) (e.g., lipid to nucleic acid vaccine compositions ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or 5:1, 6:1, 7:1, 8:1, 9:1, 10:1, or 11:1. In another example, the SNALP includes 40% 2,2-Dilinoleyl-4-dimethylaminoethyl-[1,3]-dioxolane (Lipid A), 10% dioleoylphosphatidylcholine (DSPC), 40% cholesterol, 10% polyethylene glycol (PEG)-C-DOMG (mole percent) with a particle size of 63.0±20 nm and a 0.027 nucleic acid/lipid ratio.

The nucleic acid vaccine compositions of the present disclosure may be formulated with a nucleic acid-lipid particle comprising an endosomal membrane destabilizer as disclosed in U.S. Pat. No. 7,189,705 to Lam et al., the contents of which are incorporated herein by reference in their entirety. As a non-limiting example, the endosomal membrane destabilizer may be a Ca²⁺ ion.

In some embodiments, the nucleic acid vaccine compositions of the present disclosure may be formulated with formulated lipid particles (FLiPs) disclosed in U.S. Pat. No. 8,148,344 to Akinc et al., the contents of which are herein incorporated by reference in their entirety. Akinc et al. teach that FLiPs may comprise at least one of a single or double-stranded oligonucleotide, where the oligonucleotide has been conjugated to a lipophile and at least one of an emulsion or liposome to which the conjugated oligonucleotide has been aggregated, admixed or associated. These particles have surprisingly been shown to effectively deliver oligonucleotides to heart, lung and muscle as disclosed in U.S. Pat. No. 8,148,344 to Akinc et al.

In some embodiments, the nucleic acid vaccine compositions of the present disclosure may be delivered to a cell using a composition comprising an expression vector in a lipid formulation as described in U.S. Pat. No. 6,086,913 to Tam et al., the contents of which are incorporated herein by reference in their entirety. The composition disclosed by Tam is serum-stable and comprises an expression vector comprising first and second inverted repeated sequences from an adeno associated virus (AAV), a rep gene from AAV, and a nucleic acid fragment. The expression vector in Tam is complexed with lipids.

In some embodiments, the nucleic acid vaccine compositions of the present disclosure may be formulated with a lipid formulation disclosed in US Pub. No. US 20120270921 to de Fougerolles et al., the contents of which are incorporated herein by reference in their entirety. In one non-limiting example, the lipid formulation may include a cationic lipid having the formula A described in US 20120270921. In another non-limiting example, the compositions of exemplary nucleic acid-lipid particles disclosed in Table A of US20120270921 may be used with the nucleic acid vaccine compositions of the present disclosure.

In some embodiments, the nucleic acid vaccine compositions of the present disclosure may be fully encapsulated in a lipid particle disclosed in US Pub. No. US 20120276207 to Maurer et al., the contents of which are incorporated herein by reference in their entirety. The particles may comprise a lipid composition comprising preformed lipid vesicles, a charged therapeutic agent, and a destabilizing agent to form a mixture of preformed vesicles and therapeutic agent in a destabilizing solvent, wherein the destabilizing solvent is effective to destabilize the membrane of the preformed lipid vesicles without disrupting the vesicles.

In some embodiments, the nucleic acid vaccine compositions of the present disclosure may be formulated with a conjugated lipid. In a non-limiting example, the conjugated lipid may have a formula such as described in US Pub. No. US 20120264810 to Lin et al., the contents of which are incorporated herein by reference in their entirety. The conjugate lipid may form a lipid particle which further comprises a cationic lipid, a neutral lipid, and a lipid capable of reducing aggregation.

In some embodiments, the nucleic acid vaccine compositions of the present disclosure may be formulated in a neutral liposomal formulation such as disclosed in US Pub. No. US 20120244207 to Fitzgerald et al., the contents of which are incorporated herein by reference in their entirety. The phrase “neutral liposomal formulation” refers to a liposomal formulation with a near neutral or neutral surface charge at a physiological pH. Physiological pH can be, e.g., about 7.0 to about 7.5, or, e.g., about 7.5, or, e.g., 7.0, 7.1, 7.2, 7.3, 7.4, or 7.5, or, e.g., 7.3, or, e.g., 7.4. An example of a neutral liposomal formulation is an ionizable lipid nanoparticle (iLNP). A neutral liposomal formulation can include an ionizable cationic lipid, e.g., DLin-KC2-DMA.

In some embodiments, the nucleic acid vaccine compositions of the present disclosure may be formulated with a charged lipid or an amino lipid. As used herein, the term “charged lipid” is meant to include those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.” In a non-limiting example, the amino lipid may be any amino lipid described in US Pub. No. US20110256175 to Hope et al., the contents of which are incorporated herein by reference in their entirety. For example, the amino lipids may have the structure disclosed in Tables 3-7 of Hope, such as structure (II), DLin-K-C2-DMA, DLin-K2-DMA, DLin-K6-DMA, etc. The resulting pharmaceutical preparations may be lyophilized according to Hope. In another non-limiting example, the amino lipids may be any amino lipid described in US 20110117125 to Hope et al., the contents of which are incorporated herein by reference in their entirety, such as a lipid of structure (I), DLin-K-DMA, DLin-C-DAP, DLin-DAC, DLin-MA, DLin-S-DMA, etc. In another non-limiting example, the amino lipid may have the structure (I), (II), (III), or (IV), or 4-(R)-DLin-K-DMA (VI), 4-(S)-DLin-K-DMA (V) as described in PCT Patent Application Publication No. WO2009132131 to Manoharan et al., the contents of which are incorporated herein by reference in their entirety. In another non-limiting example, the charged lipid used in any of the formulations described herein may be any charged lipid described in EP2509636 to Manoharan et al., the contents of which are incorporated herein by reference in their entirety.

In some embodiments, the nucleic acid vaccine composition s of the present disclosure may be formulated with an association complex. In a non-limiting example, the association complex comprises one or more compounds each having a structure defined by formula (I), a PEG-lipid having a structure defined by formula (XV), a steroid and a nucleic acid disclosed in U.S. Pat. No. 8,034,376 to Manoharan et al., the contents of which are incorporated herein by reference in their entirety. The nucleic acid vaccine compositions may be formulated with any association complex described in U.S. Pat. No. 8,034,376., the contents of which are herein incorporated by reference in its entirety.

In some embodiments, the nucleic acid vaccine compositions of the present disclosure may be formulated with reverse head group lipids. As a non-limiting example, the nucleic acid vaccine compositions may be formulated with a zwitterionic lipid comprising a headgroup wherein the positive charge is located near the acyl chain region and the negative charge is located at the distal end of the head group, such as a lipid having structure (A) or structure (I) described in PCT Patent Application Publication No. WO2011056682 to Leung et al., the contents of which are incorporated herein by reference in their entirety.

In some embodiments, the nucleic acid vaccine compositions of the present disclosure may be formulated in a lipid bilayer carrier. As a non-limiting example, the nucleic acid vaccine compositions may be combined with a lipid-detergent mixture comprising a lipid mixture of an aggregation-preventing agent in an amount of about 5 mol % to about 20 mol %, a cationic lipid in an amount of about 0.5 mol % to about 50 mol %, and a fusogenic lipid and a detergent, to provide a nucleic acid-lipid-detergent mixture; and then dialyzing the nucleic acid-lipid-detergent mixture against a buffered salt solution to remove the detergent and to encapsulate the nucleic acid in a lipid bilayer carrier and provide a lipid bilayer-nucleic acid composition, wherein the buffered salt solution has an ionic strength sufficient to encapsulate of from about 40% to about 80% of the nucleic acid, described in PCT Patent Application Publication No. WO1999018933 to Cullis et al., the contents of which are incorporated herein by reference in their entirety.

In some embodiments, the nucleic acid vaccine compositions of the present disclosure may comprise (a) a nucleic acid; (b) 1.0 mole % to 45 mole % of a cationic lipid; (c) 0.0 mole % to 90 mole % of another lipid; (d) 1.0 mole % to 10 mole % of a bilayer stabilizing component; (e) 0.0 mole % to 60 mole % cholesterol; and (f) 0.0 mole % to 10 mole % of cationic polymer lipid as described in EP1328254 to Cullis et al., the contents of which are incorporated herein by reference in their entirety.

In some embodiments, the nucleic acid vaccine may be delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 um, less than 950 μm, less than 975 μm.

In another embodiment, nucleic acid vaccine may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 nm to about 50 nm, from about 20 nm to about 50 nm, from about 30 nm to about 50 nm, from about 40 nm to about 50 nm, from about 20 nm to about 60 nm, from about 30 nm to about 60 nm, from about 40 nm to about 60 nm, from about 20 nm to about 70 nm, from about 30 nm to about 70 nm, from about 40 nm to about 70 nm, from about 50 nm to about 70 nm, from about 60 nm to about 70 nm, from about 20 nm to about 80 nm, from about 30 nm to about 80 nm, from about 40 nm to about 80 nm, from about 50 nm to about 80 nm, from about 60 nm to about 80 nm, from about 20 nm to about 90 nm, from about 30 nm to about 90 nm, from about 40 nm to about 90 nm, from about 50 nm to about 90 nm, from about 60 nm to about 90 nm and/or from about 70 nm to about 90 nm.

In some embodiments, the nucleic acid vaccine may be formulated in lipid nanoparticles having a diameter from about 10 nm to about 100 nm such as, but not limited to, about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm about 50 nm to about 80 nm, about 50 nm to about 90 nm, about 50 nm to about 100 nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm and/or about 90 nm to about 100 nm.

In some embodiments, the nucleic acid vaccine may be formulated in lipid nanoparticles having a diameter from 10-1000 nm. The nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm.

In some embodiments, the lipid nanoparticles may have a diameter from about 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

### Polymers, Biodegradable Nanoparticles, and Core-Shell Nanoparticles

The nucleic acid vaccine compositions of the disclosure can be formulated using natural and/or synthetic polymers. Non-limiting examples of polymers which may be used for delivery include, but are not limited to, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as, but not limited to, PHASERX® (Seattle, WA).

A non-limiting example of chitosan-based formulation includes a core of positively charged chitosan and an outer portion of negatively charged substrate (U.S. Pub. No. US20120258176; the contents of which are herein incorporated by reference in their entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In some embodiments, the polymers used in the present disclosure have undergone processing to reduce and/or inhibit the attachment of unwanted substances such as, but not limited to, bacteria, to the surface of the polymer. The polymer may be processed by methods known and/or described in the art and/or described in PCT Patent Application Publication No. WO2012150467; the contents of which are herein incorporated by reference in their entirety.

A non-limiting example of PLGA based formulations include, but are not limited to, PLGA-based injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space. The PLGA-based injectable depots may be long-acting.

Many of these polymer approaches have demonstrated efficacy in delivering oligonucleotides in vivo into the cell cytoplasm (reviewed in de Fougerolles Hum Gene Ther. 2008, 19:125-132; the contents of which are herein incorporated by reference in their entirety). Two polymer approaches that have yielded robust in vivo delivery of nucleic acids, i.e., in the case of small interfering RNA (siRNA), are dynamic polyconjugates and cyclodextrin-based nanoparticles. The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007, 104:12982-12887; the contents of which are herein incorporated by reference in their entirety). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (See again, Rozema et al., Proc Natl Acad Sci USA. 2007, 104:12982-12887). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005, 65: 8984-8982; herein incorporated by reference in its entirety) and siRNA formulated in these nanoparticles was well tolerated in non-human primates (Heidel et al., Proc Natl Acad Sci USA 2007, 104:5715-21; herein incorporated by reference in its entirety). Both of these delivery strategies incorporate rational approaches using both targeted delivery and endosomal escape mechanisms.

The polymer formulation can permit the sustained or delayed release of nucleic acid vaccine compositions (e.g., following intramuscular, subcutaneous, intraparenchymal, intrathecal, intracerebroventricular administration). The altered release profile for the nucleic acid vaccine compositions can result in, for example, translation of an encoded protein, or polypeptide or peptide over an extended period of time. Biodegradable polymers have been previously used to protect nucleic acids from degradation and been shown to result in sustained release of payloads in vivo (Rozema et al., Proc Natl Acad Sci USA. 2007, 104:12982-12887; Sullivan et al., Expert Opin Drug Deliv. 2010, 7:1433-1446; Convertine et al., Biomacromolecules. 2010, Oct. 1; Chu et al., Acc Chem Res. 2012, Jan. 13; Manganiello et al., Biomaterials. 2012, 33:2301-2309; Benoit et al., Biomacromolecules. 2011, 12:2708-2714; Singha et al., Nucleic Acid Ther. 2011, 2:133-147; de Fougerolles Hum Gene Ther. 2008, 19:125-132; Schaffert and Wagner, Gene Ther. 2008, 16:1131-1138; Chaturvedi et al., Expert Opin Drug Deliv. 2011, 8:1455-1468; Davis, Mol Pharm. 2009, 6:659-668; Davis, Nature, 2010, 464:1067-1070; the contents of each of which are herein incorporated by reference in their entirety).

In some embodiments, the nucleic acid vaccines of the present disclosure may be formulated for controlled release. One form of controlled-release formulation contains the therapeutic compound or its salt dispersed or encapsulated in a slowly degrading, non-toxic, non-antigenic polymer such as copoly(lactic/glycolic) acid, as described in the pioneering work of Kent et al., U.S. Pat. No. 4,675,189, the contents of which are incorporated by reference herein in their entirety. The compounds, or their salts, may also be formulated in cholesterol or other lipid matrix pellets, or silastomer matrix implants. As a non-limiting example, the nucleic acid vaccines of the present disclosure may be dispersed or encapsulated in the polymers disclosed in U.S. Pat. No. 4,675,189 for controlled release. An additional form of controlled-release formulation comprises a solution of biodegradable polymer, such as copoly(lactic/glycolic acid) or block copolymers of lactic acid and PEG, which is injected subcutaneously or intramuscularly to achieve a depot formulation for controlled release.

In some embodiments, the pharmaceutical compositions may be sustained release formulations. In further embodiments, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

As a non-limiting example, nucleic acid vaccine compositions may be formulated in PLGA microspheres by preparing the PLGA microspheres with tunable release rates (e.g., days and weeks) and encapsulating the nucleic acid vaccine compositions in the PLGA microspheres while maintaining the integrity of the nucleic acid vaccine compositions during the encapsulation process. EVAc are non-biodegradable, biocompatible polymers which are used extensively in pre-clinical sustained release implant applications. Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011, 12:2708-2714; Rozema et al., Proc Natl Acad Sci USA. 2007, 104:12982-12887; Davis, Mol Pharm. 2009, 6:659-668; Davis, Nature, 2010 464:1067-1070; the contents of each of which are herein incorporated by reference in their entirety).

The nucleic acid vaccine compositions of the disclosure may be formulated with or in a polymeric compound. The polymeric compound may include at least one polymer such as, but not limited to, polyethenes, polyethylene glycol (PEG), poly(1-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the nucleic acid vaccine compositions of the disclosure may be formulated with the polymeric compound of PEG grafted with PLL as described in U.S. Pat. No. 6,177,274; herein incorporated by reference in its entirety. The formulation may be used for transfecting cells in vitro or for in vivo delivery of the nucleic acid vaccine compositions. In another example, the nucleic acid vaccine compositions may be suspended in a solution or medium with a cationic polymer, in a dry pharmaceutical composition or in a solution that is capable of being dried as described in U.S. Pub. Nos. US20090042829 and US20090042825; the contents of each of which are herein incorporated by reference in their entirety.

As another non-limiting example, the nucleic acid vaccine compositions of the disclosure may be formulated with a PLGA-PEG block copolymer (see US Pub. No. US20120004293 and U.S. Pat. No. 8,236,330, herein incorporated by reference in their entireties) or PLGA-PEG-PLGA block copolymers (See U.S. Pat. No. 6,004,573, herein incorporated by reference in its entirety). As a non-limiting example, the nucleic acid vaccine compositions of the disclosure may be formulated with a diblock copolymer of PEG and PLA or PEG and PLGA (see U.S. Pat. No. 8,246,968, herein incorporated by reference in its entirety).

In some embodiments, the nucleic acid vaccines compositions may be formulated with branched PEG molecules as described in or made by the methods described in PCT Patent Application Publication No. WO20180126084; the contents of which are herein incorporated by reference in their entirety. As a non-limiting example, the branched PEG which may be used in the formulations described herein may have the formula I, formula II, formula III, formula IV, formula V, formula VI of PCT Publication No. WO20180126084, the contents of which are herein incorporated by reference in their entirety.

A polyamine derivative may be used to deliver nucleic acids or to treat and/or prevent a disease or to be included in an implantable or injectable device (U.S. Pub. No. US20100260817; the contents of which are herein incorporated by reference in their entirety). As a non-limiting example, the nucleic acid vaccine compositions of the present disclosure may be formulated using the polyamine derivative described in U.S. Pub. No. US20100260817; the contents of which are incorporated herein by reference in their entirety. As another non-limiting example, the nucleic acid vaccine compositions of the present disclosure may be delivered using a polyamide polymer such as, but not limited to, a polymer comprising a 1,3-dipolar addition polymer prepared by combining a carbohydrate diazide monomer with a dialkyne unite comprising oligoamines (U.S. Pat. No. 8,236,280; the contents of which are herein incorporated by reference in their entirety).

In some embodiments, the nucleic acid vaccine compositions of the present disclosure may be formulated with at least one polymer and/or derivatives thereof described in PCT Patent Application Publication Nos. WO2011115862, WO2012082574 and WO2012068187 and U.S. Pub. No. US20120283427, the contents of each of which are herein incorporated by reference in their entireties. The nucleic acid vaccine compositions of the present disclosure may be formulated with a polymer of formula Z as described in WO2011115862; the contents of which are herein incorporated by reference in their entirety. The nucleic acid vaccine compositions may be formulated with a polymer of formula Z, Z′ or Z″ as described in PCT Patent Application Publication Nos. WO2012082574 or WO2012068187 and U.S. Pub. No. US2012028342; the contents of each of which are herein incorporated by reference in their entireties. The polymers formulated with the nucleic acid vaccine compositions of the present disclosure may be synthesized by the methods described in PCT Patent Application Publication Nos. WO2012082574 or WO2012068187, the contents of each of which are herein incorporated by reference in their entireties.

The nucleic acid vaccine compositions of the disclosure may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

Formulations of nucleic acid vaccine compositions of the disclosure may include at least one amine-containing polymer such as, but not limited to polylysine, polyethylene imine, poly(amidoamine) dendrimers or combinations thereof.

For example, the nucleic acid vaccine compositions of the disclosure may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, and U.S. Pub. Nos. US20030073619 and US20040142474; the contents of each of which are herein incorporated by reference in their entireties. The poly(alkylene imine) may be made using methods known in the art and/or as described in U.S. Pub. No. US20100004315, which is herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of each of which are each incorporated herein by reference in their entirety. The linear biodegradable copolymer may be made using methods known in the art and/or as described in U.S. Pat. No. 6,652,886; the contents of which are each incorporated herein by reference in their entirety. The PAGA polymer may be made using methods known in the art and/or as described in U.S. Pat. No. 6,217,912; the contents of which are herein incorporated by reference in their entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. US2012009145; the contents of each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Pub. No. US20100004315 or U.S. Pat. Nos. 6,267,987 and 6,217,912; the contents of each of which are herein incorporated by reference in their entireties.

The nucleic acid vaccine compositions of the disclosure may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

The nucleic acid vaccine compositions of the disclosure may be formulated with at least one crosslinkable polyester. Crosslinkable polyesters include those known in the art and described in US Pub. No. US20120269761; the contents of which herein are incorporated by reference in their entirety.

In some embodiments, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present disclosure include those described in PCT Patent Application Publication No. WO2008103276; the contents of which are herein incorporated by reference in their entirety. The polymers may be conjugated using a ligand conjugate such as, but not limited to, the conjugates described in U.S. Pat. No. 8,273,363; the contents of which are herein incorporated by reference in their entirety.

In some embodiments, the nucleic acid vaccine compositions described herein may be conjugated with another compound. Non-limiting examples of conjugates are described in U.S. Pat. Nos. 7,964,578 and 7,833,992; the contents of each of which are herein incorporated by reference in their entireties. In some embodiments, the nucleic acid vaccine compositions of the present disclosure may be conjugated with conjugates of formula 1-122 as described in U.S. Pat. Nos. 7,964,578 and 7,833,992; the contents of each of which are herein incorporated by reference in their entireties. The nucleic acid vaccine compositions described herein may be conjugated with a metal such as, but not limited to, gold. (See e.g., Giljohann et al. Journ. Amer. Chem. Soc. 2009, 131(6): 2072-2073; the contents of which are herein incorporated by reference in their entirety). In some embodiments, the nucleic acid vaccine compositions described herein may be conjugated and/or encapsulated in gold-nanoparticles (PCT Application Publication No. WO201216269 and U.S. Pub. No. US20120302940; the contents of each of which are herein incorporated by reference in their entirety).

As described in U.S. Pub. No. US20100004313, a gene delivery composition may include a nucleotide sequence and a poloxamer. As a non-limiting example, the nucleic acid vaccine compositions of the present disclosure may be used in a gene delivery composition with the poloxamer described in U.S. Pub. No. US20100004313; the contents of which are each incorporated herein by reference in their entirety.

In some embodiments, the polymer formulations comprising the nucleic acid vaccines of the present disclosure may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. US20090042829; the contents of which are herein incorporated by reference in their entirety.

The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B—[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

In some embodiments, the nucleic acid vaccine compositions of the disclosure may be formulated in a polyplex of one or more polymers (U.S. Pub. Nos. US20120237565 and US20120270927; the contents of each of which are herein incorporated by reference in their entirety). In some embodiments, the polyplex comprises two or more cationic polymers. The cationic polymer may comprise a poly(ethylene imine) (PEI) such as linear PEI.

The nucleic acid vaccine compositions of the disclosure can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so delivery of the nucleic acid vaccine compositions may be enhanced (Wang et al., Nat Mater. 2006, 5:791-796; Fuller et al., Biomaterials. 2008, 29:1526-1532; DeKoker et al., Adv Drug Deliv Rev. 2011, 63:748-761; Endres et al., Biomaterials. 2011, 32:7721-7731; Su et al., MolPharm. 2011; 8(3):774-87; the contents of each of which are herein incorporated by reference in their entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (PCT Application Publication No. WO20120225129; the contents of which are herein incorporated by reference in their entirety).

Biodegradable calcium phosphate nanoparticles in combination with lipids and/or polymers may be used to deliver nucleic acid vaccine compositions in vivo. In some embodiments, a lipid coated calcium phosphate nanoparticle, which may also contain a targeting ligand such as anisamide, may be used to deliver the nucleic acid vaccine compositions of the present disclosure. For example, to effectively deliver siRNA in a mouse metastatic lung model a lipid coated calcium phosphate nanoparticle was used (Li et al., J Contr Rel. 2010, 142: 416-421; Li et al., J Contr Rel. 2012, 158:108-114; Yang et al., Mol Ther. 2012, 20:609-615; the contents of each of which are herein incorporated by reference in their entirety). This delivery system combines both a targeted nanoparticle and a component to enhance the endosomal escape, calcium phosphate, in order to improve delivery of the siRNA.

In some embodiments, calcium phosphate with a PEG-polyanion block copolymer may be used to delivery nucleic acid vaccine compositions of the disclosure (Kazikawa et al., J Contr Rel. 2004, 97:345-356; Kazikawa et al., J Contr Rel. 2006, 111:368-370; the contents of each of which are herein incorporated by reference in their entirety).

In some embodiments, a PEG-charge-conversional polymer (Pitella et al., Biomaterials. 2011, 32:3106-3114; the contents of which are herein incorporated by reference in their entirety) may be used to form a nanoparticle to deliver the nucleic acid vaccine compositions of the present disclosure. The PEG-charge-conversional polymer may improve upon the PEG-polyanion block copolymers by being cleaved into a polycation at acidic pH, thus enhancing endosomal escape.

In some embodiments, a core-shell nanoparticle may be used to form a nanoparticle to deliver the nucleic acid vaccine compositions of the present disclosure. The use of core-shell nanoparticles has additionally focused on a high-throughput approach to synthesize cationic cross-linked nanogel cores and various shells (Siegwart et al., Proc Natl Acad Sci USA. 2011, 108:12996-13001; the contents of which are herein incorporated by reference in their entirety). The complexation, delivery, and internalization of the polymeric nanoparticles can be precisely controlled by altering the chemical composition in both the core and shell components of the nanoparticle. For example, the core-shell nanoparticles may efficiently deliver nucleic acid vaccine compositions to mouse hepatocytes after they covalently attach cholesterol to the nanoparticle.

In some embodiments, the nanoparticles described herein may be nanoparticles which include at least one ligand, and the ligand may be a peptide, a nucleic acid aptamer, which is a small molecular weight (8-13 Kda) single-stranded RNA or DNA with low nanomolar binding affinities toward their targets, a peptide aptamer, an antibody, a small molecule ligand such as, but not limited to, folate, anisamide, and galactose. (Leng et al. Journal of Drug Delivery. 2017, 17, Article ID 6971297; the contents of which are herein incorporated by reference in their entirety).

In some embodiments, a hollow lipid core comprising a middle PLGA layer and an outer neutral lipid layer containing PEG may be used to delivery of the nucleic acid vaccine compositions of the present disclosure. As a non-limiting example, the lipid-polymer-lipid hybrid nanoparticle may be used to deliver the nucleic acid vaccine compositions described herein (Shi et al, Angew Chem Int Ed. 2011, 50:7027-7031; the contents of which are herein incorporated by reference in their entirety).

Core-shell nanoparticles for use with the nucleic acid vaccine compositions of the present disclosure may be formed by the methods described in U.S. Pat. No. 8,313,777; the contents of which are herein incorporated by reference in their entirety.

In some embodiments, the core-shell nanoparticles may comprise a core of the nucleic acid vaccine compositions described herein and a polymer shell. The polymer shell may be any of the polymers described herein and are known in the art. In an additional embodiment, the polymer shell may be used to protect the nucleic acid vaccine compositions in the core. (see, e.g., US Publication No. 20120321719; the contents of which are herein incorporated by reference in their entirety).

In some embodiments, the polymer used with the formulations described herein may be a modified polymer (such as, but not limited to, a modified polyacetal) as described in PCT Application Publication No. WO2011120053; the contents of which are herein incorporated by reference in their entirety.

In some embodiments, the nucleic acid vaccine compositions may be delivered to the cell or cytosol of a targeT-cell by contacting the cell with a membrane-destabilizing polymer and a conjugate of the nucleic acid vaccine composition, a targeting ligand and an optional linker. Non-limiting examples of membrane-destabilizing polymers are taught in International PCT Application Publication No. WO2020093061, the contents of which are herein incorporated by reference in their entirety, such as, but not limited to, the membrane-destabilizing polymers of formula XX therein.

### Excipients

In some embodiments, pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, anti-oxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21″ Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, crosslinked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chon-drux, cholesterol, xanthan, pectin, gelatin, egg yolk, casein, wool fat, cholesterol, wax, and lecithin), colloidal clays (e.g. bentonite (aluminum silicate) and VEEGUM® (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (TWEEN@20), polyoxyethylene sorbitan (TWEEN@60), polyoxyethylene sorbitan monooleate (TWEEN@80), sorbitan monopalmitate (SPAN@40), sorbitan monostearate (SPAN@60), sorbitan tristearate (SPAN®65), glyceryl monooleate, sorbitan monooleate (SPAN@80)), polyoxyethylene esters (e.g. polyoxyethylene monostearate (MYRJ@45), polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether (BRIJ@30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenyl ethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®!!, NEOLONE™, KATHON™, and/or EUXYL®.

In some embodiments, the pH of the pharmaceutical solutions are maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

In some embodiments of the present disclosure, the nucleic acid vaccine compositions described herein may comprise at least one nucleic acid vaccine that is formulated in a lipid nanoparticle (LNP) and at least one excipient. As non-limiting examples, the excipient may be a sugar such as sucrose.

### Adjuvants

Adjuvants may also be administered with or in combination with one or more of the nucleic acid vaccines described herein, e.g., the mRNA vaccine. Adjuvants may be used to enhance the immunogenicity of the nucleic acid vaccine, modify the immune response, reduce the amount of nucleic acid vaccine needed for immunization, reduced the frequency of additional or “booster” immunizations needed or to create an improved immune response in those with weakened or immunocompromised immune systems or the elderly. The adjuvants may be a component of the formulation containing the nucleic acid vaccine or they may be co-administered with the nucleic acid vaccines compositions. Co-administration of the adjuvant may be any method known in the art or described herein such as, but not limited to, intravenous (IV), intramuscular (IM), subcutaneous (SC) or intradermal (ID).

In some embodiments, the adjuvant is natural or synthetic. The adjuvants may also be organic or inorganic.

In some embodiments, the adjuvant used with the nucleic acid vaccine is from a class of adjuvants such as, but not limited to carbohydrates, microorganisms, mineral salts (e.g., aluminum hydroxide, aluminum phosphate gel, or calcium phosphate gel), emulsions (e.g., oil emulsion, surfactant based emulsion, purified saponin, and oil-in water emulsion), inert vehicles, particulate adjuvants (e.g., unilamellar liposomal vehicles such as virosomes or a structured complex of saponions and lipids such as polylactide co-glycolide (PLG)), microbial derivatives, endogenous human immunomodulators, and tensoactive compounds. Listings of adjuvants which may be used with the nucleic acid vaccines described herein may be found on the web-based vaccine adjuvant database Vaxjo (see e.g., violinet.org/vaxjo or Sayers et al., Journal ofBiomedicine and Biotechnology. 2012; 2012:831486. PMID: 22505817; the contents of which are herein incorporated by reference in their entirety).

Adjuvants may be selected for use with the nucleic acid vaccines by one of ordinary skill in the art. Adjuvants may be interferons, TNF-alpha, TNF-beta, chemokines (e.g., CCL21, eotaxin, HMGB1, SA100-8alpha, GCSF, GMCSF, granulysin, lactoferrin, ovalbumin, CD40L, CD28 agonists, PD1, soluble PD1, PDL1, PDL2) or interleukins (e.g., IL1, IL2, IL4, IL6, IL7, IL10, IL12, IL13, IL15, IL17, IL18, IL21, and IL23). Non-limiting examples of adjuvants include Abisco-100 vaccine adjuvant, Adamantylamide Dipeptide Vaccine Adjuvant, Adjumer™, AF03, Albumin-heparin microparticles vaccine adjuvant, Algal Glucan, Algammulin, alhydrogel, aluminum hydroxide vaccine adjuvant, aluminum phosphate vaccine adjuvant, aluminum potassium sulfate adjuvant, Aluminum vaccine adjuvant, amorphous aluminum hydroxyphosphate sulfate adjuvant, Arlacel A, ASO, AS04, AS03, AS-2 vaccine adjuvant, Avridine®, B7-2 vaccine adjuvant, Bay R1005, Bordetella pertussis component Vaccine Adjuvant, Bupivacaine vaccine adjuvant, Calcium Phosphate Gel, Calcium phosphate vaccine adjuvant, Cationic Liposomal Vaccine Adjuvant, cationic liposome-DNA complex JVRS-100, Cholera toxin, Cholera toxin B subunit, Corynebacterium-derived P40 Vaccine Adjuvant, CpG DNA Vaccine Adjuvant, CRL1005, CTA1-DD gene fusion protein, DDA Adjuvant, DHEA vaccine adjuvant, DL-PGL (Polyester poly (DL-lactide-co-glycolide)) vaccine adjuvant, DOC/Alum Complex, E. coli heat-labile toxin, Etx B subunit Adjuvant, Flagellin, Freund's Complete Adjuvant, Freund's Incomplete Adjuvant, Gamma Inulin, Gerbu Adjuvant, GM-CSF, GMDP, Imiquimod, Immunoliposomes Containing Antibodies to Costimulatory Molecules, ISCOM(s)™ ISCOMA-TRIX®, Killed Corynebacterium parvum Vaccine Adjuvant, Lipopolysaccharide, Liposomes, Loxoribine, LTK63 Vaccine Mutant Adjuvant, LTK72 vaccine adjuvant, LTR192G Vaccine Adjuvant, Matrix-S, MF59, Montanide Incomplete Seppic Adjuvant, Montanide ISA 51, Montanide ISA 720 Adjuvant, MPL-SE vaccine adjuvant, MPL™ Adjuvant, MTP-PE Liposomes, Murametide, Muramyl Dipeptide Adjuvant, Murapalmitine, D-Murapalmitine, NAGO, nanoemulsion vaccine adjuvant, Non-Ionic Surfactant Vesicles, non-toxic mutant E112K of Cholera Toxin mCT-E112K, PMMA, Poly(LC), Polygen Vaccine Adjuvant, Protein Cochleates, QS-21, Quil-A vaccine adjuvant, RC529 vaccine adjuvant, Recombinant hlFN-gamma/Interferon-g, Rehydragel EV, Rehydragel HPA, Resiquimod, Ribi Vaccine Adjuvant, SAF-1, Saponin Vaccine Adjuvant, Sclavo peptide, Sendai Proteoliposomes, Sendai-containing Lipid Matrices, Specol, SPT (Antigen Formulation), Squalene-based Adjuvants, Stearyl Tyrosine, Theramide®, Threonyl muramyl dipeptide (TMDP), Titer-Max Gold Adjuvant, Ty Particles vaccine adjuvant, and VSA-3 Adjuvant.

In some embodiments, the nucleic acid vaccines described herein may be used as a vaccine and may further comprise an adjuvant which may enable the vaccine to elicit a higher immune response. As a non-limiting example, the adjuvant could be a sub-micron oil-in-water emulsion which can elicit a higher immune response in human pediatric populations (see e.g., the adjuvanted vaccines described in US Patent Publication No. US20120027813 and U.S. Pat. No. 8,506,966, the contents of each of which are herein incorporated by reference in their entirety).

### Dosing and Administration

The present disclosure encompasses the delivery of nucleic acid vaccine compositions including, for example, nucleic acid vaccine for COVID-19 for any therapeutic, prophylactic, pharmaceutical, diagnostic or imaging use by any appropriate route taking into consideration likely advances in the sciences of drug delivery. Delivery may be naked or formulated.

The nucleic acid vaccine compositions of the present disclosure may be delivered to a cell naked. As used herein in, “naked” refers to delivering nucleic acid vaccine compositions free from agents which promote transfection. For example, the nucleic acid vaccine compositions delivered to the cell may contain no modifications. The naked nucleic acid vaccine compositions may be delivered to the cell using routes of administration known in the art and described herein.

The nucleic acid vaccine compositions of the present disclosure may be formulated, using the formulation components and methods described herein. The formulations may contain nucleic acid vaccine compositions which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated nucleic acid vaccine compositions may be delivered to the cell using routes of administration known in the art and described herein.

The nucleic acid vaccine compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like. The nucleic acid vaccine compositions of the present disclosure may also be cloned into a retroviral replicating vector (RRV) and transduced to cells.

### Dosing

Provided herein also include methods comprising administering the nucleic acid vaccines described herein to a subject in need thereof. The exact amount required will vary from subject to subject, depending on the species, age, health, and general condition of the subject, the severity of the disease, the particular composition, its mode of administration, its mode of activity, and the like. Compositions are typically formulated in dosage unit form for ease of administration and uniformity of dosage. It will be understood, however, that the total daily usage of the compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The present disclosure contemplates dosage levels of between about 0.001 and about 500 mg nucleic acid vaccine (e.g., nucleic acid vaccine for COVID-19, e.g., mRNA vaccine for COVID-19)/kg body weight per day, about 0.001 and about 200 mg/kg, about 0.001 and about 100 mg/kg, 0.01 and about 100 mg/kg, preferably between about 0.005 and about 50 mg/kg, 0.01 and about 50 mg/kg, 0.01 and about 40 mg/kg, 0.01 and about 30 mg/kg, 0.01 and about 10 mg/kg, 0.05 and about 50 mg/kg, 0.05 and about 30 mg/kg, 0.05 and about 10 mg/kg, 0.05 and about 5 mg/kg, 0.1 and about 50 mg/kg, 0.1 and about 30 mg/kg, 0.1 and about 10 mg/kg, 0.1 and about 1 mg/kg, 1.0 and about 50 mg/kg, 1.0 and about 40 mg/kg, 1.0 to about 30 mg/kg, 10 to about 50 mg/kg body weight. Other embodiments contemplate a dosage of between about 0.001-0.010, 0.010-0.050, 0.050-0.100, 0.1-0.5, 0.5-1.0, 1.0-5.0, 5.0-10, 10-50 mg/kg, 10-100 mg/kg body weight. The dosages may be administered about hourly, multiple times per day, daily, every other day, weekly, every other week, monthly, every other month, or on an as-needed basis.

In some embodiments, compositions of the nucleic acid vaccines may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, from about 1 mg/kg to about 25 mg/kg, from about 1 mg/kg to about 50 mg/kg, from about 10 mg/kg to about 100 mg/kg, from about 10 mg/kg to about 50 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

In some embodiments, compositions of the nucleic acid vaccines described herein may be administered at dosage levels sufficient to deliver to a subject, about 1 μg, 10 μg. 15 μg, 20 μg, 25 μg, 30 μg, 35 μg, 40 μg, 50 μg, 60 μg, 70 μg, 80 μg, 90 μg, or 100 μg of the nucleic acid composition.

In some embodiments, the nucleic acid vaccines may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24-hour period. It may be administered as a single unit dose. In some embodiments, the nucleic acid vaccines described herein are administered to a subject in split doses. The nucleic acid vaccines may be formulated in buffer only or in a formulation described herein.

In some embodiments, the nucleic acid vaccine compositions described herein may be administered to a subject in two separate phases, a loading dosing phase and a maintenance dosing phase. The dosing regimen may comprise an initial higher loading dose of the nucleic acid vaccine that is given to the subject first time at the beginning of a course of prevention, alleviation and/or treatment, e.g., first dose for preventing COVID-19, and a lower maintenance dose following the first loading dose. In some embodiments, the loading dose and the maintenance dose have the same amount of the nucleic acid vaccines of the present disclosure. In some embodiments, more than one maintenance doses are administered to the subject. The multiple maintenance doses may be administered biweekly, every three weeks, every four weeks, monthly, bimonthly, every three months, every four months, every five months, or every six months. In the context of vaccination for prevention of a disorder, e.g., the nucleic acid (e.g., mRNA) vaccine for COVID-19, the maintenance doses of the nucleic acid vaccines may also be referred to as booster doses. As used herein, a “booster dose” (or “booster shot) is an extra or supplemental dose of a vaccine after an initial primer dose. The booster dose may have the same amount of the nucleic acid vaccine as the initial loading dose. Alternatively, the booster dose has an amount of the nucleic acid vaccine that is smaller than the original amount of the nucleic acid vaccine in the initial dose. In some embodiments, the subject may receive one, two, three, four or more booster doses.

Such administration can be used as a chronic or acute treatment or prevention of a clinic-concerning condition. The amount of drug that may be combined with the carrier to produce a single dosage form will vary depending upon the host treated and the particular mode of administration. A typical preparation will contain from about 5% to about 95% active compound (w/w). Preferably, such preparations contain from about 20% to about 80%, 30% to about 70%, 40% to about 60%, or about 50% active compound. In other embodiments, the preparations used in the present disclosure will be about 5-10%, 10-20%, 20-30%, 30-40%, 40-50%, 50-60%, 60-70%, 70-80%, 80-90%, 90-99%, or greater than 99% of the active ingredient.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

### Delivery

In some embodiments, the delivery of the nucleic acid vaccines may be naked or formulated.

In some embodiments, the nucleic acid vaccines described herein may be delivered to a cell naked. As used herein in, “naked” refers to delivering nucleic acid vaccines free from agents which promote transfection. For example, the nucleic acid vaccines delivered to the cell may contain no modifications. The naked nucleic acid vaccines may be delivered to the cell using routes of administration known in the art and described herein.

In some embodiments, the nucleic acid vaccines described herein may be formulated, using the methods described herein. The formulations may further include, but are not limited to, cell penetration agents, a pharmaceutically acceptable carrier, a delivery agent, a bioerodible or biocompatible polymer, a solvent, and a sustained-release delivery depot. The formulated nucleic acid vaccines may be delivered to the cell using routes of administration known in the art and described herein.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Administration

In some embodiments, the nucleic acid vaccine compositions of the present disclosure may be administered by any route which results in a prophylactic or therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electroosmosis, endocervical, endosinusial, endotracheal, extracorporeal, hemodialysis, infiltration, interstitial, intraabdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique, ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them to cross the blood-brain barrier, vascular barrier, or other epithelial barrier.

Delivery of the nucleic acid vaccines described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

In some embodiments, the nucleic acid vaccines described herein may be administered intranasally similar to the administration of live vaccines. In another aspect the polynucleotide may be administered intramuscularly or intradermally similarly to the administration of inactivated vaccines known in the art.

In certain embodiments for transdermal administration, delivery across the barrier of the skin would be enhanced using electrodes (e.g., iontophoresis), electroporation, or the application of short, high-voltage electrical pulses to the skin, radiofrequencies, ultrasound (e.g. sonophoresis), microprojections (e.g. microneedles), jet injectors, thermal ablation, magnetophoresis, lasers, velocity, or photomechanical waves. The drug can be included in single-layer drug-in-adhesive, multi-layer drug-in-adhesive, reservoir, matrix, or vapor style patches, or could utilize patchless technology. Delivery across the barrier of the skin could also be enhanced using encapsulation, a skin lipid fluidizer, or a hollow or solid microstructured transdermal system (MTS, such as that manufactured by 3M), jet injectors. Additives to the formulation to aid in the passage of therapeutic compounds through the skin include prodrugs, chemicals, surfactants, cell penetrating peptides, permeation enhancers, encapsulation technologies, enzymes, enzyme inhibitors, gels, nanoparticles and peptide or protein chaperones.

Additional slow release, depot implant or injectable formulations will be apparent to the skilled artisan. See, for example, Sustained and Controlled Release Drug Delivery Systems, JR Robinson ed., Marcel Dekker Inc., New York, 1978; and Controlled Release of Biologically Active Agents, R W Baker, John Wiley & Sons, New York, 1987. The foregoing are incorporated by reference in their entirety.

Mixing of the nucleic acid vaccines described herein with a polymeric formulation comprising biodegradable polymers that can form a depot formulation upon administration, is suitable to achieve very long duration of action formulations.

When formulated for nasal administration, the absorption across the nasal mucous membrane may be further enhanced by surfactants, such as, for example, glycocholic acid, cholic acid, taurocholic acid, ethocholic acid, deoxycholic acid, chenodeoxycholic acid, dehdryocholic acid, glycodeoxycholic acid, cycledextrins and the like in an amount in the range of between about 0.1 and 15 weight percent, between about 0.5 and 4 weight percent, or about 2 weight percent. An additional class of absorption enhancers reported to exhibit greater efficacy with decreased irritation is the class of alkyl maltosides, such as tetradecylmaltoside (Arnold, J J et al., J Pharm Sci, 2004, 93: 2205-13; Ahsan, F et al., Pharm Res, 2001,18:1742-46) and references therein, all of which are hereby incorporated by reference in their entirety.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of the present disclosure may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of present disclosure may also be administered in the form of suppositories for rectal administration. These compositions can be prepared by mixing the active ingredient the present disclosure with a suitable non-irritating excipient that is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of the present disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of the present disclosure may also be topically applied to the lower intestinal tract by rectal suppository formulation or in a suitable enema formulation. Topical transdermal patches are also included in the present disclosure.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When formulated for delivery by inhalation, a number of formulations offer advantages. Adsorption of the therapeutic agents to readily dispersed solids such as diketopiperazines (for example, Technosphere particles (Pfutzner, A and Forst, T, 2005, Expert Opin Drug Deliv 2:1097-1106) or similar structures gives a formulation that results in rapid initial uptake of the therapeutic compound. Lyophilized powders, especially glassy particles, containing the therapeutic compound and an excipient are useful for delivery to the lung with good bioavailability, for example, see Exubera® (inhaled insulin, Pfizer, Inc. and Aventis Pharmaceuticals Inc.) and Afrezza® (inhaled insulin, Mannkind, Corp.).

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMO-PHOR®, alcohols, oils, modified oils, glycols, polysorbates, cyclodextrins, polymers, and/or combinations thereof.

### Injectable Forms

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the nucleic acid vaccine then depends upon its rate of dissolution which, in turn, may depend upon crystal size and crystalline form. Alternatively, delayed absorption of a parenterally administered nucleic acid vaccine may be accomplished by dissolving or suspending the nucleic acid vaccine in an oil vehicle. Injectable depot forms are made by forming microencapsule matrices of the nucleic acid vaccine in biodegradable polymers such as polylactide-polyglycolide. Depending upon the ratio of nucleic acid vaccine to polymer and the nature of the particular polymer employed, the rate of polynucleotides release can be controlled. Examples of other biodegradable polymers include, but are not limited to, poly(orthoesters) and poly(anhydrides). Depot injectable formulations may be prepared by entrapping the nucleic acid vaccine in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, e.g., by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005.

### Solid Dosage Forms: Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Properties of the Pharmaceutical Compositions

The nucleic acid vaccine pharmaceutical compositions described herein may be characterized using one or more of bioavailability, therapeutic window, volume of distribution, biological effect and detection of polynucleotides by mass spectrometry.

### Bioavailability

The nucleic acid vaccines, when formulated into a composition with a delivery agent as described herein, can exhibit an increase in bioavailability as compared to a composition lacking a delivery agent as described herein. As used herein, the term “bioavailability” refers to the systemic availability of a given amount of nucleic acid vaccines administered to a mammal. Bioavailability can be assessed by measuring the area under the curve (AUC) or the maximum serum or plasma concentration of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modem Pharmaceutics, Drugs and the Pharmaceutical Sciences, v. 72, Marcel Dekker, N.Y, Inc., 1996, herein incorporated by reference in its entirety.

The Cmax value is the maximum concentration of the compound achieved in the serum or plasma of a mammal following administration of the compound to the mammal. The Cmax value of a particular compound can be measured using methods known to those of ordinary skill in the art. The phrases “increasing bioavailability” or “improving the pharmacokinetics,” as used herein mean that the systemic availability of a first nucleic acid vaccine, measured as AUC, Cmax, or Cmin, in a mammal is greater, when co-administered with a delivery agent as described herein, than when such co-administration does not take place. In some embodiments, the bioavailability of the nucleic acid vaccines can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

In some embodiments, liquid formulations of nucleic acid vaccines may have various in vivo half-life, requiring modulation of doses to yield a prophylactic or therapeutic effect. To address this, in some embodiments, nucleic acid vaccine formulations may be designed to improve bioavailability and/or prophylactic or therapeutic effect during repeat administrations. Such formulations may enable sustained release of nucleic acid vaccines and/or reduce nucleic acid vaccine degradation rates by nucleases. In some embodiments, suspension formulations are provided comprising nucleic acid vaccines, water immiscible oil depots, surfactants and/or co-surfactants and/or co-solvents. Combinations of oils and surfactants may enable suspension formulation with nucleic acid vaccines. Delivery of nucleic acid vaccines in a water immiscible depot may be used to improve bioavailability through sustained release of polynucleotides from the depot to the surrounding physiologic environment and/or prevent polynucleotide degradation by nucleases.

In some embodiments, cationic nanoparticles comprising combinations of divalent and monovalent cations may be formulated with nucleic acid vaccines. Such nanoparticles may form spontaneously in solution over a given period (e.g. hours, days, etc.). Such nanoparticles do not form in the presence of divalent cations alone or in the presence of monovalent cations alone. The delivery of nucleic acid vaccines in cationic nanoparticles or in one or more depot comprising cationic nanoparticles may improve nucleic acid vaccine bioavailability by acting as a long-acting depot and/or reducing the rate of degradation by nucleases.

### Therapeutic Window

The nucleic acid vaccines, when formulated into a composition with a delivery agent as described herein, can exhibit an increase in the therapeutic window of the administered nucleic acid vaccine composition as compared to the therapeutic window of the administered nucleic acid vaccine composition lacking a delivery agent as described herein. As used herein “therapeutic window” refers to the range of plasma concentrations, or the range of levels of therapeutically active substance at the site of action, with a high probability of eliciting a prophylactic or therapeutic effect. In some embodiments, the therapeutic window of the nucleic acid vaccines when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

The nucleic acid vaccines, when formulated into a composition with a delivery agent as described herein, can exhibit an improved volume of distribution (Vdist), e.g., reduced or targeted, relative to a composition lacking a delivery agent as described herein. The volume of distribution (Vdist) relates the amount of the drug (e.g., nucleic acid vaccine of the present disclosure) in the body to the concentration of the drug in the blood or plasma. As used herein, the term “volume of distribution” refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, Vdist can be used to determine a loading dose to achieve a steady state concentration. In some embodiments, the volume of distribution of the nucleic acid vaccines when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In some embodiments, the biological effect of the nucleic acid vaccine delivered to the animals may be categorized by analyzing the protein expression in the animals. The protein expression may be determined from analyzing a biological sample collected from a mammal administered the nucleic acid vaccine described herein. Detection of Polynucleotides by Mass Spectrometry

Mass spectrometry (MS) is an analytical technique that can provide structural and molecular mass/concentration information on molecules after their conversion to ions. The molecules are first ionized to acquire positive or negative charges and then they travel through the mass analyzer to arrive at different areas of the detector according to their mass/charge (m/z) ratio.

Mass spectrometry is performed using a mass spectrometer which includes an ion source for ionizing the fractionated sample and creating charged molecules for further analysis. For example, ionization of the sample may be performed by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

After the sample has been ionized, the positively charged or negatively charged ions thereby created may be analyzed to determine a mass-to-charge ratio (i.e., m/z). Suitable analyzers for determining mass-to-charge ratios include quadropole analyzers, ion traps analyzers, and time-of-flight analyzers. The ions may be detected using several detection modes. For example, selected ions may be detected (i.e., using a selective ion monitoring mode (SIM)), or alternatively, ions may be detected using a scanning mode, e.g., multiple reaction monitoring (MRM) or selected reaction monitoring (SRM).

Liquid chromatography-multiple reaction monitoring (LC-MS/MRM) coupled with stable isotope labeled dilution of peptide standards has been shown to be an effective method for protein verification (e.g., Keshishian et al., Mol Cell Proteomics, 2009, 8: 2339-2349; Kuhn et al., Clin Chem 2009, 55:1108-1117; Lopez et al., Clin Chem, 2010, 56:281-290; the contents of each of which are herein incorporated by reference in their entirety). Unlike untargeted mass spectrometry frequently used in biomarker discovery studies, targeted MS methods are peptide sequence-based modes of MS that focus the full analytical capacity of the instrument on tens to hundreds of selected peptides in a complex mixture. By restricting detection and fragmentation to only those peptides derived from proteins of interest, sensitivity and reproducibility are improved dramatically compared to discovery-mode MS methods. This method of mass spectrometry based multiple reaction monitoring (MRM) quantitation of proteins can dramatically impact the discovery and quantitation of biomarkers via rapid, targeted, multiplexed protein expression profiling of clinical samples.

In some embodiments, the biological sample, once obtained from the subject, may be subjected to enzyme digestion. As used herein, the term “digest” means to break apart into shorter peptides. As used herein, the phrase “treating a sample to digest proteins” means manipulating a sample in such a way as to break down proteins in a sample. These enzymes include, but are not limited to, trypsin, endoproteinase Glu-C and chymotrypsin.

In some embodiments, a biological sample may be analyzed for protein using electrospray ionization. Electrospray ionization (ESI) mass spectrometry (ESIMS) uses electrical energy to aid in the transfer of ions from the solution to the gaseous phase before they are analyzed by mass spectrometry. Samples may be analyzed using methods known in the art (e.g., Ho et al., Cin Biochem Rev. 2003, 24(1):3-12; herein incorporated by reference in its entirety). The ionic species contained in solution may be transferred into the gas phase by dispersing a fine spray of charge droplets, evaporating the solvent and ejecting the ions from the charged droplets to generate a mist of highly charged droplets. The mist of highly charged droplets may be analyzed using at least 1, at least 2, at least 3 or at least 4 mass analyzers such as, but not limited to, a quadropole mass analyzer. Further, the mass spectrometry method may include a purification step. As a non-limiting example, the first quadrapole may be set to select a single m/z ratio so it may filter out other molecular ions having a different m/z ratio which may eliminate complicated and time-consuming sample purification procedures prior to MS analysis.

In some embodiments, a biological sample may be analyzed for protein in a tandem ESIMS system (e.g., MS/MS). As non-limiting examples, the droplets may be analyzed using a product scan (or daughter scan) a precursor scan (parent scan) a neutral loss or a multiple reaction monitoring.

In some embodiments, a biological sample may be analyzed using matrix-assisted laser desorption/ionization (MALDI) mass spectrometry (MALDIMS). MALDI provides for the nondestructive vaporization and ionization of both large and small molecules, such as proteins. In MALDI analysis, the analyte is first co-crystallized with a large molar excess of a matrix compound, which may also include, but is not limited to, an ultraviolet absorbing weak organic acid. Non-limiting examples of matrices used in MALDI are a-cyano-4-hy-droxycinnamic acid, 3,5-dimethoxy-4-hydroxycinnamic acid and 2,5-dihydroxybenzoic acid. Laser radiation of the analyte-matrix mixture may result in the vaporization of the matrix and the analyte. The laser induced desorption provides high ion yields of the intact analyte and allows for measurement of compounds with high accuracy. Samples may be analyzed using methods known in the art (e.g., Lewis, Wei and Siuzdak, Encyclopedia ofAnalytical Chemistry 2000:5880-5894; the contents of which are herein incorporated by reference in their entirety). As non-limiting examples, mass analyzers used in the MALDI analysis may include a linear time-of-flight (TOF), a TOF reflectron or a Fourier transform mass analyzer.

### Expression Systems

In some embodiments, nucleic acid vaccines described herein may be operably linked to one or more regulatory nucleotide sequences and encoded in an expression construct. Regulatory nucleotide sequences will generally be appropriate for a hosT-cell used for expression. Numerous types of appropriate expression vectors and suitable regulatory sequences are known in the art for a variety of hosT-cells. Typically, the one or more regulatory nucleotide sequences may include, but are not limited to, promoter sequences, leader or signal sequences, transcriptional start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed hosT-cells. Certain embodiments include an expression vector encoding a nucleic acid vaccine for COVID-19 sequence operably linked to at least one regulatory sequence. Regulatory sequences for use herein include promoters, enhancers, and other expression control elements. In certain embodiments, an expression vector is designed considering the choice of the hosT-cell to be transformed, the particular nucleic acid vaccine sequence to be expressed, the vector's copy number, the ability to control that copy number, or the expression of other proteins encoded by the vector, such as antibiotic markers.

In some embodiments, the nucleic acids described herein may be expressed in microorganisms. As a non-limiting example, the nucleic acid may be expressed in a bacterial system, for example, in Bacillus brevis, Bacillus megaterium, Bacillus subtilis, Caulobacter crescentus, Escherichia coli and their derivatives. Exemplary promoters include the 1-arabinose inducible araBAD promoter (PBAD), the lac promoter, the 1-rhamnose inducible rhaP BAD promoter, the T7 RNA polymerase promoter, the trc and tac promoter, the lambda phage promoter Pl, and the anhydrotetracycline-inducible tetA promoter/operator.

In some embodiments, the nucleic acids described herein may be expressed in a yeast expression system. Non-limiting examples of promoters which may be used in yeast vectors include the promoters for 3-phosphoglycerate kinase (Hitzeman et al., J. Biol. Chem. 255:2073 (1980)); other glycolytic enzymes (Hess et al., J. Adv. Enzyme Res. 7:149 (1968); Holland et al., Biochemistry 17:4900 (1978). Others promoters are from, e.g., enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, glucokinase alcohol oxidase I (AOX1), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Certain yeast expression systems are commercially available, for example, from Clontech Laboratories, Inc. (Palo Alto, Calif., e.g., Pyex 4T family of vectors for S. cerevisiae), Invitrogen (Carlsbad, Calif, e.g. Ppicz series Easy Select Pichia Expression Kit) and Stratagene (La Jolla, Calif., e.g. ESP™ Yeast Protein Expression and Purification System for S. pombe and Pesc vectors for S. cerevisiae).

In some embodiments, the nucleic acids described herein may be expressed in mammalian expression systems. Non-limiting examples of mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-1 promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In a specific embodiment, a yeast alcohol oxidase promoter is used.

In some embodiments, promoters for use in mammalian hosT-cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

In some embodiments, the nucleic acids described herein may be expressed in insecT-cell expression systems. Eukaryotic expression systems employing insecT-cell hosts may rely on either plasmid or baculoviral expression systems. Typical insect hosT-cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedron promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silkworm (Bombyx mori). Numerous baculovirus expression systems are commercially available, for example, from Thermo Fisher (Bac-N-Blue™ k or BAC-TO-BAC™ Systems), Clontech (BacPAK™ Baculovirus Expression System), Novagen (Bac Vector System™), or others from Pharmingen or Quantum Biotechnologies. Another insecT-cell host is the common fruit fly, Drosophila melanogaster, for which a transient or stable plasmid-based transfection kit is offered commercially by Thermo Fisher (The DES™ System).

In some embodiments, cells are transformed with vectors that express a nucleic acid described herein. Transformation techniques for inserting new genetic material into eukaryotic cells, including animal and planT-cells, are well known. Viral vectors may be used for inserting expression cassettes into hosT-cell genomes. Alternatively, the vectors may be transfected into the hosT-cells. Transfection may be accomplished by methods as described in the art such as, but not limited to, calcium phosphate precipitation, electroporation, optical transfection, protoplast fusion, impalefection, and hydrodynamic delivery.

### IV. Methods of Use

One aspect of the present disclosure provides methods of using nucleic acid vaccines of the present disclosure and pharmaceutical compositions and formulations comprising the nucleic acid vaccines and at least one pharmaceutically acceptable carrier. Provided herein are compositions, methods, kits, and reagents for diagnosis, treatment, alleviation or prevention of a disease or condition in humans or other mammals where the active agent is the nucleic acid vaccine, cells containing the nucleic acid vaccine or polypeptides translated from nucleic acid vaccine polynucleotides.

In some embodiments, the methods of use can be assessed using any endpoint indicating a benefit to the subject, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

### Therapeutic or Prophylactic Uses

The nucleic acid vaccines described herein may be used to protect, treat or cure infection arising from contact with an infectious agent such as, but not limited to, viruses, bacteria, fungi, parasites and protozoa. As a non-limiting example, the infectious agent is a virus and the virus is SARS-CoV-2 and/or a variant thereof. In some embodiments, the variants of SARS-CoV-2 are VOI, VOC and VOHC variants.

The nucleic acid vaccines described herein may be used as prophylactic agents where the nucleic acid vaccines are administered to a subject, and wherein the nucleic acid vaccine polynucleotide is translated in vivo to produce one or more proteins, peptides, fragments or variants thereof of SARS-CoV-2 for the prevention of COVID-19.

The nucleic acid vaccines described herein may be used as therapeutic agents where the nucleic acid vaccines are administered to a subject, and wherein the nucleic acid vaccine polynucleotide is translated in vivo to produce one or more proteins, peptides, fragments or variants thereof of SARS-CoV-2 for the alleviation of one or more symptoms of COVID-19.

In some embodiments, provided are methods for treating or preventing a viral infection and/or a disease, disorder, or condition associate with a viral infection or a symptom thereof, in a subject, by administering a nucleic acid vaccine comprising one or more polynucleotides encoding a viral polypeptide. The administration may be in combination with an anti-viral or anti-bacterial agent or a small molecule compound described herein or known in the art.

In some embodiments, the nucleic acid vaccines described herein may be used to protect against and/or prevent the transmission of an emerging or engineered threat which may be known or unknown.

In some embodiments, provided herein are methods of inducing translation of a polypeptide (e.g., one or more proteins, peptides, fragments or variants thereof of SARS-CoV-2 in a cell, tissue or organism using the nucleic acid polynucleotides described herein. The translated polypeptide may be used for the prevention, alleviation and/or treatment of COVID-19. Such translation can be in vitro, in vivo, ex vivo, or in culture. The cell, tissue or organism may be contacted with an effective amount of a composition or pharmaceutical composition containing the nucleic acid vaccine which includes a polynucleotide with at least one region encoding the polypeptide of interest (e.g., one or more proteins, polypeptides, peptides, fragments or variants thereof of SARS-CoV-2 for the treatment and/or prevention of COVID-19.

In some embodiments, the effective amount of the nucleic acid vaccine described herein provided to a cell, a tissue or a subject may be enough for immune prophylaxis.

An “effective amount” of the composition of the nucleic acid vaccine is provided based, at least in part, on the target tissue, targeT-cell type, means of administration, physical characteristics of the polynucleotide (e.g., size, and the number of unmodified and modified nucleosides) and other components of the nucleic acid vaccine. An effective amount of the composition containing the nucleic acid vaccine described herein is one that provides an induced or boosted immune response as a function of production in the cell of one or more proteins, polypeptides, peptides, fragments or variants thereof of SARS-CoV-2 as compared to an untreated cell. Increased production may be demonstrated by increased cell transfection (i.e., the percentage of cells transfected with the nucleic acid vaccine), increased protein translation from the polynucleotide or altered innate immune response of the hosT-cell.

Provided herein are directed to methods of inducing in vivo translation of one or more proteins, polypeptides, peptides, fragments or variants thereof of SARS-CoV-2 in a mammalian subject in need thereof. An effective amount of a nucleic acid vaccine composition containing a polynucleotide that has at least one translatable region encoding the polypeptide (e.g., one or more proteins, polypeptides, peptides, fragments or variants thereof of SARS-CoV-2) is administered to the subject using the delivery methods described herein. The polynucleotide is provided in an amount and under other conditions such that the polynucleotide is translated in the cell. The cell in which the polynucleotide is localized, or the tissue in which the cell is present, may be targeted with one or more rounds of nucleic acid vaccine administration.

In certain embodiments, the administered nucleic acid vaccine comprising polynucleotides directs production of one or more polypeptides that provide a functional immune system-related activity which is substantially absent in the cell, tissue or organism in which the polypeptide is translated. For example, the missing functional activity may be enzymatic, structural, or gene regulatory in nature. In related embodiments, the administered polynucleotides direct production of one or more polypeptides that increases a functional activity related to the immune system which is present but substantially deficient in the cell in which the polypeptide is translated.

Additionally, the polypeptide translated from the nucleic acid vaccine may antagonize, directly or indirectly, the activity of a biological moiety present in, on the surface of, or secreted from the cell. Non-limiting examples of biological moieties that may be antagonized include a nucleic acid, a carbohydrate, a protein toxin such as shiga and tetanus toxins, lipids (e.g., cholesterol), a lipoprotein (e.g., low density lipoprotein), or a small molecule toxin (e.g., cholera, botulinum, and diphtheria toxins). In some embodiments, the biological molecule which may be antagonized may be an endogenous protein that may have an undesirable activity such as, but not limited to, cytotoxic or cytostatic activity. The proteins described herein may be engineered for localization within the cell, potentially within a specific compartment such as the cytoplasm or nucleus, or are engineered for secretion from the cell or translocation to the plasma membrane of the cell.

In some embodiments, the polynucleotides of the nucleic acid vaccines and their encoded polypeptides may be used for treatment of any of a variety of diseases, disorders, and/or conditions, including but not limited to viral infections (e.g., infections caused by SARS-CoV-2 and/or a variant thereof).

The subject to whom the nucleic acid vaccine may be administered suffers from or may be at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

The agents (e.g., compositions of nucleic acid vaccines and any additional moieties) can be administered simultaneously, for example in a combined unit dose (e.g., providing simultaneous delivery of both agents). The agents can also be administered at a specified time interval, such as, but not limited to, an interval of minutes, hours, days or weeks. Generally, the agents may be concurrently bioavailable, e.g., detectable, in the subject. In some embodiments, the agents may be administered essentially simultaneously, for example two unit dosages administered at the same time, or a combined unit dosage of the two agents. In other embodiments, the agents may be delivered in separate unit dosages. The agents may be administered in any order, or as one or more preparations that includes two or more agents. In a preferred embodiment, at least one administration of one of the agents, e.g., the first agent, may be made within minutes, one, two, three, or four hours, or even within one or two days of the other agent, e.g., the second agent. In some embodiments, combinations can achieve synergistic results, e.g., greater than additive results, e.g., at least 25, 50, 75, 100, 200, 300, 400, or 500% greater than additive results.

In some embodiments, the nucleic acid vaccine described herein may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 7 days, 8 days, 9 days, 10 days, 14 days, 21 days, 28 days, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some embodiments, the nucleic acid vaccines may be formulated by the methods described herein. In one aspect, the formulation may comprise a nucleic acid vaccine or polynucleotide which can have a therapeutic and/or prophylactic effect on more than one disease, disorder or condition. As a non-limiting example, the formulation may comprise polynucleotides encoding one or more proteins, polypeptide, peptides, fragments or variants thereof of SARS-CoV-2 for the treatment and/or prevention of COVID-19.

In some embodiments, the nucleic acid vaccines described herein may be used for research in many applications, such as, but not limited to, identifying and locating intracellular and extracellular proteins, protein interaction, signal pathways and cell biology.

### Modulation of the Immune Response

In some embodiments, the nucleic acid vaccines comprising the polynucleotides described herein may act as a single composition as a vaccine. As used herein, a “vaccine” refers to a composition, a substance or preparation that stimulates, induces, causes or improves immunity in an organism, e.g., an animal organism, for example, a mammalian organism (e.g., a human). Preferably, a vaccine provides immunity against one or more diseases or disorders in the organism, including prophylactic and/or therapeutic immunity. Exemplary vaccines include one or more agents that resembles an infectious agent, e.g., a disease-causing microorganism, and can be made, for example, from live, attenuated, modified, weakened or killed forms of disease-causing microorganisms, or antigens derived therefrom, including combinations of antigenic components. In exemplary embodiments, a vaccine stimulates, induces causes or improves immunity in an organism or causes or mimics infection in the organism without inducing any disease or disorder. A vaccine introduces an antigen into the tissues, extracellular space or cells of a subject and elicits an immune response, thereby protecting the subject from a particular disease or pathogen infection. The nucleic acid vaccines described herein may encode an antigen and when the polynucleotides are expressed in cells, a desired immune response is achieved. As a non-limiting example, the nucleic acid vaccines described herein may encode one or more proteins, polypeptides, peptides, fragments or variants thereof of SARS-CoV-2 and when the polynucleotides are expressed in cells, a desired immune response against SARS-CoV-2 is achieved to treat and/or prevent COVID-19.

Nucleic acid vaccines may be administered prophylactically or therapeutically as part of an active immunization scheme to healthy individuals or early in infection during the incubation phase or during active infection after onset of symptoms.

The nucleic acid vaccines described herein may also be administered as a second line treatment after the standard first line treatments such as antibiotics and antivirals have failed to induce passive immunity. In this regard, the nucleic acid vaccines described herein are useful in settings where resistance to first line treatments has developed and disease persists and induces chronic disease.

Nucleic acid vaccines may be administered as part of a treatment regimen for latent viral infections, such as SARS-CoV-2 infections. In this embodiment, one or more polynucleotides are administered which ultimately produce proteins which result a desired immune response against SARS-CoV-2 is achieved to treat and/or prevent COVID-19.

The use of RNA in or as a vaccine overcomes the disadvantages of conventional genetic vaccination involving incorporating DNA into cells in terms of safeness, feasibility, applicability, and effectiveness to generate immune responses. RNA molecules are considered to be significantly safer than DNA vaccines, as RNAs are more easily degraded. They are cleared quickly out of the organism and cannot integrate into the genome and influence the cell's gene expression in an uncontrollable manner. It is also less likely for RNA vaccines to cause severe side effects like the generation of autoimmune disease or anti-DNA antibodies (Bringmann A. et al., Journal of Biomedicine and Biotechnology (2010), vol. 2010, article ID623687). Transfection with RNA requires only insertion into the cell's cytoplasm, which is easier to achieve than into the nucleus. However, RNA is susceptible to RNase degradation and other natural decomposition in the cytoplasm of cells.

Various attempts to increase the stability and shelf life of RNA vaccines. US Pub. No. US 20050032730 to Von Der Mulbe et al. discloses improving the stability of mRNA vaccine compositions by increasing G(guanosine)/C(cytosine) content of the mRNA molecules. U.S. Pat. No. 5,580,859 to Feigner et al. teaches incorporating polynucleotide sequences coding for regulatory proteins that binds to and regulates the stabilities of mRNA. While not wishing to be bound by theory, it is believed that the nucleic acid vaccines described herein may result in improved stability and therapeutic efficacy due at least in part to the specificity, purity and selectivity of the construct designs. Additionally, modified nucleosides, or combinations thereof, may be introduced into the nucleic acid vaccines described herein to activate the innate immune response. Such activating molecules are useful as adjuvants when combined with polypeptides and/or other vaccines. In certain embodiments, the activating molecules contain a translatable region which encodes for a polypeptide sequence useful as a vaccine, thus providing the ability to be a self-adjuvant.

In some embodiments, the nucleic acid vaccines described herein may be used in the prevention, treatment and diagnosis of diseases and physical disturbances caused by infectious agents such as, but not limited to, SARS-CoV-2, or a VOC, VOI or VOHC of SARS-CovV-2. The nucleic acid vaccines described herein may encode at least one polypeptide of interest (e.g., one or more proteins, polypeptides, peptides, fragments or variants thereof of SARS-CoV-2) and may be provided to an individual in order to stimulate the immune system to protect against the disease-causing agents. As a non-limiting example, the biological activity and/or effect from an infectious agent may be inhibited and/or abolished by providing neutralizing antibodies which have the ability to bind and neutralize the infectious agent; the neutralizing antibodies produced from the immune system stimulated by the polypeptides translated from the nucleic acid vaccine.

As a non-limiting example, the polynucleotides encoding an immunogen may be delivered to cells to trigger multiple innate response pathways (see PCT Patent Application Publication Nos. WO2012006377 and US Patent Publication No. US20130177639; the contents of each of which are herein incorporated by reference in their entirety). As another non-limiting example, the nucleic acid vaccines described herein may be delivered to a vertebrate in a dose amount large enough to be immunogenic to the vertebrate (see PCT Patent Application Publication Nos. WO2012006372 and WO2012006369 and US Publication Nos. US20130149375 and US20130177640; the contents of each of which are herein incorporated by reference in their entirety).

In some embodiments, the nucleic acid vaccines described herein may be delivered to a mammal (e.g., human) in a dose amount large enough to be immunogenic for stimulating an immune response in the mammal. The immune response can defend a viral infection, thereby, prevent and/or treat a disease. As a non-limiting example, the nucleic acid vaccines described herein may treat and/or prevent infectious diseases including viral infectious diseases such as COVID-19 caused by SARS-CoV-2, or a VOC, VOI or VOHC of SARS-CoV-2.

Nucleic acid vaccines described herein may be utilized in various settings depending on the prevalence of the infection or the degree or level of unmet medical need. As a non-limiting example, the nucleic acid vaccines described herein may be utilized to treat and/or prevent COVID-19 infection, including the diseases and conditions related to COVID-19 infection (including infection by the original and mutated versions of SARS-CoV-2).

Symptoms of COVID-19 infection are changing as more is learned about the disease but the current symptoms include fever or chills, cough, shortness of breath or difficulty breathing, fatigue, body aches, muscle aches, headaches, sore throat, congestion or runny nose, nausea and/or vomiting, diarrhea, and a new loss of taste or smell.

In some embodiments, the nucleic acid vaccines described herein may be better designed, as compared to current anti-viral treatments, to produce the appropriate protein conformation on translation as the nucleic acid vaccines co-opt natural cellular machinery. Unlike traditional vaccines which are manufactured ex vivo and may trigger unwanted cellular responses, the nucleic acid vaccines are presented to the cellular system in a more native fashion. In some embodiments, the nucleic acid vaccines described herein are a tailored active vaccine for COVID-19 that not only can prevent infection by SARS-CoV-2 but can limit transmission of SARS-CoV-2.

In some embodiments, the nucleic acid vaccines described herein may be used to prevent pandemic COVID-19 by reacting to emerging new strains with the very rapid nucleic acid based vaccine production process.

In some embodiments, a single injection of a nucleic acid vaccine may provide protection for an entire season.

In some embodiments, the nucleic acid vaccines described herein may be immunostimulatory. The polynucleotide sequence of the nucleic acid vaccine may further comprise a sequence region encoding a cytokine that promotes the immune response, such as a monokine, lymphokine, interleukin or chemokine, such as IL-1, IL-2, IL-3, IL-4, IL-5, IL-6, IL-7, IL-8, IL-9, IL-10, IL-12, INF-a, INF-γ, GM-CFS, LT-a, or growth factors such as hGH.

Treatment and/or Prevention of COVID-19

In some embodiments, the nucleic acid vaccines described herein encode one or more proteins, polypeptides, peptides, fragments or variants thereof of SARS-CoV-2 and may be used for the treatment and/or prevention of COVID-19

In some embodiments, the nucleic acid vaccines described herein can produce much higher neutralizing antibody titers and they may produce responses early than commercially available anti-virals. As a non-limiting example, the nucleic acid vaccines described herein can produce 10 times, or 9×, or 8×, or 7×, or 6×, or 5×, or 4×, or 3× more neutralizing antibody titers than other vaccines.

In some embodiments, the nucleic acid vaccines described herein co-opt the natural cellular machinery to produce the appropriate protein conformation on translation. Unlike traditional vaccines which are manufactured ex vivo and may trigger unwanted cellular responses, the nucleic acid vaccines described herein are introduced to the cellular system in a way that is closer to the native way or the way normal cellular processing occurs. Additionally, formulations may be used to shield or target delivery of the nucleic acid vaccines to specific cells or tissues in the subject.

In some embodiments, nucleic acid vaccines described herein represent a targeted active vaccine that not only can prevent infection but can limit transmission of COVID-19.

In some embodiments, the nucleic acid vaccines may be used to prevent pandemic SARS-CoV-2 infection or COVID-19 by reacting to emerging new strains with the very rapid vaccine production process.

In some embodiments a single injection of nucleic acid vaccines encoding one or more proteins, polypeptides, peptides, fragments or variants thereof of SARS-CoV-2 may provide protection for at least 6 months, at least 1 year, at least 2 years, at least 3 years, at least 4 years, at least 5 years, at least 6 years, at least 7 years, at least 8 years, at least 9 years, at least 10 years, at least 11 years, at least 12 years, at least 13 years, at least 15 years or more than 15 years.

The nucleic acid vaccines described herein may also be used to maintain or restore antigenic memory in a subject or population as part of a vaccination plan for COVID-19 or other diseases caused by SARS-CoV-2.

In some embodiments, nucleic acid vaccines compositions may be created which include polynucleotides that encode one or more proteins, polypeptides, peptides, fragments or variants thereof of SARS-CoV-2 which are showing prevalence increased infection rates for the year. The protein sequences of SARS-CoV-2 have been shown to change or mutate over time, wherein some of the mutations have shown increased infection rates. As a non-limiting example, the nucleic acid vaccines compositions may be created which include polynucleotides that encode one or more proteins, polypeptides, peptides, fragments or variants thereof of SARS-CoV-2 which are showing prevalence increased infection rates for the year such as, but not limited to the D614G mutation in the spike protein.

In some embodiments, the nucleic acid vaccines may be used to induce neutralizing antibodies in a subject. The neutralization activity of the neutralizing antibodies induced by the present nucleic acid vaccines may correlate to the resulting effectiveness (e.g., immune protection) of the vaccines described herein. In some aspects, the immune protection provided by the present nucleic acid vaccines may be greater than the immune protection provided in convalescent subjects. The induced neutralization activity from the present nucleic acid vaccines may increase the recovery rate of those exposed to SARS-CoV-2 or whom are in convalescence. As compared to the other mRNA vaccines available such as BNT162b2 and mRNA-1273, the present nucleic acid vaccines induce higher neutralizing antibody titers in treated subjects. In some embodiments, the nucleic acid vaccines include at least 10 times, 9 times, 8 times, 7 times, 6 times, 5 times or 4 times more neutralizing antibody titers than other vaccines for COVID-19.

In some embodiments, a vaccination scheme or plan is developed which allows for not only ongoing vaccination in the current year but memory booster vaccinations across years, strains, or groups thereof to establish and maintain memory in a population. In this manner, a population is less likely to succumb to any pandemic or outbreak involving recurrence of older strains. Any combination of a prior vaccine component strain can be utilized to create or design a memory booster vaccine.

In some embodiments, nucleic acid vaccines which are memory booster vaccines are administered to boost memory across a time period of 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more than 50 years.

In some embodiments, nucleic acid vaccines which are memory booster vaccines are administered to boost memory for alternating historic years including every other year from the past vaccine component strains relative to a current year. In some embodiments the selection of the vaccine components can be from every 2nd, 3rd, 4th, 5th, 6th, 7th, 8th, 9th, 10th or more years.

In some embodiments, nucleic acid vaccines which are memory booster vaccines are administered to boost memory over ten-year periods.

In some embodiments, the nucleic acid booster vaccine may be used in a population either once or periodically to create herd immunity which means greater than 30% of a population is protected.

In some embodiments, the nucleic acid booster vaccine may be used in a population either once or periodically to create herd immunity against COVID-19 which means greater than 30% of a population is protected.

In some embodiments, the nucleic acid vaccines are used to target at risk populations for COVID-19 such as those having pre-existing conditions including, but not limited to, cancer, chronic kidney disease, chronic obstructive pulmonary disease (COPD), immunocompromised state (weakened immune system) from solid organ transplant, blood or bone marrow transplant, immune deficiencies, HIV, and use of corticosteroids or other immune weaking medicines, obesity (body mass index (BMI) of 30 or higher), heart conditions such as heart failure, coronary artery disease, or cardiomyopathies, sickle cell disease, type 1 or type 2 diabetes mellitus, asthma (moderate-to-severe), cerebrovascular disease, cystic fibrosis, hypertension or high blood pressure, neurological conditions such as dementia, liver disease, pregnancy, pulmonary fibrosis, smoking, and thalassemia.

In some embodiments, the nucleic acid vaccines are used to protect healthcare workers who are at risk of being exposed to SARS-CoV-2.

As a non-limiting example, the nucleic acid vaccine of the present disclosure comprises a LNP formulated polynucleotide encoding the full-length S protein with D614G (SEQ ID NO: 27) (as referred to as “PTX-B”). Methods for use of PTX-B vaccine to induce a protective immune response in a subject is provided. The protective immune response can protect a subject against a viral infection, such as infection by SAR-CoV-2 original strain and its variant thereof. The SARS-CoV-2 variant can be any VOC, VOI and/or VOHC strain. As non-limiting examples, the variant is an Alpha variant, a Beta variant, or a Delta variant.

In some embodiments, the nucleic acid vaccine PTX-B is used to protect against an Alpha variant.

In some embodiments, the nucleic acid vaccine PTX-B is used to protect against a Beta variant.

In some embodiments, the nucleic acid vaccine PTX-B is used to protect against a Delta variant.

In some embodiments, the nucleic acid vaccine PTX-B is used to induce neutralizing antibodies in a subject. The nucleic acid vaccine PTX-B induces antibodies with high neutralization titers. For example, the neutralization titers of induced antibodies are as least 100 fold, 90 fold, 80 fold, 70 fold, 60 fold, 50 fold, 40 fold, 30 fold, 25 fold, 20 fold, 15 fold, 10 fold or 5 fold of the titers in sera from infected subjects or convalescent subjects.

In some embodiments, a dosing regimen of the nucleic acid vaccine PTX-B is provided. The dose of PTX-B ranges from 1 μg to 500 μg, from 1 μg to 1 mg, from 1 mg to 10 mg, from 1 mg to 100 mg, or from 10 mg to 100 mg/kg of a subject body weight. In some examples, a dose of PTX-B can achieve a dose level of about 10 μg, 20 μg, 25 μg, 30 μg, 35 μg, 40 μg, 45 μg, 50 μg, 60 μg, 70 μg, 80 μg, 90 μg, or 100 μg of the nucleic acid vaccine. At least one dose of the nucleic acid vaccine PTX-B is administered. In some embodiments, at least one booster dose of PTX-B is administered. The booster dose may be administered to the subject, one month, two months, three months, four months, six months, or one year or greater after the subject receives the first dose of PTX-B In some instances, more than one booster dose, e.g., two, three, four or more, is administered to the subject.

In another embodiment, the nucleic acid vaccine of the present disclosure comprises a LNP formulated polynucleotide encoding the full-length S protein of SARS-CoV-2 variant B.1.351 (South African variant (SEQ ID NO: 43).

### V. Kits and Devices

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

In some embodiments, the present disclosure provides kits for modulating the expression of genes in vitro or in vivo, comprising nucleic acid vaccine compositions of the present disclosure or a combination of nucleic acid vaccine compositions of the present disclosure, nucleic acid vaccine compositions modulating other genes, siRNAs, miRNAs or other oligonucleotide molecules.

The kit may further comprise packaging and instructions and/or a delivery agent to form a formulation, e.g., for administration to a subject in need of treatment using the nucleic acid vaccine compositions described herein. The delivery agent may comprise a saline, a buffered solution, a lipidoid, a dendrimer or any suitable delivery agent.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated or it may be lyophilized. The amount of each component may be varied to enable consistent, reproducible higher concentration saline or simple buffer formulations. The components may also be varied in order to increase the stability of nucleic acid vaccine compositions in the buffer solution over a period of time and/or under a variety of conditions.

### Devices

The present disclosure provides for devices which may incorporate nucleic acid vaccine compositions of the present disclosure. These devices can contain a stable formulation available to be immediately delivered to a subject in need thereof, such as a human patient.

Non-limiting examples of the devices include a pump, a catheter, a needle, a transdermal patch, a pressurized olfactory delivery device, electroporation devices, iontophoresis devices, multi-layered microfluidic devices. The devices may be employed to deliver nucleic acid vaccine compositions of the present disclosure according to single, multi- or split-dosing regiments. The devices may be employed to deliver nucleic acid vaccine compositions of the present disclosure across biological tissue, intradermal, subcutaneously, or intramuscularly. More examples of devices suitable for delivering oligonucleotides are disclosed in International Publication WO 2013/090648, the contents of which are incorporated herein by reference in their entirety.

### VI. Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means+/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Adjuvant: As used herein, the term “adjuvant” means a substance that enhances a subject's immune response to an antigen. The nucleic acid vaccines described herein may optionally comprise one or more adjuvants.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigen: As defined herein, the term “antigen” refers to a composition, for example, a substance or agent which causes an immune response in an organism, e.g., causes the immune response of the organism to produce antibodies against the substance or agent in particular, which provokes an adaptive immune response in an organism. Antigens can be any immunogenic substance including, in particular, proteins, polypeptides, polysaccharides, nucleic acids, lipids and the like. Exemplary antigens are derived from infectious agents. Such agents can include parts or subunits of infectious agents, for example, coats, coat components, e.g., coat protein or polypeptides, surface components, e.g., surface proteins or polypeptides, capsule components, cell wall components, flagella, fimbrae, and/or toxins or toxoids) of infectious agents, for example, bacteria, viruses, and other microorganisms. Certain antigens, for example, lipids and/or nucleic acids are antigenic, preferably, when combined with proteins and/or polysaccharides.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide described herein may be considered biologically active if even a portion of the polynucleotides is biologically active or mimics an activity considered biologically relevant.

Chimera: As used herein, “chimera” is an entity having two or more incongruous or heterogeneous parts or regions.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Encoded protein cleavage signal: As used herein, “encoded protein cleavage signal” refers to the nucleotide sequence which encodes a protein cleavage signal.

Engineered: As used herein, embodiments of the nucleic acid vaccines are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Exosome: As used herein, “exosome” is a vesicle secreted by mammalian cells or a complex involved in RNA degradation.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a polynucleotide of a nucleic acid vaccine and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules.

Calculation of the percent identity of two polynucleotide sequences, for example, can be performed by aligning the two sequences for optimal comparison purposes (e.g., gaps can be introduced in one or both of a first and a second nucleic acid sequences for optimal alignment and nonidentical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M, ed., Oxford University Press, N.Y., 1988; Biocomputing: Informatics and Genome Projects, Smith, D. W., ed., Academic Press, N.Y, 1993; Sequence Analysis in Molecular Biology, von Heinje, G., Academic Press, 1987; Computer Analysis of Sequence Data, Part I, Griffin, A. M, and Griffin, H. G., eds., Humana Press, N.J., 1994; and Sequence Analysis Primer, Gribskov, M. and Devereux, J., eds., M Stockton Press, N.Y, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al, J. Molec. Biol., 215, 403 (1990)).

Infectious Agent: As used herein, the phrase “infectious agent” means an agent capable of producing an infection in an organism, for example, in an animal. An infectious agent may refer to any microorganism, virus, infectious substance, or biological product that may be engineered as a result of biotechnology, or any naturally occurring or bioengineered component of any such microorganism, virus, infectious substance, or biological product, can cause emerging and contagious disease, death or other biological malfunction in a human, an animal, a plant or another living organism.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence.

Modified: As used herein “modified” refers to a changed state or structure of a molecule described herein. Molecules may be modified in many ways including chemically, structurally, and functionally.

Mucus: As used herein, “mucus” refers to the natural substance that is viscous and comprises mucin glycoproteins.

Naturally occurring: As used herein, “naturally occurring” means existing in nature without artificial aid.

Neutralizing antibody: As used herein, a “neutralizing antibody” refers to an antibody which binds to its antigen and defends a cell from an antigen or infectious agent by neutralizing or abolishing any biological activity it has.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid Vaccine: As used herein, “nucleic acid vaccine” refers to a vaccine or vaccine composition which includes a nucleic acid or nucleic acid molecule (e.g., a polynucleotide) encoding an antigen (e.g., an antigenic protein or polypeptide.) In exemplary embodiments, a nucleic acid vaccine includes a ribonucleic (“RNA”) polynucleotide, ribonucleic acid (“RNA”) or ribonucleic acid (“RNA”) molecule. Such embodiments can be referred to as ribonucleic acid (“RNA”) vaccines.

Off-target: As used herein, “off target” refers to any unintended effect on any one or more target, gene, or cellular transcript.

Open reading frame: As used herein, the term “open reading frame” or “ORF” refers to a continuous polynucleotide sequence, for example, a DNA sequence or RNA sequence (e.g., an mRNA sequence), comprising a start codon, a subsequent region comprising a plurality of amino acid-encoding codons, and a terminal stop codon, wherein the region comprising the plurality of amino acid-encoding codons contains no stop codons.

Operably linked: As used herein, the phrase “operably linked” refers to a functional connection between two or more molecules, constructs, transcripts, entities, moieties or the like.

Part: As used herein, a “part” or “region” of a polynucleotide is defined as any portion of the polynucleotide which is less than the entire length of the polynucleotide.

Peptide: As used herein, “peptide” is less than or equal to 50 amino acids long, e.g., about 5, 10, 15, 20, 25, 30, 35, 40, 45, or 50 amino acids long.

Paratope: As used herein, a “paratope” refers to the antigen-binding site of an antibody.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (com), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Beige et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound described herein wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidi-none (NMP), dimethyl sulfoxide (DMSO), N,N′-dimethyl-formamide (DMF), N,N′-dimethylacetamide (DMAC), 1,3-dimethyl-2-imidazolidinone (DMEU), 1,3-dimethyl-3,4,5, 6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Polypeptide per unit drug (PUD): As used herein, a PUD or product per unit drug, is defined as a subdivided portion of total daily dose, usually 1 mg, pg, kg, etc., of a product (such as a polypeptide) as measured in body fluid or tissue, usually defined in concentration such as pmol/mL, mmol/mL, etc. divided by the measure in the body fluid.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Purified: As used herein, “purify,” “purified,” “purification” means to make substantially pure or clear from unwanted components, material defilement, admixture or imperfection.

Repeated transfection: As used herein, the term “repeated transfection” refers to transfection of the same cell culture with a polynucleotide a plurality of times. The cell culture can be transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition may be administered, e.g., for experimental, diagnostic, prophylactic, and/or therapeutic purposes. Typical subjects include animals (e.g., mammals such as mice, rats, rabbits, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules described herein may be chemical or enzymatic.

Vaccine: As used herein, a vaccine is a compound or composition which comprises at least one polynucleotide encoding at least one antigen.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr. period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Translation: As used herein “translation” is the process by which a polynucleotide molecule is processed by a ribosome or ribosomal-like machinery, e.g., cellular or artificial, to produce a peptide or polypeptide.

Transcription: As used herein “transcription” is the process by which a polynucleotide molecule is processed by a polymerase or other enzyme to produce a polynucleotide, e.g., an RNA polynucleotide.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. Treatment may be administered to a subject who does not exhibit signs of a disease, infection, disorder, and/or condition and/or to a subject who exhibits only early signs of a disease, infection, disorder, and/or condition for the purpose of decreasing the risk of developing pathology associated with the disease, infection, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vaccine: As used herein, the phrase “vaccine” refers to a biological preparation that improves immunity in the context of a particular disease, disorder or condition.

Viral protein: As used herein, the phrase “viral protein” means any protein originating from a virus.

### EXAMPLES

### Example 1. In Vivo Study of LNP Formulated mRNA

Five (5) groups of female C57bl/6 mice, 8 mice/group (6 weeks old), were administered formulations as described in Table 6. On day 0 and 21 the mice were bled before receiving 20 micrograms (“μg”) of one of the formulations in Table 6 via intramuscular administration on day 1 and 22. On day 43 the mice were euthanized, and blood was collected by cardiac puncture, the spleen was harvested and splenocytes isolated.

TABLE 6
Formulation Table

| Group | Description of Formulation |
|---|---|
| 1 | LNP formulated TdTomato mRNA negative control |
| | (SEQ ID NO: 46) |
| 2 | LNP formulated S protein receptor binding domain |
| | (RBD) mRNA (SEQ ID NO: 25) |
| 3 | LNP formulated full-length S protein mRNA (“PTX-B”) |
| | (SEQ ID NO: 27) |
| 4 | LNP formulated full-length S protein with mutated furin site |
| | mRNA (SEQ ID NO: 26) |
| 5 | Dulbecco's Phosphate-Buffered Saline (DPBS) control |

A clinical isolate virus neutralization assay was performed on the pooled samples from the live bleed from day 21. At termination (Day 43), sera antibody binding to SARS-CoV-2 RBD and S protein, pseudovirus neutralization, clinical isolate virus neutralization and T-cell response (determined by enzyme-linked immunospot (ELIspot) and flow cytometry) were measured.

There were no apparent adverse reactions from the mice. Data from the initial preliminary bleed showed that all three constructs had neutralizing activity. Group 3 formulation (full-length S protein mRNA) was the best, followed closely by the Group 4 formulation (full-length S protein with mutated furin site); the Group 2 formulation (S protein RBD domain) was the lowest performer; and no activity was seen in either negative control treated groups.

Splenocytes were stimulated with SARS-CoV antigens (RBD peptide pool plus S protein), and antigen-specific T-cell responses were measured by counting IFN-γ secreting T-cells in ELISpot, or Th1 cytokine (IFN-γ/TNF-α/IL-2) and Th2 cytokine (IL-4/IL-5) producing T-cells in flow cytometry. Th1 cytokine (IFN-γ/TNF-α/IL-2) and Th2 cytokine (IL-4/IL-5) were also measured in the supernatant of the SARS-CoV antigen-stimulated T-cells by a multiplex mouse cytokine assay.

### ELISpot Assay

The ELISpot assay demonstrated that the splenocytes from the mice treated with Group 2, Group 3 and Group 4 formulations produced T-cells that were secreting IFN-γ in response to a peptide pool which contained overlapping peptides within the RBD and conserved S2 regions of the S protein. The responses in Group 2, Group 3, and Group 4 were similar and neither of the two control groups showed a significant response to the peptide pool. Flow cytometry assay did not detect a significant Th1 or Th2 responses in the three groups as compared to the two control groups.

A direct binding ELISA was used to determine if any antibodies were elicited to bind to the S protein. Either the RBD domain or the full-length S protein was bound to plates and different dilutions of sera from the treated mice were incubated in the plates before being washed and detected by an anti-mouse antibody. Sera from Group 2, Group 3 and Group 4 were positive for antibodies that could bind both the RBD and the full-length S protein and the controls were negative for binding.

### SARS-CoV-2 Neutralization Assay

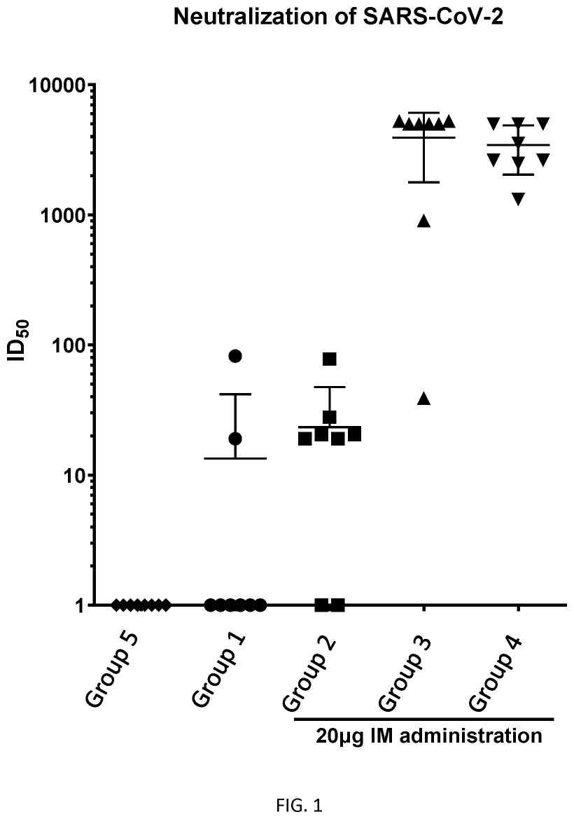

For determining whether the antibodies elicited from the Group 2, Group 3 or Group 4 formulations were neutralizing, two different assays were used. The first assay used a SARS-CoV-2 virus that was isolated from one of the first COVID-19 patients in Ontario and the readout for this assay is a microscopic reading on the health of Vero2 E6 cells that have been incubated with live virus and different dilutions of the sera from the treated mice. This assay has been used to characterize sera from convalescent patients where the ID50 values ranged from 1:80 to 1:320, therefore the dilution series chosen for the test of these mouse sera was between 1:20 and 1:2560. The sera from Group 1 mice had minimal detectable neutralization activity. Group 2 showed some moderate activity with 6 of the 8 samples with ID50s between 1:20 and a 1:80. Group 3 and Group 4 showed strong neutralizing activity with most sera samples retaining 100% neutralization activity even at the highest dilution of 1:2560. Results are provided in FIG. 1.

### Pseudovirus Neutralization Assay

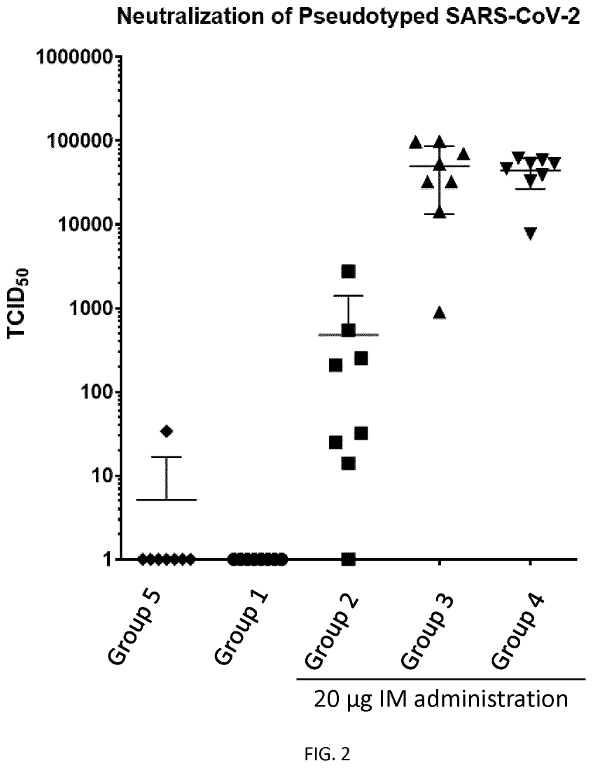

The second neutralization assay consisted of a pseudovirus assay. This assay utilizes a SARS-CoV-2 S protein pseudotype lentivirus that encodes a luciferase gene and can infect HEK293T-cells made to express hACE2 and TMPRSS2 for better transduction efficiency. This assay has been characterized by determining the titer of sera from ˜50 convalescent patients where the sera had an average ID50 of ˜1:500 with a range of 1:1 to ˜1:10,000, but for this study, a range of dilutions from 1:40 to 1:24,400 was used. The sera from both of the control groups (Group 1 and Group 5) had minimal to no activity. Group 2 had significant but low neutralization activity. The activity of Group 3 and Group 4 was above the quantitative range of the assay. Values were extrapolated and the average ID50 values were ˜1:50,000 (Group 3) and ˜1:45,000 (Group 4). Results are provided in FIG. 2.

### Conclusion

This study demonstrates that the LNP formulated mRNA vaccines when injected into mice intramuscularly twice over a three-week period were able to elicit T-cell responses and antibodies that can bind the S protein of SARS-CoV-2. The treated mice produced antibodies that could neutralize a clinical isolate of SARS-CoV-2 as well as a SARS-CoV-2 pseudotyped lentivirus. This was particularly true of Group 3 and Group 4 formulations which resulted in titers above the quantitative range in each assay.

### Example 2. Neutralizing Antibody Study in Mice

The vaccine candidate, LNP formulated vaccine encoding full-length S protein with D614G mutation (SEQ ID NO: 27; coding region provided as SEQ ID NO: 7) (vaccine formulation applied to Group 3 in Table 6, referred to hereafter as “PTX-B”), was chosen as the candidate for further study. The ability of PTX-B to produce neutralizing antibodies and T-cell response in mice was evaluated. Three (3) groups of female C57BL/6 mice (10 mice/group) were vaccinated on Days 1 and 22 as follows:
Group 1: 10 μg LNP formulated tdTomato mRNA (negative control)
Group 2: 1 μg PTX-B
Group 3: 10 μg PTX-B

Parameters evaluated in this study included clinical isolate virus neutralization assay and pseudovirus neutralization assay on pooled samples from live bleed three weeks (Day 22) after the first immunization; and the following assessments at termination (Day 43) after the second immunization: sera antibody binding to S protein from SARS-CoV-2, pseudovirus neutralization, clinical isolate virus neutralization, splenocyte T-cell responses by ELISpot and flow cytometry, and cytokine secretion.

The live phase of the experiment showed no apparent adverse reactions in the mice. Data from the initial preliminary bleed on Day 22 showed that the 10 μg dose level produced neutralizing antibodies while the 1 μg dose level was only marginally different from the negative control group.

At termination, three weeks after the second immunization (booster), the 1p g and 10 μg dose groups showed approximately equal T-cell responses in the ELISpot assay, but the 10 μg dose level group performed much better in the antibody-based assays with evidence of high levels of IgG isotypes (total IgG, IgG1, IgG2b and IgG2c). The levels of IgM were higher in the mice dosed with 1 μg than those dosed with 10 μg, perhaps due to an early class switching due to a stronger stimulus in the 10 μg group. There was evidence of IgGA, especially at the 10 μg dose, but this isotype was not induced to as high a level as the IgG isotypes. Both 1 and 10 μg PTX-B elicited very strong S-specific IgG, IgG1, IgG2b, IgG2c (end-point titers for 1 and 10 μg PTX-B are, respectively: 2.7±0.9E6, 3.0±0.5E7 for IgG; 1.1±0.2E6, 2.8±0.8E6 for IgG1; 9.4±2.0E5, 9.7±3.4E6 for IgG2b; 3.5±1.8E7, 1.95±0.0E8 for IgG2c). Both 1 and 10 μg PTX-B also elicited strong S-specific IgA (end-point titer for 1 and 10 μg PTX-B is, respectively: 3.3±3.1E4, 1.7±0.6E7), although the titers were lower than those of the IgG. The dose of g PTX-B usually induced higher S-specific binding antibody than the dose of 1 μg. The preponderance of the Th1 antibody (IgG2b and IgG2c) over the Th2 antibody (IgG1) also indicated that PTX-B induced a Th1-biased antibody response. Very low or little S-specific binding antibodies were detected in the sera of the control mice receiving the tdTomato mRNA.

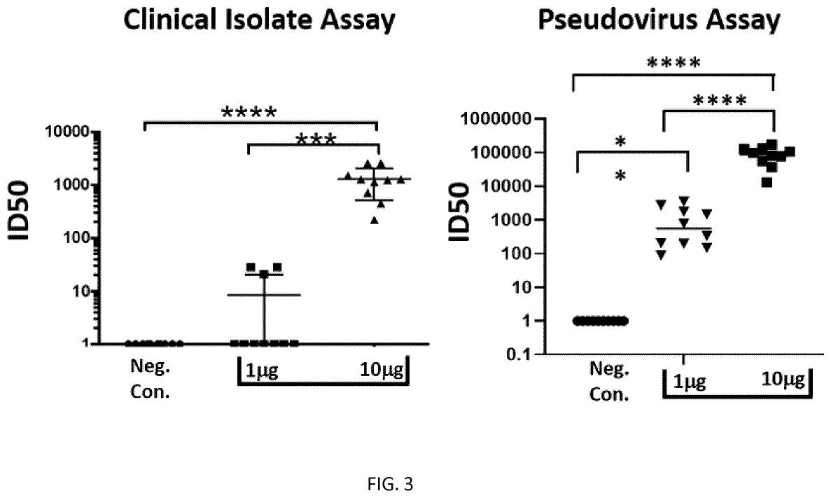

As in Example 1, the first neutralization assay used a SARS-CoV-2 virus that was isolated from one of the first COVID-19 patients in Ontario and the second assay was a pseudovirus neutralization assay using a SARS-CoV-2 S protein pseudotyped lentivirus. In both antibody neutralization assays the 10 μg dose group greatly outperformed the 1 μg dose group, though this dose group did show considerable neutralizing activity (comparable to that seen with sera from convalescent patients). FIG. 3 shows the ID50 (dilution at which 50% inhibition of infectivity is seen) for both the SARS-CoV-2 clinical isolate and pseudovirus neutralization assays. The sera from the negative control group showed no activity in either assay. There was a dose-responsive effect with sera from the 10 μg group demonstrating considerably more neutralization activity, especially in the SARS-CoV-2 clinical isolate assay. Statistics were performed by Kruskal-Wallis test using multiple comparisons; in FIG. 3, **=P<0.01, ***=P<0.001 ****=P<0.0001. There was no significant activity in the negative control group, moderate activity in the 1 μg dose group and very strong neutralizing activity in the 10 μg group with ID50s up to 1:90,000 in the pseudovirus assay.

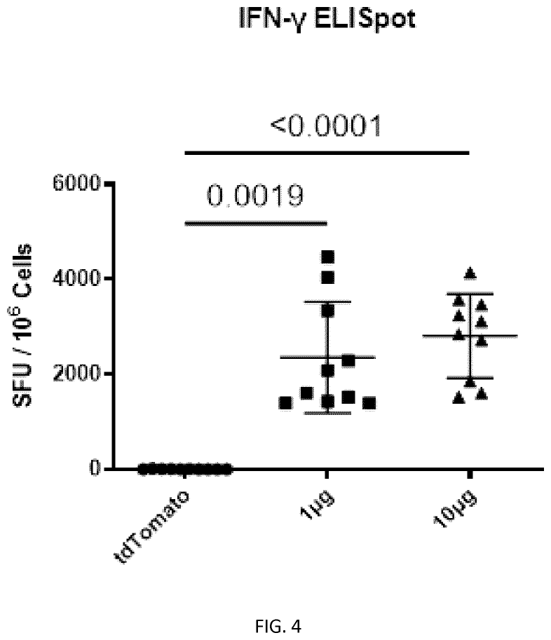

IFN-γ analysis by ELISpot was performed to determine the T-cell response to immunization with the vaccine. Splenocytes from mice were stimulated with peptide pools of SARS-CoV-2 S protein (315 15mer peptides with 1 lmer overlap). IFN-γ producing T-cells were measured by ELISpot analysis. A higher frequency of T-cells from PTX-B-immunized mice produced IFN-γ compared with those from mice vaccinated with the negative control (FIG. 4). Mice were vaccinated with a prime and booster of PTX-B at Days 1 and 22. Mice were sacrificed at Day 43 and splenocytes were stimulated in the presence of SARS-CoV-2 peptide pool overnight on a 96 well ELISpot plate precoated with anti-IFN-γ antibodies. Following incubation, the plates were washed stained and treated with an anti-IFN-γ HRP antibody and read on an ELISpot reader. Statistics were performed using Kruskal-Wallis test with multiple comparison analysis.

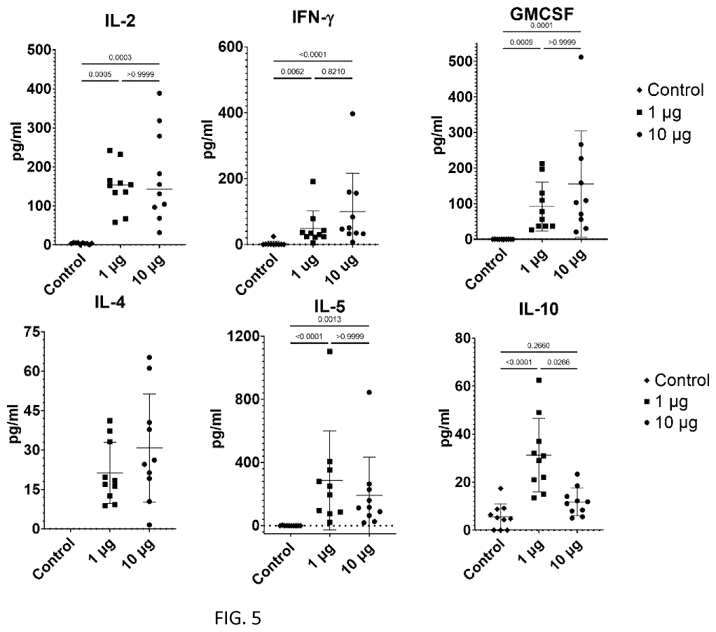

Cytokine profiling by Luminex showed that mice immunized with PTX-B produced in a dose-dependent manner high levels of IL-2, IFN-γ, and GM-CSF but low levels of IL-4 and IL-10 (FIG. 5). Mice were vaccinated with a prime and booster of PTX-B at Days 1 and 22. Mice were sacrificed at Day 43 and splenocytes were stimulated in the presence of SARS-CoV-2 peptide pool overnight. Supernatants were analyzed by Luminex for the presence of IL-2, IFN-γ, GM-CSF, IL-4, IL-5, and IL-10. Statistics were performed by Kruskal-Wallis test by multiple comparisons. The levels of TNF-α were not detectable in the assay for mice immunized with the PTX-B or control. Interestingly, levels of IL-5 were detectable in PTX-B-immunized mice but did not increase with vaccination.

### Cellular Immune Response

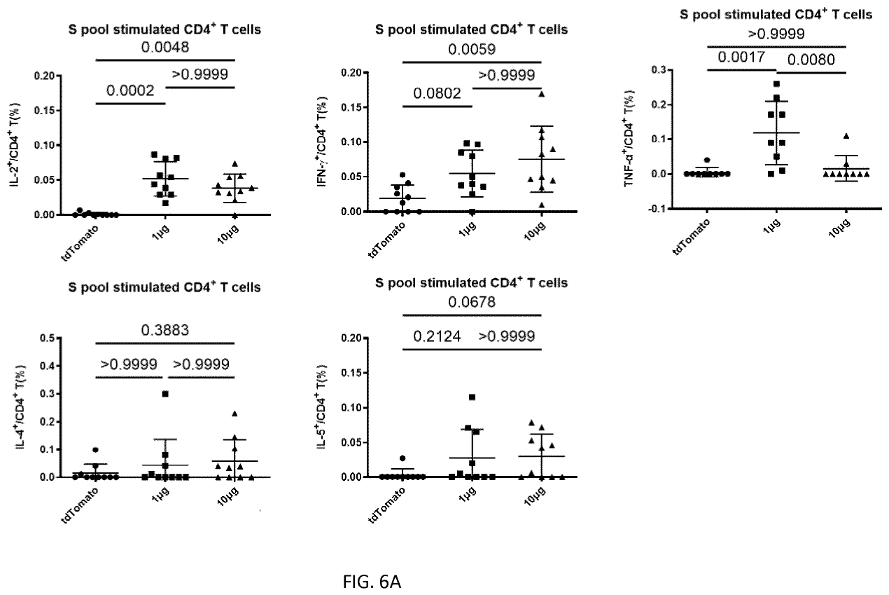

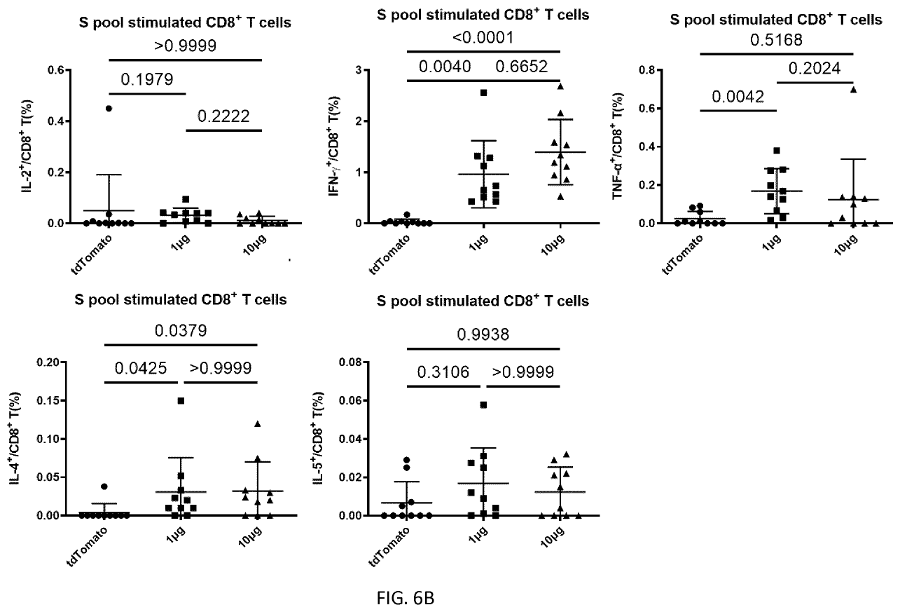

PTX-B also elicited a strong cellular immune response. Mouse splenocytes were prepared at 3 weeks after the boost vaccination, stimulated with a S peptide pool, and the S-specific cellular responses were measured by IFN-γ/IL-4 ELISpot, flow cytometry analysis of cytokine production by CD4+ and CD8+ T-cells, and a multiplex immunoassay to detect the cytokines in the supernatant of the stimulated splenocytes. These assays showed that both 1 μg and 10 μg PTX-B induced robust S-specific cellular immune responses, which is Th1-biased as indicated by the predominant Th1 cytokine (IFN-γ/TNF-α/IL-2) production over Th2 cytokine (IL-4/IL-5) from CD4+ T-cells. Of note, significant amount of S-specific CD8+ T-cells were induced by PTX-B. In contrast to the humoral response, especially the nAb response, the cellular responses elicited by 1 g and 10 μg PTX-B were usually comparable. Cytokine profiling by flow cytometry showed significant proportions of CD4+(FIG. 6A) and CD8+(FIG. 6B) cells producing IL-2 and IFN-γ detected in PTX-B-immunized mice, especially CD8+ IFN-γ producing cells. In contrast, IL-4 and IL-5 producing cells were not significantly different in immunized mice compared to the control mice. Mice were vaccinated with a prime and booster of PTX-B at Days 1 and 22. Mice were sacrificed at Day 43 and splenocytes were stimulated in the presence of SARS-CoV-2 peptide pool overnight. Following overnight stimulation, cells were surface stained for anti-CD3, anti-CD4 and anti-CD8 antibodies. Cells were then fixed and permeabilized and stained for IL-2, IFN-γ, TNF-α, IL-4 and IL-5. Cells were evaluated using flow cytometry. FIG. 6A and FIG. 6B show that TNF-α producing cells were slightly higher than control mice but not consistently high in a dose dependent manner. These results demonstrate that vaccination with PTX-B induced an S protein specific Th1 response.

It was determined that immunizations with either 1 μg or 10 μg PTX-B led to similar T-cell response, both well above the background in the negative control group. For the antibody-based assays (antibody levels and neutralization ability), the 10 μg dose outperformed the 1 μg dose.

### Example 3. Mouse AAV6-hACE2 Challenge Model

A non-GLP challenge study was conducted in AAV6-hACE2 (receptor for SARS-CoV-2) transfected C57BL/6 mice to investigate the protective efficacy of PTX-B. Four groups of female C57BL/6 mice (12 mice/group) were vaccinated with PTX-B on Days 1 and 22 as follows:
Group 1: Formulation buffer (negative control)
Group 2: 20 μg PTX-B
Group 3: 4 μg PTX-B
Group 4: 1 μg PTX-B

On Day 29, the animals were transduced with 1×10¹¹ vector genome copies of AAV-hACE2 per mouse and then challenged intranasally with 2.5×10⁴ TCID50 with SARS-CoV-2 per mouse on Day 38. Study termination was on Day 42. The parameters evaluated in this study included infectivity of lung homogenates, viral RNA levels in the lung, and lung histopathology. Mice were euthanized and one lung was taken for histology while the second lung was split in half for homogenization in media for infectivity test and homogenization in RNA extracting buffer for viral load determination.

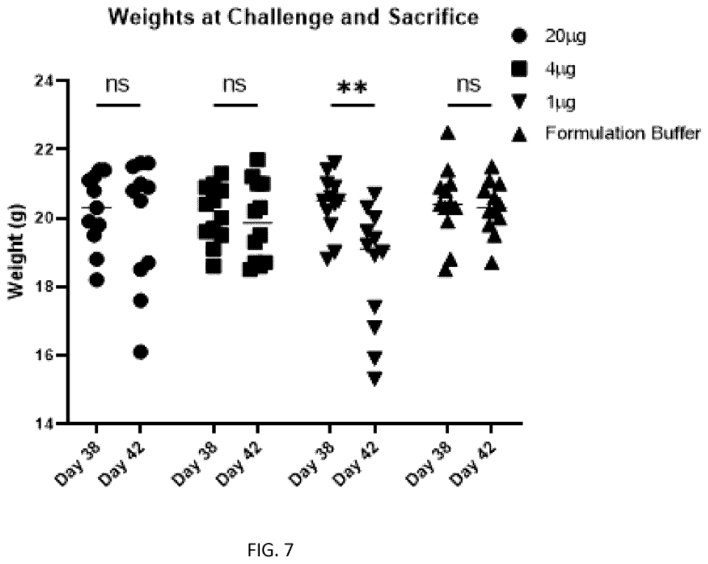

The live phase of the experiment showed no apparent adverse reactions in the mice. Body weights were measured on Day 38, at the time of challenge and then again on Day 42, immediately prior to sacrifice. There was a statistically significant weight loss observed in the 1 μg PTX-B vaccination group (20.39 vs 18.54, 9.07% p=0.0016) (FIG. 7). C57BL/6 mice were immunized with PTX-B prime-booster and transduced with AAV6-hACE2. Body weights were measured at time of challenge (Day 38) and immediately before sacrifice (Day 42). Analysis performed by 2-way ANOVA with multiple comparisons. In FIG. 7, ** p<0.01. No significant weight loss was observed in the 20 μg or 4 μg groups, or in the formulation control group.

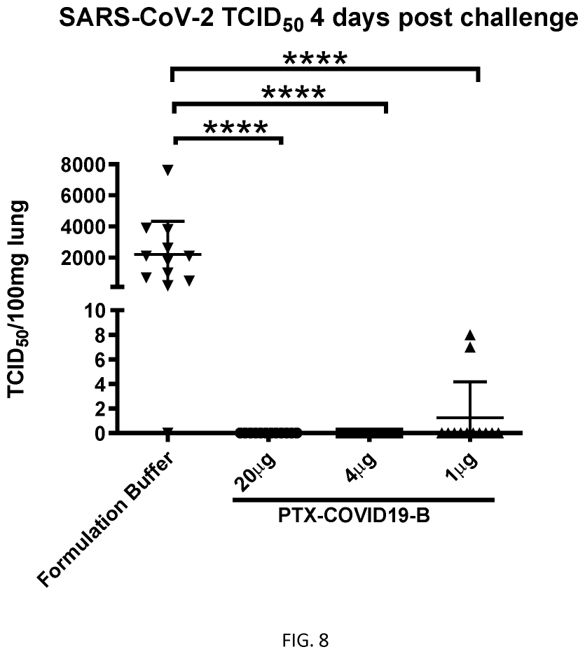

As shown in FIG. 8, PTX-B provided protective efficacy at all three dose levels tested. No infective virus was found in the mice immunized with 20 or 4 μg of vaccine (TCID50=0) and 10 of 12 mice immunized with 1 μg were also free of infective virus (mean TCID50=1.25±2.93) while 11 of the 12 mice in the formulation buffer negative control group had easily detectable infectious SARS-CoV-2. In FIG. 8, TCID50 means tissue culture 50% infectious dose. As shown, PTX-B neutralizes SARS-CoV-2. TCID50 were measured in AAV6-hACE2 transduced C57BL/6 mice that were immunized by prime-booster with PTX-B at 3 different doses or a formulation buffer negative control. Mice were transduced with AAV6-hACE2 at 7 days post booster and challenged 9 days later. All mice were sacrificed at 4 days post challenge with SARS-CoV-2 and virus measured from lung homogenates (n=12 per group) (**** p<0.0001).

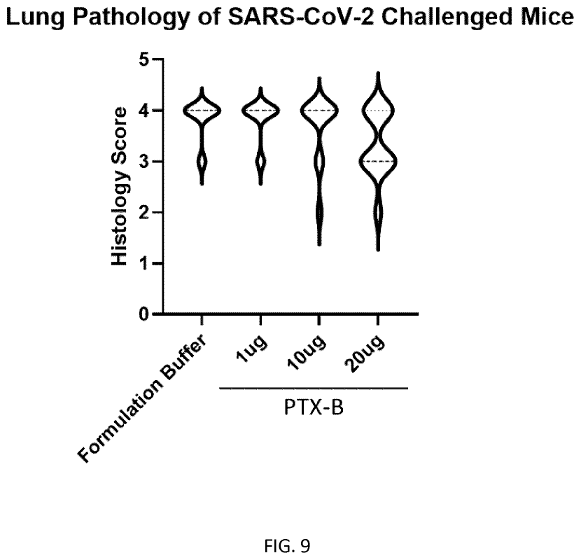

Additionally, detection of viral RNA in the lungs by PCR demonstrated a dose-responsive reduction with more than 100-fold difference between the averages of the high dose and negative control groups. Sections of one lung were graded for lung histopathology in infected mice. All mice demonstrated significant histopathology. It is not clear how much of the pathology was due to SARS-CoV-2 and how much was due to the AAV6 virus used to express hACE2; however, there was a trend to lower histopathology scores in the groups of mice treated with the two higher dose levels of PTX-B (FIG. 9). To summarize, mice were immunized with the indicated amount of PTX-B, transduced with AAV6-hACE2 and nine days later challenged with SARS-CoV-2. Four days after challenge, mice were immunized and the left lung was fixed in formalin, processed for histology and examined under the microscope by a certified pathologist who was blinded to the treatment conditions. Each sample was assigned a histology score from 1-5 with the lowest being normal. A trend to lower pathology was seen with increasing doses of the vaccine.

In conclusion, administration of PTX-B (1, 4 and 20 μg) conferred protection against SARS-CoV-2 infection using the AAV6-hACE2 transduction mouse model and had positive effects on lung pathology suggesting a protective or damage preventing feature of PTX-B. There was also a reduced total amount of SARS-CoV-2 mRNA in the lungs at euthanasia. A post-challenge weight loss was observed in the low dose (1 μg) vaccination group.

### Example 4. Hamster Challenge Model

A challenge study was performed in 6-8-week-old male Syrian Golden hamsters challenged with SARS-CoV-2 to determine if the vaccine protected from infection.

The Syrian golden hamster is susceptible to SARS-CoV-2 infection and has demonstrated utility for evaluating candidate vaccines.

Group 1 hamsters received 20 μg LNP formulated full-length S protein mRNA (PTX-B).

Group 2 hamsters received 4 μg LNP formulated full-length S protein mRNA (PTX-B).

Group 3 hamsters received 1 μg LNP formulated full-length S protein mRNA (PTX-B).

Group 4 hamsters received formulation buffer (PBS sham/negative vaccine control group).

On Day 0, all hamsters were pre-bled for baseline analysis. On Day 1 all hamsters received the first intramuscular injection (vaccine or control, according to Group 1-Group 4). Animals were allowed to acclimatize for 7 days prior to receiving the first vaccine dose. On Day 21 all hamsters were subjected to live bleeding. On Day 22 all hamsters received the second (booster) vaccination according to Group 1-Group 4. On Day 29 all hamsters received AAV6-hACE2 (see, e.g., Example 3) intranasally to facilitate SARS-CoV-2 infection. On Day 38, all hamsters were infected with SARS-CoV-2 via intranasal infection. All animals received a total dose of 7.5×10\hat{ }5 TCID₅₀ as determined by back-titration. Following this, animals were monitored daily for weight loss and signs of disease or distress. Additionally, viral shedding was monitored by collecting oral swabs on every second day. Hamsters were euthanized on Day 42 for endpoint analysis: (i) infectivity of lung homogenates; and (ii) viral RNA level in lung; (iii) lung histopathology. Animals were monitored during the study for any observable clinical signs during the vaccination phase. There were no apparent adverse reactions observed. Full experimental design is illustrated in Table 7.

TABLE 7
Experimental Overview

| | |
|---|---|
| Route of vaccination | Hamsters were vaccinated at two sites |
| | intramuscularly in the hind leg, with 200 uL |
| | (100 ul per site) total using 23-25 gauge, |
| | ⅜-1-inch needles. |
| Time of vaccination | 2 doses on days 1 and 22, challenge on day 43 |
| Challenge virus | SARS-CoV-2 |
| Dose | 7.5 × 10⁵ TCID50 |
| Route | Intranasal |

At 4 days and 8 days post-infection (dpi), four animals from each group were selected at random and euthanized. Tissues were collected for viral load by qRT-PCR and infectious virus titre levels as well as for histopathology. At the terminal point of the experiment (8 dpi), blood was also collected from animals in both groups to evaluate titers of neutralizing antibodies.

Animals in the vaccinated groups showed on average no weight loss during the course of the experiment. By contrast, hamsters in the sham-vaccine group showed moderate average weight loss beginning at 3 dpi. Overall, the average weight loss was 11% by terminal point of the experiment. No other significant clinical signs of disease were reported for either group.

Following euthanization of the animals at 4 dpi and 8 dpi, half of the lung was placed into formalin for tissue fixation. Tissues subsequently underwent H&E staining and were evaluated by a pathologist who was blinded to the groups. Pathology scores were significantly higher at both timepoints in the control group (sham vaccination) compared to the vaccinated group. This suggested more severe disease in the unvaccinated group.

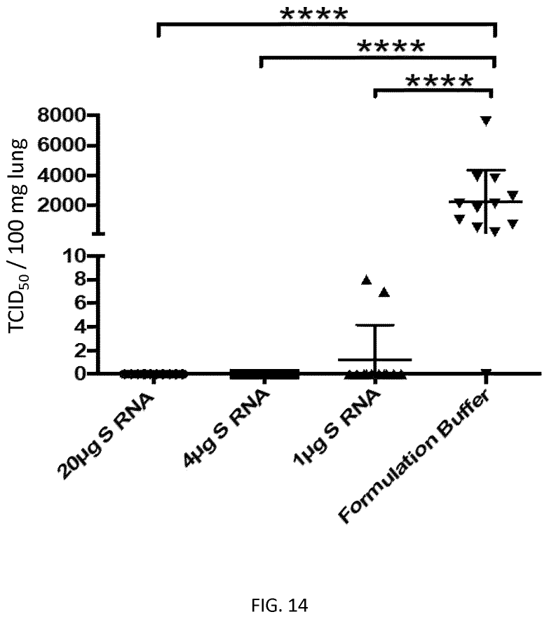

Collection of oral swabs over the course of the experiment was used to evaluate viral shedding. Interestingly, while viral RNA was detected in both groups throughout the experiment, the levels of actual infectious virus was significantly lower in vaccinated animals (ranging from 2-3 log reduction, as illustrated in FIG. 14). This suggests that viral shedding was lower in the vaccinated group throughout the course of the experiment and that PTX-B can reduce viral shedding thereby providing a therapeutic benefit.

Examination of the viral burden in nasal turbinates demonstrated significantly lower quantities of infectious virus at 4 dpi in the vaccinated group and undetectable levels of infectious virus at 8 dpi. A similar trend was seen in the lungs, although a notable difference was that infectious virus was not detected at either timepoint in the lungs of vaccinated animals. Viral RNA was detected in lungs in both vaccinated and unvaccinated groups at both timepoints.

These data indicate that vaccination with PTX-B conferred protection against an intranasal challenge with SARS-CoV2-2 in the hamster model of infection.

### Example 5. Immunogenicity and Local Tolerance Study in Mice

The goal of this 2-dose immunogenicity and tolerability study was to obtain basic safety data pertinent to mRNA vaccines in addition to immunological data in a different strain of mice than used for the other preclinical experiments. PTX-B was administered to groups of BALB/c mice by IM injection on Days 1 and 22 at dose levels of 0, 4, or 20 μg as outlined in Table 8. Main study animals were evaluated for clinical signs, body weight changes, and dermal observations by modified Draize scoring. The hematology cohort of animals was sacrificed on Day 24 (two days after second dose), blood was drawn for hematology and organ weights were recorded, gross pathology evaluated, and liver, spleen, and injection site tissues were examined microscopically. The cytokine cohort was sacrificed on Day 22 for determination of serum cytokine concentrations. The main study animals were terminated on Day 43 (three weeks after the second dose) and assessed for immunogenicity end points, hematology, clinical chemistry, liver function tests, gross pathology, and organ weights.

TABLE 8
Immunogenicity and Local Tolerance Study in Mice - Study Design

| | | | Dose | Dose | No. of Animals | | | | | |
|---|---|---|---|---|---|---|---|---|---|---|
| Group | Test | Dose | Volume | Concentration | Main Study | | Hematology | | Cytokine | |
| No. | Material | (μg) | (μl) | (μg/mL) | Males | Females | Males | Females | Males | Females |
| 1 | Control | 0 | 50 | 0 | 10 | 10 | 5 | 5 | 5 | 5 |
| 2 | PTX-B | 4 | 50 | 0.08 | 10 | 10 | 5 | 5 | 5 | 5 |
| 3 | PTX-B | 20 | 50 | 0.4 | 10 | 10 | 5 | 5 | 5 | 5 |

### Safety-Related Endpoints

Transient, slight body weight loss was observed in both sexes after the second dose of 20 μg PTX-B; however, no difference in the average body weights among the groups was apparent at the end of the study (data not shown).

Test material-related findings at the injection site were noted upon clinical observation, and gross and microscopic examination; all findings were reversible. Based on Draize scoring, occasional findings of redness and/or swelling were observed at the injection site at both 4 and 20 μg and erected fur was seen in a minority of female mice for one to two days after the first dose, but these were not considered significant findings. Upon termination two days after the second dose, histopathological findings at the injection site included minimal to moderate mixed cell inflammation at 4 and 20 μg in both sexes; the finding was accompanied by edema and, in one female at 20 μg, by mineralized material. A dose relationship in the incidence and severity of the finding was noted in females. Inflammation correlated grossly with firm abnormal consistency and swelling. In addition, minimal to mild hemorrhage was noted in a few animals at both doses, correlating grossly with dark focus of the injection site or subcutis; no dose relationship was evident. At the end of the study, no macroscopic findings were observed at the injection site.

Serum cytokine analysis 6 hours after the second dose (Day 22) was performed to monitor for cytokine release syndrome, which is a known potential side effect of LNP-formulated mRNAs. IFN-γ, IL-1β, IL-6, IL-10, MCP-1, and TNF-α were analyzed using a validated immunoassay method. PTX-B-related increases in serum concentrations of IL-6 (up to 53-fold and 266-fold of control in males and females respectively) and MCP-1 (up to 20-fold and 15-fold of control in males and females respectively) were observed in both sexes at the two dose levels. In general, the magnitude of the responses was dose related. For MCP-1, the response had no meaningful sex-related difference. For IL-6, the increase was greater in females than in males. Mild increases (up to 2.5-fold) in serum concentrations of IFN-γ were observed in some animals of both sexes. No PTX-B-related changes in IL-10, IL-10, and TNF-α were apparent. The pattern of cytokine changes observed was not consistent with cytokine release syndrome.

Body weight was determined weekly during the study. A slight dip in body weight was seen in both males and females in the 20 μg group. The body weight of each group had recovered by the end of the study (data not shown.)

Hematological parameters were determined two days (Day 24) and three weeks (Day 43) after the second vaccination. At the first time point, the only changes that changed dose-responsively in both sexes were leukocytes (males 264% and 420% of control at 4 and 20 μg respectively, females 329% and 514% of control at 4 and 20 μg respectively) and reticulocytes (males 69% and 41% of control at 4 and 20 μg respectively, females 53% and 27% of control at 4 and 20 μg respectively); no effect on red cell parameters was observed. All hematological parameters were within normal ranges on Day 43 (leukocytes: males 77% and 77% of control at 4 and 20 μg respectively, females 67% and 75% of control at 4 and 20 μg respectively, reticulocytes (males 107% and 117% of control at 4 and 20 μg respectively, females 122% and 129% of control at 4 and 20 μg respectively).

At two days (Day 24) after the second dose, the liver of females at 20 μg showed minimal hepatocellular cytoplasmic alteration, characterized by accumulation of glycogen-like material, was noted. The change correlated with increased weights (absolute and relative to brain weight, 24% to 27%) and pale discoloration. However, at Day 43, all liver function tests were within normal ranges and not significantly different from the control group.

On Day 24, increased spleen weights (absolute and relative to brain weight, 32% to 49%) were noted in both sexes at 4 and 20 μg. This increase was statistically significant and correlated grossly with enlargement in females. No microscopic correlate could be established.

By the end of the study (Day 43, three weeks after the second dose), no PTX-B-related gross findings were noted. Although absolute spleen weights remained increased (14% to 18%) at 20 μg, the magnitude of the increase was substantially lower than at two days after the second dose and all hematological parameters were normal.

In summary, PTX-B administered to BALB/c mice by IM injection on Days 1 and 22 at dose levels of 4 and 20 μg was well tolerated. Findings observed after the second dose were limited primarily increases in serum concentrations of IL-6 and MCP-1, dose-related increases in leukocytes and decreases in reticulocytes, dose-related injection site reactions, and increased spleen weight with no microscopic correlate. Additional findings noted only at 20 ug/dose included slight body weight loss and hepatocellular cytoplasmic alteration. All findings were fully or partially reversible; by Day 43, test material-related effects were limited to slight increase in spleen weights at 20 ug/dose.

### Immunogenicity-Related Endpoints

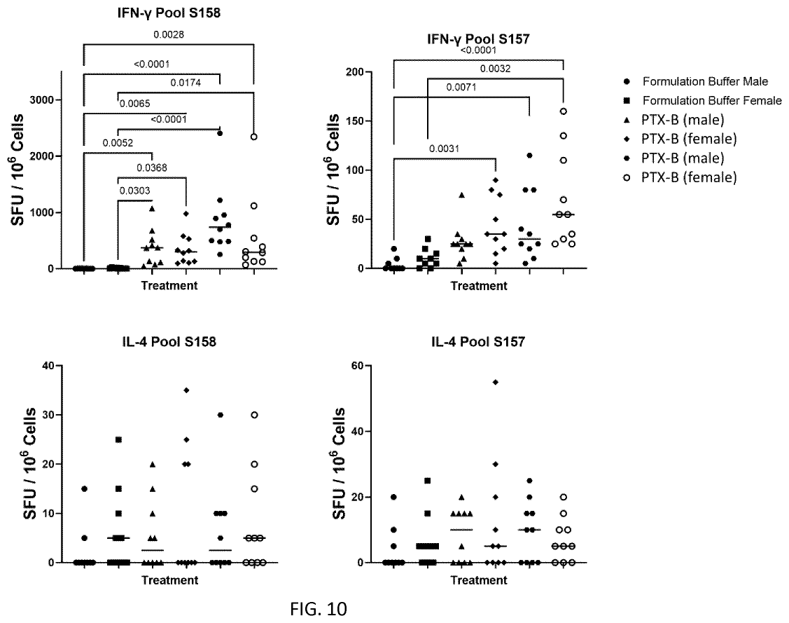

Splenocytes were collected at end of study for analysis by ELISpot (FIG. 10, IFN-γ and IL-4 ELISpots of splenocytes from PTX-B immunized mice). Splenocytes collected from PTX-B mice were stimulated in the presence of SARS-CoV-2 S protein peptide pools S158 and S157 (available, e.g., from JPT Peptide Technologies, Berlin, Germany) on IFN-γ and IL-4 multiplexed ELISpot plate. Spots were counted after overnight stimulation. Statistics performed by two-way ANOVA. As shown in FIG. 10, a significant increase in expression of IFN-γ was observed from both male and female mice stimulated with the S158 peptide pool. Similarly, the S157 peptide pool induced significant increase in IFN-γ expression in male mice. IL-4 expression was not significantly increased by stimulation with either peptide pool. This combination of results indicates a Th1 skewed response.

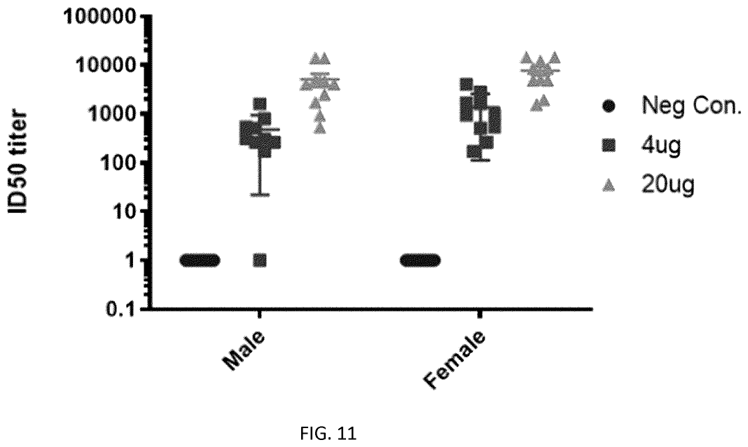

In the SARS-CoV-2 neutralization assay, sera from both female and male mice treated with formulation buffer as a negative control provided no protection from infection with a SARS-CoV-2 clinical isolate at any dilution tested (FIG. 11). Conversely, sera from female and male mice immunized with 4 μg of PTX-B gave mean ID50 titers of 1353 and 480, respectively. Sera from mice immunized with 20 μg of PTX-B provided even greater protection with mean ID50 titers of 7645 (females) and 5118 (males) demonstrating a dose-responsive effect (FIG. 11).

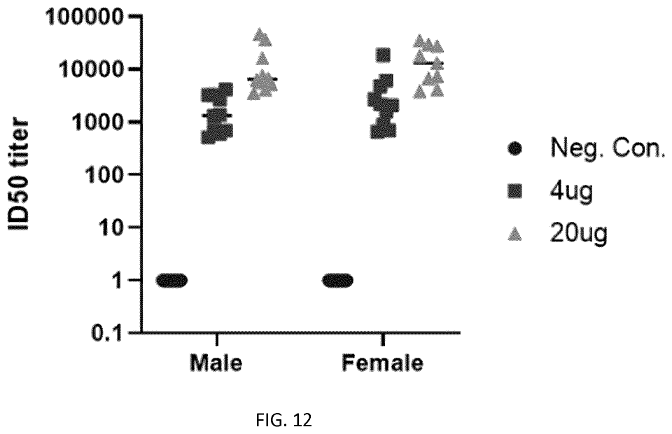

To confirm the strong neutralizing effect of the sera from mice treated with PTX-B, a second independent pseudovirus neutralization assay was performed. Sera from the negative control group showed no neutralizing capacity (FIG. 12). Sera from the female and male mice treated with 4 μg of PTX-B provided protective activity with ID50 values of 4048 and 1863, respectively. Sera from mice immunized with 20 μg of PTX-B showed mean ID50 values of 16390 (females) and 1414 (males).

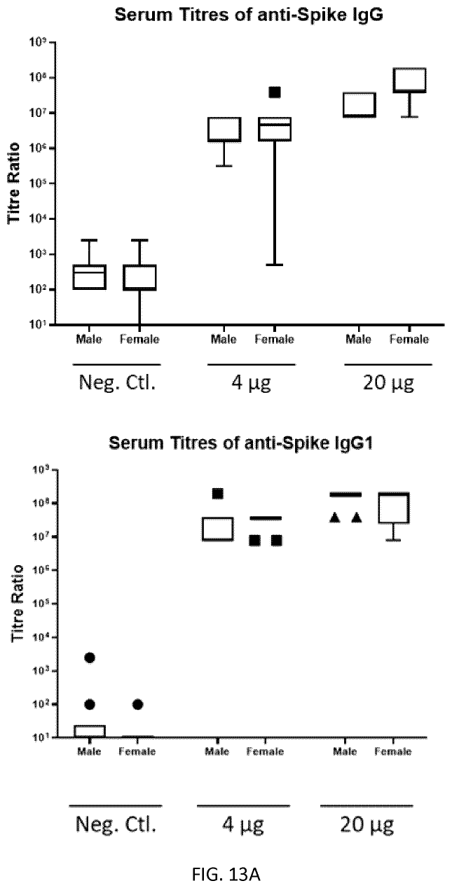

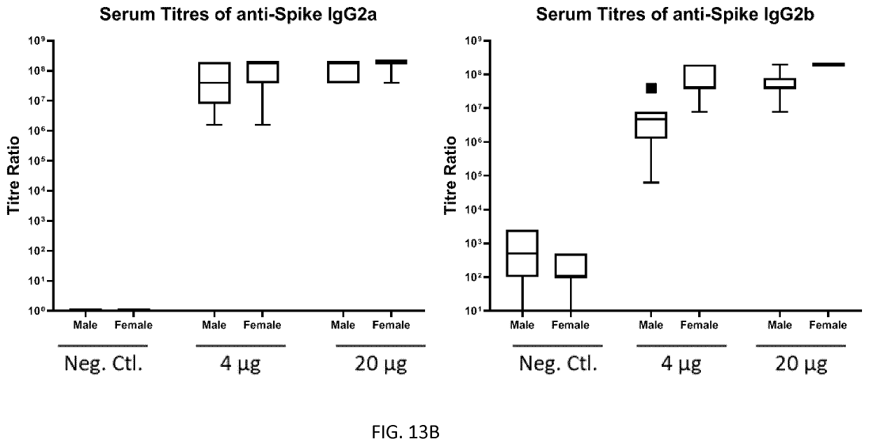

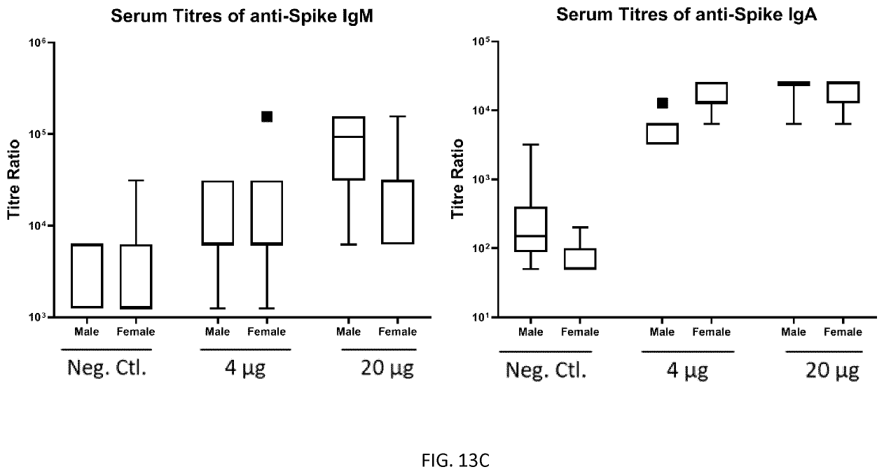

Immunization with PTX-B, at both 4 and 20 μg, resulted in production of a strong neutralizing antibody response in BALB/c mice (FIG. 13A-FIG. 13C). Serial dilutions of sera from PTX-B treated mice were performed and the anti-SARS-CoV-2 Spike IgG, IgG1, IgG2a, IgG2b, IgM and IgA were measured using anti-isotype HRP antibodies. Median values are represented using boxplots with whiskers representing the Tukey analysis of the Q1 and Q3 of the interquartile range with statistical outliers represented with individual dots. The results were dose-responsive and consistent with what was demonstrated in C57BL/6 mice in previous experiments. The anti-SARS-CoV-2 anti-Spike protein antibody profile induced by prime-booster with PTX-B showed that this formulated vaccine promoted seroconversion against SARS-CoV-2 Spike protein. SARS-CoV-2 spike protein specific IgG (FIG. 13A, left panel), IgG1 (FIG. 13A, right panel), IgG2a (FIG. 13B, left panel), IgG2b (FIG. 13B, right panel), IgM (FIG. 13C, left panel) and IgA (FIG. 13C, right panel) were induced at both dose levels tested.

### Example 6. Efficacy of SARS-CoV-2 N, M, N/M Protein Nucleic Acid Vaccines

A co-culture assay was performed by mixing fluorescent T-cells (labelled with CFSE) with activated dendritic cells (DC) transfected with mRNA of N, M or N/M hybrids with or without a secretory signal as described in Table 9 below. Specifically, monocytes were isolated on Day 1 from peripheral blood mononuclear cells (PBMCs) from COVID-19 convalescent patients. Monocyte-derived dendritic cells (MDDCs) were also differentiated from PBMCs from the same convalescent patient. MDDCs were transfected with RNA vaccine on Day 4 and transfected MDDCs were co-cultured with PBMCs to induce autologous stimulation of T-cells in the co-culture.

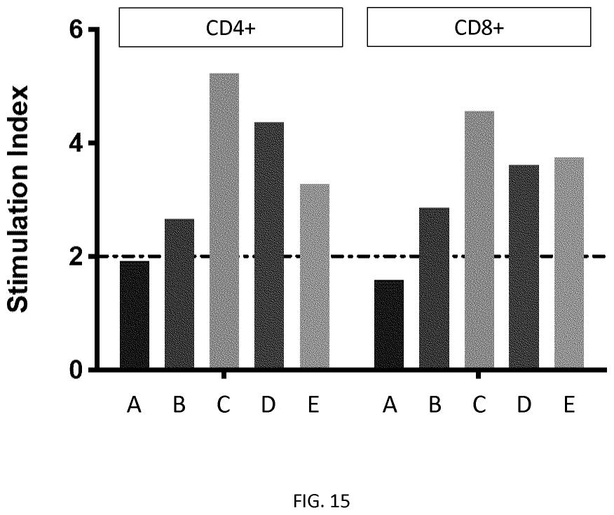

TABLE 9
Description of Nucleic Acid Vaccines
Used in Example 6 & FIG. 15

| | | SEQ |
|---|---|---|
| Group | mRNA Vaccine Description | ID NO |
| A | tdTomato (neg. control) | 46 |
| B | N - sequence encoding N protein | 30 |
| C | M - sequence encoding M protein | 28 |
| D | NM - sequence encoding N and M proteins | 29 |
| E | sNM - sequence with signal peptide and encoding | 31 |
| | N and M proteins | |

Dendritic cells in co-culture were matured using a cytokine cocktail comprising TNF-a, IL-1b, IL-6, and PGE2 factors. On Day 5, cells were subject to labeling with carboxyfluorescein succinimidyl ester (CFSE) to monitor lymphocyte proliferation (see, e.g., Lyons A B, Parish C R (May 1994). “Determination of lymphocyte division by flow cytometry”. Journal of Immunological Methods. 171 (1): 131-7, incorporated herein by reference).

Subsequently, on Day 11, cells were stained using fluorescent antibody for CD4 or CD8 to identify CD4+/CD8+ T-cells and amount of CFSE label in antibody-labelled cells was determined to ascertain T-cell proliferation.

Cells that were stimulated by the DC had low levels of CSFE due to multiple cellular divisions. A threshold was established to identify low CFSE-stained cells, i.e., cells having undergone 1 or more division (e.g., CFSE signal reduced to 12 in cells having divided 1 time; CFSE signal reduced by more than 12 in cells having divided more than 1 time). Typically, cells having divided between 1 and 7 times were identified as “low CFSE” cells. Thus, the ratio of [low CFSE staining in mRNA transfected cells]-to-[low CFSE staining in control cells] provides a T-cell stimulation index (SI). Increased SI values indicate extent to which the mRNA vaccine stimulated a T-cell response.

Stimulation of T-cells, i.e., stimulation index (SI) was calculated as follows: SI=[Proliferation (% CFSE-lo) of T-cells stimulated with DC transfected with mRNA]divided by [Proliferation (% CFSE-lo) of T-cells stimulated with DC control (DC with medium)]. An SI value greater than 2 was deemed to indicate biologically significant proliferation. Data are shown in FIG. 15.

### Example 7. Pseudovirus Neutralization Assay Using PTX-B Against VOCs

Neutralization capacity of various patient sera was tested using a pseudovirus neutralization assay (see, e.g., Example 2) employing pseudovirus pseudotyped to S proteins of wild type SARS-CoV-2 variants of concern (VOCs) and other variants as described in Table 10.

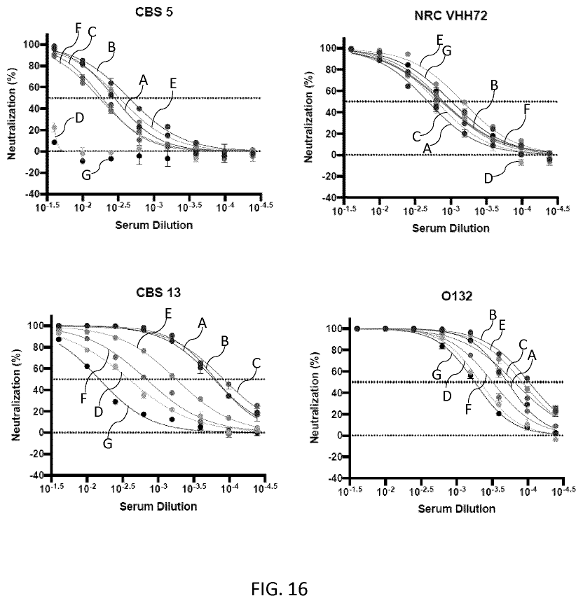

TABLE 10
Description of Pseudovirus-Encoded S
Protein in Example 7 & FIGs. 16-18

| Group | Pseudovirus ID | Description of Encoded S Protein |
|---|---|---|
| A | WT | S protein from original strain from |
| | | Wuhan, China |
| B | D614G | WT S protein with D614G mutation |
| C | UK variant | PANGO lineage B.1.17 S protein |
| D | SA variant | PANGO lineage B.1.351 S protein |
| E | D614G + L452R | WT S protein with D614G and L452R |
| F | UK + L452R | PANGO lineage B.1.17 S protein with |
| | | L452R mutation |
| G | SA + L452R | PANGO lineage B.1.351 S protein with |
| | | L452R mutation |
| H | BR variant | PANGO lineage P.1 S protein |

Sera tested included CBS 13 and CBS 5 (Canadian Blood Services samples from COVID-19 patients), 0132 (serum from a doubly mRNA-vaccinated patient), serum containing NRC VHH72 antibody (targeting S protein RBD). As shown in FIG. 16, neutralization capacity of the various tested sera was reduced against pseudovirus encoding different SARS-CoV-2 variant S proteins. Generally, neutralization capacity dropped against variants encoding the UK and South African S protein variants as compared to WT and D614G variant. The South African variant S protein and the South African variant S protein including the L452R mutation consistently showed the lowest levels of neutralization.

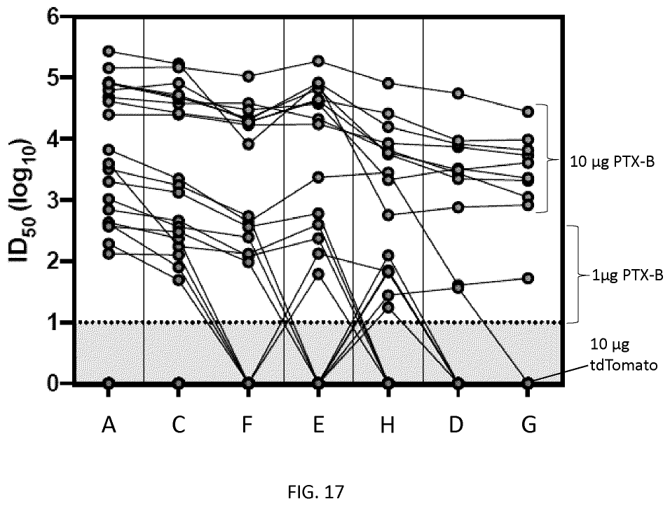

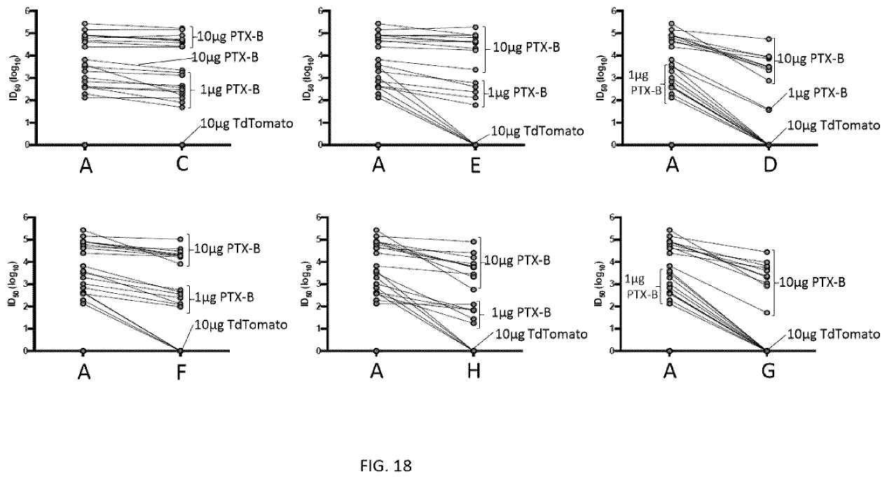

Next, sera from mice vaccinated with 1 μg or 10 μg of PTX-B or 10 μg of control mRNA vaccine encoding TdTomato was tested against VOC pseudoviruses and ID50 was measured. As shown in FIG. 17, sera from mice vaccinated with control mRNA vaccine (TdTomato) showed no protection from infection. Sera from mice vaccinated with 1 μg PTX-B showed protection from infection with a dose-dependent increase in protection efficiency seen for the 10 μg dose. Generally, ID50 values between 2 and 3 in FIG. 17 are likely protective against infection and ID values greater than 3 are presumed protective. As can be seen, highest levels of protection are demonstrated against WT (A), UK (C), UK+L452R (F), and D614G+L452R (E) pseudovirus S protein variants, with reductions in protection efficacy observed for BR (H), SA (D), and SA+L452R (G) pseudovirus S protein variants. In FIG. 17, lines connecting dots indicate sera from the same mouse. FIG. 18 shows a comparison of protection efficiency of each variant (C, F, E, H, D, and G as provided in Table 10) against WT SARS-CoV-2 pseudotyped pseudovirus (A in Table 10) and confirms the trends shown in FIG. 17.

### Example 8. Safety, Tolerability, and Immunogenicity in Humans

### Study Overview

A Phase Ia/Ib, First-in-Human, Observer-Blinded, Randomized, Placebo Controlled, Ascending Dose Study was conducted to evaluate the safety, tolerability, and immunogenicity of PTX-B Vaccine in healthy seronegative adults aged 18-64 and >65.

Objectives of the study were to evaluate the safety and tolerability of 2 doses of PTX-B vaccine in healthy seronegative adults 18-64 years of age and >65 years of age and to evaluate the immunogenicity of 2 doses of PTX-B vaccine in healthy seronegative adults 18-64 years of age and >65 years of age.

Safety and tolerability endpoints evaluated were: occurrence of events during the follow-up after each vaccination using both the Per Protocol (PP) and Safety Populations, including: vital signs and administration site reactions (e.g., arm check evaluations including pain, tenderness, erythema/redness, induration/swelling) during the follow-up after each vaccination; and daily solicited adverse events (AEs; e.g., fever, chills, nausea/vomiting, diarrhea, headache, fatigue, myalgia) through the third day post each vaccination.

Overall safety was analyzed using both the Modified Intent-to-Treat (mITT) and Safety Populations. Analysis using the mITT Population was not performed if it differed from the PP Population by ≤5% of the subjects for each of the treatment groups. Unsolicited adverse events from Day 1 through Day 56 were analyzed, along with medically attended AEs (Day 1 through Day 56), new onset chronic disease (NOCD), serious adverse events [SAEs], adverse events of special interest [AESIs], and potential immune mediated medical conditions (PIMMCs) from Day 1 through to Day 395 (approximately 1 year after the last vaccination). Findings from targeted physical examinations, vital sign assessments, and clinical safety laboratory testing were also recorded.

Immune response endpoints evaluated included: Immunogenicity analysis using both the mITT and Safety Populations. Analysis using the mITT Population was not performed if it differed from the PP Population by ≤5% of the subjects for each of the treatment groups. Antibodies (immunoglobulin (Ig) M, IgG, IgA [enzyme-linked immunosorbent assay]; and neutralization) were analyzed and cell-mediated immunity using blood/peripheral blood mononuclear cells (PBMCs [Flow Cytometry, Enzyme Linked Immunospot Assay]) was analyzed.

The study was designed with age- and dose-escalations and performed in seronegative adult subjects without evidence of recent of exposure to Severe Acute Respiratory Syndrome (SARS)-CoV-2 or viral respiratory disease not identified as influenza or respiratory syncytial virus (RSV) (febrile or lower respiratory tract infection).

The study was performed in 2 phases as follows:

Phase 1a (˜60 subjects) subjects received 2 doses 4 weeks apart of PTX-B intramuscular (IM) vaccine or placebo.

Cohort 1 included 20 healthy subjects 18 to 64 years of age administered 16 μg PTX-B IM vaccine or placebo; 5 sentinel subjects (4 PTX-B:1 placebo), followed by remaining 11 PTX-B:4 placebo (cohort expansion) subjects.

Cohort 2 included 20 healthy subjects 18 to 64 years of age administered 40 μg PTX-B IM vaccine or placebo; 5 sentinel subjects (4 PTX-B:1 placebo), followed by remaining 11 PTX-B:4 placebo (cohort expansion) subjects.

Cohort 3 included 20 healthy subjects 18 to 64 years of age administered 100 μg PTX-B IM vaccine or placebo; 5 sentinel subjects (4 PTX-B:1 placebo), followed by remaining 11 PTX-B:4 placebo (cohort expansion) subjects.

Assessment of the sentinel cohorts included a safety follow-up assessment (including solicited and unsolicited AEs and safety laboratory evaluations) through the third day post vaccination.

Phase 1b (˜60 subjects) subjects received 2 doses 4 weeks apart of PTX-B vaccine (15 received Placebo and 45 active treatment).

Cohort 4 included 20 healthy subjects ≥65 years of age; 15 subjects were administered PTX-B IM vaccine with 1 dose level determined based on previous results from Phase 1a and 5 subjects were administered placebo.

Cohort 5 included 20 healthy subjects ≥65 years of age; 15 subjects were administered PTX-B IM vaccine with 1 dose level to be determined based on previous results from Phase 1a and 5 subjects were administered placebo.

Cohort 6 included 20 healthy subjects ≥65 years of age administered PTX-B IM vaccine, 1 dose level was determined based on previous results and 5 subjects were administered placebo.

About 14 days elapsed between each cohort for independent safety review before enrolling the next cohort.

Subjects visit the clinical site for screening (Days−21 to−1) and on Days 1, 8, 28±2, 36, 56±2, 90±3, 180±5, and 395-14. Safety phone calls to subjects were performed on Days 2 and 29. Screening procedures included informed consent, evaluation of entry criteria, demographics, height, weight, body mass index, medical and surgical histories, safety evaluations (including SAE evaluations, recording prior medications and procedures, physical examinations, vital sign assessments, clinical blood and urine samples, nasopharyngeal [NP] swab with the option for the clinic to confirm eligibility via an additional point-of-care test), and blood samples for immunogenicity analysis (antibodies). On Day 1, randomization was performed (overall 15:5 investigational vaccine:placebo vaccine for Phase 1a and Phase 1b cohorts). On Day 1 and Day 28, eligibility was confirmed and safety evaluations, immunogenicity and cell mediated immunity blood sampling, paper diary training and distribution, and vaccine or placebo administration was performed. Safety evaluations, blood and urine samples for safety evaluations, and blood samples for immunogenicity analysis and cell mediated immunity were performed through the end of study visit. Assessments of unsolicited AEs were performed on Day 1 through Day 56. Medically attended AE assessments were performed when subjects were in house on Days 1 through 56. Assessments of NOCDs, AESIs (including COVID 19 cases for enhanced disease), and PIMMCs were performed from Day 1 through the end of study. The SAEs will be assessed throughout the study.

Vaccine and placebo were prepared by an unblinded site pharmacist and administered by IM injection in the upper arm deltoid muscle of the non-dominant side at the clinical research site by unblinded CPU personnel. Subjects were observed for immediate AEs and/or reactogenicity for approximately 1 hour after administration of vaccine. Subjects were provided with a Diary Card and trained to record specifically elicited systemic and local symptoms daily, as well as any additional AEs, during the follow-up period after each vaccination. Subjects were requested to take a photo of completed Diary Card and text/email the photo to the site to ensure close oversight of reactions.

The duration of ongoing monitoring of subjects is approximately 14 months for each subject.

### Inclusion Criteria for Study

Subjects were required to meet all inclusion criteria (number 1-8 below) to be eligible for study participation. In addition, racial and ethnic minorities were sought to obtain a diverse study population.

1. Subject has read, understood, and signed the informed consent form.

2. Healthy adult males and females 18 to 64 years of age (Phase 1a), or ≥65 years of age (Phase 1b), inclusive, at screening.

3. Seronegative to SARS-CoV-2 and reverse transcription-polymerase chain reaction (RT-PCR)-negative at screening, without evidence of recent of exposure or viral respiratory disease not identified as influenza or RSV (febrile or lower respiratory tract infection).

4. Body mass index of ≥18 and ≤30 kg/m2 at screening.

5. Must be in general good health before study participation with no clinically relevant abnormalities that could interfere with study assessments. Older (≥65 years of age) participants can have stable comorbidities (no change in medications or exacerbations in past 3 months).

6. Women of childbearing potential (WOCBP) and men whose sexual partners are WOCBP must be able and willing to use at least 1 highly effective method of contraception (i.e., including hysterectomy, bilateral salpingectomy, and bilateral oophorectomy, hormonal oral [in combination with male condoms with spermicide], transdermal, implant, or injection, barrier [i.e., condom, diaphragm with spermicide]; intrauterine device; vasectomized partner [6 months minimum], clinically sterile partner; or abstinence) during the study. A female subject was considered a WOCBP after menarche and until she is in a postmenopausal state for 12 consecutive months (without an alternative medical cause) or otherwise permanently sterile. Subjects not of childbearing potential are not required to use any other forms of contraception during the study. Non-childbearing potential is defined as subject confirmed: Surgical sterilization (e.g., bilateral oophorectomy, bilateral salpingectomy, bilateral occlusion by cautery [Essure System® is not acceptable], hysterectomy, or tubal ligation), postmenopausal (defined as permanent cessation of menstruation for at least 12 consecutive months prior to screening); if postmenopausal status is unclear, pregnancy tests was performed prior to vaccinations.

7. Women of childbearing potential must have a negative pregnancy test before each vaccination. If menopausal status is unclear, a pregnancy test is required.

8. Must be able to attend all visits (scheduled and unscheduled, as applicable) for the duration of the study and comply with all study procedures, including daily completion of the Diary Card after each injection.

### Exclusion Criteria for Study

Subjects including subjects ≥65 years (for phase 1b) were not eligible for study participation if they met any of the exclusion criteria (numbered 1-35 below), or were discontinued at the discretion of the investigator if they developed any of the exclusion criteria during the study.

1. History of an acute or chronic medical condition including dementia that, in the opinion of the investigator, would render vaccination unsafe or would interfere with the evaluation of responses.

2. History of any medical conditions that place subjects at higher risk for severe illness due to SARS-CoV-2 will be excluded including: Chronic kidney disease; COPD (chronic obstructive pulmonary disease); Heart conditions, such as heart failure, coronary artery disease, or cardiomyopathies; Any Immunocompromised state including from transplantation, history immunodeficiency, HIV, immunosuppressive drug intake; Sickle cell disease; Current smoker or history of >5 pack/years of smoking; Type 2 diabetes mellitus.

Subjects with history of any of the following conditions who might be at an increased risk of complications from COVID-19 were excluded: Asthma (moderate-to-severe); Cerebrovascular disease (affects blood vessels and blood supply to the brain); Cystic fibrosis; Hypertension or high blood pressure; Neurologic conditions, such as dementia; Liver disease; Pulmonary fibrosis (having damaged or scarred lung tissues); Thalassemia (a type of blood disorder); Type 1 diabetes mellitus.

3. History of ongoing clinical condition or medication or treatments that may adversely affect the immune system.

4. Individuals who are seropositive or RT-PCR positive for SARS-CoV-2, including prior to a second dose of PTX-B vaccine.

5. Individuals who are at increased risk of exposure to SARS-CoV-2 (e.g., healthcare workers, emergency responders).

6. Close contact of anyone known to have SARS-CoV-2 infection within 30 days prior to vaccine administration.

7. Living in a group setting or group care facility (e.g., dormitory, assisted living or nursing home).

8. Individuals with any elevated (Grade 1 or higher) laboratory test assessed as clinically significant for age/sex by the investigator at screening.

9. Individuals with any elevated for age/sex (Grade 1 or higher) liver function enzyme at screening, regardless of the appraisal of clinical significance (one retest permitted). The criteria for excluding subjects with elevated liver enzymes are as follows: Alkaline phosphatase, alanine aminotransferase, aspartate aminotransferase, or gamma-glutamyl transferase >1.5× upper limit of normal (ULN); Total bilirubin >1.5×ULN.

10. Active neoplastic disease (excluding nonmelanoma skin cancer that was successfully treated) or a history of any hematological malignancy. “Active” is defined as having received treatment within the past 5 years.

11. Long-term (>2 weeks) use of oral or parenteral steroids or high-dose inhaled steroids (>800 μg/day of beclomethasone dipropionate or equivalent) within 6 months before screening (nasal and topical steroids are allowed).

12. History of autoimmune, inflammatory disease, or PIMMCs (Appendix B).

13. Women currently pregnant, lactating, or planning a pregnancy between enrollment and 181 days after randomization.

14. History of Guillain-Barre Syndrome or any degenerative neurology disorder.

15. History of anaphylactic-type reaction to any injected vaccines.

16. Known or suspected hypersensitivity to 1 or more of the components of the vaccine.

17. History of alcohol abuse, illicit drug use, physical dependence to any opioid, or any history of drug abuse or addiction within 12 months of screening.

18. Acute illness or fever (temperature >37.5C) within 3 days before study enrollment (enrollment may be delayed for full recovery if acceptable to the investigator).

19. Individuals currently participating or planning to participate in a study that involves an experimental agent (vaccine, drug, biologic, device, or medication); or who have received an experimental agent within 1 month (3 months for immunoglobulins) before enrollment in this study; or who expect to receive another experimental agent during participation in this study.

20. Receipt of immunoglobulin or another blood product within the 3 months before enrollment in this study or those who expect to receive immunoglobulin or another blood product during this study.

21. Individuals who intend to donate blood within 6 months after the first vaccination.

22. Individuals using prescription medications for prophylaxis of SARS-CoV-2.

23. Individuals who plan to receive another vaccine within the first 3 months of the study except influenza vaccine which should not be given within 2 weeks of vaccine.

24. Receipt of any other SARS-CoV-2 or other experimental coronavirus (Middle East Respiratory Syndrome, SARS etc.) vaccine at any time prior to or during the study.

25. Receipt of any investigational vaccine or investigational drug within 1 month of enrollment and through the end of the study (1 year after the last vaccination).

26. Plan to travel outside Canada from enrollment through Day 56.

27. History of surgery or major trauma within 12 weeks of screening, or surgery planned during the study.

28. Significant blood loss (>400 mL) or has donated 1 or more units of blood or plasma within 6 weeks prior to study participation.

29. Strenuous activity or significant alcohol intake (as assessed by the investigator) within 72 hours prior to safety laboratory sample collection.

30. Positive urine drugs of abuse screen or alcohol breathalyzer test result.

31. Positive screen for human immunodeficiency virus-1 and -2 antibodies, hepatitis B surface antigen, or hepatitis C virus antibody.

32. Involved in the planning or conduct of this study.

33. Unwilling or unlikely to comply with the requirements of the study.

34. Subjects is an employee, contractor, or friend or relative of any employee of sponsor, CRO, study site, or site affiliate.

35. Subjects oximetry is <90%.

### Study Compositions

The vaccine product was presented as a 0.2 mg/mL, 2 mL fill in a 3 mL United States Pharmacopeia/European Pharmacopeia Type I borosilicate glass vial with a fluoro-resin laminated bromobutyl rubber stopper and an aluminum coverseal with a red, plastic, flip-off cap. PTX-B messenger ribonucleic acid Humoral Vaccine was an injectable IM 0.2 mg/mL 0.5 mL solution, with multiple dose levels as described.

The placebo was commercially available sodium chloride 0.9% injectable IM solution 0.0 mg/mL 0.5 mL.

### Summary of Study Results

This Phase 1 first-in-human observer-blinded, randomized, placebo —controlled, ascending dose study evaluated the safety, tolerability, and immunogenicity of PTX-B vaccine in healthy seronegative adults aged 18 to 64. The study was designed with dose-escalations and was performed in seronegative adult subjects without evidence of recent of exposure to SARS-CoV-2.

Safety Population included all subjects who provided consent, were randomized, and received any amount of vaccine/placebo. The Safety Population was used for all safety analyses and analysis of immunogenicity and analyzed as actually treated.

Per Protocol Population included all subjects in the Safety Population who received the assigned doses of the vaccine/placebo according to protocol, had serology results, and no major protocol deviations affecting the primary immunogenicity outcomes, as determined by the Sponsor before database lock and unblinding. The PP Population was the primary population used for the analysis of safety endpoints.

Modified Intent-to-Treat Population included all subjects in the Safety Population who provided any serology data. The mITT Population was used for the analysis of immunogenicity endpoints. Analysis using the mITT Population was performed if it differed from the PP Population by ≤5% of the subjects for each of the treatment groups.

Immunogenicity data for each cohort and study phase was listed and summarized by phase, cohort, and time point using appropriate descriptive statistics.

Vital signs, clinical laboratory tests, and physical examination findings were listed and summarized by study phase, cohort, and time point using appropriate descriptive statistics.

The number and percentage of subjects reporting any treatment-emergent adverse event (TEAE) or reactogenicity were summarized by study phase and cohort and tabulated by system organ class and preferred term (coded using MedDRA). The TEAEs were further classified by severity and relationship and for SAEs, medically attended AEs, NOCD, and AESIs.

Additionally, number of subjects who became infected after vaccination and whether being vaccinated made the disease less or more severe were presented.

Subjects were randomized to receive either PTX-B vaccine or placebo in a 3:1 ratio. Dosing occurred in cohorts starting with the 16 μg, dose followed by 40 μg and then 100 μg. Each cohort was started with a 5-subject sentinel group dosed first followed by the rest of the cohort. The adverse events were collected at frequent intervals and an independent Safety Review Committee (iSRC) comprising of infectious disease experts and statisticians met at frequent intervals to review and authorize dosing of the next group.

A total of 60 subjects were enrolled and 58 subjects received both doses of study medications. Two subjects dropped out of the study after receiving one dose of study medication due to personal reasons unrelated to study drug. Overall, the male to female ratio was exactly 50:50, with 83.3% being white, 13.3% Asian and 3.3% noted other.

Adverse events collected were graded according to the industry standard FDA guidance on vaccine reactions as either local (at the site of the injection) or systemic. Overall PTX-B was safe and well tolerated at all three dose levels of 16 μg, 40 μg and 100 μg. There were no Serious Adverse Events. The only local adverse event recorded was pain at the injection site as would be expected. There was no redness or swelling after either dose of 40 μg of PTX-B. This compares favorably with other mRNA vaccines where redness and swelling has been recorded in clinical trials as well as in general use. Systemic reactions included fatigue, chills and fever and were generally mild to moderate and well tolerated with headache being the most common reaction occurring up to 60% after the second dose. These results compare very favorably with published adverse events to approved emergency use mRNA vaccines.

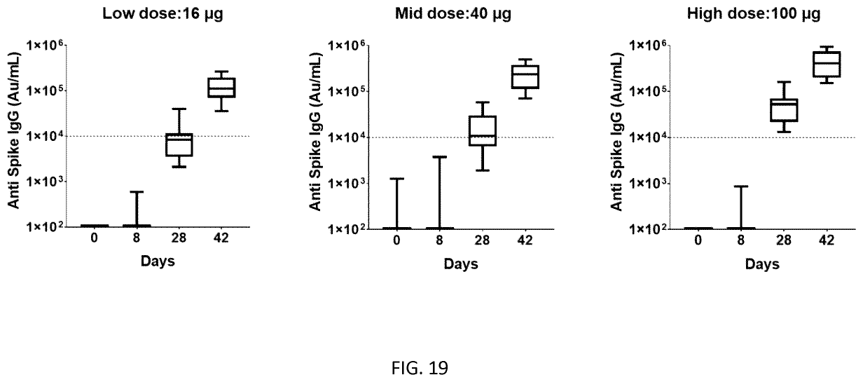

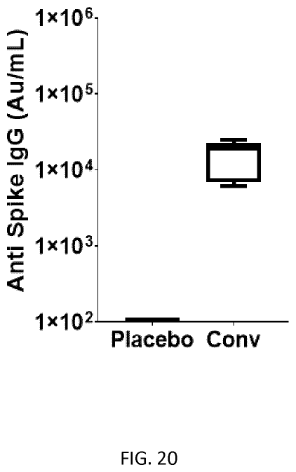

PTX-B vaccination induced high anti-S IgG antibodies: Participants in the clinical trial were vaccinated on day zero and day twenty-eight. Plasma samples were collected on day zero (pre-screen), on days 8, 28 (before the 2nd dose) and 42 to determine levels of IgG anti-S protein using multiplex sandwich-based immunoassays with an electrochemiluminescence (ECL) readout from the Meso Scale Discovery (MSD) platform. All study participants in all three vaccine dose cohorts (16, 40 and 100 μg) developed a strong IgG antibody response against Spike protein that was detected by day 28 and enhanced by day 42. No antibodies against S protein were detected in participants injected with placebo. Also, the highest levels of antibodies were found in the mid and high doses. By day 42, PTX-B vaccinated participants had more than one log higher antibody levels than convalescent subjects' plasma which was evaluated in the same assay. Total IgG levels were analyzed in subjects. High levels of both anti-Spike (shown in FIG. 19) and anti-RBD were induced at all dose levels after the just the first dose (Day 28) (FIG. 19). The levels increased even higher two weeks after the second dose (Day 42) to levels more than 10-fold higher than the average values from 5 serum samples from convalescent patients (FIG. 19). Anti-Spike IgG (Au/mL) was also measured in SARS-CoV-2 convalescent patients (FIG. 20).

The levels of antibodies were comparable to the ones published in a recent report from Stanford University, where IgG responses in individuals vaccinated with the COVID-19 mRNA vaccine compared to SARS-CoV-2 infected patients were evaluated (Roltgen et al. (Apr. 7, 2021). mRNA Vaccination Compared to Infection Elicits an IgG-Predominant Response with Greater SARS-CoV-2 Specificity and Similar Decrease in Variant Spike Recognition. Pre-print downloaded May 11, 2021 from doi.org/10.1101/2021.04.05.21254952).

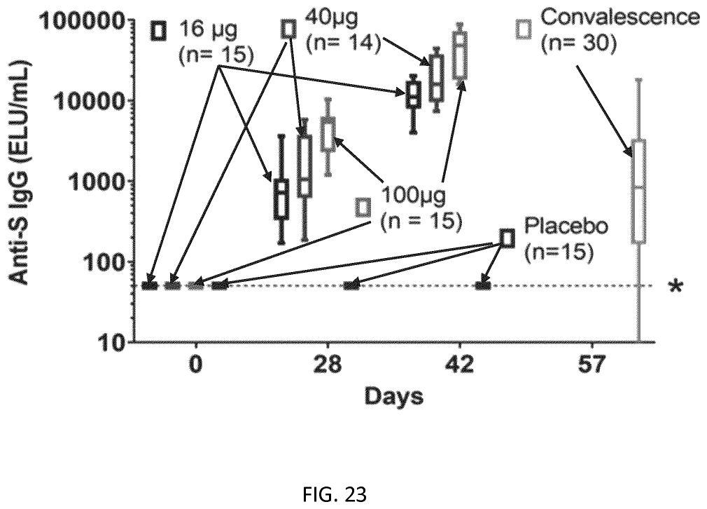

The high levels of anti-S protein IgG antibodies induced at all three different doses were further confirmed using ELISA. Serum samples from vaccinated subjects were added to spike protein coated ELISA plates and bound IgG antibodies were then detected using peroxidase labeled secondary anti-IgG antibody. The IgG antibody concentrations, which were determined by interpolation on the calibration curve, are up to about 1000 ELISA UNITS/milliliter after a single dose of PTX-B (Day 28) (FIG. 23). The IgG concentrations continue to increase after the second dose, over 10,000 ELISA UNITS/milliliter (FIG. 23).

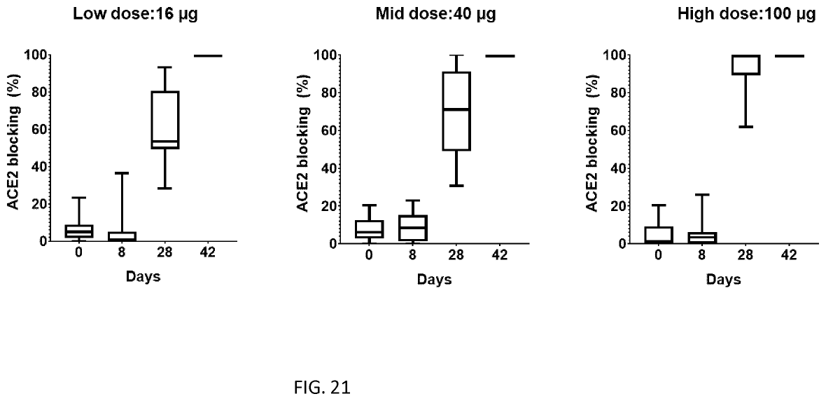

PTX-B vaccination induced high neutralizing antibody levels: Neutralizing activity from the study participants' plasma was evaluated by S-ACE2 blocking MSD assay. Here the antibodies block the interaction between S protein with the ACE2 receptor and the decrease in ECL signal is used to calculate percentage inhibition of the plasma at the same dilution. All participants in the study from the 3 dose levels showed blocking activity by day 28 and all of them reached 100% blocking activity by day 42 with samples diluted 1:100 or greater. These results indicate that PTX-B induced a strong neutralizing antibody response. Moreover, the quantification of the antibody levels in ng/mL with a reference standard showed that all participants produced neutralizing antibodies by day 28 with the first immunization and increase ten-fold by day 42, two weeks after the second immunization. Neutralizing antibodies were analyzed in subjects by assessing the ability to block interaction between Spike protein and hACE2 in vitro. PTX-B vaccinated participants showed high levels of neutralization activity in plasma at day 28 and day 42 using an S:ACE2 receptor blocking assay based on MSD technology (FIG. 21). When percentage inhibition was evaluated in samples diluted at or more than 100-fold, all the participants had 100% inhibition by day 42. Data published by Roltgen et al, where individuals were vaccinated with the BioNTech/Pfizer mRNA vaccine, showed a 75% average inhibition at day 42 (1:100 dilution) using the same MSD assay. These data indicate that PTX-B has a stronger neutralization capacity at all doses.

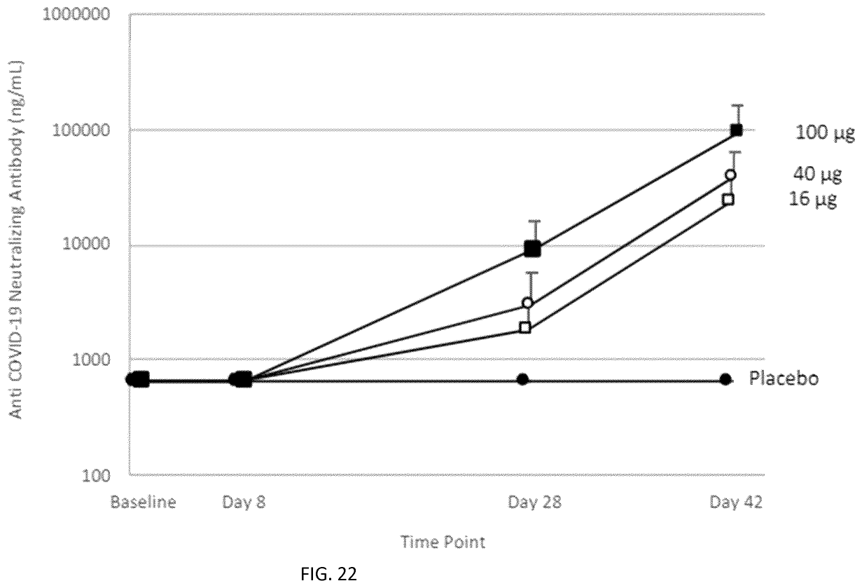

Neutralizing activity was evaluated by S-ACE2 blocking MSD assay. Quantification of the antibody levels in ng/mL is based on the activity of a reference standard. All participants produced neutralizing antibodies by day 28 with the first immunization, and increased ten-fold by day 42, two weeks after the second immunization (FIG. 22).

### Example 9. Pseudovirus Neutralization of PTX-B Induced Anti-COVID19 Antibodies in Human

Neutralizing activity was further evaluated by pseudovirus neutralization assay. Spike-pseudotyped AG-luciferase rVSV viruses were produced in ES-293 cells following the protocol reported by Bewley K. R (Bewley et al., Quantification of SARS-CoV-2 neutralizing antibody by wild-type plaque reduction neutralization, microneutralization and pseudotyped virus neutralization assays; Nature Protocols, 2021, 16, 3114-3140). Stocks of pseudovirus were subjected to the sighting procedure to obtain the optimal pseudovirus dilution.

Serum samples from PTX-B vaccinated subjects were heat treated to inactive components. The inactivated serum samples and reference sera were serially diluted. The prediluted test sera and reference sera were incubated with pseudovirus diluent at a 1:1 ratio for 1 hour at 37° C. The serum and pseudovirus mixtures were then transferred to plated Vero E6 cells and incubated for an additional 18-22 hours at 37° C. and under 5% CO2.

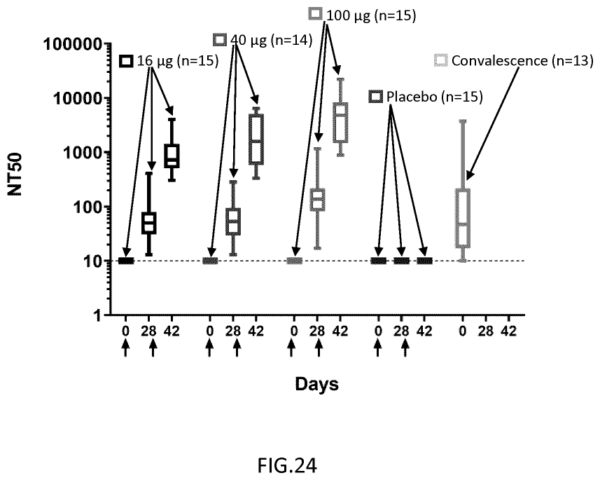

ONE-Glo EX luciferase assay reagent was added to the cell plates. The reaction plates were incubated for 3 minutes at room temperature and read luciferase levels. The midpoints of each curve were determined using the SoftMax Pro protocol using 4PL regression and outputs were reported as each sample's median neutralization titer (NT₅₀). The PTX-B vaccine leads to a neutralization titer that is comparable to that obtained from patients in convalescence, after a first dose (Day 28). The neutralization titers are further increased two weeks after a second dose of PTX-B (Day 42), which is higher than that obtained from patients in convalescence (FIG. 24).

The median neutralization level can reach to 0.3 (16 μg dose and 40 g dose) and 0.8 (100 μg dose) fold of the level in convalescent patients, respectively, after the first dose (on Day 28; before receiving the second dose). After two weeks after the second dose (on Day 42), the median neutralization levels increase to 4.0-fold (16 μg dose), 8.5 fold (40 μg dose) and 23.0 fold (100 μg dose) of the level in convalescent patients, respectively. The 100 μg dose induced higher binding and neutralizing antibody titers than the lower doses.

The neutralization activity of neutralizing antibodies induced by PTX-B vaccine was compared with COVID19 mRNA vaccines BNT162b2 and mRNA-1273. As shown in Tables 11 and 12, the mean neutralization level of neutralizing antibodies induced by PTX-B vaccine is comparable to that of BNT162b2.

TABLE 11
Mean neutralization level of PTX-B
(fold of convalescence (n = 33))

| | 16 μg | 40 μg | 100 μg |
|---|---|---|---|
| Day | (n = 15) | (n = 14) | (n = 15) |
| 28 | 0.3 | 0.3 | 0.8 |
| 42 | 4.0 | 8.6 | 23.0 |

TABLE 12
Mean neutralization level of BNT162b2* (fold of convalescence)

| Day | 10 μg | 20 μg | 30 μg |
|---|---|---|---|
| 21 | 0.2 | 0.2 | 0.1 |
| 28 | 1.7 | 3.9 | 3.8 |
| 35 | 1.0 | 3.1 | 1.7 |

*The data are from Walsh, E. E. et al., Safety and immunogenicity of two RNA based COVID-19 vaccine candidates, N Engl. J. Med., 2020, 383(25): 2439-2450.

Similarly, the neutralization activity of neutralizing antibodies induced by PTX-B vaccine is comparable to those induced by mRNA-1273 vaccine as compared to the data reported by Anderson E. J., et al., Safety and immunogenicity of SARS-CoV-2 mRNA 1273 vaccine in older adults. N Engl. J. Med., 2020, 383: 2427-2438.

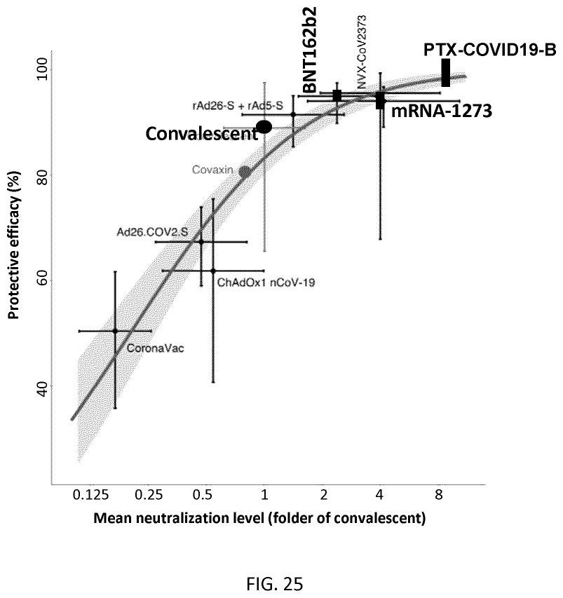

The prediction of the protective efficacy of PTX-B vaccine using the modeling developed by Khoury, D. S., et al, (Khoury, D. S., et al, Neutralizing antibody levels are highly predictive of immune protection from symptomatic SARS-CoV-2 infection. Nat. Med., 2021, 27: 1205-1211), suggests that PTX-B is comparable to the nucleic acid vaccines BNT162b2 and mRNA-1273 (FIG. 25). The neutralization activity of PTX-B vaccine is higher than BNT162b2 and mRNA-1273 vaccines (FIG. 25). The results suggest that PTX-B can elicit a stronger response in human than the BNT162b2 and mRNA-1273 vaccines.

### Example 10: Neutralization Capacity for Different SAR-CoV-2 Variants of Concern (VOCs)

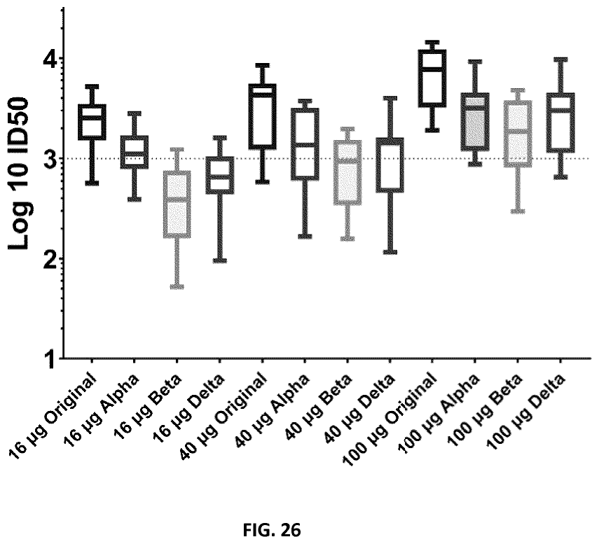

The same sera from groups of subjects receiving 2 doses (day 0 and da 28) of 16 μg, 40 μg or 100 μg PTX-B vaccine were tested for their pseudoviral neutralization activity against viral variants. Pseudovirus of SAR-CoV-2 original virus and its variants, Alpha, Beta and Delta VOCs were prepared for neutralization assays. The results as shown in FIG. 26 suggest that PTX-B vaccine induces comparable neutralization activity against the SAR-CoV-2 original strain, and the Alpha, Beta and Delta VOCs in all three dose cohorts (FIG. 26). The neutralization activity induced by the high dose of PTX-B (100 μg) in general is higher than that induced by the low dose (16 μg) and mid-dose (40 μg) of PTX-B. The neutralization capacity against the original strain, Alpha and Delta variants is also comparable to that obtained by the BNT162b2 vaccination reported by Ade K. T. et al., (Ade K. T et al., Neutralizing antibody responses to SARS-CoV-2 variants in vaccinated Ontario long-term care home residents and workers, Aug. 8, 2021; MedRxiv preprint doi: https://doi.org/10.1101/2021.08.06.2126172).

### Equivalents and Scope

Those skilled in the art will recognize, or be able to ascertain using no more than routine experimentation, many equivalents to the specific embodiments described herein. The scope is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments, to the tenth of the unit of the lower limit of the range, unless the context clearly dictates otherwise.

In addition, it is to be understood that any particular embodiment that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions described herein (e.g., any therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A polynucleotide encoding at least one structural protein of SARS-CoV-2 or a variant thereof, wherein the at least one structural protein is the spike (S) protein and wherein the polynucleotide comprises a first sequence region, said first sequence region comprising a nucleic acid sequence having at least 95% identity to a member of the group consisting of SEQ ID NO: 7, 20, 26, 27, and 32.

2. The polynucleotide of claim 1, wherein said first sequence region is at least 95% identical or at least 99% identical to SEQ ID NO: 7.

3. (canceled)

4. The polynucleotide of claim 3, wherein said first sequence region consists of SEQ ID NO: 7.

5. The polynucleotide of claim 4, wherein the encoded spike protein has a protein sequence of SEQ ID NO: 2.

6. The polynucleotide of claim 1, wherein at least 50% of the polynucleotide sequence is codon optimized.

7.-8. (canceled)

9. The polynucleotide of claim 1, wherein the polynucleotide is an mRNA.

10. The polynucleotide of claim 9, comprising a 5′UTR and a 3′UTR, wherein said 5′UTR comprises SEQ ID NO: 47 and said 3′UTR comprises SEQ ID NO: 48.

11. The polynucleotide of claim 10, wherein at least one uracil nucleoside is modified to be N1-methylpseudouridine.

12. The polynucleotide of claim 11, wherein all uracil nucleosides are modified to be N1-methylpseudouridine.

13. A nucleic acid vaccine comprising the polynucleotide of claim 12.

14. The nucleic acid vaccine of claim 13, formulated in lipid nanoparticle (LNP).

15. A pharmaceutical composition comprising the nucleic acid vaccine of claim 14 and a pharmaceutically acceptable excipient.

16. The pharmaceutical composition of claim 15, wherein the polynucleotide of the nucleic acid vaccine is an mRNA, and wherein the pharmaceutical composition comprises about 0.2 mg/mL of the mRNA.

17. The pharmaceutical composition of claim 16 which is suitable for intramuscular (IM) injection.

18.-26. (canceled)

27. A method of preventing COVID-19 or mitigating or ameliorating the physiologic effects or symptoms of COVID-19 in a subject, said method comprising administering the nucleic acid vaccine of claim 13 to said subject.

28. (canceled)

29. The method of claim 27, wherein the polynucleotide of the nucleic acid vaccine is an mRNA, and wherein the dose administered to the subject is:
i. from about 5 μg to about 100 μg of the mRNA;
ii. about 16 μg of the mRNA;
iii. about 40 μg of the mRNA; or
iv. about 100 μg of the mRNA.

30.-34. (canceled)

35. The method of claim 27, comprising administering a second dose of the nucleic acid vaccine after between about 1 and about 5 weeks of a first dose, or after about 4 weeks of a first dose.

36. (canceled)

37. The method of claim 27, wherein:
i. anti-Spike protein IgG antibodies are detected in the subject by day 28 after receiving a first dose of the nucleic acid vaccine;
ii. anti-Spike protein IgG antibodies are present in an enhanced amount in the subject by day 42 after receiving a second dose of the nucleic acid vaccine; and/or
iii. anti-Spike protein IgG antibodies in the subject are greater than 10-fold the values of anti-Spike protein IgG antibodies in serum samples from SARS-CoV-2 convalescent patients.

38.-39. (canceled)

40. A method of inducing SARS-CoV-2 neutralizing antibody production in a subject comprising administering the nucleic acid vaccine of claim 13 to said subject.

41. The method of claim 40, wherein;
i. the SARS-CoV-2 neutralizing antibodies can be detected in the subject by day 28 after administration,
ii. the SARS-CoV-2 neutralizing antibody production is enhanced in the subject at day 42 after receiving a second dose of the nucleic acid vaccine; and/or
iii. the level of neutralizing antibodies to SARS-CoV-2 is increased ten-fold by day 42.

42.-43. (canceled)


SEQUENCE LISTING (53 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 5 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 1273 aa, Severe acute respiratory syndrome coronavirus 2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - RNA, 3822 nt, Artificial Sequence
  source: /note=“Description of Artificial Sequence Synthetic polynucleotide”
auguuuguguuucugguccuccugccccuggugucaagccaaugcgucaaccugacuacacggacccagcugcccccugcuuauaccaacucauuuacuagagggguguacuacccagauaaaguguucagaucaagcgugcuccacagcacccaggaccucuuccugcccuucuuuucaaacgugaccugguuccaugccauccaugugagcggaaccaacgggaccaagcgguucgauaauccagugcuccccuucaacgaugggguguauuuugcaagcacugaaaagucaaauauuauccgcggauggauuuucgggacaacacucgauagcaagacccagucacugcucaucgugaacaacgcuacaaauguggucaucaaggugugcgaguuucaguucugcaaugacccuuuucucggcgucuacuaccacaagaacaacaagagcuggauggaaucagaauuccggguguauagcucagcuaauaauugcaccuucgaguaugugagccagccuuuccucauggaccuggaggggaagcagggcaacuucaagaaucugagagaauucguguuuaaaaacaucgauggauauuucaaaaucuauagcaagcacacuccuaucaaccucguccgggaccugccacaaggcuuuagcgcacuggagccccucguggaccugcccaucgggaucaauaucaccagauuucagacucugcuggcucuccaccgcagcuaccugacuccaggggauagcucaagcggcuggaccgcuggagccgcugccuauuacgugggguaccugcagccuaggacuuuucugcugaaguacaaugagaacgggacaauuacugacgcugucgauugugcacucgacccccucagcgagacaaaauguacccugaaaagcuuuacuguggaaaaagggauuuaccagacuagcaauuucagaguccagccaacagagucaaucgugcgguuuccuaacaucacaaaccucugcccauucggggaggucuucaaugccacccgcuucgccagcguguacgcuuggaauagaaagcggaucagcaacugcguggccgacuacagcgugcucuacaauagcgcuagcuucucaacauuuaaaugcuacggggugagccccacaaaacugaaugaccuguguuucaccaauguguaugccgauagcuucgugauuagaggcgacgaggugcgccaaaucgcaccuggccaaaccggaaagauugcagauuauaacuauaagcugcccgaugauuuuacuggcugugucaucgcuuggaacagcaacaaccucgauucaaaagucggaggaaacuauaacuaccuguauagacuguucagaaagagcaaccugaagcccuuugaaagggauaucagcacagaaauuuaccaggccgggagcaccccaugcaacggggucgaaggguuuaacuguuauuucccccugcagagcuacggguuucaacccaccaauggggugggguaucagccuuaccgcguggucgugcugagcuucgaacugcugcacgcccccgccaccgugugcggcccuaaaaagagcacaaaccucgucaagaacaagugcgugaacuucaauuucaacggccugacuggcacaggcguccucaccgaaagcaauaaaaaguuccucccuuuccagcaauucggcagagacauugcagacacaacugaugcugugagggacccccagacacuggagauccuggauauuacccccuguucauucggcggagugucagucaucaccccuggcacuaacacaucaaaucagguggcaguccuguaucagggcgugaacugcaccgaagugccugucgccauucaugcagaccagcugacccccacuuggagaguguacucaacuggcagcaaugucuuucaaaccagagccggcugucucaucggcgcagaacaugucaacaacagcuaugagugugacaucccaaucggagcuggcaucugugccagcuaucagacacagaccaacagcccucggagggcuagaagcguggccagccagagcauuaucgcuuacaccaugucacugggcgccgagaauagcguggccuauagcaacaauagcaucgccauucccaccaauuuuaccaucagcgugaccacagagauccucccagugagcaugaccaagacaagcgucgauugcacaauguacauuugcggcgacagcacugaauguagcaaccuccugcugcaguacgggagcuuuuguacccaacugaaucgggcacucaccgggaucgccguggagcaggauaaaaacacucaggaggucuuugcucaggucaaacagaucuacaaaaccccaccaauuaaagacuucggaggcuucaacuucucacagauucugccagacccuucaaagccaagcaagcgcagcuucauugaagaucuccuguucaacaaggugacucuggcugacgcuggguucaucaagcaguauggagacugccugggggacaucgcagcaagggaucugaucugugcucagaaguuuaauggccucacagugcugcccccucuccugacugacgagaugauugcucaguacaccagcgcccugcuggcuggaacuaucacaucaggguggacauucggggccggggcugcucugcagauuccuuucgcaaugcaaauggcauauagguucaacgggaucggcgucacacagaacgugcuguaugagaaucagaaacugaucgcaaaucaauuuaauucagcaaucgggaaaauccaggacucacugucaucaaccgcaucagcccugggcaagcugcaggacgucgucaaccaaaacgcccaggcacucaacacacuggugaagcagcugucaagcaauuucggagcaaucagcagcguccugaaugauauccugagcaggcuggauaagguggaggcagaagugcagaucgaccgccugaucaccggccggcuccagagccugcaaaccuaugucacucagcaacugaucagagcugcagaaauccgggcaagcgccaaucuggccgccaccaagaugucagaaugcgugcucggccagagcaagaggguggacuucugcggcaaaggcuaccaccugaugagcuucccucagagcgccccccacggcguggucuuccuccaugugacuuaugugcccgcacaggaaaagaacuucacuacugccccagccaucugccacgacggaaaggcucauuucccacgcgaggggguguucgucagcaauggaacccacugguucgugacccagcggaacuucuaugagccucagauuauuaccacagacaacacauuugugucaggaaauugcgaugucgugaucgggaucgucaacaauacugucuaugacccccuccagccagaacucgauagcuucaaagaggaacucgauaaguauuuuaaaaaucacaccagcccugauguggaucugggcgauaucagcggaaucaaugcuagcguggugaacauccagaaggagauugacaggcucaacgaagucgcaaagaaccugaacgagucacugaucgaccuccaggaacugggcaaguacgaacaguacaucaaguggcccugguacaucuggcucggguucaucgcuggccugauugcuaucgugauggugacaaucaugcucugcuguaugaccagcugcugcagcugccugaagggaugcugcucaugcggcagcugcuguaaguucgaugaggacgacucagagcccgugcucaaaggcgugaaacugcauuauacuuga

SEQ ID NO: 8 - DNA, 1084 bp, Artificial Sequence
  source: /note=“Description of Artificial Sequence Synthetic polynucleotide”
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatggccgactcaaacggcacaattaccgtggaggagctgaagaagctcctggagcagtggaatctggtcattggctttctgttcctgacctggatctgcctgctgcagttcgcttacgctaaccgcaaccggttcctctatatcattaaactcatttttctgtggctgctctggcctgtgacactggcctgtttcgtgctcgccgccgtctaccgcattaactggatcacaggagggattgctatcgctatggcttgcctggtcgggctgatgtggctctcatacttcatcgcaagctttaggctctttgcccggactcggagcatgtggtcattcaaccccgagacaaacattctgctcaatgtgcctctgcacgggaccatcctgaccaggcccctcctggagagcgagctggtgatcggggccgtgatcctgaggggacatctgaggatcgccggccatcatctgggcaggtgtgacatcaaggatctccctaaggagatcactgtggcaactagccggacactgagctattacaaactcggcgcaagccagcgcgtcgctggggactcaggcttcgcagcttacagcagatataggatcgggaactacaagctgaataccgatcacagctcaagcagcgataacatcgctctgctcgtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 1555 bp, Artificial Sequence
  source: /note=“Description of Artificial Sequence Synthetic polynucleotide”
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 1675 bp, Artificial Sequence
  source: /note=“Description of Artificial Sequence Synthetic polynucleotide”
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgtcagataacggcccacagaatcagagaaacgccccacggatcacttttggcgggcctagcgattcaaccgggtcaaatcagaatggggaacgcagcggggcccggagcaagcagagacggccccaagggctgcctaacaatacagcatcatggttcacagccctgacccagcatggcaaggaagatctcaagtttcctcgcggacagggcgtgcctattaacaccaattcaagccctgacgatcagatcggctactacagaagggctactcgcagaatccgcggaggcgatggaaaaatgaaagacctgagcccaaggtggtacttctattacctgggaactggccctgaggcaggactgccctatggcgccaacaaggatggcattatttgggtcgcaactgaaggagcactgaacacaccaaaggatcacatcggaacaaggaaccccgcaaacaatgccgctatcgtgctccagctgcctcaagggactaccctgcccaaagggttttatgccgaagggagcaggggcgggagccaggcaagcagcagaagctcatcacggtcaagaaattcaagcaggaatagcaccccaggaagctcaaggggaacaagcccagcccggatggcaggcaacggcggggatgcagcactcgcactgctcctgctggaccgcctgaatcagctggaatcaaaaatgagcgggaagggacagcaacagcagggacaaactgtcactaagaagtcagccgccgaggcaagcaagaagcccaggcaaaagcgcactgcaaccaaggcttacaatgtcactcaagcattcggcagaaggggcccagaacagacccagggcaatttcggcgaccaggagctgattagacaaggcactgattacaaacactggcctcagatcgctcagtttgcccccagcgcttcagccttttttgggatgagccggatcggcatggaggtgacaccatcagggacctggctcacctacactggagccattaaactggatgacaaagacccaaacttcaaagatcaagtgatcctgctcaacaagcacattgatgcatataaaacttttcctcctactgagcctaagaaggacaagaagaagaaagcagatgagacacaggctctgccccagcgccagaagaagcagcagactgtgacactgctgcccgctgcagatctggacgacttcagcaaacagctgcaacaatcaatgagctcagctgactcaactcaggcttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 1621 bp, Artificial Sequence
  source: /note=“Description of Artificial Sequence Synthetic polynucleotide”
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatggacatgagagctccagcccagatctttggctttctgctcctgctgtttccaggcactaggtgcgacaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 4238 bp, Artificial Sequence
  source: /note=“Description of Artificial Sequence Synthetic polynucleotide”
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccaatgtttgtgtttctggtcctcctgcccctggtgtcaagccaatgcgtcaacttcactacacggacccagctgccccctgcttataccaactcatttactagaggggtgtactacccagataaagtgttcagatcaagcgtgctccacagcacccaggacctcttcctgcccttcttttcaaacgtgacctggttccatgccatccatgtgagcggaaccaacgggaccaagcggttcgccaatccagtgctccccttcaacgatggggtgtattttgcaagcactgaaaagtcaaatattatccgcggatggattttcgggacaacactcgatagcaagacccagtcactgctcatcgtgaacaacgctacaaatgtggtcatcaaggtgtgcgagtttcagttctgcaatgacccttttctcggcgtctactaccacaagaacaacaagagctggatggaatcagaattccgggtgtatagctcagctaataattgcaccttcgagtatgtgagccagcctttcctcatggacctggaggggaagcagggcaacttcaagaatctgagagaattcgtgtttaaaaacatcgatggatatttcaaaatctatagcaagcacactcctatcaacctcgtccggggcctgccacaaggctttagcgcactggagcccctcgtggacctgcccatcgggatcaatatcaccagatttcagactctgctggctctccacatcagctacctgactccaggggatagctcaagcggctggaccgctggagccgctgcctattacgtggggtacctgcagcctaggacttttctgctgaagtacaatgagaacgggacaattactgacgctgtcgattgtgcactcgaccccctcagcgagacaaaatgtaccctgaaaagctttactgtggaaaaagggatttaccagactagcaatttcagagtccagccaacagagtcaatcgtgcggtttcctaacatcacaaacctctgcccattcggggaggtcttcaatgccacccgcttcgccagcgtgtacgcttggaatagaaagcggatcagcaactgcgtggccgactacagcgtgctctacaatagcgctagcttctcaacatttaaatgctacggggtgagccccacaaaactgaatgacctgtgtttcaccaatgtgtatgccgatagcttcgtgattagaggcgacgaggtgcgccaaatcgcacctggccaaaccggaaacattgcagattataactataagctgcccgatgattttactggctgtgtcatcgcttggaacagcaacaacctcgattcaaaagtcggaggaaactataactacctgtatagactgttcagaaagagcaacctgaagccctttgaaagggatatcagcacagaaatttaccaggccgggagcaccccatgcaacggggtcaaagggtttaactgttatttccccctgcagagctacgggtttcaacccacctatggggtggggtatcagccttaccgcgtggtcgtgctgagcttcgaactgctgcacgcccccgccaccgtgtgcggccctaaaaagagcacaaacctcgtcaagaacaagtgcgtgaacttcaatttcaacggcctgactggcacaggcgtcctcaccgaaagcaataaaaagttcctccctttccagcaattcggcagagacattgcagacacaactgatgctgtgagggacccccagacactggagatcctggatattaccccctgttcattcggcggagtgtcagtcatcacccctggcactaacacatcaaatcaggtggcagtcctgtatcagggcgtgaactgcaccgaagtgcctgtcgccattcatgcagaccagctgacccccacttggagagtgtactcaactggcagcaatgtctttcaaaccagagccggctgtctcatcggcgcagaacatgtcaacaacagctatgagtgtgacatcccaatcggagctggcatctgtgccagctatcagacacagaccaacagccctcggagggctagaagcgtggccagccagagcattatcgcttacaccatgtcactgggcgtcgagaatagcgtggcctatagcaacaatagcatcgccattcccaccaattttaccatcagcgtgaccacagagatcctcccagtgagcatgaccaagacaagcgtcgattgcacaatgtacatttgcggcgacagcactgaatgtagcaacctcctgctgcagtacgggagcttttgtacccaactgaatcgggcactcaccgggatcgccgtggagcaggataaaaacactcaggaggtctttgctcaggtcaaacagatctacaaaaccccaccaattaaagacttcggaggcttcaacttctcacagattctgccagacccttcaaagccaagcaagcgcagcttcattgaagatctcctgttcaacaaggtgactctggctgacgctgggttcatcaagcagtatggagactgcctgggggacatcgcagcaagggatctgatctgtgctcagaagtttaatggcctcacagtgctgccccctctcctgactgacgagatgattgctcagtacaccagcgccctgctggctggaactatcacatcagggtggacattcggggccggggctgctctgcagattcctttcgcaatgcaaatggcatataggttcaacgggatcggcgtcacacagaacgtgctgtatgagaatcagaaactgatcgcaaatcaatttaattcagcaatcgggaaaatccaggactcactgtcatcaaccgcatcagccctgggcaagctgcaggacgtcgtcaaccaaaacgcccaggcactcaacacactggtgaagcagctgtcaagcaatttcggagcaatcagcagcgtcctgaatgatatcctgagcaggctggataaggtggaggcagaagtgcagatcgaccgcctgatcaccggccggctccagagcctgcaaacctatgtcactcagcaactgatcagagctgcagaaatccgggcaagcgccaatctggccgccaccaagatgtcagaatgcgtgctcggccagagcaagagggtggacttctgcggcaaaggctaccacctgatgagcttccctcagagcgccccccacggcgtggtcttcctccatgtgacttatgtgcccgcacaggaaaagaacttcactactgccccagccatctgccacgacggaaaggctcatttcccacgcgagggggtgttcgtcagcaatggaacccactggttcgtgacccagcggaacttctatgagcctcagattattaccacagacaacacatttgtgtcaggaaattgcgatgtcgtgatcgggatcgtcaacaatactgtctatgaccccctccagccagaactcgatagcttcaaagaggaactcgataagtattttaaaaatcacaccagccctgatgtggatctgggcgatatcagcggaatcaatgctagcgtggtgaacatccagaaggagattgacaggctcaacgaagtcgcaaagaacctgaacgagtcactgatcgacctccaggaactgggcaagtacgaacagtacatcaagtggccctggtacatctggctcgggttcatcgctggcctgattgctatcgtgatggtgacaatcatgctctgctgtatgaccagctgctgcagctgcctgaagggatgctgctcatgcggcagctgctgtaagttcgatgaggacgactcagagcccgtgctcaaaggcgtgaaactgcattatacttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 128 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgata

SEQ ID NO: 14 - DNA, 164 bp, Artificial Sequence
  source: /note=“Description of Artificial Sequence Synthetic polynucleotide”
ctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 15 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1273 aa, Severe acute respiratory syndrome coronavirus 2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1273 aa, Severe acute respiratory syndrome coronavirus 2
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - RNA, 3822 nt, Artificial Sequence
  source: /note=“Description of Artificial Sequence Synthetic polynucleotide”
auguuuguguuucugguccuccugccccuggugucaagccaaugcgucaacuucacuacacggacccagcugcccccugcuuauaccaacucauuuacuagagggguguacuacccagauaaaguguucagaucaagcgugcuccacagcacccaggaccucuuccugcccuucuuuucaaacgugaccugguuccaugccauccaugugagcggaaccaacgggaccaagcgguucgccaauccagugcuccccuucaacgaugggguguauuuugcaagcacugaaaagucaaauauuauccgcggauggauuuucgggacaacacucgauagcaagacccagucacugcucaucgugaacaacgcuacaaauguggucaucaaggugugcgaguuucaguucugcaaugacccuuuucucggcgucuacuaccacaagaacaacaagagcuggauggaaucagaauuccggguguauagcucagcuaauaauugcaccuucgaguaugugagccagccuuuccucauggaccuggaggggaagcagggcaacuucaagaaucugagagaauucguguuuaaaaacaucgauggauauuucaaaaucuauagcaagcacacuccuaucaaccucguccggggccugccacaaggcuuuagcgcacuggagccccucguggaccugcccaucgggaucaauaucaccagauuucagacucugcuggcucuccacaucagcuaccugacuccaggggauagcucaagcggcuggaccgcuggagccgcugccuauuacgugggguaccugcagccuaggacuuuucugcugaaguacaaugagaacgggacaauuacugacgcugucgauugugcacucgacccccucagcgagacaaaauguacccugaaaagcuuuacuguggaaaaagggauuuaccagacuagcaauuucagaguccagccaacagagucaaucgugcgguuuccuaacaucacaaaccucugcccauucggggaggucuucaaugccacccgcuucgccagcguguacgcuuggaauagaaagcggaucagcaacugcguggccgacuacagcgugcucuacaauagcgcuagcuucucaacauuuaaaugcuacggggugagccccacaaaacugaaugaccuguguuucaccaauguguaugccgauagcuucgugauuagaggcgacgaggugcgccaaaucgcaccuggccaaaccggaaacauugcagauuauaacuauaagcugcccgaugauuuuacuggcugugucaucgcuuggaacagcaacaaccucgauucaaaagucggaggaaacuauaacuaccuguauagacuguucagaaagagcaaccugaagcccuuugaaagggauaucagcacagaaauuuaccaggccgggagcaccccaugcaacggggucaaaggguuuaacuguuauuucccccugcagagcuacggguuucaacccaccuauggggugggguaucagccuuaccgcguggucgugcugagcuucgaacugcugcacgcccccgccaccgugugcggcccuaaaaagagcacaaaccucgucaagaacaagugcgugaacuucaauuucaacggccugacuggcacaggcguccucaccgaaagcaauaaaaaguuccucccuuuccagcaauucggcagagacauugcagacacaacugaugcugugagggacccccagacacuggagauccuggauauuacccccuguucauucggcggagugucagucaucaccccuggcacuaacacaucaaaucagguggcaguccuguaucagggcgugaacugcaccgaagugccugucgccauucaugcagaccagcugacccccacuuggagaguguacucaacuggcagcaaugucuuucaaaccagagccggcugucucaucggcgcagaacaugucaacaacagcuaugagugugacaucccaaucggagcuggcaucugugccagcuaucagacacagaccaacagcccucggagggcuagaagcguggccagccagagcauuaucgcuuacaccaugucacugggcgucgagaauagcguggccuauagcaacaauagcaucgccauucccaccaauuuuaccaucagcgugaccacagagauccucccagugagcaugaccaagacaagcgucgauugcacaauguacauuugcggcgacagcacugaauguagcaaccuccugcugcaguacgggagcuuuuguacccaacugaaucgggcacucaccgggaucgccguggagcaggauaaaaacacucaggaggucuuugcucaggucaaacagaucuacaaaaccccaccaauuaaagacuucggaggcuucaacuucucacagauucugccagacccuucaaagccaagcaagcgcagcuucauugaagaucuccuguucaacaaggugacucuggcugacgcuggguucaucaagcaguauggagacugccugggggacaucgcagcaagggaucugaucugugcucagaaguuuaauggccucacagugcugcccccucuccugacugacgagaugauugcucaguacaccagcgcccugcuggcuggaacuaucacaucaggguggacauucggggccggggcugcucugcagauuccuuucgcaaugcaaauggcauauagguucaacgggaucggcgucacacagaacgugcuguaugagaaucagaaacugaucgcaaaucaauuuaauucagcaaucgggaaaauccaggacucacugucaucaaccgcaucagcccugggcaagcugcaggacgucgucaaccaaaacgcccaggcacucaacacacuggugaagcagcugucaagcaauuucggagcaaucagcagcguccugaaugauauccugagcaggcuggauaagguggaggcagaagugcagaucgaccgccugaucaccggccggcuccagagccugcaaaccuaugucacucagcaacugaucagagcugcagaaauccgggcaagcgccaaucuggccgccaccaagaugucagaaugcgugcucggccagagcaagaggguggacuucugcggcaaaggcuaccaccugaugagcuucccucagagcgccccccacggcguggucuuccuccaugugacuuaugugcccgcacaggaaaagaacuucacuacugccccagccaucugccacgacggaaaggcucauuucccacgcgaggggguguucgucagcaauggaacccacugguucgugacccagcggaacuucuaugagccucagauuauuaccacagacaacacauuugugucaggaaauugcgaugucgugaucgggaucgucaacaauacugucuaugacccccuccagccagaacucgauagcuucaaagaggaacucgauaaguauuuuaaaaaucacaccagcccugauguggaucugggcgauaucagcggaaucaaugcuagcguggugaacauccagaaggagauugacaggcucaacgaagucgcaaagaaccugaacgagucacugaucgaccuccaggaacugggcaaguacgaacaguacaucaaguggcccugguacaucuggcucggguucaucgcuggccugauugcuaucgugauggugacaaucaugcucugcuguaugaccagcugcugcagcugccugaagggaugcugcucaugcggcagcugcuguaaguucgaugaggacgacucagagcccgugcucaaaggcgugaaacugcauuauacuuga

SEQ ID NO: 21 - RNA, 669 nt, Artificial Sequence
  source: /note=“Description of Artificial Sequence Synthetic polynucleotide”
auggccgacucaaacggcacaauuaccguggaggagcugaagaagcuccuggagcaguggaaucuggucauuggcuuucuguuccugaccuggaucugccugcugcaguucgcuuacgcuaaccgcaaccgguuccucuauaucauuaaacucauuuuucuguggcugcucuggccugugacacuggccuguuucgugcucgccgccgucuaccgcauuaacuggaucacaggagggauugcuaucgcuauggcuugccuggucgggcugauguggcucucauacuucaucgcaagcuuuaggcucuuugcccggacucggagcauguggucauucaaccccgagacaaacauucugcucaaugugccucugcacgggaccauccugaccaggccccuccuggagagcgagcuggugaucggggccgugauccugaggggacaucugaggaucgccggccaucaucugggcaggugugacaucaaggaucucccuaaggagaucacuguggcaacuagccggacacugagcuauuacaaacucggcgcaagccagcgcgucgcuggggacucaggcuucgcagcuuacagcagauauaggaucgggaacuacaagcugaauaccgaucacagcucaagcagcgauaacaucgcucugcucguccaguga

SEQ ID NO: 22 - RNA, 1140 nt, Artificial Sequence
  source: /note=“Description of Artificial Sequence Synthetic polynucleotide”
augaggccccaggggcugccaaacaacaccgcaagcugguucacugcccugacucaacacggaaaggaggaccucaaguuccccaggggacagggggugccuauuaacacuaacucaagcccugacgaucagaucggcuauuaucgcagggcuacaaggcgcauuagaggaggagaugggaaaaugaaggaccugagcccucggugguauuuuuauuaccugggcacugggccagaggccggacugccauacggcgcuaauaaagacgggauuaucuggguggccacugaaggcgcucugaauacccccaaggaucacauugggacucggaacccagccaacaacgccgcuaucgugcuccagcucccucagggcaccacucugcccaagggguucuacgccgagggcucaagaggggggagccaggccucaagcagaagcggcagcggccccaggcagaagagaacagcuacuaaagcauauaaugugacacaggccuucggccgcagagggcccgaacaaacccagggaaacuucggcgaucaggagcugauccggcagggcacugauuacaagcacuggccucagauugcccaguucgcuccaucagccagcgcuuucuucggaaugagccggaucgggauggaggugaccccuucagggacauggcugacuuauaccggagcaaucaagcuggaugacaaggaccccaauuucaaagaccaggugauccugcugaacaaacauauugaugccuacaaagggucaggcagacucuucgcccgcacuagaucaauguggucauuuaacccagaaacuaacauccugcugaacgugccucugcauggcacaauucucaccagaccccugcuggaaagcgaacuggucauuggcgcugugauccugagaggccaccugagaauugcuggacaccaucucggcagaugugauauuaaggaucugccaaaggagaucaccguggcuacaagcagaacucucagcuacuacaagcugggggccagccagcgggucgcaggggauagcggauuugccgccuacucaagauauagaaucggaaacuauaaacucaacaccgaccauucaagcagcagcgacaacauugcacugcugguccaguga

SEQ ID NO: 23 - RNA, 1260 nt, Artificial Sequence
  source: /note=“Description of Artificial Sequence Synthetic polynucleotide”
augucagauaacggcccacagaaucagagaaacgccccacggaucacuuuuggcgggccuagcgauucaaccgggucaaaucagaauggggaacgcagcggggcccggagcaagcagagacggccccaagggcugccuaacaauacagcaucaugguucacagcccugacccagcauggcaaggaagaucucaaguuuccucgcggacagggcgugccuauuaacaccaauucaagcccugacgaucagaucggcuacuacagaagggcuacucgcagaauccgcggaggcgauggaaaaaugaaagaccugagcccaaggugguacuucuauuaccugggaacuggcccugaggcaggacugcccuauggcgccaacaaggauggcauuauuugggucgcaacugaaggagcacugaacacaccaaaggaucacaucggaacaaggaaccccgcaaacaaugccgcuaucgugcuccagcugccucaagggacuacccugcccaaaggguuuuaugccgaagggagcaggggcgggagccaggcaagcagcagaagcucaucacggucaagaaauucaagcaggaauagcaccccaggaagcucaaggggaacaagcccagcccggauggcaggcaacggcggggaugcagcacucgcacugcuccugcuggaccgccugaaucagcuggaaucaaaaaugagcgggaagggacagcaacagcagggacaaacugucacuaagaagucagccgccgaggcaagcaagaagcccaggcaaaagcgcacugcaaccaaggcuuacaaugucacucaagcauucggcagaaggggcccagaacagacccagggcaauuucggcgaccaggagcugauuagacaaggcacugauuacaaacacuggccucagaucgcucaguuugcccccagcgcuucagccuuuuuugggaugagccggaucggcauggaggugacaccaucagggaccuggcucaccuacacuggagccauuaaacuggaugacaaagacccaaacuucaaagaucaagugauccugcucaacaagcacauugaugcauauaaaacuuuuccuccuacugagccuaagaaggacaagaagaagaaagcagaugagacacaggcucugccccagcgccagaagaagcagcagacugugacacugcugcccgcugcagaucuggacgacuucagcaaacagcugcaacaaucaaugagcucagcugacucaacucaggcuuga

SEQ ID NO: 24 - RNA, 1206 nt, Artificial Sequence
  source: /note=“Description of Artificial Sequence Synthetic polynucleotide”
auggacaugagagcuccagcccagaucuuuggcuuucugcuccugcuguuuccaggcacuaggugcgacaggccccaggggcugccaaacaacaccgcaagcugguucacugcccugacucaacacggaaaggaggaccucaaguuccccaggggacagggggugccuauuaacacuaacucaagcccugacgaucagaucggcuauuaucgcagggcuacaaggcgcauuagaggaggagaugggaaaaugaaggaccugagcccucggugguauuuuuauuaccugggcacugggccagaggccggacugccauacggcgcuaauaaagacgggauuaucuggguggccacugaaggcgcucugaauacccccaaggaucacauugggacucggaacccagccaacaacgccgcuaucgugcuccagcucccucagggcaccacucugcccaagggguucuacgccgagggcucaagaggggggagccaggccucaagcagaagcggcagcggccccaggcagaagagaacagcuacuaaagcauauaaugugacacaggccuucggccgcagagggcccgaacaaacccagggaaacuucggcgaucaggagcugauccggcagggcacugauuacaagcacuggccucagauugcccaguucgcuccaucagccagcgcuuucuucggaaugagccggaucgggauggaggugaccccuucagggacauggcugacuuauaccggagcaaucaagcuggaugacaaggaccccaauuucaaagaccaggugauccugcugaacaaacauauugaugccuacaaagggucaggcagacucuucgcccgcacuagaucaauguggucauuuaacccagaaacuaacauccugcugaacgugccucugcauggcacaauucucaccagaccccugcuggaaagcgaacuggucauuggcgcugugauccugagaggccaccugagaauugcuggacaccaucucggcagaugugauauuaaggaucugccaaaggagaucaccguggcuacaagcagaacucucagcuacuacaagcugggggccagccagcgggucgcaggggauagcggauuugccgccuacucaagauauagaaucggaaacuauaaacucaacaccgaccauucaagcagcagcgacaacauugcacugcugguccaguga

SEQ ID NO: 25 - RNA, 1144 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauggacaugcgggcucccgcccagaucuucggguuccugcugcuccuguuccccggcaccagaugcgaucgcgugcagcccaccgagagcaucguccgcuuccccaacauuaccaaccugugccccuucggcgaggucuuuaacgccacaagauucgcaucaguguaugccuggaacaggaagagaaucucaaacuguguggcagacuauucaguccuguauaauagcgcaagcuuuucaacauucaaaugcuauggcgucagccccaccaaacucaaugaucugugcuuuaccaaugucuacgcagacagcuucgucauuaggggagaugaagugagacagauugcccccggccagaccggcaagaucgcugacuacaauuacaagcuccccgacgacuuuacagggugugucauugccuggaacagcaacaaccuggauucaaaagugggcggcaacuacaacuaucuguaccggcucuuuagaaaaagcaaccugaagccuuucgagagggacauuagcacagaaaucuaucaggcuggcagcaccccaugcaacggaguggaaggcuucaacugcuauuuuccucugcagagcuacggcuuccagcccacuaauggcgucggcuaucagcccuacaggguggucguccucagcuuugaacugcugcaugcuccugcuacaguguguggacccaagaagucaaccaaucuggucaagaacaagugugugaauuuuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 4213 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuuguguuucugguccuccugccccuggugucaagccaaugcgucaaccugacuacacggacccagcugcccccugcuuauaccaacucauuuacuagagggguguacuacccagauaaaguguucagaucaagcgugcuccacagcacccaggaccucuuccugcccuucuuuucaaacgugaccugguuccaugccauccaugugagcggaaccaacgggaccaagcgguucgauaauccagugcuccccuucaacgaugggguguauuuugcaagcacugaaaagucaaauauuauccgcggauggauuuucgggacaacacucgauagcaagacccagucacugcucaucgugaacaacgcuacaaauguggucaucaaggugugcgaguuucaguucugcaaugacccuuuucucggcgucuacuaccacaagaacaacaagagcuggauggaaucagaauuccggguguauagcucagcuaauaauugcaccuucgaguaugugagccagccuuuccucauggaccuggaggggaagcagggcaacuucaagaaucugagagaauucguguuuaaaaacaucgauggauauuucaaaaucuauagcaagcacacuccuaucaaccucguccgggaccugccacaaggcuuuagcgcacuggagccccucguggaccugcccaucgggaucaauaucaccagauuucagacucugcuggcucuccaccgcagcuaccugacuccaggggauagcucaagcggcuggaccgcuggagccgcugccuauuacgugggguaccugcagccuaggacuuuucugcugaaguacaaugagaacgggacaauuacugacgcugucgauugugcacucgacccccucagcgagacaaaauguacccugaaaagcuuuacuguggaaaaagggauuuaccagacuagcaauuucagaguccagccaacagagucaaucgugcgguuuccuaacaucacaaaccucugcccauucggggaggucuucaaugccacccgcuucgccagcguguacgcuuggaauagaaagcggaucagcaacugcguggccgacuacagcgugcucuacaauagcgcuagcuucucaacauuuaaaugcuacggggugagccccacaaaacugaaugaccuguguuucaccaauguguaugccgauagcuucgugauuagaggcgacgaggugcgccaaaucgcaccuggccaaaccggaaagauugcagauuauaacuauaagcugcccgaugauuuuacuggcugugucaucgcuuggaacagcaacaaccucgauucaaaagucggaggaaacuauaacuaccuguauagacuguucagaaagagcaaccugaagcccuuugaaagggauaucagcacagaaauuuaccaggccgggagcaccccaugcaacggggucgaaggguuuaacuguuauuucccccugcagagcuacggguuucaacccaccaauggggugggguaucagccuuaccgcguggucgugcugagcuucgaacugcugcacgcccccgccaccgugugcggcccuaaaaagagcacaaaccucgucaagaacaagugcgugaacuucaauuucaacggccugacuggcacaggcguccucaccgaaagcaauaaaaaguuccucccuuuccagcaauucggcagagacauugcagacacaacugaugcugugagggacccccagacacuggagauccuggauauuacccccuguucauucggcggagugucagucaucaccccuggcacuaacacaucaaaucagguggcaguccuguaucaggacgugaacugcaccgaagugccugucgccauucaugcagaccagcugacccccacuuggagaguguacucaacuggcagcaaugucuuucaaaccagagccggcugucucaucggcgcagaacaugucaacaacagcuaugagugugacaucccaaucggagcuggcaucugugccagcuaucagacacagaccauccugagaagcguggccagccagagcauuaucgcuuacaccaugucacugggcgccgagaauagcguggccuauagcaacaauagcaucgccauucccaccaauuuuaccaucagcgugaccacagagauccucccagugagcaugaccaagacaagcgucgauugcacaauguacauuugcggcgacagcacugaauguagcaaccuccugcugcaguacgggagcuuuuguacccaacugaaucgggcacucaccgggaucgccguggagcaggauaaaaacacucaggaggucuuugcucaggucaaacagaucuacaaaaccccaccaauuaaagacuucggaggcuucaacuucucacagauucugccagacccuucaaagccaagcaagcgcagcuucauugaagaucuccuguucaacaaggugacucuggcugacgcuggguucaucaagcaguauggagacugccugggggacaucgcagcaagggaucugaucugugcucagaaguuuaauggccucacagugcugcccccucuccugacugacgagaugauugcucaguacaccagcgcccugcuggcuggaacuaucacaucaggguggacauucggggccggggcugcucugcagauuccuuucgcaaugcaaauggcauauagguucaacgggaucggcgucacacagaacgugcuguaugagaaucagaaacugaucgcaaaucaauuuaauucagcaaucgggaaaauccaggacucacugucaucaaccgcaucagcccugggcaagcugcaggacgucgucaaccaaaacgcccaggcacucaacacacuggugaagcagcugucaagcaauuucggagcaaucagcagcguccugaaugauauccugagcaggcuggauaagguggaggcagaagugcagaucgaccgccugaucaccggccggcuccagagccugcaaaccuaugucacucagcaacugaucagagcugcagaaauccgggcaagcgccaaucuggccgccaccaagaugucagaaugcgugcucggccagagcaagaggguggacuucugcggcaaaggcuaccaccugaugagcuucccucagagcgccccccacggcguggucuuccuccaugugacuuaugugcccgcacaggaaaagaacuucacuacugccccagccaucugccacgacggaaaggcucauuucccacgcgaggggguguucgucagcaauggaacccacugguucgugacccagcggaacuucuaugagccucagauuauuaccacagacaacacauuugugucaggaaauugcgaugucgugaucgggaucgucaacaauacugucuaugacccccuccagccagaacucgauagcuucaaagaggaacucgauaaguauuuuaaaaaucacaccagcccugauguggaucugggcgauaucagcggaaucaaugcuagcguggugaacauccagaaggagauugacaggcucaacgaagucgcaaagaaccugaacgagucacugaucgaccuccaggaacugggcaaguacgaacaguacaucaaguggcccugguacaucuggcucggguucaucgcuggccugauugcuaucgugauggugacaaucaugcucugcuguaugaccagcugcugcagcugccugaagggaugcugcucaugcggcagcugcuguaaguucgaugaggacgacucagagcccgugcucaaaggcgugaaacugcauuauacuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - RNA, 4225 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuuguguuucugguccuccugccccuggugucaagccaaugcgucaaccugacuacacggacccagcugcccccugcuuauaccaacucauuuacuagagggguguacuacccagauaaaguguucagaucaagcgugcuccacagcacccaggaccucuuccugcccuucuuuucaaacgugaccugguuccaugccauccaugugagcggaaccaacgggaccaagcgguucgauaauccagugcuccccuucaacgaugggguguauuuugcaagcacugaaaagucaaauauuauccgcggauggauuuucgggacaacacucgauagcaagacccagucacugcucaucgugaacaacgcuacaaauguggucaucaaggugugcgaguuucaguucugcaaugacccuuuucucggcgucuacuaccacaagaacaacaagagcuggauggaaucagaauuccggguguauagcucagcuaauaauugcaccuucgaguaugugagccagccuuuccucauggaccuggaggggaagcagggcaacuucaagaaucugagagaauucguguuuaaaaacaucgauggauauuucaaaaucuauagcaagcacacuccuaucaaccucguccgggaccugccacaaggcuuuagcgcacuggagccccucguggaccugcccaucgggaucaauaucaccagauuucagacucugcuggcucuccaccgcagcuaccugacuccaggggauagcucaagcggcuggaccgcuggagccgcugccuauuacgugggguaccugcagccuaggacuuuucugcugaaguacaaugagaacgggacaauuacugacgcugucgauugugcacucgacccccucagcgagacaaaauguacccugaaaagcuuuacuguggaaaaagggauuuaccagacuagcaauuucagaguccagccaacagagucaaucgugcgguuuccuaacaucacaaaccucugcccauucggggaggucuucaaugccacccgcuucgccagcguguacgcuuggaauagaaagcggaucagcaacugcguggccgacuacagcgugcucuacaauagcgcuagcuucucaacauuuaaaugcuacggggugagccccacaaaacugaaugaccuguguuucaccaauguguaugccgauagcuucgugauuagaggcgacgaggugcgccaaaucgcaccuggccaaaccggaaagauugcagauuauaacuauaagcugcccgaugauuuuacuggcugugucaucgcuuggaacagcaacaaccucgauucaaaagucggaggaaacuauaacuaccuguauagacuguucagaaagagcaaccugaagcccuuugaaagggauaucagcacagaaauuuaccaggccgggagcaccccaugcaacggggucgaaggguuuaacuguuauuucccccugcagagcuacggguuucaacccaccaauggggugggguaucagccuuaccgcguggucgugcugagcuucgaacugcugcacgcccccgccaccgugugcggcccuaaaaagagcacaaaccucgucaagaacaagugcgugaacuucaauuucaacggccugacuggcacaggcguccucaccgaaagcaauaaaaaguuccucccuuuccagcaauucggcagagacauugcagacacaacugaugcugugagggacccccagacacuggagauccuggauauuacccccuguucauucggcggagugucagucaucaccccuggcacuaacacaucaaaucagguggcaguccuguaucagggcgugaacugcaccgaagugccugucgccauucaugcagaccagcugacccccacuuggagaguguacucaacuggcagcaaugucuuucaaaccagagccggcugucucaucggcgcagaacaugucaacaacagcuaugagugugacaucccaaucggagcuggcaucugugccagcuaucagacacagaccaacagcccucggagggcuagaagcguggccagccagagcauuaucgcuuacaccaugucacugggcgccgagaauagcguggccuauagcaacaauagcaucgccauucccaccaauuuuaccaucagcgugaccacagagauccucccagugagcaugaccaagacaagcgucgauugcacaauguacauuugcggcgacagcacugaauguagcaaccuccugcugcaguacgggagcuuuuguacccaacugaaucgggcacucaccgggaucgccguggagcaggauaaaaacacucaggaggucuuugcucaggucaaacagaucuacaaaaccccaccaauuaaagacuucggaggcuucaacuucucacagauucugccagacccuucaaagccaagcaagcgcagcuucauugaagaucuccuguucaacaaggugacucuggcugacgcuggguucaucaagcaguauggagacugccugggggacaucgcagcaagggaucugaucugugcucagaaguuuaauggccucacagugcugcccccucuccugacugacgagaugauugcucaguacaccagcgcccugcuggcuggaacuaucacaucaggguggacauucggggccggggcugcucugcagauuccuuucgcaaugcaaauggcauauagguucaacgggaucggcgucacacagaacgugcuguaugagaaucagaaacugaucgcaaaucaauuuaauucagcaaucgggaaaauccaggacucacugucaucaaccgcaucagcccugggcaagcugcaggacgucgucaaccaaaacgcccaggcacucaacacacuggugaagcagcugucaagcaauuucggagcaaucagcagcguccugaaugauauccugagcaggcuggauaagguggaggcagaagugcagaucgaccgccugaucaccggccggcuccagagccugcaaaccuaugucacucagcaacugaucagagcugcagaaauccgggcaagcgccaaucuggccgccaccaagaugucagaaugcgugcucggccagagcaagaggguggacuucugcggcaaaggcuaccaccugaugagcuucccucagagcgccccccacggcguggucuuccuccaugugacuuaugugcccgcacaggaaaagaacuucacuacugccccagccaucugccacgacggaaaggcucauuucccacgcgaggggguguucgucagcaauggaacccacugguucgugacccagcggaacuucuaugagccucagauuauuaccacagacaacacauuugugucaggaaauugcgaugucgugaucgggaucgucaacaauacugucuaugacccccuccagccagaacucgauagcuucaaagaggaacucgauaaguauuuuaaaaaucacaccagcccugauguggaucugggcgauaucagcggaaucaaugcuagcguggugaacauccagaaggagauugacaggcucaacgaagucgcaaagaaccugaacgagucacugaucgaccuccaggaacugggcaaguacgaacaguacaucaaguggcccugguacaucuggcucggguucaucgcuggccugauugcuaucgugauggugacaaucaugcucugcuguaugaccagcugcugcagcugccugaagggaugcugcucaugcggcagcugcuguaaguucgaugaggacgacucagagcccgugcucaaaggcgugaaacugcauuauacuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - RNA, 1072 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauggccgacucaaacggcacaauuaccguggaggagcugaagaagcuccuggagcaguggaaucuggucauuggcuuucuguuccugaccuggaucugccugcugcaguucgcuuacgcuaaccgcaaccgguuccucuauaucauuaaacucauuuuucuguggcugcucuggccugugacacuggccuguuucgugcucgccgccgucuaccgcauuaacuggaucacaggagggauugcuaucgcuauggcuugccuggucgggcugauguggcucucauacuucaucgcaagcuuuaggcucuuugcccggacucggagcauguggucauucaaccccgagacaaacauucugcucaaugugccucugcacgggaccauccugaccaggccccuccuggagagcgagcuggugaucggggccgugauccugaggggacaucugaggaucgccggccaucaucugggcaggugugacaucaaggaucucccuaaggagaucacuguggcaacuagccggacacugagcuauuacaaacucggcgcaagccagcgcgucgcuggggacucaggcuucgcagcuuacagcagauauaggaucgggaacuacaagcugaauaccgaucacagcucaagcagcgauaacaucgcucugcucguccagugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - RNA, 1543 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugaggccccaggggcugccaaacaacaccgcaagcugguucacugcccugacucaacacggaaaggaggaccucaaguuccccaggggacagggggugccuauuaacacuaacucaagcccugacgaucagaucggcuauuaucgcagggcuacaaggcgcauuagaggaggagaugggaaaaugaaggaccugagcccucggugguauuuuuauuaccugggcacugggccagaggccggacugccauacggcgcuaauaaagacgggauuaucuggguggccacugaaggcgcucugaauacccccaaggaucacauugggacucggaacccagccaacaacgccgcuaucgugcuccagcucccucagggcaccacucugcccaagggguucuacgccgagggcucaagaggggggagccaggccucaagcagaagcggcagcggccccaggcagaagagaacagcuacuaaagcauauaaugugacacaggccuucggccgcagagggcccgaacaaacccagggaaacuucggcgaucaggagcugauccggcagggcacugauuacaagcacuggccucagauugcccaguucgcuccaucagccagcgcuuucuucggaaugagccggaucgggauggaggugaccccuucagggacauggcugacuuauaccggagcaaucaagcuggaugacaaggaccccaauuucaaagaccaggugauccugcugaacaaacauauugaugccuacaaagggucaggcagacucuucgcccgcacuagaucaauguggucauuuaacccagaaacuaacauccugcugaacgugccucugcauggcacaauucucaccagaccccugcuggaaagcgaacuggucauuggcgcugugauccugagaggccaccugagaauugcuggacaccaucucggcagaugugauauuaaggaucugccaaaggagaucaccguggcuacaagcagaacucucagcuacuacaagcugggggccagccagcgggucgcaggggauagcggauuugccgccuacucaagauauagaaucggaaacuauaaacucaacaccgaccauucaagcagcagcgacaacauugcacugcugguccagugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 1663 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccaugucagauaacggcccacagaaucagagaaacgccccacggaucacuuuuggcgggccuagcgauucaaccgggucaaaucagaauggggaacgcagcggggcccggagcaagcagagacggccccaagggcugccuaacaauacagcaucaugguucacagcccugacccagcauggcaaggaagaucucaaguuuccucgcggacagggcgugccuauuaacaccaauucaagcccugacgaucagaucggcuacuacagaagggcuacucgcagaauccgcggaggcgauggaaaaaugaaagaccugagcccaaggugguacuucuauuaccugggaacuggcccugaggcaggacugcccuauggcgccaacaaggauggcauuauuugggucgcaacugaaggagcacugaacacaccaaaggaucacaucggaacaaggaaccccgcaaacaaugccgcuaucgugcuccagcugccucaagggacuacccugcccaaaggguuuuaugccgaagggagcaggggcgggagccaggcaagcagcagaagcucaucacggucaagaaauucaagcaggaauagcaccccaggaagcucaaggggaacaagcccagcccggauggcaggcaacggcggggaugcagcacucgcacugcuccugcuggaccgccugaaucagcuggaaucaaaaaugagcgggaagggacagcaacagcagggacaaacugucacuaagaagucagccgccgaggcaagcaagaagcccaggcaaaagcgcacugcaaccaaggcuuacaaugucacucaagcauucggcagaaggggcccagaacagacccagggcaauuucggcgaccaggagcugauuagacaaggcacugauuacaaacacuggccucagaucgcucaguuugcccccagcgcuucagccuuuuuugggaugagccggaucggcauggaggugacaccaucagggaccuggcucaccuacacuggagccauuaaacuggaugacaaagacccaaacuucaaagaucaagugauccugcucaacaagcacauugaugcauauaaaacuuuuccuccuacugagccuaagaaggacaagaagaagaaagcagaugagacacaggcucugccccagcgccagaagaagcagcagacugugacacugcugcccgcugcagaucuggacgacuucagcaaacagcugcaacaaucaaugagcucagcugacucaacucaggcuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - RNA, 1609 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauggacaugagagcuccagcccagaucuuuggcuuucugcuccugcuguuuccaggcacuaggugcgacaggccccaggggcugccaaacaacaccgcaagcugguucacugcccugacucaacacggaaaggaggaccucaaguuccccaggggacagggggugccuauuaacacuaacucaagcccugacgaucagaucggcuauuaucgcagggcuacaaggcgcauuagaggaggagaugggaaaaugaaggaccugagcccucggugguauuuuuauuaccugggcacugggccagaggccggacugccauacggcgcuaauaaagacgggauuaucuggguggccacugaaggcgcucugaauacccccaaggaucacauugggacucggaacccagccaacaacgccgcuaucgugcuccagcucccucagggcaccacucugcccaagggguucuacgccgagggcucaagaggggggagccaggccucaagcagaagcggcagcggccccaggcagaagagaacagcuacuaaagcauauaaugugacacaggccuucggccgcagagggcccgaacaaacccagggaaacuucggcgaucaggagcugauccggcagggcacugauuacaagcacuggccucagauugcccaguucgcuccaucagccagcgcuuucuucggaaugagccggaucgggauggaggugaccccuucagggacauggcugacuuauaccggagcaaucaagcuggaugacaaggaccccaauuucaaagaccaggugauccugcugaacaaacauauugaugccuacaaagggucaggcagacucuucgcccgcacuagaucaauguggucauuuaacccagaaacuaacauccugcugaacgugccucugcauggcacaauucucaccagaccccugcuggaaagcgaacuggucauuggcgcugugauccugagaggccaccugagaauugcuggacaccaucucggcagaugugauauuaaggaucugccaaaggagaucaccguggcuacaagcagaacucucagcuacuacaagcugggggccagccagcgggucgcaggggauagcggauuugccgccuacucaagauauagaaucggaaacuauaaacucaacaccgaccauucaagcagcagcgacaacauugcacugcugguccagugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - RNA, 4225 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauguuuguguuucugguccuccugccccuggugucaagccaaugcgucaacuucacuacacggacccagcugcccccugcuuauaccaacucauuuacuagagggguguacuacccagauaaaguguucagaucaagcgugcuccacagcacccaggaccucuuccugcccuucuuuucaaacgugaccugguuccaugccauccaugugagcggaaccaacgggaccaagcgguucgccaauccagugcuccccuucaacgaugggguguauuuugcaagcacugaaaagucaaauauuauccgcggauggauuuucgggacaacacucgauagcaagacccagucacugcucaucgugaacaacgcuacaaauguggucaucaaggugugcgaguuucaguucugcaaugacccuuuucucggcgucuacuaccacaagaacaacaagagcuggauggaaucagaauuccggguguauagcucagcuaauaauugcaccuucgaguaugugagccagccuuuccucauggaccuggaggggaagcagggcaacuucaagaaucugagagaauucguguuuaaaaacaucgauggauauuucaaaaucuauagcaagcacacuccuaucaaccucguccggggccugccacaaggcuuuagcgcacuggagccccucguggaccugcccaucgggaucaauaucaccagauuucagacucugcuggcucuccacaucagcuaccugacuccaggggauagcucaagcggcuggaccgcuggagccgcugccuauuacgugggguaccugcagccuaggacuuuucugcugaaguacaaugagaacgggacaauuacugacgcugucgauugugcacucgacccccucagcgagacaaaauguacccugaaaagcuuuacuguggaaaaagggauuuaccagacuagcaauuucagaguccagccaacagagucaaucgugcgguuuccuaacaucacaaaccucugcccauucggggaggucuucaaugccacccgcuucgccagcguguacgcuuggaauagaaagcggaucagcaacugcguggccgacuacagcgugcucuacaauagcgcuagcuucucaacauuuaaaugcuacggggugagccccacaaaacugaaugaccuguguuucaccaauguguaugccgauagcuucgugauuagaggcgacgaggugcgccaaaucgcaccuggccaaaccggaaacauugcagauuauaacuauaagcugcccgaugauuuuacuggcugugucaucgcuuggaacagcaacaaccucgauucaaaagucggaggaaacuauaacuaccuguauagacuguucagaaagagcaaccugaagcccuuugaaagggauaucagcacagaaauuuaccaggccgggagcaccccaugcaacggggucaaaggguuuaacuguuauuucccccugcagagcuacggguuucaacccaccuauggggugggguaucagccuuaccgcguggucgugcugagcuucgaacugcugcacgcccccgccaccgugugcggcccuaaaaagagcacaaaccucgucaagaacaagugcgugaacuucaauuucaacggccugacuggcacaggcguccucaccgaaagcaauaaaaaguuccucccuuuccagcaauucggcagagacauugcagacacaacugaugcugugagggacccccagacacuggagauccuggauauuacccccuguucauucggcggagugucagucaucaccccuggcacuaacacaucaaaucagguggcaguccuguaucagggcgugaacugcaccgaagugccugucgccauucaugcagaccagcugacccccacuuggagaguguacucaacuggcagcaaugucuuucaaaccagagccggcugucucaucggcgcagaacaugucaacaacagcuaugagugugacaucccaaucggagcuggcaucugugccagcuaucagacacagaccaacagcccucggagggcuagaagcguggccagccagagcauuaucgcuuacaccaugucacugggcgucgagaauagcguggccuauagcaacaauagcaucgccauucccaccaauuuuaccaucagcgugaccacagagauccucccagugagcaugaccaagacaagcgucgauugcacaauguacauuugcggcgacagcacugaauguagcaaccuccugcugcaguacgggagcuuuuguacccaacugaaucgggcacucaccgggaucgccguggagcaggauaaaaacacucaggaggucuuugcucaggucaaacagaucuacaaaaccccaccaauuaaagacuucggaggcuucaacuucucacagauucugccagacccuucaaagccaagcaagcgcagcuucauugaagaucuccuguucaacaaggugacucuggcugacgcuggguucaucaagcaguauggagacugccugggggacaucgcagcaagggaucugaucugugcucagaaguuuaauggccucacagugcugcccccucuccugacugacgagaugauugcucaguacaccagcgcccugcuggcuggaacuaucacaucaggguggacauucggggccggggcugcucugcagauuccuuucgcaaugcaaauggcauauagguucaacgggaucggcgucacacagaacgugcuguaugagaaucagaaacugaucgcaaaucaauuuaauucagcaaucgggaaaauccaggacucacugucaucaaccgcaucagcccugggcaagcugcaggacgucgucaaccaaaacgcccaggcacucaacacacuggugaagcagcugucaagcaauuucggagcaaucagcagcguccugaaugauauccugagcaggcuggauaagguggaggcagaagugcagaucgaccgccugaucaccggccggcuccagagccugcaaaccuaugucacucagcaacugaucagagcugcagaaauccgggcaagcgccaaucuggccgccaccaagaugucagaaugcgugcucggccagagcaagaggguggacuucugcggcaaaggcuaccaccugaugagcuucccucagagcgccccccacggcguggucuuccuccaugugacuuaugugcccgcacaggaaaagaacuucacuacugccccagccaucugccacgacggaaaggcucauuucccacgcgaggggguguucgucagcaauggaacccacugguucgugacccagcggaacuucuaugagccucagauuauuaccacagacaacacauuugugucaggaaauugcgaugucgugaucgggaucgucaacaauacugucuaugacccccuccagccagaacucgauagcuucaaagaggaacucgauaaguauuuuaaaaaucacaccagcccugauguggaucugggcgauaucagcggaaucaaugcuagcguggugaacauccagaaggagauugacaggcucaacgaagucgcaaagaaccugaacgagucacugaucgaccuccaggaacugggcaaguacgaacaguacaucaaguggcccugguacaucuggcucggguucaucgcuggccugauugcuaucgugauggugacaaucaugcucugcuguaugaccagcugcugcagcugccugaagggaugcugcucaugcggcagcugcuguaaguucgaugaggacgacucagagcccgugcucaaaggcgugaaacugcauuauacuugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacauucuagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - DNA, 3822 bp, Artificial Sequence
  source: /note=“Description of Artificial Sequence Synthetic polynucleotide”
atgtttgtgtttctggtcctcctgcccctggtgtcaagccaatgcgtcaacttcactacacggacccagctgccccctgcttataccaactcatttactagaggggtgtactacccagataaagtgttcagatcaagcgtgctccacagcacccaggacctcttcctgcccttcttttcaaacgtgacctggttccatgccatccatgtgagcggaaccaacgggaccaagcggttcgccaatccagtgctccccttcaacgatggggtgtattttgcaagcactgaaaagtcaaatattatccgcggatggattttcgggacaacactcgatagcaagacccagtcactgctcatcgtgaacaacgctacaaatgtggtcatcaaggtgtgcgagtttcagttctgcaatgacccttttctcggcgtctactaccacaagaacaacaagagctggatggaatcagaattccgggtgtatagctcagctaataattgcaccttcgagtatgtgagccagcctttcctcatggacctggaggggaagcagggcaacttcaagaatctgagagaattcgtgtttaaaaacatcgatggatatttcaaaatctatagcaagcacactcctatcaacctcgtccggggcctgccacaaggctttagcgcactggagcccctcgtggacctgcccatcgggatcaatatcaccagatttcagactctgctggctctccacatcagctacctgactccaggggatagctcaagcggctggaccgctggagccgctgcctattacgtggggtacctgcagcctaggacttttctgctgaagtacaatgagaacgggacaattactgacgctgtcgattgtgcactcgaccccctcagcgagacaaaatgtaccctgaaaagctttactgtggaaaaagggatttaccagactagcaatttcagagtccagccaacagagtcaatcgtgcggtttcctaacatcacaaacctctgcccattcggggaggtcttcaatgccacccgcttcgccagcgtgtacgcttggaatagaaagcggatcagcaactgcgtggccgactacagcgtgctctacaatagcgctagcttctcaacatttaaatgctacggggtgagccccacaaaactgaatgacctgtgtttcaccaatgtgtatgccgatagcttcgtgattagaggcgacgaggtgcgccaaatcgcacctggccaaaccggaaacattgcagattataactataagctgcccgatgattttactggctgtgtcatcgcttggaacagcaacaacctcgattcaaaagtcggaggaaactataactacctgtatagactgttcagaaagagcaacctgaagccctttgaaagggatatcagcacagaaatttaccaggccgggagcaccccatgcaacggggtcaaagggtttaactgttatttccccctgcagagctacgggtttcaacccacctatggggtggggtatcagccttaccgcgtggtcgtgctgagcttcgaactgctgcacgcccccgccaccgtgtgcggccctaaaaagagcacaaacctcgtcaagaacaagtgcgtgaacttcaatttcaacggcctgactggcacaggcgtcctcaccgaaagcaataaaaagttcctccctttccagcaattcggcagagacattgcagacacaactgatgctgtgagggacccccagacactggagatcctggatattaccccctgttcattcggcggagtgtcagtcatcacccctggcactaacacatcaaatcaggtggcagtcctgtatcagggcgtgaactgcaccgaagtgcctgtcgccattcatgcagaccagctgacccccacttggagagtgtactcaactggcagcaatgtctttcaaaccagagccggctgtctcatcggcgcagaacatgtcaacaacagctatgagtgtgacatcccaatcggagctggcatctgtgccagctatcagacacagaccaacagccctcggagggctagaagcgtggccagccagagcattatcgcttacaccatgtcactgggcgtcgagaatagcgtggcctatagcaacaatagcatcgccattcccaccaattttaccatcagcgtgaccacagagatcctcccagtgagcatgaccaagacaagcgtcgattgcacaatgtacatttgcggcgacagcactgaatgtagcaacctcctgctgcagtacgggagcttttgtacccaactgaatcgggcactcaccgggatcgccgtggagcaggataaaaacactcaggaggtctttgctcaggtcaaacagatctacaaaaccccaccaattaaagacttcggaggcttcaacttctcacagattctgccagacccttcaaagccaagcaagcgcagcttcattgaagatctcctgttcaacaaggtgactctggctgacgctgggttcatcaagcagtatggagactgcctgggggacatcgcagcaagggatctgatctgtgctcagaagtttaatggcctcacagtgctgccccctctcctgactgacgagatgattgctcagtacaccagcgccctgctggctggaactatcacatcagggtggacattcggggccggggctgctctgcagattcctttcgcaatgcaaatggcatataggttcaacgggatcggcgtcacacagaacgtgctgtatgagaatcagaaactgatcgcaaatcaatttaattcagcaatcgggaaaatccaggactcactgtcatcaaccgcatcagccctgggcaagctgcaggacgtcgtcaaccaaaacgcccaggcactcaacacactggtgaagcagctgtcaagcaatttcggagcaatcagcagcgtcctgaatgatatcctgagcaggctggataaggtggaggcagaagtgcagatcgaccgcctgatcaccggccggctccagagcctgcaaacctatgtcactcagcaactgatcagagctgcagaaatccgggcaagcgccaatctggccgccaccaagatgtcagaatgcgtgctcggccagagcaagagggtggacttctgcggcaaaggctaccacctgatgagcttccctcagagcgccccccacggcgtggtcttcctccatgtgacttatgtgcccgcacaggaaaagaacttcactactgccccagccatctgccacgacggaaaggctcatttcccacgcgagggggtgttcgtcagcaatggaacccactggttcgtgacccagcggaacttctatgagcctcagattattaccacagacaacacatttgtgtcaggaaattgcgatgtcgtgatcgggatcgtcaacaatactgtctatgaccccctccagccagaactcgatagcttcaaagaggaactcgataagtattttaaaaatcacaccagccctgatgtggatctgggcgatatcagcggaatcaatgctagcgtggtgaacatccagaaggagattgacaggctcaacgaagtcgcaaagaacctgaacgagtcactgatcgacctccaggaactgggcaagtacgaacagtacatcaagtggccctggtacatctggctcgggttcatcgctggcctgattgctatcgtgatggtgacaatcatgctctgctgtatgaccagctgctgcagctgcctgaagggatgctgctcatgcggcagctgctgtaagttcgatgaggacgactcagagcccgtgctcaaaggcgtgaaactgcattatacttga

SEQ ID NO: 34 - DNA, 669 bp, Artificial Sequence
  source: /note=“Description of Artificial Sequence Synthetic polynucleotide”
atggccgactcaaacggcacaattaccgtggaggagctgaagaagctcctggagcagtggaatctggtcattggctttctgttcctgacctggatctgcctgctgcagttcgcttacgctaaccgcaaccggttcctctatatcattaaactcatttttctgtggctgctctggcctgtgacactggcctgtttcgtgctcgccgccgtctaccgcattaactggatcacaggagggattgctatcgctatggcttgcctggtcgggctgatgtggctctcatacttcatcgcaagctttaggctctttgcccggactcggagcatgtggtcattcaaccccgagacaaacattctgctcaatgtgcctctgcacgggaccatcctgaccaggcccctcctggagagcgagctggtgatcggggccgtgatcctgaggggacatctgaggatcgccggccatcatctgggcaggtgtgacatcaaggatctccctaaggagatcactgtggcaactagccggacactgagctattacaaactcggcgcaagccagcgcgtcgctggggactcaggcttcgcagcttacagcagatataggatcgggaactacaagctgaataccgatcacagctcaagcagcgataacatcgctctgctcgtccagtga

SEQ ID NO: 35 - DNA, 1140 bp, Artificial Sequence
  source: /note=“Description of Artificial Sequence Synthetic polynucleotide”
atgaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtga

SEQ ID NO: 36 - DNA, 1260 bp, Artificial Sequence
  source: /note=“Description of Artificial Sequence Synthetic polynucleotide”
atgtcagataacggcccacagaatcagagaaacgccccacggatcacttttggcgggcctagcgattcaaccgggtcaaatcagaatggggaacgcagcggggcccggagcaagcagagacggccccaagggctgcctaacaatacagcatcatggttcacagccctgacccagcatggcaaggaagatctcaagtttcctcgcggacagggcgtgcctattaacaccaattcaagccctgacgatcagatcggctactacagaagggctactcgcagaatccgcggaggcgatggaaaaatgaaagacctgagcccaaggtggtacttctattacctgggaactggccctgaggcaggactgccctatggcgccaacaaggatggcattatttgggtcgcaactgaaggagcactgaacacaccaaaggatcacatcggaacaaggaaccccgcaaacaatgccgctatcgtgctccagctgcctcaagggactaccctgcccaaagggttttatgccgaagggagcaggggcgggagccaggcaagcagcagaagctcatcacggtcaagaaattcaagcaggaatagcaccccaggaagctcaaggggaacaagcccagcccggatggcaggcaacggcggggatgcagcactcgcactgctcctgctggaccgcctgaatcagctggaatcaaaaatgagcgggaagggacagcaacagcagggacaaactgtcactaagaagtcagccgccgaggcaagcaagaagcccaggcaaaagcgcactgcaaccaaggcttacaatgtcactcaagcattcggcagaaggggcccagaacagacccagggcaatttcggcgaccaggagctgattagacaaggcactgattacaaacactggcctcagatcgctcagtttgcccccagcgcttcagccttttttgggatgagccggatcggcatggaggtgacaccatcagggacctggctcacctacactggagccattaaactggatgacaaagacccaaacttcaaagatcaagtgatcctgctcaacaagcacattgatgcatataaaacttttcctcctactgagcctaagaaggacaagaagaagaaagcagatgagacacaggctctgccccagcgccagaagaagcagcagactgtgacactgctgcccgctgcagatctggacgacttcagcaaacagctgcaacaatcaatgagctcagctgactcaactcaggcttga

SEQ ID NO: 37 - DNA, 1206 bp, Artificial Sequence
  source: /note=“Description of Artificial Sequence Synthetic polynucleotide”
atggacatgagagctccagcccagatctttggctttctgctcctgctgtttccaggcactaggtgcgacaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtga

SEQ ID NO: 38 - DNA, 1072 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggccgactcaaacggcacaattaccgtggaggagctgaagaagctcctggagcagtggaatctggtcattggctttctgttcctgacctggatctgcctgctgcagttcgcttacgctaaccgcaaccggttcctctatatcattaaactcatttttctgtggctgctctggcctgtgacactggcctgtttcgtgctcgccgccgtctaccgcattaactggatcacaggagggattgctatcgctatggcttgcctggtcgggctgatgtggctctcatacttcatcgcaagctttaggctctttgcccggactcggagcatgtggtcattcaaccccgagacaaacattctgctcaatgtgcctctgcacgggaccatcctgaccaggcccctcctggagagcgagctggtgatcggggccgtgatcctgaggggacatctgaggatcgccggccatcatctgggcaggtgtgacatcaaggatctccctaaggagatcactgtggcaactagccggacactgagctattacaaactcggcgcaagccagcgcgtcgctggggactcaggcttcgcagcttacagcagatataggatcgggaactacaagctgaataccgatcacagctcaagcagcgataacatcgctctgctcgtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - DNA, 1543 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - DNA, 1663 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgtcagataacggcccacagaatcagagaaacgccccacggatcacttttggcgggcctagcgattcaaccgggtcaaatcagaatggggaacgcagcggggcccggagcaagcagagacggccccaagggctgcctaacaatacagcatcatggttcacagccctgacccagcatggcaaggaagatctcaagtttcctcgcggacagggcgtgcctattaacaccaattcaagccctgacgatcagatcggctactacagaagggctactcgcagaatccgcggaggcgatggaaaaatgaaagacctgagcccaaggtggtacttctattacctgggaactggccctgaggcaggactgccctatggcgccaacaaggatggcattatttgggtcgcaactgaaggagcactgaacacaccaaaggatcacatcggaacaaggaaccccgcaaacaatgccgctatcgtgctccagctgcctcaagggactaccctgcccaaagggttttatgccgaagggagcaggggcgggagccaggcaagcagcagaagctcatcacggtcaagaaattcaagcaggaatagcaccccaggaagctcaaggggaacaagcccagcccggatggcaggcaacggcggggatgcagcactcgcactgctcctgctggaccgcctgaatcagctggaatcaaaaatgagcgggaagggacagcaacagcagggacaaactgtcactaagaagtcagccgccgaggcaagcaagaagcccaggcaaaagcgcactgcaaccaaggcttacaatgtcactcaagcattcggcagaaggggcccagaacagacccagggcaatttcggcgaccaggagctgattagacaaggcactgattacaaacactggcctcagatcgctcagtttgcccccagcgcttcagccttttttgggatgagccggatcggcatggaggtgacaccatcagggacctggctcacctacactggagccattaaactggatgacaaagacccaaacttcaaagatcaagtgatcctgctcaacaagcacattgatgcatataaaacttttcctcctactgagcctaagaaggacaagaagaagaaagcagatgagacacaggctctgccccagcgccagaagaagcagcagactgtgacactgctgcccgctgcagatctggacgacttcagcaaacagctgcaacaatcaatgagctcagctgactcaactcaggcttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 41 - DNA, 1609 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggacatgagagctccagcccagatctttggctttctgctcctgctgtttccaggcactaggtgcgacaggccccaggggctgccaaacaacaccgcaagctggttcactgccctgactcaacacggaaaggaggacctcaagttccccaggggacagggggtgcctattaacactaactcaagccctgacgatcagatcggctattatcgcagggctacaaggcgcattagaggaggagatgggaaaatgaaggacctgagccctcggtggtatttttattacctgggcactgggccagaggccggactgccatacggcgctaataaagacgggattatctgggtggccactgaaggcgctctgaatacccccaaggatcacattgggactcggaacccagccaacaacgccgctatcgtgctccagctccctcagggcaccactctgcccaaggggttctacgccgagggctcaagaggggggagccaggcctcaagcagaagcggcagcggccccaggcagaagagaacagctactaaagcatataatgtgacacaggccttcggccgcagagggcccgaacaaacccagggaaacttcggcgatcaggagctgatccggcagggcactgattacaagcactggcctcagattgcccagttcgctccatcagccagcgctttcttcggaatgagccggatcgggatggaggtgaccccttcagggacatggctgacttataccggagcaatcaagctggatgacaaggaccccaatttcaaagaccaggtgatcctgctgaacaaacatattgatgcctacaaagggtcaggcagactcttcgcccgcactagatcaatgtggtcatttaacccagaaactaacatcctgctgaacgtgcctctgcatggcacaattctcaccagacccctgctggaaagcgaactggtcattggcgctgtgatcctgagaggccacctgagaattgctggacaccatctcggcagatgtgatattaaggatctgccaaaggagatcaccgtggctacaagcagaactctcagctactacaagctgggggccagccagcgggtcgcaggggatagcggatttgccgcctactcaagatatagaatcggaaactataaactcaacaccgaccattcaagcagcagcgacaacattgcactgctggtccagtgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 4225 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgtttgtgtttctggtcctcctgcccctggtgtcaagccaatgcgtcaacctgactacacggacccagctgccccctgcttataccaactcatttactagaggggtgtactacccagataaagtgttcagatcaagcgtgctccacagcacccaggacctcttcctgcccttcttttcaaacgtgacctggttccatgccatccatgtgagcggaaccaacgggaccaagcggttcgataatccagtgctccccttcaacgatggggtgtattttgcaagcactgaaaagtcaaatattatccgcggatggattttcgggacaacactcgatagcaagacccagtcactgctcatcgtgaacaacgctacaaatgtggtcatcaaggtgtgcgagtttcagttctgcaatgacccttttctcggcgtctactaccacaagaacaacaagagctggatggaatcagaattccgggtgtatagctcagctaataattgcaccttcgagtatgtgagccagcctttcctcatggacctggaggggaagcagggcaacttcaagaatctgagagaattcgtgtttaaaaacatcgatggatatttcaaaatctatagcaagcacactcctatcaacctcgtccgggacctgccacaaggctttagcgcactggagcccctcgtggacctgcccatcgggatcaatatcaccagatttcagactctgctggctctccaccgcagctacctgactccaggggatagctcaagcggctggaccgctggagccgctgcctattacgtggggtacctgcagcctaggacttttctgctgaagtacaatgagaacgggacaattactgacgctgtcgattgtgcactcgaccccctcagcgagacaaaatgtaccctgaaaagctttactgtggaaaaagggatttaccagactagcaatttcagagtccagccaacagagtcaatcgtgcggtttcctaacatcacaaacctctgcccattcggggaggtcttcaatgccacccgcttcgccagcgtgtacgcttggaatagaaagcggatcagcaactgcgtggccgactacagcgtgctctacaatagcgctagcttctcaacatttaaatgctacggggtgagccccacaaaactgaatgacctgtgtttcaccaatgtgtatgccgatagcttcgtgattagaggcgacgaggtgcgccaaatcgcacctggccaaaccggaaagattgcagattataactataagctgcccgatgattttactggctgtgtcatcgcttggaacagcaacaacctcgattcaaaagtcggaggaaactataactacctgtatagactgttcagaaagagcaacctgaagccctttgaaagggatatcagcacagaaatttaccaggccgggagcaccccatgcaacggggtcgaagggtttaactgttatttccccctgcagagctacgggtttcaacccaccaatggggtggggtatcagccttaccgcgtggtcgtgctgagcttcgaactgctgcacgcccccgccaccgtgtgcggccctaaaaagagcacaaacctcgtcaagaacaagtgcgtgaacttcaatttcaacggcctgactggcacaggcgtcctcaccgaaagcaataaaaagttcctccctttccagcaattcggcagagacattgcagacacaactgatgctgtgagggacccccagacactggagatcctggatattaccccctgttcattcggcggagtgtcagtcatcacccctggcactaacacatcaaatcaggtggcagtcctgtatcagggcgtgaactgcaccgaagtgcctgtcgccattcatgcagaccagctgacccccacttggagagtgtactcaactggcagcaatgtctttcaaaccagagccggctgtctcatcggcgcagaacatgtcaacaacagctatgagtgtgacatcccaatcggagctggcatctgtgccagctatcagacacagaccaacagccctcggagggctagaagcgtggccagccagagcattatcgcttacaccatgtcactgggcgccgagaatagcgtggcctatagcaacaatagcatcgccattcccaccaattttaccatcagcgtgaccacagagatcctcccagtgagcatgaccaagacaagcgtcgattgcacaatgtacatttgcggcgacagcactgaatgtagcaacctcctgctgcagtacgggagcttttgtacccaactgaatcgggcactcaccgggatcgccgtggagcaggataaaaacactcaggaggtctttgctcaggtcaaacagatctacaaaaccccaccaattaaagacttcggaggcttcaacttctcacagattctgccagacccttcaaagccaagcaagcgcagcttcattgaagatctcctgttcaacaaggtgactctggctgacgctgggttcatcaagcagtatggagactgcctgggggacatcgcagcaagggatctgatctgtgctcagaagtttaatggcctcacagtgctgccccctctcctgactgacgagatgattgctcagtacaccagcgccctgctggctggaactatcacatcagggtggacattcggggccggggctgctctgcagattcctttcgcaatgcaaatggcatataggttcaacgggatcggcgtcacacagaacgtgctgtatgagaatcagaaactgatcgcaaatcaatttaattcagcaatcgggaaaatccaggactcactgtcatcaaccgcatcagccctgggcaagctgcaggacgtcgtcaaccaaaacgcccaggcactcaacacactggtgaagcagctgtcaagcaatttcggagcaatcagcagcgtcctgaatgatatcctgagcaggctggataaggtggaggcagaagtgcagatcgaccgcctgatcaccggccggctccagagcctgcaaacctatgtcactcagcaactgatcagagctgcagaaatccgggcaagcgccaatctggccgccaccaagatgtcagaatgcgtgctcggccagagcaagagggtggacttctgcggcaaaggctaccacctgatgagcttccctcagagcgccccccacggcgtggtcttcctccatgtgacttatgtgcccgcacaggaaaagaacttcactactgccccagccatctgccacgacggaaaggctcatttcccacgcgagggggtgttcgtcagcaatggaacccactggttcgtgacccagcggaacttctatgagcctcagattattaccacagacaacacatttgtgtcaggaaattgcgatgtcgtgatcgggatcgtcaacaatactgtctatgaccccctccagccagaactcgatagcttcaaagaggaactcgataagtattttaaaaatcacaccagccctgatgtggatctgggcgatatcagcggaatcaatgctagcgtggtgaacatccagaaggagattgacaggctcaacgaagtcgcaaagaacctgaacgagtcactgatcgacctccaggaactgggcaagtacgaacagtacatcaagtggccctggtacatctggctcgggttcatcgctggcctgattgctatcgtgatggtgacaatcatgctctgctgtatgaccagctgctgcagctgcctgaagggatgctgctcatgcggcagctgctgtaagttcgatgaggacgactcagagcccgtgctcaaaggcgtgaaactgcattatacttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - DNA, 4225 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgtttgtgtttctggtcctcctgcccctggtgtcaagccaatgcgtcaacttcactacacggacccagctgccccctgcttataccaactcatttactagaggggtgtactacccagataaagtgttcagatcaagcgtgctccacagcacccaggacctcttcctgcccttcttttcaaacgtgacctggttccatgccatccatgtgagcggaaccaacgggaccaagcggttcgccaatccagtgctccccttcaacgatggggtgtattttgcaagcactgaaaagtcaaatattatccgcggatggattttcgggacaacactcgatagcaagacccagtcactgctcatcgtgaacaacgctacaaatgtggtcatcaaggtgtgcgagtttcagttctgcaatgacccttttctcggcgtctactaccacaagaacaacaagagctggatggaatcagaattccgggtgtatagctcagctaataattgcaccttcgagtatgtgagccagcctttcctcatggacctggaggggaagcagggcaacttcaagaatctgagagaattcgtgtttaaaaacatcgatggatatttcaaaatctatagcaagcacactcctatcaacctcgtccggggcctgccacaaggctttagcgcactggagcccctcgtggacctgcccatcgggatcaatatcaccagatttcagactctgctggctctccacatcagctacctgactccaggggatagctcaagcggctggaccgctggagccgctgcctattacgtggggtacctgcagcctaggacttttctgctgaagtacaatgagaacgggacaattactgacgctgtcgattgtgcactcgaccccctcagcgagacaaaatgtaccctgaaaagctttactgtggaaaaagggatttaccagactagcaatttcagagtccagccaacagagtcaatcgtgcggtttcctaacatcacaaacctctgcccattcggggaggtcttcaatgccacccgcttcgccagcgtgtacgcttggaatagaaagcggatcagcaactgcgtggccgactacagcgtgctctacaatagcgctagcttctcaacatttaaatgctacggggtgagccccacaaaactgaatgacctgtgtttcaccaatgtgtatgccgatagcttcgtgattagaggcgacgaggtgcgccaaatcgcacctggccaaaccggaaacattgcagattataactataagctgcccgatgattttactggctgtgtcatcgcttggaacagcaacaacctcgattcaaaagtcggaggaaactataactacctgtatagactgttcagaaagagcaacctgaagccctttgaaagggatatcagcacagaaatttaccaggccgggagcaccccatgcaacggggtcaaagggtttaactgttatttccccctgcagagctacgggtttcaacccacctatggggtggggtatcagccttaccgcgtggtcgtgctgagcttcgaactgctgcacgcccccgccaccgtgtgcggccctaaaaagagcacaaacctcgtcaagaacaagtgcgtgaacttcaatttcaacggcctgactggcacaggcgtcctcaccgaaagcaataaaaagttcctccctttccagcaattcggcagagacattgcagacacaactgatgctgtgagggacccccagacactggagatcctggatattaccccctgttcattcggcggagtgtcagtcatcacccctggcactaacacatcaaatcaggtggcagtcctgtatcagggcgtgaactgcaccgaagtgcctgtcgccattcatgcagaccagctgacccccacttggagagtgtactcaactggcagcaatgtctttcaaaccagagccggctgtctcatcggcgcagaacatgtcaacaacagctatgagtgtgacatcccaatcggagctggcatctgtgccagctatcagacacagaccaacagccctcggagggctagaagcgtggccagccagagcattatcgcttacaccatgtcactgggcgtcgagaatagcgtggcctatagcaacaatagcatcgccattcccaccaattttaccatcagcgtgaccacagagatcctcccagtgagcatgaccaagacaagcgtcgattgcacaatgtacatttgcggcgacagcactgaatgtagcaacctcctgctgcagtacgggagcttttgtacccaactgaatcgggcactcaccgggatcgccgtggagcaggataaaaacactcaggaggtctttgctcaggtcaaacagatctacaaaaccccaccaattaaagacttcggaggcttcaacttctcacagattctgccagacccttcaaagccaagcaagcgcagcttcattgaagatctcctgttcaacaaggtgactctggctgacgctgggttcatcaagcagtatggagactgcctgggggacatcgcagcaagggatctgatctgtgctcagaagtttaatggcctcacagtgctgccccctctcctgactgacgagatgattgctcagtacaccagcgccctgctggctggaactatcacatcagggtggacattcggggccggggctgctctgcagattcctttcgcaatgcaaatggcatataggttcaacgggatcggcgtcacacagaacgtgctgtatgagaatcagaaactgatcgcaaatcaatttaattcagcaatcgggaaaatccaggactcactgtcatcaaccgcatcagccctgggcaagctgcaggacgtcgtcaaccaaaacgcccaggcactcaacacactggtgaagcagctgtcaagcaatttcggagcaatcagcagcgtcctgaatgatatcctgagcaggctggataaggtggaggcagaagtgcagatcgaccgcctgatcaccggccggctccagagcctgcaaacctatgtcactcagcaactgatcagagctgcagaaatccgggcaagcgccaatctggccgccaccaagatgtcagaatgcgtgctcggccagagcaagagggtggacttctgcggcaaaggctaccacctgatgagcttccctcagagcgccccccacggcgtggtcttcctccatgtgacttatgtgcccgcacaggaaaagaacttcactactgccccagccatctgccacgacggaaaggctcatttcccacgcgagggggtgttcgtcagcaatggaacccactggttcgtgacccagcggaacttctatgagcctcagattattaccacagacaacacatttgtgtcaggaaattgcgatgtcgtgatcgggatcgtcaacaatactgtctatgaccccctccagccagaactcgatagcttcaaagaggaactcgataagtattttaaaaatcacaccagccctgatgtggatctgggcgatatcagcggaatcaatgctagcgtggtgaacatccagaaggagattgacaggctcaacgaagtcgcaaagaacctgaacgagtcactgatcgacctccaggaactgggcaagtacgaacagtacatcaagtggccctggtacatctggctcgggttcatcgctggcctgattgctatcgtgatggtgacaatcatgctctgctgtatgaccagctgctgcagctgcctgaagggatgctgctcatgcggcagctgctgtaagttcgatgaggacgactcagagcccgtgctcaaaggcgtgaaactgcattatacttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - RNA, 99 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - DNA, 68 bp, Artificial Sequence
  source: /note=“Description of Artificial Sequence Synthetic polynucleotide”
atggacatgagagctccagcccagatctttggctttctgctcctgctgtttccaggcactaggtgcga

SEQ ID NO: 46 - RNA, 1729 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccaccauggucagcaaaggggaggaggucauuaaagaauucaugagauuuaaagucagaauggagggcagcaugaacggccacgaguucgaaaucgagggcgaaggagagggccgcccauaugaagggacccagacagccaagcugaaagugaccaagggcggaccacugccuuucgcuugggacauccugucaccccaauuuauguacgggucaaaggccuacgugaaacauccagccgacaucccagauuacaagaaacugagcuuuccugaaggguuuaagugggaacgggucaugaacuucgaggauggcggacuggugacugucacccaagauagcagccuccaggacggcacucugaucuauaaggugaagaugcgcggaacuaacuuuccacccgacgggccagucaugcagaagaagacaaugggcugggaagcuucaaccgaaaggcucuaucccagagaugggguccugaagggcgaaauccaccaggcccugaagcucaaggauggagggcacuaccugguggaguuuaaaaccaucuauauggccaaaaagcccgugcaacugccaggauauuauuacguggacacaaagcucgauaucaccagccacaacgaagauuacacaauugucgaacaguaugagcggucagaaggaaggcaucaccucuuucugggacaugggaccggcucaacuggcagcggcucaucaggcaccgccucaucagaagacaacaauauggccgugaucaaggaguucaugagauucaaggugagaauggaggggagcaugaacggccacgaauucgaaaucgagggcgagggggagggcagacccuaugaaggcacccagacugcaaaacugaaggucacaaaaggcggcccccugccauucgcuugggacauccugagcccccaauucauguauggaucaaaggccuaugugaagcacccugccgauauuccagauuauaagaagcucagcuuccccgagggcuuuaagugggagagggugaugaacuucgaggauggcggccucgugaccgucacccaggauucaagccugcaggacggaacacugaucuacaaagucaagaugagaggaaccaacuuucccccagauggccccgugaugcaaaaaaaaacaaugggcugggaggcuagcacugaacggcuguacccacgcgacggggugcugaaaggggaaauucaccaggcucugaaacugaaagauggaggacauuaccucguggaguucaagaccauuuacauggcuaaaaagccugugcaacugcccggcuauuauuauguggacacaaaacuggauauuaccucacacaacgaagacuauacaauuguggaacaguaugagaggagcgaggggcgccaccaccuguuucucuacggcauggaugaacucuacaagugacucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacau

SEQ ID NO: 47 - RNA, 128 nt, Artificial Sequence
  source: /note=“Description of Artificial Sequence Synthetic polynucleotide”
ucaacacaacauauacaaaacaaacgaaucucaagcaaucaagcauucuacuucuauugcagcaauuuaaaucauuucuuuuaaagcaaaagcaauuuucugaaaauuuucaccauuuacgaacgaua

SEQ ID NO: 48 - RNA, 164 nt, Artificial Sequence
  source: /note=“Description of Artificial Sequence Synthetic polynucleotide”
cucgagcuagugacugacuaggaucugguuaccacuaaaccagccucaagaacacccgaauggagucucuaagcuacauaauaccaacuuacacuuacaaaauguugucccccaaaauguagccauucguaucugcuccuaauaaaaagaaaguuucuucacau

SEQ ID NO: 49 - RNA, 68 nt, Artificial Sequence
  source: /note=“Description of Artificial Sequence Synthetic polynucleotide”
auggacaugagagcuccagcccagaucuuuggcuuucugcuccugcuguuuccaggcacuaggugcga

SEQ ID NO: 50 - DNA, 1144 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggacatgcgggctcccgcccagatcttcgggttcctgctgctcctgttccccggcaccagatgcgatcgcgtgcagcccaccgagagcatcgtccgcttccccaacattaccaacctgtgccccttcggcgaggtctttaacgccacaagattcgcatcagtgtatgcctggaacaggaagagaatctcaaactgtgtggcagactattcagtcctgtataatagcgcaagcttttcaacattcaaatgctatggcgtcagccccaccaaactcaatgatctgtgctttaccaatgtctacgcagacagcttcgtcattaggggagatgaagtgagacagattgcccccggccagaccggcaagatcgctgactacaattacaagctccccgacgactttacagggtgtgtcattgcctggaacagcaacaacctggattcaaaagtgggcggcaactacaactatctgtaccggctctttagaaaaagcaacctgaagcctttcgagagggacattagcacagaaatctatcaggctggcagcaccccatgcaacggagtggaaggcttcaactgctattttcctctgcagagctacggcttccagcccactaatggcgtcggctatcagccctacagggtggtcgtcctcagctttgaactgctgcatgctcctgctacagtgtgtggacccaagaagtcaaccaatctggtcaagaacaagtgtgtgaatttttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - DNA, 4213 bp, Artificial Sequence
  source: /note=“Description of Artificial Sequence Synthetic polynucleotide”
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgtttgtgtttctggtcctcctgcccctggtgtcaagccaatgcgtcaacctgactacacggacccagctgccccctgcttataccaactcatttactagaggggtgtactacccagataaagtgttcagatcaagcgtgctccacagcacccaggacctcttcctgcccttcttttcaaacgtgacctggttccatgccatccatgtgagcggaaccaacgggaccaagcggttcgataatccagtgctccccttcaacgatggggtgtattttgcaagcactgaaaagtcaaatattatccgcggatggattttcgggacaacactcgatagcaagacccagtcactgctcatcgtgaacaacgctacaaatgtggtcatcaaggtgtgcgagtttcagttctgcaatgacccttttctcggcgtctactaccacaagaacaacaagagctggatggaatcagaattccgggtgtatagctcagctaataattgcaccttcgagtatgtgagccagcctttcctcatggacctggaggggaagcagggcaacttcaagaatctgagagaattcgtgtttaaaaacatcgatggatatttcaaaatctatagcaagcacactcctatcaacctcgtccgggacctgccacaaggctttagcgcactggagcccctcgtggacctgcccatcgggatcaatatcaccagatttcagactctgctggctctccaccgcagctacctgactccaggggatagctcaagcggctggaccgctggagccgctgcctattacgtggggtacctgcagcctaggacttttctgctgaagtacaatgagaacgggacaattactgacgctgtcgattgtgcactcgaccccctcagcgagacaaaatgtaccctgaaaagctttactgtggaaaaagggatttaccagactagcaatttcagagtccagccaacagagtcaatcgtgcggtttcctaacatcacaaacctctgcccattcggggaggtcttcaatgccacccgcttcgccagcgtgtacgcttggaatagaaagcggatcagcaactgcgtggccgactacagcgtgctctacaatagcgctagcttctcaacatttaaatgctacggggtgagccccacaaaactgaatgacctgtgtttcaccaatgtgtatgccgatagcttcgtgattagaggcgacgaggtgcgccaaatcgcacctggccaaaccggaaagattgcagattataactataagctgcccgatgattttactggctgtgtcatcgcttggaacagcaacaacctcgattcaaaagtcggaggaaactataactacctgtatagactgttcagaaagagcaacctgaagccctttgaaagggatatcagcacagaaatttaccaggccgggagcaccccatgcaacggggtcgaagggtttaactgttatttccccctgcagagctacgggtttcaacccaccaatggggtggggtatcagccttaccgcgtggtcgtgctgagcttcgaactgctgcacgcccccgccaccgtgtgcggccctaaaaagagcacaaacctcgtcaagaacaagtgcgtgaacttcaatttcaacggcctgactggcacaggcgtcctcaccgaaagcaataaaaagttcctccctttccagcaattcggcagagacattgcagacacaactgatgctgtgagggacccccagacactggagatcctggatattaccccctgttcattcggcggagtgtcagtcatcacccctggcactaacacatcaaatcaggtggcagtcctgtatcaggacgtgaactgcaccgaagtgcctgtcgccattcatgcagaccagctgacccccacttggagagtgtactcaactggcagcaatgtctttcaaaccagagccggctgtctcatcggcgcagaacatgtcaacaacagctatgagtgtgacatcccaatcggagctggcatctgtgccagctatcagacacagaccatcctgagaagcgtggccagccagagcattatcgcttacaccatgtcactgggcgccgagaatagcgtggcctatagcaacaatagcatcgccattcccaccaattttaccatcagcgtgaccacagagatcctcccagtgagcatgaccaagacaagcgtcgattgcacaatgtacatttgcggcgacagcactgaatgtagcaacctcctgctgcagtacgggagcttttgtacccaactgaatcgggcactcaccgggatcgccgtggagcaggataaaaacactcaggaggtctttgctcaggtcaaacagatctacaaaaccccaccaattaaagacttcggaggcttcaacttctcacagattctgccagacccttcaaagccaagcaagcgcagcttcattgaagatctcctgttcaacaaggtgactctggctgacgctgggttcatcaagcagtatggagactgcctgggggacatcgcagcaagggatctgatctgtgctcagaagtttaatggcctcacagtgctgccccctctcctgactgacgagatgattgctcagtacaccagcgccctgctggctggaactatcacatcagggtggacattcggggccggggctgctctgcagattcctttcgcaatgcaaatggcatataggttcaacgggatcggcgtcacacagaacgtgctgtatgagaatcagaaactgatcgcaaatcaatttaattcagcaatcgggaaaatccaggactcactgtcatcaaccgcatcagccctgggcaagctgcaggacgtcgtcaaccaaaacgcccaggcactcaacacactggtgaagcagctgtcaagcaatttcggagcaatcagcagcgtcctgaatgatatcctgagcaggctggataaggtggaggcagaagtgcagatcgaccgcctgatcaccggccggctccagagcctgcaaacctatgtcactcagcaactgatcagagctgcagaaatccgggcaagcgccaatctggccgccaccaagatgtcagaatgcgtgctcggccagagcaagagggtggacttctgcggcaaaggctaccacctgatgagcttccctcagagcgccccccacggcgtggtcttcctccatgtgacttatgtgcccgcacaggaaaagaacttcactactgccccagccatctgccacgacggaaaggctcatttcccacgcgagggggtgttcgtcagcaatggaacccactggttcgtgacccagcggaacttctatgagcctcagattattaccacagacaacacatttgtgtcaggaaattgcgatgtcgtgatcgggatcgtcaacaatactgtctatgaccccctccagccagaactcgatagcttcaaagaggaactcgataagtattttaaaaatcacaccagccctgatgtggatctgggcgatatcagcggaatcaatgctagcgtggtgaacatccagaaggagattgacaggctcaacgaagtcgcaaagaacctgaacgagtcactgatcgacctccaggaactgggcaagtacgaacagtacatcaagtggccctggtacatctggctcgggttcatcgctggcctgattgctatcgtgatggtgacaatcatgctctgctgtatgaccagctgctgcagctgcctgaagggatgctgctcatgcggcagctgctgtaagttcgatgaggacgactcagagcccgtgctcaaaggcgtgaaactgcattatacttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 125 bp, Artificial Sequence
  source: /note=“Description of Artificial Sequence Synthetic polynucleotide”
aaccagcctcaagaacacccgaatggagtctctaagctacataataccaacttacacttacaaaatgttgtcccccaaaatgtagccattcgtatctgctcctaataaaaagaaagtttcttcac

SEQ ID NO: 53 - RNA, 125 nt, Artificial Sequence
  source: /note=“Description of Artificial Sequence Synthetic polynucleotide”
aaccagccucaagaacacccgaauggagucucuaagcuacauaauaccaacuuacacuuacaaaauguugucccccaaaauguagccauucguaucugcuccuaauaaaaagaaaguuucuucac